(19) United States
(12) Patent Application Publication
(10) Pub. No.: US 2025/0311539 A1
(43) Pub. Date: Oct. 2, 2025

(54) LIGHT-EMITTING ELEMENT, AMINE COMPOUND FOR LIGHT-EMITTING ELEMENT, DISPLAY DEVICE INCLUDING THE LIGHT-EMITTING ELEMENT, AND ELECTRONIC APPARATUS INCLUDING THE DISPLAY DEVICE

(71) Applicant: Samsung Display Co., Ltd., Yongin-si (KR)

(72) Inventors: Taku IMAIZUMI, Yokohama (JP); Ichinori TAKADA, Yokohama (JP)

(21) Appl. No.: 19/090,226

(22) Filed: Mar. 25, 2025

(30) Foreign Application Priority Data
Mar. 26, 2024 (KR) 10-2024-0040759

(51) Int. Cl. H10K 50/15; H10K 50/17; H10K 85/30; H10K 85/60

(52) U.S. Cl. CPC H10K 50/15; H10K 50/17; H10K 85/324; H10K 85/342; H10K 85/346; H10K 85/622; H10K 85/623; H10K 85/626; H10K 85/633; H10K 85/636; H10K 85/654; H10K 85/6572; H10K 85/6576

(57) ABSTRACT

A light-emitting element includes a first electrode, a second electrode on the first electrode, a light-emitting layer between the first electrode and the second electrode, and a hole transport region between the first electrode and the light-emitting layer, wherein the hole transport region includes an amine compound represented by Formula 1 and Formula 2.

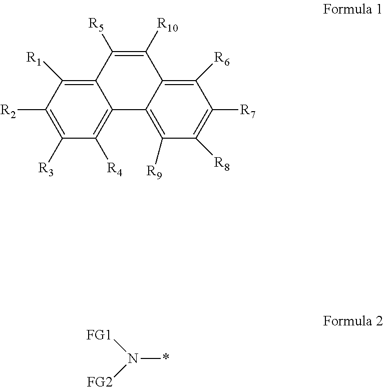

### CROSS-REFERENCE TO RELATED APPLICATION

The present application claims priority to and the benefit of Korean Patent Application No. 10-2024-0040759, filed on Mar. 26, 2024, in the Korean Intellectual Property Office, the entire content of which is incorporated herein by reference.

### BACKGROUND

### 1. Field

One or more embodiments of the present disclosure relate to a light-emitting element, an amine compound used in the light-emitting element, a display device including the light-emitting element, and an electronic apparatus including the display device.

### 2. Description of the Related Art

Recently, there has been active research and development in organic electroluminescence display devices used as image display devices. These devices include self-luminous light-emitting elements where holes and electrons injected separately from a first electrode and a second electrode recombine in a light-emitting layer. This recombination causes the light-emitting material in the light-emitting layer to emit light, enabling image display (e.g., display of images).

For the application of light-emitting elements to display devices, high luminous efficiency, long lifespan, and/or the like are desired or required. Therefore, the development of materials for light-emitting elements that may achieve these characteristics is actively pursued.

Additionally, to implement a light-emitting element with high efficiency and long lifespan, there is ongoing development of materials for the hole transport region of light-emitting element. These materials aim to improve charge transportability and material stability.

### SUMMARY

One or more aspects of embodiments of the present disclosure are directed toward a light-emitting element with (that has) enhanced (e.g., improved) luminous efficiency and lifespan.

One or more aspects of embodiments of the present disclosure are directed toward an amine compound, which is a material for a light-emitting element having characteristics of high efficiency and long lifespan.

One or more aspects of embodiments of the present disclosure are directed toward a display device including the light-emitting element having improved luminous efficiency and lifespan and exhibiting excellent or suitable display quality.

Additional aspects will be set forth in part in the description which follows and, in part, will be apparent from the description, or may be learned by practice of the presented embodiments.

According to one or more embodiments of the present disclosure, an amine compound represented by Formula 1 and Formula 2 is provided.

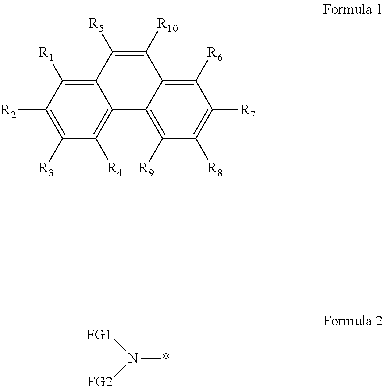

In Formula 1, at least one selected from among R₁ to R₅ is an amine group represented by Formula 2, and at least one selected from among the remaining R₁ to R₅ and R₆ to R₁₀ is deuterium, and the remaining that is not deuterium may be a substituted or unsubstituted aryl group having ring-forming carbons of 6 to 40. In Formula 2, FG1 and FG2 may each independently be a substituted or unsubstituted aryl group having ring-forming carbons of 6 to 40 except for a fluorenyl group or a substituted or unsubstituted heteroaryl group including 1 to 3 hetero atoms and having ring-forming carbons of 5 to 40, and FG1 and FG2 include no alkyl group as a substituent. If (e.g., when) R₂ is the amine group represented by Formula 2, a case where FG1 and FG2 are each a substituted or unsubstituted aryl group having ring-forming carbons of 6 to 15, at the same time, is excluded, and a case where FG1 or FG2 is benzonaphthofuranyl group substituted with an aryl group is excluded; if (e.g., when) R₃ is the amine group represented by Formula 2, a case where FG1 and FG2 are each a substituted or unsubstituted aryl group having ring-forming carbons of 6 to 9, at the same time, is excluded, and when one of FG1 and FG2 is an aryl group having a total ring-forming carbons of 26 or more, the other may be an aryl group having a total ring-forming carbons of 6 to 15 or a substituted or unsubstituted heteroaryl group having ring-forming carbons of 5 to 40; if (e.g., when) R₅ is the amine group represented by Formula 2, when FG1 or FG2 is a 4-dibenzofuranyl group or 4-dibenzothiophenyl group, at least one hydrogen of the 4-dibenzofuranyl group or 4-dibenzothiophenyl group is substituted with a substituent, and cases where FG1 or FG2 is a naphthyl group or a benzonaphthofuranyl group substituted with an aryl group are excluded.

In one or more embodiments, the amine compound may be represented by any one selected from among Formula 1A to Formula 1E:

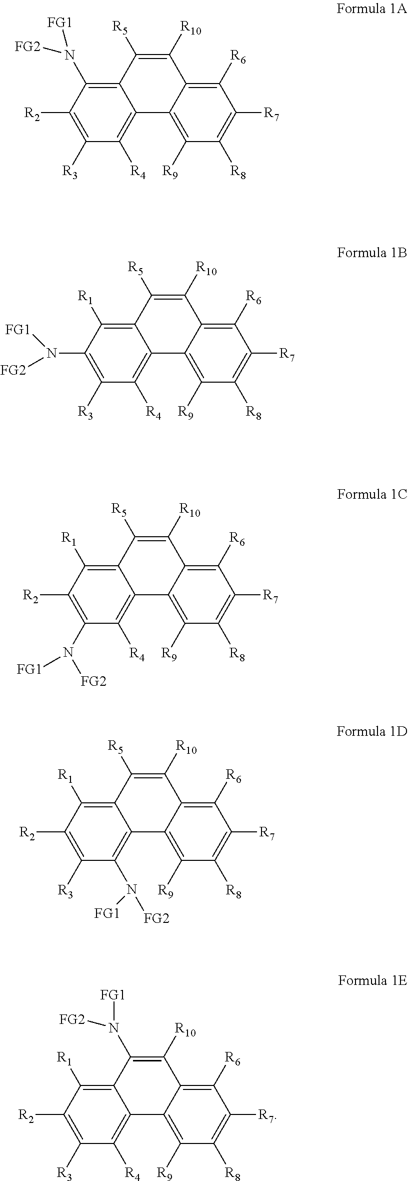

In Formula 1A to Formula 1E, R₁ to R₁₀, FG1, and FG2 may each independently be the same as defined in Formula 1 and Formula 2.

In one or more embodiments, in Formula 1, the remaining of R₁ to R₅ that are not the amine group represented by Formula 2, and R₆ to R₁₀, may all be deuterium(s), or any one selected from among the remaining of R₁ to R₅ that is not the amine group represented by Formula 2, and R₆ to R₁₀, may be a substituted or unsubstituted phenyl group, with (and) the remaining may all be deuterium(s).

In one or more embodiments, at least one hydrogen of FG1 and FG2, except those in R₁ to R₅ and R₆ to R₁₀, may be substituted with deuterium.

In one or more embodiments, the amine compound represented by Formula 1 and Formula 2 may be a monoamine compound including no additional amino group as a substituent.

In one or more embodiments, in Formula 2, FG1 and FG2 may each independently be a substituted or unsubstituted phenyl group, a substituted or unsubstituted biphenyl group, a substituted or unsubstituted terphenyl group, a substituted or unsubstituted naphthyl group, a substituted or unsubstituted phenanthryl group, a substituted or unsubstituted dibenzofuranyl group, a substituted or unsubstituted dibenzothiophenyl group, a substituted or unsubstituted benzonaphthofuranyl group, or a substituted or unsubstituted benzonaphthothiophenyl group.

According to one or more embodiments of the disclosure, a light-emitting element includes a first electrode, a second electrode on (e.g., arranged on) the first electrode, a light-emitting layer between the first electrode and the second electrode, and a hole transport region between the first electrode and the light-emitting layer and including the amine compound according to one or more embodiments.

In one or more embodiments, the hole transport region may include at least one selected from among a hole injection layer, a hole transport layer, an electron-blocking layer, and an emission auxiliary layer, wherein at least one selected from among the hole injection layer, the hole transport layer, the electron-blocking layer, and the emission auxiliary layer may include the amine compound.

In one or more embodiments, the hole transport region may include a hole injection layer on (e.g., arranged on) the first electrode, and a hole transport layer on (e.g., arranged on) the hole injection layer, and the amine compound may be included in the hole transport layer.

In one or more embodiments, at least one hydrogen of the amine compound, except those in R₁ to R₅ and R₆ to R₁₀, may be substituted with deuterium.

In one or more embodiments, the light-emitting layer may include a compound represented by Formula E-1.

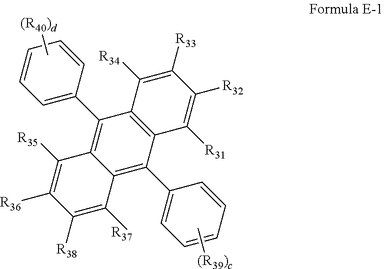

In Formula E-1, c and d may each independently be an integer of 0 to 5, R₃₁ to R₄₀ may each independently be hydrogen, deuterium, a halogen, a substituted or unsubstituted silyl group, a substituted or unsubstituted thio group, a substituted or unsubstituted oxy group, a substituted or unsubstituted alkyl group having 1 to 10 carbons, a substituted or unsubstituted alkenyl group having 2 to 10 carbons, a substituted or unsubstituted aryl group having ring-forming carbons of 6 to 30, or a substituted or unsubstituted heteroaryl group having ring-forming carbons of 2 to 30, and/or bonded to an adjacent group to form a ring.

In one or more embodiments, the light-emitting element may be to emit blue light or green light.

According to one or more embodiments of the disclosure, a display device includes a base layer, a circuit layer on (e.g., arranged on) the base layer, and a display element layer on (e.g., arranged on) the circuit layer and including a light-emitting element, wherein the light-emitting element includes a first electrode, a second electrode on (e.g., arranged on) the first electrode, a light-emitting layer between the first electrode and the second electrode, and a hole transport region between the first electrode and the light-emitting layer and including the amine compound represented by Formula 1 and Formula 2.

In one or more embodiments, the light-emitting element may be to emit blue light or green light.

In one or more embodiments, the display device may further include a light control layer including a quantum dot.

### BRIEF DESCRIPTION OF DRAWINGS

The accompanying drawings are included to provide a further understanding of the present disclosure, and are incorporated in and constitute a part of this disclosure. The drawings illustrate example embodiments of the present disclosure and, together with the description, serve to explain principles of the present disclosure. Above and/or other aspects of the disclosure should become apparent and appreciated from the following description of embodiments taken in conjunction with the accompanying drawings. In the drawings:

FIG. 1 is a plan view illustrating a display device according to one or more embodiments of the present disclosure;

FIG. 2 is a cross-sectional view illustrating a section taken along the line I-I′ of the display device of FIG. 1;

FIG. 3 is a cross-sectional view schematically illustrating a light-emitting element according to one or more embodiments of the present disclosure;

FIG. 4 is a cross-sectional view schematically illustrating a light-emitting element according to one or more embodiments of the present disclosure;

FIG. 5 is a cross-sectional view schematically illustrating a light-emitting element according to one or more embodiments of the present disclosure;

FIG. 6 is a cross-sectional view schematically illustrating a light-emitting element according to one or more embodiments of the present disclosure;

FIG. 7 is a cross-sectional view schematically illustrating a light-emitting element according to one or more embodiments of the present disclosure;

FIG. 8 is a cross-sectional view illustrating a display device according to one or more embodiments of the present disclosure;

FIG. 9 is a cross-sectional view illustrating a display device according to one or more embodiments of the present disclosure;

FIG. 10 is a cross-sectional view illustrating a display device according to one or more embodiments of the present disclosure;

FIG. 11 is a cross-sectional view illustrating a display device according to one or more embodiments of the present disclosure; and

FIG. 12 is a view illustrating an inside of a vehicle in which a display device according to one or more embodiments of the present disclosure is arranged.

### DETAILED DESCRIPTION

The present disclosure may be modified in one or more suitable manners and have many forms, and thus specific/example embodiments will be illustrated in the drawings and described in more detail in the detailed description of the present disclosure. It should be understood, however, that it is not intended to limit the disclosure to the particular forms disclosed, but rather, is intended to cover all modifications, equivalents, and alternatives falling within the spirit and scope of the disclosure.

When explaining each of drawings, like reference numbers are used for referring to like elements. In the accompanying drawings, the dimensions of each structure may be exaggeratingly illustrated for clarity of the present disclosure. It will be understood that, although the terms “first,” “second,” and/or the like, may be used herein to describe one or more suitable components, these components should not be limited by these terms. These terms are only used to distinguish one component from another. For example, a first component could be termed a second component, and, similarly, a second component could be termed a first component, without departing from the scope of example embodiments of the disclosure. As used herein, the singular forms, “a,” “an,” “one,” and “the” are intended to include the plural forms as well, unless the context clearly indicates otherwise. Further, the utilization of “may” when describing embodiments of the present disclosure refers to “one or more embodiments of the present disclosure.”

In the present disclosure, it will be understood that the terms “comprise(s)/comprising,” “include(s)/including,” “have (has)/having” and/or the like specify the presence of features, numbers, steps, operations, component, parts, and/or one or more (e.g., any suitable) combinations thereof disclosed in the disclosure, but do not exclude the possibility of presence or addition of one or more other features, numbers, steps, operations, component, parts, and/or one or more (e.g., any suitable) combinations thereof. Additionally, the terms “comprise(s)/comprising,” “include(s)/including,” “have/has/having”, or other similar terms include or support the terms “consisting of” and “consisting essentially of,” indicating the presence of stated features, numbers, steps, operations, components, and/or parts, without or essentially without the presence of other features, numbers, steps, operations, components, and/or parts thereof. As used herein, the terms “and,” “or,” and “and/or” may include any and all combinations of one or more of the associated listed items. Expressions such as “at least one of,” “one of,” and “selected from,” when preceding a list of elements, modify the entire list of elements and do not modify the individual elements of the list. For example, “at least one of a, b, or c,” “at least one selected from a, b, and c,” “at least one selected from among a to c,” etc., may indicate only a, only b, only c, both (e.g., simultaneously) a and b, both (e.g., simultaneously) a and c, both (e.g., simultaneously) b and c, all of a, b, and c, or variations thereof. The “/” utilized herein may be interpreted as “and” or as “or” depending on the situation.

In the present disclosure, if (e.g., when) a layer, a film, a region, or a plate is referred to as being “on” or “in an upper portion of” another layer, film, region, or plate, it may be not only “directly on” the layer, film, region, or plate, but one or more intervening layers, films, regions, or plates may also be present therebetween. On the contrary to this, if (e.g., when) a layer, a film, a region, or a plate is referred to as being “below”, “in a lower portion of” another layer, film, region, or plate, it can be not only directly under the layer, film, region, or plate, but one or more intervening layers, films, regions, or plates may also be present therebetween. In addition, it will be understood that if (e.g., when) a part is referred to as being “on” another part, the part may be arranged above the other part, or arranged under the other part as well. In the present disclosure, “directly on” may refer to that there are no additional layers, films, regions, plates, and/or like, between a layer, a film, a region, a plate, etc. and the other part. For example, “directly on” may refer to two layers or two members are arranged without utilizing an additional member such as an adhesive member therebetween.

In the present disclosure, the term “substituted or unsubstituted” may refer to substituted or unsubstituted with at least one substituent selected from the group consisting of deuterium, a halogen, a cyano group, a nitro group, an amino group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, a hydrocarbon ring group, an aryl group, and a heterocyclic group. In addition, each of the substituents exemplified above may be substituted or unsubstituted. For example, a biphenyl group may be interpreted as an aryl group or a phenyl group substituted with a phenyl group.

In the present disclosure, the phrase “bonded to an adjacent group to form a ring” may refer to that a group is bonded to an adjacent group to form a substituted or unsubstituted hydrocarbon ring or a substituted or unsubstituted heterocycle. The hydrocarbon ring may include an aliphatic hydrocarbon ring and/or an aromatic hydrocarbon ring. The heterocycle may include an aliphatic heterocycle and/or an aromatic heterocycle. The hydrocarbon ring and the heterocycle may each be monocyclic or polycyclic. In addition, the rings formed by adjacent groups being bonded to each other may be connected to another ring to form a spiro structure.

In the present disclosure, the term “adjacent group” may refer to a substituent substituted for an atom which is directly linked to an atom substituted with a corresponding substituent, another substituent substituted for an atom which is substituted with a corresponding substituent, or a substituent sterically positioned at the nearest position to a corresponding substituent. For example, two methyl groups in 1,2-dimethylbenzene may be interpreted as “adjacent groups” to each other and two ethyl groups in 1,1-diethylcyclopentane may be interpreted as “adjacent groups” to each other. In addition, two methyl groups in 4,5-dimethylphenanthrene may be interpreted as “adjacent groups” to each other.

In the present disclosure, examples of a halogen may include fluorine, chlorine, bromine, or iodine.

In the present disclosure, an alkyl group may be linear or branched. The number of carbons in the alkyl group may be 1 to 60, 1 to 30, 1 to 20, 1 to 15, 1 to 10, or 1 to 6. Examples of the alkyl group may include a methyl group, an ethyl group, an n-propyl group, an isopropyl group, an n-butyl group, an s-butyl group, a t-butyl group, an i-butyl group, a 2-ethylbutyl group, a 3,3-dimethylbutyl group, an n-pentyl group, an i-pentyl group, a neopentyl group, a t-pentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, an n-nonyl group, an n-decyl group, an adamantyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldocecyl group, a 2-octyldodecyl group, an n-tridecyl group, an n-tetradecyl group, an n-pentadecyl group, an n-hexadecyl group, a 2-ethylhexadecyl group, a 2-butylhexadecyl group, a 2-hexylhexadecyl group, a 2-octylhexadecyl group, an n-heptadecyl group, an n-octadecyl group, an n-nonadecyl group, an n-eicosyl group, a 2-ethyleicosyl group, a 2-butyleicosyl group, a 2-hexyleicosyl group, a 2-octyleicosyl group, an n-henicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, and/or the like, but embodiments of the present disclosure are not limited thereto.

In the present disclosure, the alkyl group may include a cycloalkyl group (cyclic alkyl group). The cycloalkyl group has 3 to 60 carbon atoms, 3 to 30 carbon atoms, or 3 to 20 carbon atoms, or 3 to 10 carbon atoms. Examples of the cycloalkyl group may include, but are not limited to, a cyclopropyl group, a cyclobutyl group, a cyclopentyl group, a cyclohexyl group, a 4-methylcyclohexyl group, a 4-t-butylcyclohexyl group, a cycloheptyl group, a cyclooctyl group, a cyclononyl group, a cyclodecyl group, a norbornyl group, a 1-adamantyl group, a 2-adamantyl group, an isobornyl group, a bicycloheptyl group, and/or the like.

In the present disclosure, an alkenyl group refers to a hydrocarbon group including at least one carbon-carbon double bond in the middle or terminal of an alkyl group having 2 or more carbon atoms. The alkenyl group may be linear or branched. The number of carbon atoms in the alkenyl group is not specifically limited, for example, may be 2 to 60, 2 to 30, 2 to 20, or 2 to 10. Examples of the alkenyl group may include a vinyl group, a 1-butenyl group, a 1-pentenyl group, a 1,3-butadienyl aryl group, a styrenyl group, a styryl vinyl group, and/or the like, but embodiments of the present disclosure are not limited thereto.

In the present disclosure, an alkynyl group refers to a hydrocarbon group including at least one carbon-carbon triple bond in the middle or terminal of an alkyl group having 2 or more carbon atoms. The alkynyl group may be linear or branched. Although the number of carbon atoms in the alkynyl group is not specifically limited, it may be 2 to 30, 2 to 20, or 2 to 10. Examples of the alkynyl group may include an ethynyl group, a propynyl group, and/or the like, but embodiments of the present disclosure are not limited thereto.

In the present disclosure, a hydrocarbon ring group refers to any functional group or substituent derived from an aliphatic hydrocarbon ring. The hydrocarbon ring group may be a saturated hydrocarbon ring group having 5 to 20 ring-forming carbon atoms.

As used herein, an aryl group refers to any functional group or a substituent derived from an aromatic hydrocarbon ring. The aryl group may be a monocyclic aryl group or a polycyclic aryl group. The number of ring-forming carbons of the aryl group may be 6 to 60, 6 to 30, 6 to 20, or 6 to 15. Examples of the aryl group may include a phenyl group, a naphthyl group, a fluorenyl group, an anthracenyl group, a phenanthryl group, a biphenylyl group, a terphenylyl group, a quarterphenylyl group, a quinquephenylyl group, a sexiphenylyl group, a triphenylenyl group, a pyrenyl group, a benzofluoranthenyl group, and a chrysenyl group, but embodiments of the present disclosure are not limited thereto.

In the present disclosure, a fluorenyl group may be substituted, and two substituents may be bonded to each other to form a spiro structure. Examples of the substituted fluorenyl group are as follows. However, embodiments of the present disclosure are not limited thereto.

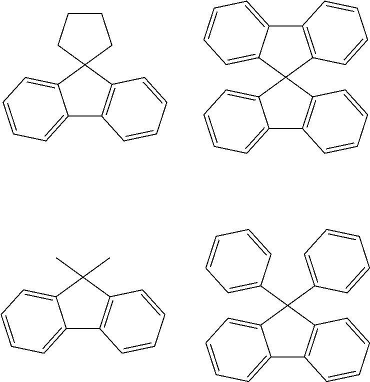

A heterocyclic group as used herein refers to any functional group or substituent derived from a ring containing at least one of B, O, N, P, S, Si, or Se as a heteroatom. The heterocyclic group includes an aliphatic heterocyclic group and/or an aromatic heterocyclic group. The aromatic heterocyclic group may be a heteroaryl group. The aliphatic heterocycle and the aromatic heterocycle may each be monocyclic or polycyclic.

In the present disclosure, the heterocyclic group may contain at least one of B, O, N, P, S, Si or Se as a heteroatom. If (e.g., when) the heterocyclic group contains two or more heteroatoms, the two or more heteroatoms may be the same as or different from each other. The heterocyclic group may be a monocyclic heterocyclic group or a polycyclic heterocyclic group, and may include a heteroaryl group. The number of ring-forming carbon atoms in the heterocyclic group may be 2 to 60, 2 to 30, 2 to 20, or 2 to 10.

In the present disclosure, the aliphatic heterocyclic group may include at least one of B, O, N, P, S, Si, or Se as a heteroatom. The number of ring-forming carbon atoms in the aliphatic heterocyclic group may be 2 to 30, 2 to 20, or 2 to 10. Examples of the aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group, and/or the like, but embodiments of the present disclosure are not limited thereto.

As used herein, a heteroaryl group may include at least one selected from among B, O, N, P, S, Si, and Se as hetero atom(s). When the heteroaryl group includes two or more hetero atoms, the two or more hetero atoms may all be the same, or may be different. The heteroaryl group may be a monocyclic heterocyclic group or a polycyclic heterocyclic group. The number of ring-forming carbons of heteroaryl group may be 2 to 60, 2 to 30, 2 to 20, or 2 to 10. Examples of the heteroaryl group may include a thienyl group, a furyl group, a pyrrolyl group, an imidazolyl group, a pyridyl group, a bipyridinyl group, a pyrimidinyl group, a triazinyl, a triazolyl group, an acridinyl group, a pyridazinyl group, a pyrazinyl group, a quinolyl group, a quinazolinyl group, a quinoxalinyl group, a phenoxazinyl group, a phthalazinyl group, a pyridopyrimidinyl group, a pyridopyrazinyl group, a pyrazinopyrazinyl group, an isoquinolinyl group, an indolyl group, a carbazolyl group, a N-arylcarbazolyl group, a N-heteroarylcarbazolyl group, a N-alkylcarbazolyl group, a benzoxazolyl group, a benzimidazolyl group, a benzothiazolyl group, a benzocarbazolyl group, a benzothiophenyl group, a dibenzothiophenyl group, a thienothiophenyl group, a benzofuranyl group, a phenanthrolinyl group, a thiazolyl group, an isoxazolyl group, an oxazolyl group, an oxadiazolyl group, a thiadiazolyl group, a phenothiazinyl group, a dibenzosilolyl group, a dibenzofuranyl group, and/or the like, but embodiments of the present disclosure are not limited thereto.

In the present disclosure, the above description of the aryl group may be applied to an arylene group except that the arylene group is a divalent group. The above description of the heteroaryl group may be applied to a heteroarylene group except that the heteroarylene group is a divalent group.

In the present disclosure, a silyl group may include an alkylsilyl group and/or an arylsilyl group. Examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and/or the like, but embodiments of the present disclosure are not limited thereto.

In the present disclosure, the number of carbon atoms in a carbonyl group is not specifically limited, for example, may be 1 to 40, 1 to 30, or 1 to 20. For example, the carbonyl group may have the following structures, but embodiments of the present disclosure are not limited thereto.

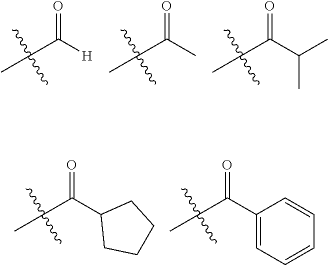

In the present disclosure, the number of carbon atoms in a sulfinyl group or a sulfonyl group is not particularly limited, for example, may be 1 to 30. The sulfinyl group may include an alkyl sulfinyl group and/or an aryl sulfinyl group. The sulfonyl group may include an alkyl sulfonyl group and/or an aryl sulfonyl group.

In the present disclosure, a thio group may include an alkylthio group and/or an arylthio group. The thio group may refer to that a sulfur atom is bonded to the alkyl group or the aryl group defined above. Examples of the thio group may include a methylthio group, an ethylthio group, a propylthio group, a pentylthio group, a hexylthio group, an octylthio group, a dodecylthio group, a cyclopentylthio group, a cyclohexylthio group, a phenylthio group, and a naphthylthio group, but embodiments of the present disclosure are not limited thereto.

In the present disclosure, an oxy group may refer to that an oxygen atom is bonded to the alkyl group or the aryl group defined above. The oxy group may include an alkoxy group and/or an aryl oxy group. The alkoxy group may be a linear chain, a branched chain, or a ring. The number of carbon atoms in the alkoxy group is not specifically limited, but may be, for example, 1 to 20 or 1 to 10. Examples of the oxy group may include methoxy, ethoxy, n-propoxy, isopropoxy, butoxy, pentyloxy, hexyloxy, octyloxy, nonyloxy, decyloxy, benzyloxy, and/or the like, but embodiments of the present disclosure are not limited thereto.

A boron group as used herein may refer to that a boron atom is bonded to the alkyl group or the aryl group defined above. The boron group may include an alkyl boron group and/or an aryl boron group. Examples of the boron group may include a dimethylboron group, a trimethylboron group, a t-butyldimethylboron group, a diphenylboron group, a phenylboron group, and/or the like, but embodiments of the present disclosure are not limited thereto.

In the present disclosure, the number of carbon atoms in an amine group is not specifically limited, for example, may be 1 to 30. The amine group may include an alkyl amine group and/or an aryl amine group. Examples of the amine group may include a methylamine group, a dimethylamine group, a phenylamine group, a diphenylamine group, a naphthylamine group, a 9-methyl-anthracenylamine group, a triphenylamine group, and/or the like, but embodiments of the present disclosure are not limited thereto. In the present disclosure, the term “amine group” may be used interchangeably with the term “amino group.”

In the present disclosure, the alkyl group among an alkylthio group, an alkylsulfoxy group, an alkylaryl group, an alkylamino group, an alkyl boron group, an alkyl silyl group, and an alkyl amine group may be the same as the examples of the alkyl group described above.

In the present disclosure, the aryl group among an aryloxy group, an arylthio group, an arylsulfoxy group, an arylamino group, an arylboron group, an arylsilyl group, and an arylamine group may be the same as the examples of the aryl group described above.

In the present disclosure, a direct linkage may refer to a single bond.

As used herein, “being deuterated” refers to that the abundance of deuterium isotope is greater than its natural abundance.

In the present disclosure,

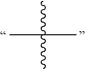

and “-*” each refer to a position to be connected.

Hereinafter, embodiments of the disclosure will be described with reference to the accompanying drawings.

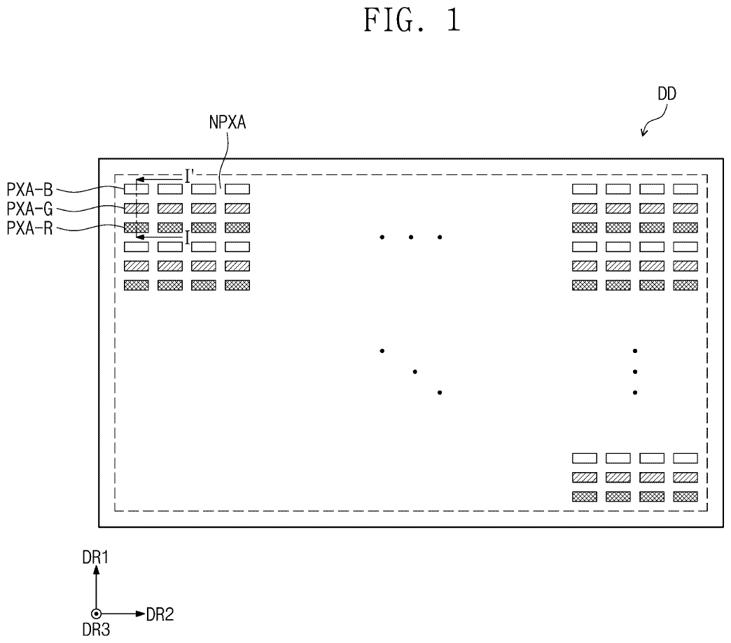

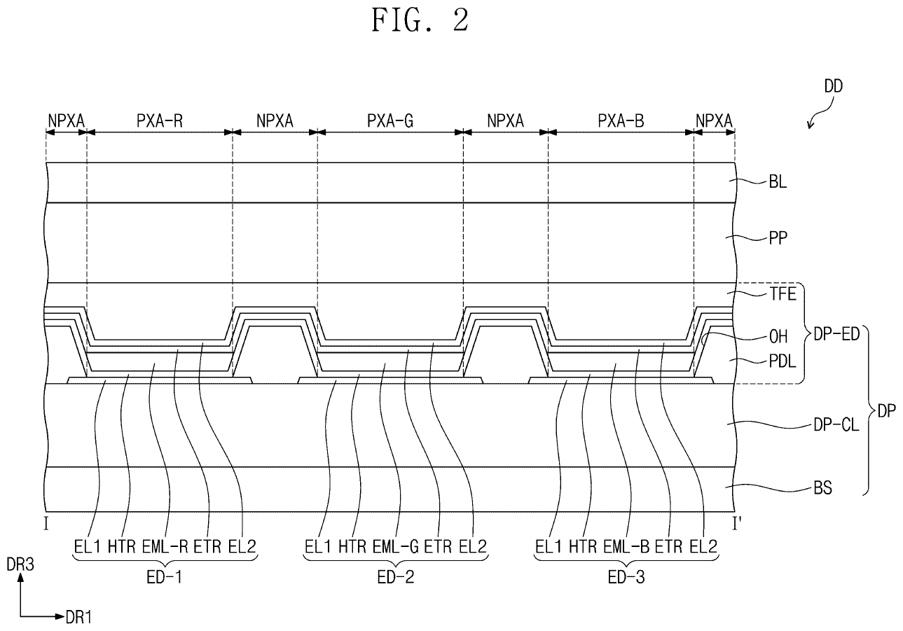

FIG. 1 is a plan view illustrating a display device DD according to one or more embodiments of the present disclosure. FIG. 2 is a cross-sectional view of the display device DD. FIG. 2 is a cross-sectional view illustrating a part taken along the line I-I′ of the display device DD of FIG. 1.

The display device DD may include a display panel DP and an optical layer PP arranged on the display panel DP. The display panel DP may include light-emitting elements ED-1, ED-2, and ED-3. The display device DD may include a plurality of light-emitting elements ED-1, ED-2, and ED-3. The optical layer PP may be arranged on the display panel DP to control reflected light in the display panel DP due to external light. The optical layer PP may include, for example, a polarization layer and/or a color filter layer. In one or more embodiments, the optical layer PP may not be provided in the display device DD.

A base substrate BL may be arranged on the optical layer PP. The base substrate BL may be a member which provides a base surface on which the optical layer PP arranged. The base substrate BL may be a glass substrate, a metal substrate, a plastic substrate, and/or the like. However, embodiments of the present disclosure are not limited thereto, and the base substrate BL may be an inorganic layer, an organic layer, or a composite material layer. In one or more embodiments, the base substrate BL may not be provided.

The display device DD according to one or more embodiments may further include a filling layer. The filling layer may be arranged between a display element layer DP-ED and the base substrate BL. The filling layer may be an organic material layer. The filling layer may include at least one of an acrylic-based resin, a silicone-based resin, or an epoxy-based resin.

The display panel DP may include a base layer BS, a circuit layer DP-CL provided on the base layer BS, and the display element layer DP-ED. The display element layer DP-ED may include a pixel defining film PDL, the light-emitting elements ED-1, ED-2, and ED-3 arranged between respective portions of the pixel defining film PDL, and an encapsulation layer TFE arranged on the light-emitting elements ED-1, ED-2, and ED-3.

The base layer BS may be a member which provides a base surface on which the display element layer DP-ED is arranged. The base layer BS may be a glass substrate, a metal substrate, a plastic substrate, and/or the like. However, embodiments of the present disclosure are not limited thereto, and the base layer BS may be an inorganic layer, an organic layer, or a composite material layer.

In one or more embodiments, the circuit layer DP-CL may be arranged on the base layer BS, and the circuit layer DP-CL may include a plurality of transistors. Each of the transistors may include a control electrode, an input electrode, and an output electrode. For example, in one or more embodiments, the circuit layer DP-CL may include switching transistor(s) and driving transistor(s) for driving the light-emitting elements ED-1, ED-2, and ED-3 of the display element layer DP-ED.

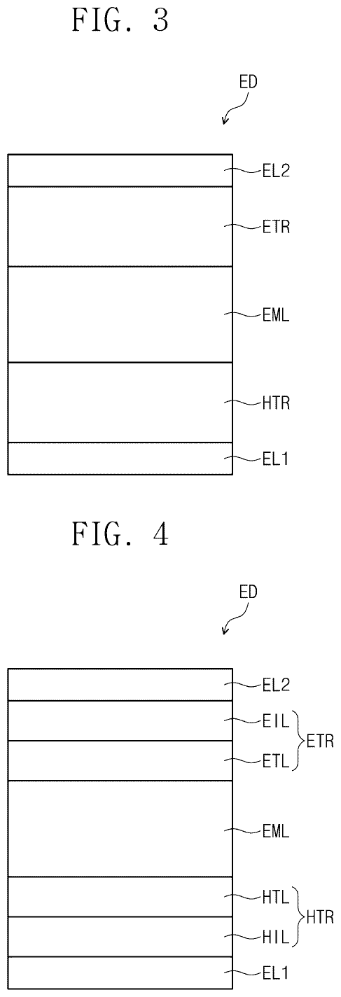

Each of the light-emitting elements ED-1, ED-2, and ED-3 may have a structure of one of light-emitting elements ED of embodiments according to FIGS. 3 to 7, which will be described in more detail later. Each of the light-emitting elements ED-1, ED-2, and ED-3 may include a first electrode EL1, a hole transport region HTR, respective light-emitting layers EML-R, EML-G, and EML-B, an electron transport region ETR, and a second electrode EL2.

FIG. 2 illustrates one or more embodiments in which the respective light-emitting layers EML-R, EML-G, and EML-B of the light-emitting elements ED-1, ED-2, and ED-3 are arranged in openings OH defined in the pixel defining film PDL, and the hole transport region HTR, the electron transport region ETR, and the second electrode EL2 are each provided as a common layer across the entire light-emitting elements ED-1, ED-2, and ED-3. However, embodiments of the present disclosure are not limited thereto, for example, in one or more embodiments, the hole transport region HTR and the electron transport region ETR may each be provided by being patterned inside the openings OH defined in the pixel defining film PDL. For example, the hole transport region HTR, the respective light-emitting layers EML-R, EML-G, and EML-B, and the electron transport region ETR of the light-emitting elements ED-1, ED-2, and ED-3 in one or more embodiments may be provided by being patterned in an inkjet printing method.

The encapsulation layer TFE may cover the light-emitting elements ED-1, ED-2, and ED-3. The encapsulation layer TFE may seal the display element layer DP-ED. The encapsulation layer TFE may be a thin film encapsulation layer. The encapsulation layer TFE may be formed by laminating one layer or a plurality of layers.

The encapsulation layer TFE may include at least one insulation layer. The encapsulation layer TFE according to one or more embodiments may include at least one inorganic film (hereinafter, an encapsulation-inorganic film). The encapsulation layer TFE according to one or more embodiments may include at least one organic film (hereinafter, an encapsulation-organic film) and at least one encapsulation-inorganic film.

The encapsulation-inorganic film protects the display element layer DP-ED from moisture/oxygen, and the encapsulation-organic film protects the display element layer DP-ED from foreign substances such as dust particles. The encapsulation-inorganic film may include silicon nitride, silicon oxynitride, silicon oxide, titanium oxide, aluminum oxide, and/or the like, but embodiments of the present disclosure are not particularly limited thereto. The encapsulation-organic film may include an acrylic-based compound, an epoxy-based compound, and/or the like. In one or more embodiments, the encapsulation-organic film may include a photopolymerizable organic material, but embodiments of the present disclosure are not particularly limited thereto.

The encapsulation layer TFE may be arranged on the second electrode EL2 and may be arranged filling the opening OH.

Referring to FIG. 1 and FIG. 2, the display device DD may include a non-light emitting region NPXA and light emitting regions PXA-R, PXA-G, and PXA-B. The light emitting regions PXA-R, PXA-G, and PXA-B may be regions in which light generated by the respective light-emitting elements ED-1, ED-2, and ED-3 is emitted.

The light emitting regions PXA-R, PXA-G, and PXA-B may be spaced and/or apart (e.g., spaced apart or separated) from one another on a plane (e.g., in a plan view).

Each of the light emitting regions PXA-R, PXA-G, and PXA-B may be a region divided by the pixel defining film PDL. The non-light emitting region NPXA may be regions between adjacent light emitting regions PXA-R, PXA-G, and PXA-B, which correspond to the pixel defining film PDL. In one or more embodiments, the light emitting regions PXA-R, PXA-G, and PXA-B may respectively correspond to pixels. The pixel defining film PDL may divide the light-emitting elements ED-1, ED-2, and ED-3. The respective light-emitting layers EML-R, EML-G, and EML-B of the light-emitting elements ED-1, ED-2, and ED-3 may be arranged in openings OH defined in the pixel defining film PDL and separated from one another.

The light emitting regions PXA-R, PXA-G, and PXA-B may be divided into a plurality of groups according to the color of light generated from the light-emitting elements ED-1, ED-2, and ED-3. In the display device DD of one or more embodiments illustrated in FIG. 1 and FIG. 2, three light emitting regions PXA-R, PXA-G, and PXA-B, which emit red light, green light, and blue light, respectively, are exemplarily illustrated. For example, the display device DD of one or more embodiments may include a red light emitting region PXA-R, a green light emitting region PXA-G, and a blue light emitting region PXA-B that are separated from one another.

In the display device DD according to one or more embodiments, the plurality of light-emitting elements ED-1, ED-2 and ED-3 may be to emit light beams having wavelengths different from one another. For example, in one or more embodiments, the display device DD may include a first light-emitting element ED-1 that is to emit red light, a second light-emitting element ED-2 that is to emit green light, and a third light-emitting element ED-3 that is to emit blue light. For example, the red light emitting region PXA-R, the green light emitting region PXA-G, and the blue light emitting region PXA-B of the display device DD may correspond to the first light-emitting element ED-1, the second light-emitting element ED-2, and the third light-emitting element ED-3, respectively.

However, embodiments of the present disclosure are not limited thereto, and the first to third light-emitting elements ED-1, ED-2, and ED-3 may be to emit light beams in substantially the same wavelength range or at least one light-emitting element may be to emit a light beam in a wavelength range different from the others. For example, in one or more embodiments, the first to third light-emitting elements ED-1, ED-2, and ED-3 may all emit blue light.

The light emitting regions PXA-R, PXA-G, and PXA-B in the display device DD according to one or more embodiments may be arranged in a stripe form.

Referring to FIG. 1, a plurality of red light emitting regions PXA-R may be arranged with each other along a second direction axis DR2, a plurality of green light emitting regions PXA-G may be arranged with each other along the second direction axis DR2, and a plurality of blue light emitting regions PXA-B may be arranged with each other along the second direction axis DR2. In addition, the red light emitting region PXA-R, the green light emitting region PXA-G, and the blue light emitting region PXA-B may be alternately arranged in this order along a first direction axis DR1.

FIG. 1 and FIG. 2 illustrate that all the light emitting regions PXA-R, PXA-G, and PXA-B have similar area, but embodiments of the present disclosure are not limited thereto. Thus, in one or more embodiments, the light emitting regions PXA-R, PXA-G, and PXA-B may have different areas from one another according to the wavelength range of the emitted light. The areas of the light emitting regions PXA-R, PXA-G, and PXA-B may refer to areas if (e.g., when) viewed on a plane defined by the first direction axis DR1 and the second direction axis DR2 (e.g., the areas in a plan view of the light emitting regions PXA-R, PXA-G, and PXA-B).

In one or more embodiments, an arrangement form of the light emitting regions PXA-R, PXA-G, and PXA-B is not limited to the configuration illustrated in FIG. 1, and the order in which the red light emitting region PXA-R, the green light emitting region PXA-G, and the blue light emitting region PXA-B are arranged may be provided in one or more suitable combinations according to the characteristics of display quality desired or required in the display device DD. For example, in one or more embodiments, the arrangement form of the light emitting regions PXA-R, PXA-G, and PXA-B may be a pentile (PENTILE©) arrangement form (for example, an RGBG matrix, an RGBG structure, or an RGBG matrix structure) or a diamond (Diamond Pixel™) arrangement form (e.g., a display (e.g., an OLED display) containing red, blue, and green (RGB) light-emitting regions arranged in the shape of diamonds). PENTILE© is a duly registered trademark of Samsung Display Co., Ltd. Diamond Pixel™ is a trademark of Samsung Display Co., Ltd.

In one or more embodiments, the areas of the light emitting regions PXA-R, PXA-G, and PXA-B may be different from one another. For example, in one or more embodiments, the area of the green light emitting region PXA-G may be smaller than that of the blue light emitting region PXA-B, but embodiments of the present disclosure are not limited thereto.

Hereinafter, FIGS. 3 to 7 are each a cross-sectional view schematically illustrating a light-emitting element according to one or more embodiments of the present disclosure.

A light-emitting element ED illustrated in FIG. 3, according to one or more embodiments, may include a first electrode EL1, a hole transport region HTR, a light-emitting layer EML, an electron transport region ETR, and a second electrode EL2, which are sequentially stacked in the stated order. Compared to FIG. 3, FIG. 4 illustrates a cross-sectional view of a light-emitting element ED according to one or more embodiments in which a hole transport region HTR includes a hole injection layer HIL and a hole transport layer HTL, and an electron transport region ETR includes an electron injection layer EIL and an electron transport layer ETL.

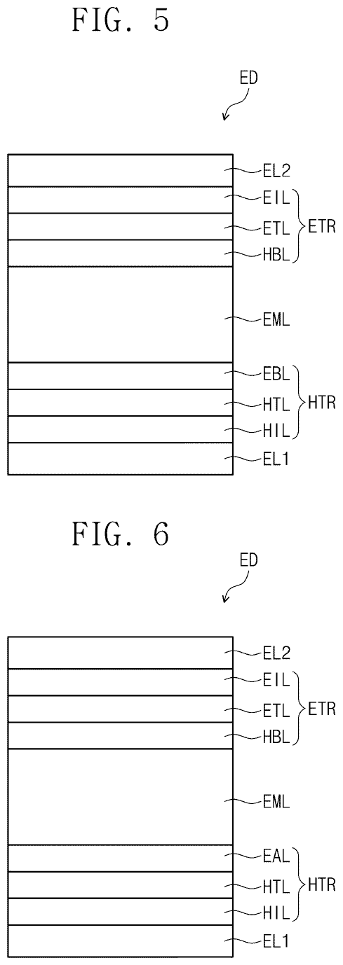

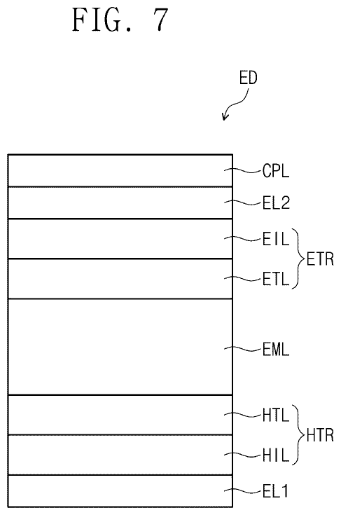

In addition, compared to FIG. 3, FIG. 5 illustrates a cross-sectional view of a light-emitting element ED according to one or more embodiments in which a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. Compared to FIG. 3, FIG. 6 illustrates a cross-sectional view of a light-emitting element ED according to one or more embodiments in which a hole transport region HTR includes a hole injection layer HIL, a hole transport layer HTL, and an auxiliary light-emitting layer EAL, and an electron transport region ETR includes an electron injection layer EIL, an electron transport layer ETL, and a hole blocking layer HBL. Compared to FIG. 4, FIG. 7 illustrates a cross-sectional view of a light-emitting element ED according to one or more embodiments in which a capping layer CPL is arranged on a second electrode EL2.

The first electrode EL1 has conductivity (e.g., is a conductor). The first electrode EL1 may be formed of a metal material, a metal alloy, and/or a conductive compound. The first electrode EL1 may be an anode or a cathode. However, embodiments of the present disclosure are not limited thereto. In one or more embodiments, the first electrode EL1 may be a pixel electrode. The first electrode EL1 may be a transmissive electrode, a transflective electrode, or a reflective electrode. The first electrode EL1 may include at least one selected from among silver (Ag), magnesium (Mg), copper (Cu), aluminum (Al), platinum (Pt), palladium (Pd), gold (Au), nickel (Ni), neodymium (Nd), iridium (Ir), chromium (Cr), lithium (Li), calcium (Ca), lithium fluoride (LiF), molybdenum (Mo), titanium (Ti), tungsten (W), indium (In), tin (Sn), and zinc (Zn), a compound of two or more selected therefrom, a mixture of two or more selected therefrom, and/or an oxide thereof.

If (e.g., when) the first electrode EL1 is a transmissive electrode, the first electrode EL1 may include a transparent metal oxide such as indium tin oxide (ITO), indium zinc oxide (IZO), zinc oxide (ZnO), and/or indium tin zinc oxide (ITZO). If (e.g., when) the first electrode EL1 is a transflective electrode or a reflective electrode, the first electrode EL1 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, W, a compound thereof, or a mixture thereof (e.g., a mixture of Ag and Mg). In one or more embodiments, the first electrode EL1 may have a multilayer structure including a reflective film or a transflective film formed of one or more of the above-described materials, and a transparent conductive film formed of ITO, IZO, ZnO, ITZO, and/or the like. For example, in one or more embodiments, the first electrode EL1 may have a three-layer structure of ITO/Ag/ITO, but embodiments of the present disclosure are not limited thereto. In one or more embodiments, the first electrode EL1 may include one of the above-described metal materials, one or more combinations of at least two metal materials of the above-described metal materials, any oxide of the above-described metal materials, and/or the like. A thickness of the first electrode EL1 may be from about 700 angströms (Å) to about 10,000 Å. For example, in one or more embodiments, the thickness of the first electrode EL1 may be from about 1,000 Å to about 3,000 Å.

In the present disclosure, “being deuterated” refers to that abundance of deuterium isotope increases compared to its natural abundance.

The hole transport region HTR may be provided on the first electrode EL1. The hole transport region HTR may be arranged between the first electrode EL1 and the light-emitting layer EML.

The hole transport region HTR may include at least one of a hole injection layer HIL, a hole transport layer HTL, an auxiliary light-emitting layer EAL, or an electron-blocking layer EBL. The auxiliary light-emitting layer EAL may be also referred to as a buffer layer. For example, in one or more embodiments, the hole transport region HTR may have a thickness of about 50 Å to about 15000 Å.

The hole transport region HTR may have a single layer composed of a single material, a single layer composed of a plurality of different materials, or a plurality of layers composed of a plurality of different materials.

For example, in one or more embodiments, the hole transport region HTR may have a single-layered structure of a hole injection layer HIL or a hole transport layer HTL, or a single-layered structure composed of a hole injection material and/or a hole transport material. In one or more embodiments, the hole transport region HTR may have a single-layered structure composed of a plurality of different materials, or a structure of a hole injection layer HIL/a hole transport layer HTL, a hole injection layer HIL/an auxiliary light-emitting layer EAL, a hole transport layer HTL/an auxiliary light-emitting layer EAL, a hole transport layer HTL/an electron-blocking layer EBL, a hole injection layer HIL/a hole transport layer HTL/an auxiliary light-emitting layer EAL, or a hole injection layer HIL/a hole transport layer HTL/an electron-blocking layer EBL sequentially stacked from the first electrode EL1 in the stated order, but embodiments of the present disclosure are not limited thereto. In one or more embodiments, the hole transport layer HTL may have a single layer or a plurality of layers.

The hole transport region HTR may be formed using one or more suitable methods such as vacuum deposition, spin coating, casting, Langmuir-Blodgett (LB), inkjet printing, laser printing, or laser induced thermal imaging (LITI).

The light-emitting element ED according to one or more embodiments may include an amine compound represented by Formula 1 and Formula 2 in the hole transport region HTR. In one or more embodiments, at least one selected from among the hole injection layer HIL, the hole transport layer HTL, the electron-blocking layer EBL, and the emission auxiliary layer EAL may include the amine compound according to one or more embodiments. For example, the light-emitting element ED according to one or more embodiments may include the amine compound according to one or more embodiments in the hole transport layer HTL.

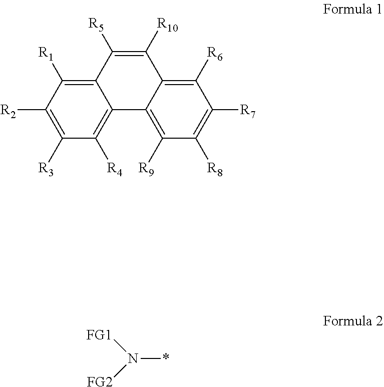

The amine compound according to one or more embodiments may include a phenanthrene group (or also referred to as a phenanthrene moiety) represented by Formula 1 and an amine group (or also referred to as an amine moiety) represented by Formula 2, and a nitrogen atom (N) of the amine group may be bonded at any position among R₁ to R₅ of the phenanthrene group represented by Formula 1. For example, any one selected from among R₁ to R₅ of the phenanthrene group may be the amine group represented by Formula 2. The amine group represented by Formula 2 may be directly bonded to the phenanthrene group represented by Formula 1. In addition, in the amine compound according to one or more embodiments, in the phenanthrene group represented by Formula 1, at least one selected from among the remaining, in R₁ to R₅, that is not substituted with the amine group represented by Formula 2 may be substituted with deuterium. For example, in one or more embodiments, the phenanthrene group represented by Formula 1 may be deuterated.

In addition, in the amine compound according to one or more embodiments, the amine group represented by Formula 2 may be an aromatic amine. FG1 and FG2 in FIG. 2 may each independently be an aryl group or a heteroaryl group. In one or more embodiments, the amine compound according to one or more embodiments may be a monoamine compound including no additional amino substituent.

The amine compound according to one or more embodiments may essentially include a deuterated phenanthrene group and an aromatic amine group, and a nitrogen atom of the aromatic amine group may be characterized by being directly bonded to the phenanthrene group. In addition, the types (kinds) of aromatic groups contained in the aromatic amine group may vary depending on a location at which the amine group is bonded to the phenanthrene group. The amine compound according to one or more embodiments may include a deuterated phenanthrene group and an aromatic amine group, thereby having high planarity and sufficient electron density and thus having excellent or suitable hole transport ability. As a result, the light-emitting element including the amine compound according to one or more embodiments may exhibit characteristics of high efficiency and long lifespan.

According to one or more embodiments of the present disclosure, in Formula 1, any one selected from among R₁ to R₅ may be an amine group represented by Formula 2, at least one selected from among the remaining of R₁ to R₅, and R₆ to R₁₀ may be deuterium, and the remaining that are not deuterium may be a substituted or unsubstituted aryl group having ring-forming carbons of 6 to 40. For example, in the amine compound according to one or more embodiments, the amine group represented by Formula 2 is bonded at any location of R₁ to R₅, at least one selected from among the remaining of R₁ to R₅ in which the amine group represented by Formula 2 is not bonded, and R₆ to R₁₀ may be deuterium(s), and the remaining among R₁ to R₅ and R₆ to R₁₀ that are not the amine group represented by Formula 2 or deuterium may be a substituted or unsubstituted aryl group having ring-forming carbons of 6 to 40.

In the amine compound according to one or more embodiments, the remaining that are unsubstituted with the amine group, of the phenanthrene group represented by Formula 1, may all be substituted with deuterium, or at least one selected from among the remaining that are unsubstituted with the amine group may be substituted with deuterium, and the remaining that are unsubstituted with deuterium may be a substituted or unsubstituted aryl group. For example, in one or more embodiments, the remaining among R₁ to R₅ that are not the amine group represented by Formula 2 and R₆ to R₁₀ may all be deuterium(s), or the remaining among R₁ to R₅ that are not the amine group represented by Formula 2 and any one selected from among R₆ to R₁₀ may be a substituted or unsubstituted phenyl group, and the rest may be deuterium. For example, in the amine compound according to one or more embodiments, any one selected from among R₁ to R₅ may be the amine group represented by Formula 2, the remaining among R₁ to R₅ to which the amine group of Formula 2 is not bonded may be deuterium(s), R₆ to R₁₀ may all be deuterium(s), or at least one selected from among R₆ to R₁₀ may be deuterium, and the rest may be a substituted or unsubstituted aryl group.

In Formula 2, FG1 and FG2 may each independently be a substituted or unsubstituted aryl group having ring-forming carbons of 6 to 40 or a substituted or unsubstituted heteroaryl group having ring-forming carbons of 5 to 40. If (e.g., when) FG1 or FG2 is an aryl group, a case where the aryl group is a fluorenyl group is excluded. In one or more embodiments, if (e.g., when) FG1 or FG2 is a heteroaryl group, FG1 or FG2 may include one to three ring-forming hetero atoms. In addition, FG1 and FG2 may include no alkyl group as a substituent, for example, FG1 and FG2 may exclude an alkyl group as a substituent. FG1 and FG2 may not include (e.g., may exclude) a linear chain alkyl group, a branched chain alkyl group, and a cyclic alkyl group, as a substituent. For example, when FG1 or FG2 is a heteroaryl group, FG1 and FG2 may include one to three ring-forming hetero atoms. Additionally, FG1 and FG2 may exclude any alkyl group as a substituent. Specifically, FG1 and FG2 may exclude any linear chain alkyl group, branched chain alkyl group, and cyclic alkyl group as substituents. Also, for example, the amine group represented by Formula 2 may be an amine group including FG1 and FG2, which are aromatic groups.

For example, in one or more embodiments, FG1 and FG2 may each independently be a substituted or unsubstituted phenyl group, a substituted or unsubstituted biphenylyl group, a substituted or unsubstituted terphenylyl group, a substituted or unsubstituted naphthyl group, a substituted or unsubstituted phenanthryl group, a substituted or unsubstituted dibenzofuranyl group, a substituted or unsubstituted dibenzothiophenyl group, a substituted or unsubstituted benzonaphthofuranyl group, or a substituted or unsubstituted benzonaphthothiophenyl group.

In Formula 2, “-*” is a portion which is bonded to one of R₁ to R₅ in Formula 1. The nitrogen atom of the amine group represented by Formula 2 may be directly bonded to one selected from among R₁ to R₅.

If (e.g., when) R₂ in Formula 1 is the amine group represented by Formula 2, a case where FG1 and FG2 are each a substituted or unsubstituted aryl group having ring-forming carbons of 6 to 15 at the same time is excluded. In addition, if (e.g., when) R₂ is the amine group represented by Formula 2, a case where FG1 or FG2 is a benzonaphthofuranyl group substituted with an aryl group is excluded. For example, if (e.g., when) R₂ in Formula 1 is the amine group represented by Formula 2, at least one selected from among FG1 and FG2 may be a substituted or unsubstituted aryl group having ring-forming carbons of 16 to 40 or a substituted or unsubstituted heteroaryl group having ring-forming carbons of 5 to 40. In addition, if (e.g., when) R₂ in Formula is the amine group represented by Formula 2, FG1 and FG2 include no benzonaphthofuranyl group substituted with an aryl group.

If (e.g., when) R₃ in Formula 1 is the amine group represented by Formula 2, a case where FG1 and FG2 are each a substituted or unsubstituted aryl group having ring-forming carbons of 6 to 9 at the same time is excluded, and if (e.g., when) one of FG1 and FG2 is an aryl group having total ring-forming carbons of 26 or greater, the other may be an aryl group having a total ring-forming carbons of 6 to 15 or a heteroaryl group having ring-forming carbons of 5 to 40.

For example, in the case where R₃ in Formula 1 is the amine group represented by Formula 2, if (e.g., when) one among FG1 and FG2 is a substituted or unsubstituted aryl group having ring-forming carbons of 6 to 9, and the other may be a substituted or unsubstituted aryl group having ring-forming carbons of 10 to 40 or a substituted or unsubstituted heteroaryl group having ring-forming carbons of 5 to 40. In addition, in the case where R₃ in Formula 1 is the amine group represented by Formula 2, if (e.g., when) one among FG1 and FG2 is an aryl group and the number of total ring-forming carbons of entire rings constituting the aryl group is 26 or greater, the other may be an aryl group having total ring-forming carbons of entire rings of 6 to 15 or a substituted or unsubstituted heteroaryl group having ring-forming carbons of 5 to 40.

In a case where R₅ in Formula 1 is the amine group represented by Formula 2, if (e.g., when) FG1 or FG2 is a 4-dibenzofuranyl group or a 4-dibenzothiophenyl group, at least one hydrogen of the 4-dibenzofuranyl group or the 4-dibenzothiophenyl group is substituted with a substituent, and cases where FG1 or FG2 is a naphthyl group or a benzonaphthofuranyl group substituted with an aryl group are excluded.

For example, in the case where R₅ in Formula 1 is the amine group represented by Formula 2, if (e.g., when) FG1 or FG2 is the 4-dibenzofuranyl group or the 4-dibenzothiophenyl group, as represented by Formula A, at least one hydrogen of the bonded 4-dibenzofuranyl group or 4-dibenzothiophenyl group may be substituted. In addition, if (e.g., when) R₅ in Formula 1 is the amine group represented by Formula 2, cases where FG1 or FG2 is a benzonaphthofuranyl group substituted or unsubstituted a naphthyl group or an aryl group are excluded.

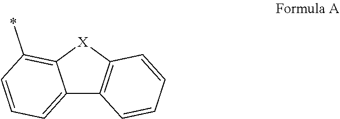

In Formula A, X may be O or S, and “-*” is a portion where the N atom of the amine group represented by Formula 2 is bonded.

If (e.g., when) R₁ in Formula 1 is the amine group represented by Formula 2, FG1 and FG2 may each independently be, except for a fluorenyl group, a substituted or unsubstituted aryl group having ring-forming carbons of 6 to 40 or a substituted or unsubstituted heteroaryl group including one to three hetero atoms and having ring-forming carbons of 5 to 40, and FG1 and FG2 include no alkyl group as a substituent.

In addition, if (e.g., when) R₄ in Formula 1 is the amine group represented by Formula 2, FG1 and FG2 may each independently be, except for a fluorenyl group, a substituted or unsubstituted aryl group having ring-forming carbons of 6 to 40 or a substituted or unsubstituted heteroaryl group including one to three hetero atoms and having ring-forming carbons of 5 to 40, and FG1 and FG2 include no alkyl group as a substituent.

In the amine compound according to one or more embodiments including a phenanthrene group represented by Formula 1 and an amine group represented by Formula 2, at least one hydrogen may be substituted with deuterium. For example, in the amine compound according to one or more embodiments in which a phenanthrene group represented by Formula 1 and deuterated, and an aromatic amine group represented by Formula 2 are bonded, at least one hydrogen of the amine group may be represented by deuterium. For example, at least one hydrogen of FG1 and FG2 in Formula 2 may be substituted with deuterium.

The amine compound according to one or more embodiments including the phenanthrene group represented by Formula 1 and the amine group represented by Formula 2 may be a monoamine compound including no additional amino group.

The amine compound according to one or more embodiments may be represented by any one selected from among Formulae 1A to 1E.

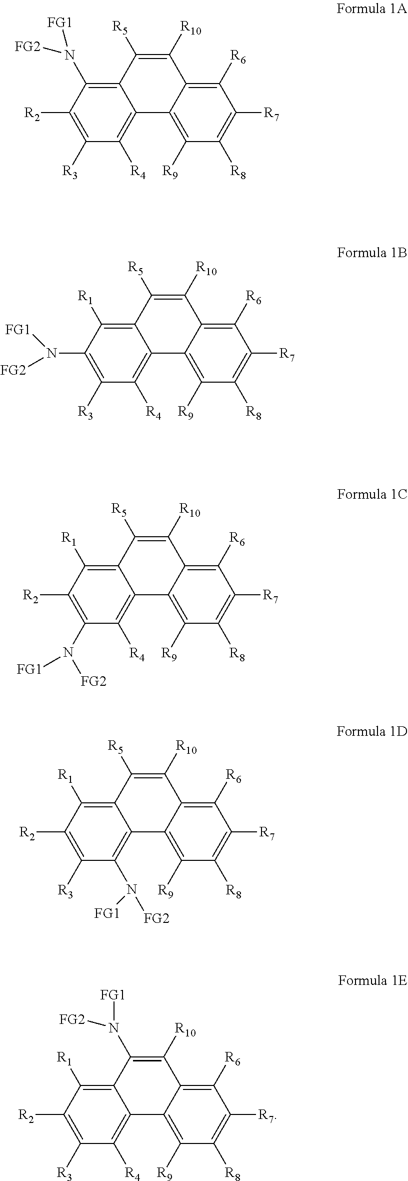

In Formula 1A to Formula 1E, the descriptions of the amine compound represented by Formula 1 and Formula 2 may be similarly applied to R₁ to R₁₀, FG1, and FG2.

The amine compound according to one or more embodiments may be any one selected from among compounds in Compound Group 1. The hole transport region HTR of the light-emitting element ED according to one or more embodiments may include at least one selected from among the amine compounds disclosed in Compound Group 1. In the following Compound Group 1, D is deuterium.

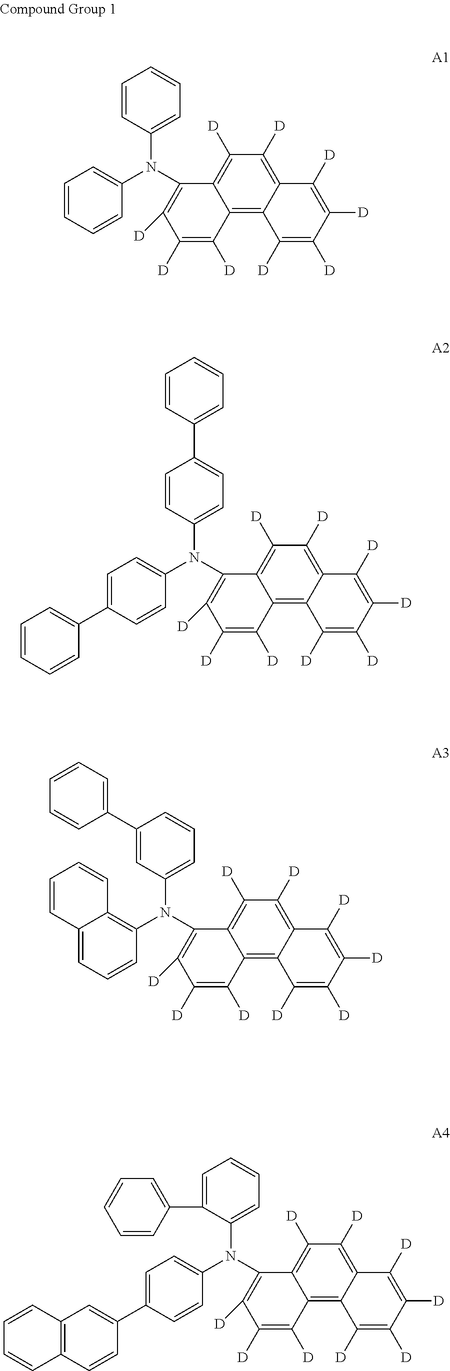 [SMILES: [2H]c1c([2H])c([2H])c2c(c1[2H])c([2H])c([2H])c1c(N(c3ccccc3)c3ccccc3)c([2H])c([2H])c([2H])c12 ; [2H]c1c([2H])c([2H])c2c(c1[2H])c([2H])c([2H])c1c(N(c3ccc(-c4ccccc4)cc3)c3ccc(-c4ccccc4)cc3)c([2H])c([2H])c([2H])c12 ; [2H]c1c([2H])c([2H])c2c(c1[2H])c([2H])c([2H])c1c(N(c3cccc(-c4ccccc4)c3)c3cccc4ccccc34)c([2H])c([2H])c([2H])c12 ; [2H]c1c([2H])c([2H])c2c(c1[2H])c([2H])c([2H])c1c(N(c3ccc(-c4ccc5ccccc5c4)cc3)c3ccccc3-c3ccccc3)c([2H])c([2H])c([2H])c12]

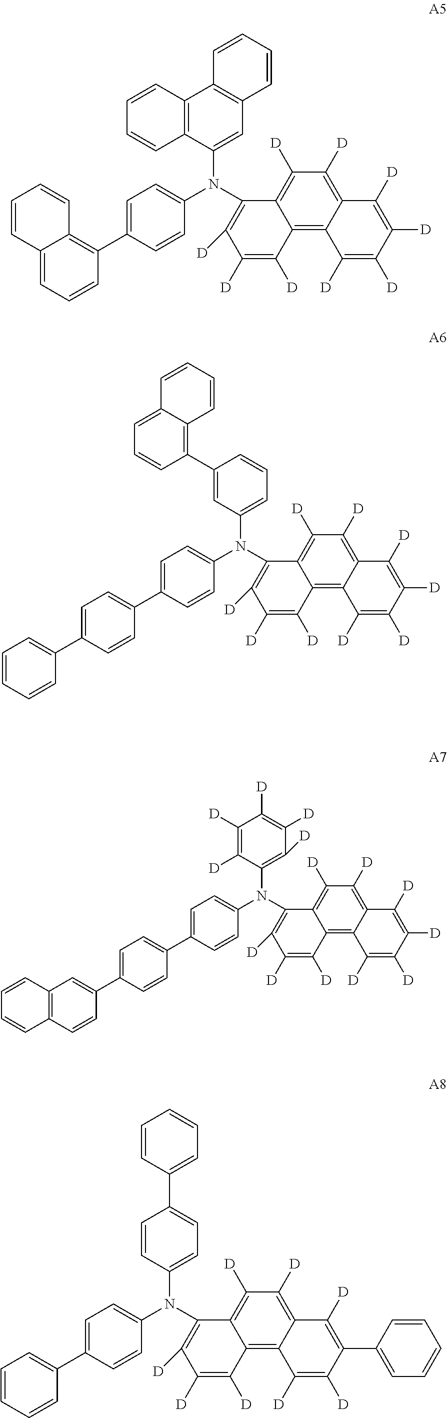

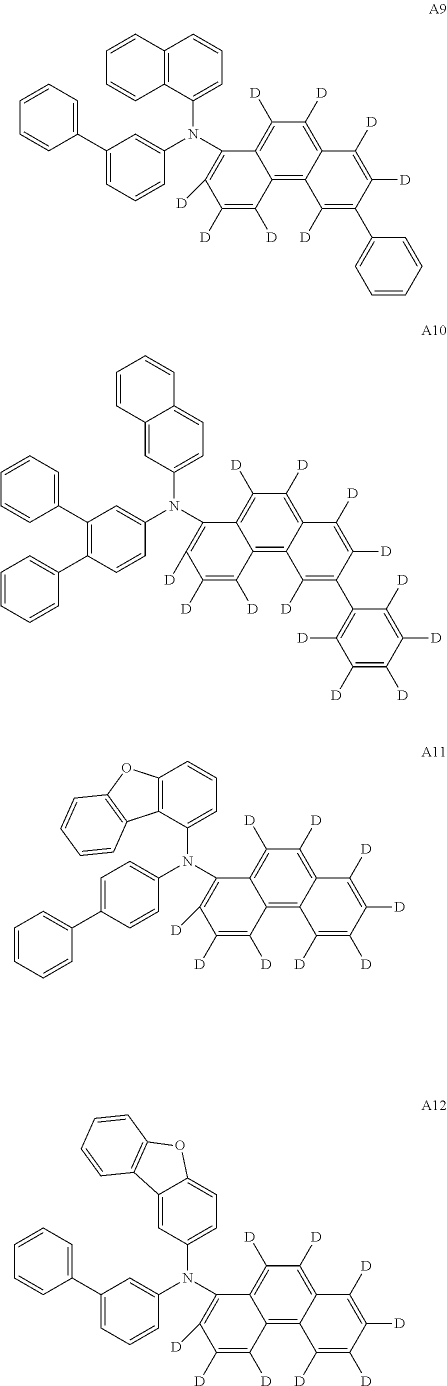

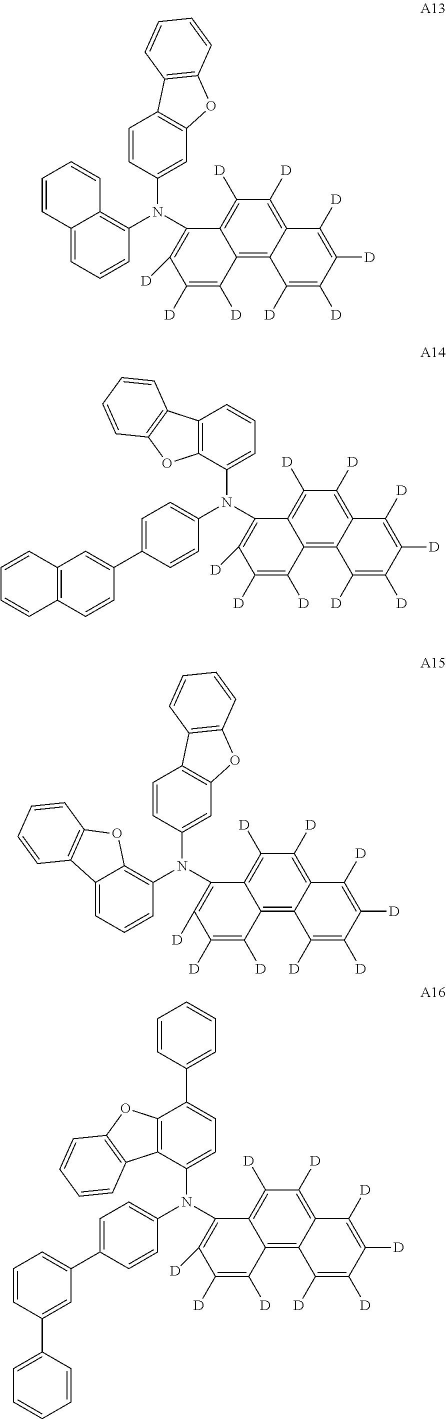

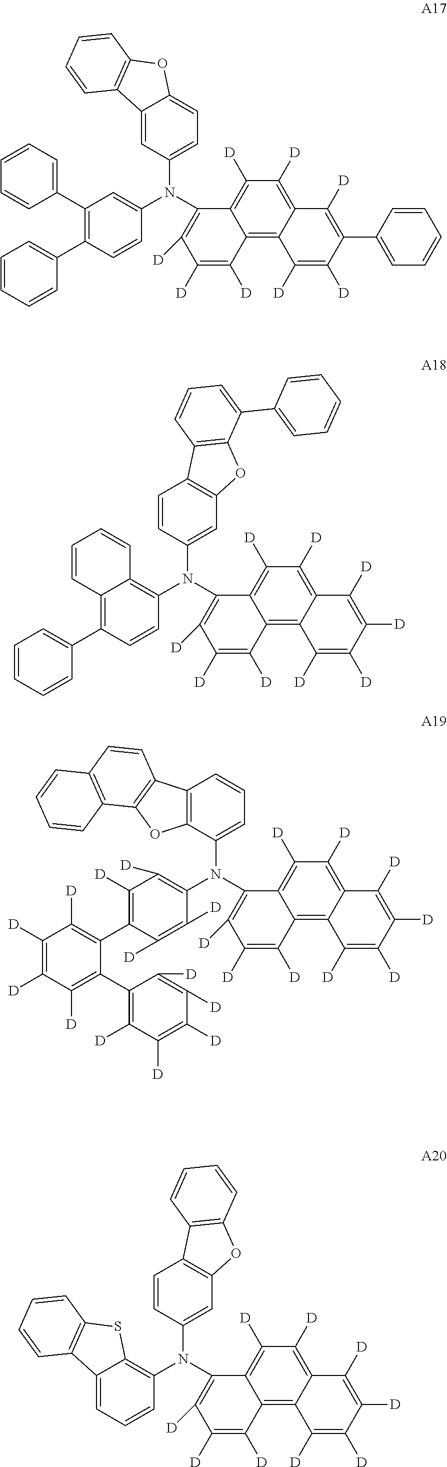

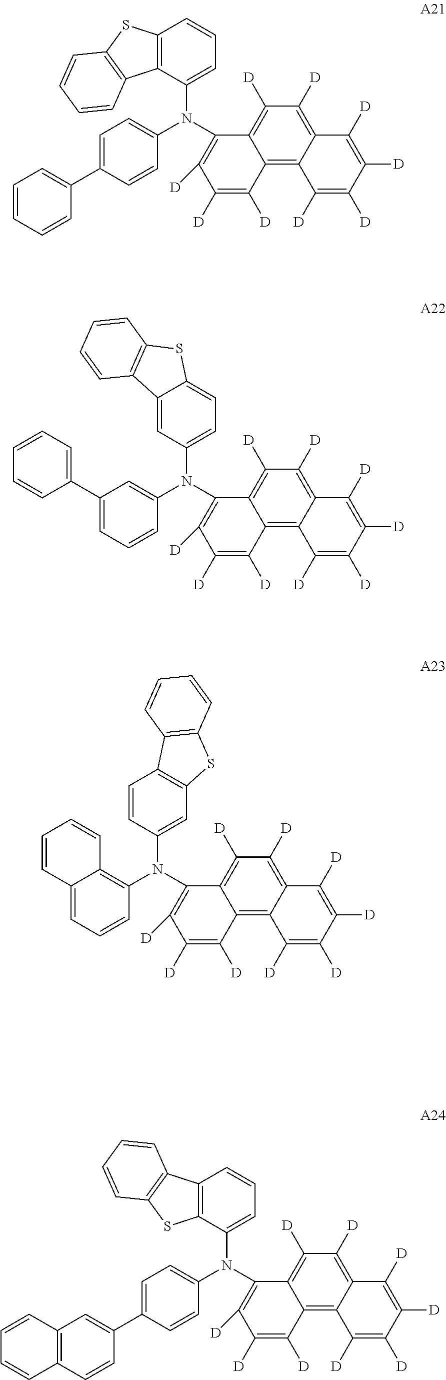 [SMILES: [2H]c1c([2H])c([2H])c2c(c1[2H])c([2H])c([2H])c1c(N(c3ccc(-c4ccccc4)cc3)c3cccc4sc5ccccc5c34)c([2H])c([2H])c([2H])c12 ; [2H]c1c([2H])c([2H])c2c(c1[2H])c([2H])c([2H])c1c(N(c3cccc(-c4ccccc4)c3)c3ccc4sc5ccccc5c4c3)c([2H])c([2H])c([2H])c12 ; [2H]c1c([2H])c([2H])c2c(c1[2H])c([2H])c([2H])c1c(N(c3ccc4c(c3)sc3ccccc34)c3cccc4ccccc34)c([2H])c([2H])c([2H])c12 ; [2H]c1c([2H])c([2H])c2c(c1[2H])c([2H])c([2H])c1c(N(c3ccc(-c4ccc5ccccc5c4)cc3)c3cccc4c3sc3ccccc34)c([2H])c([2H])c([2H])c12]

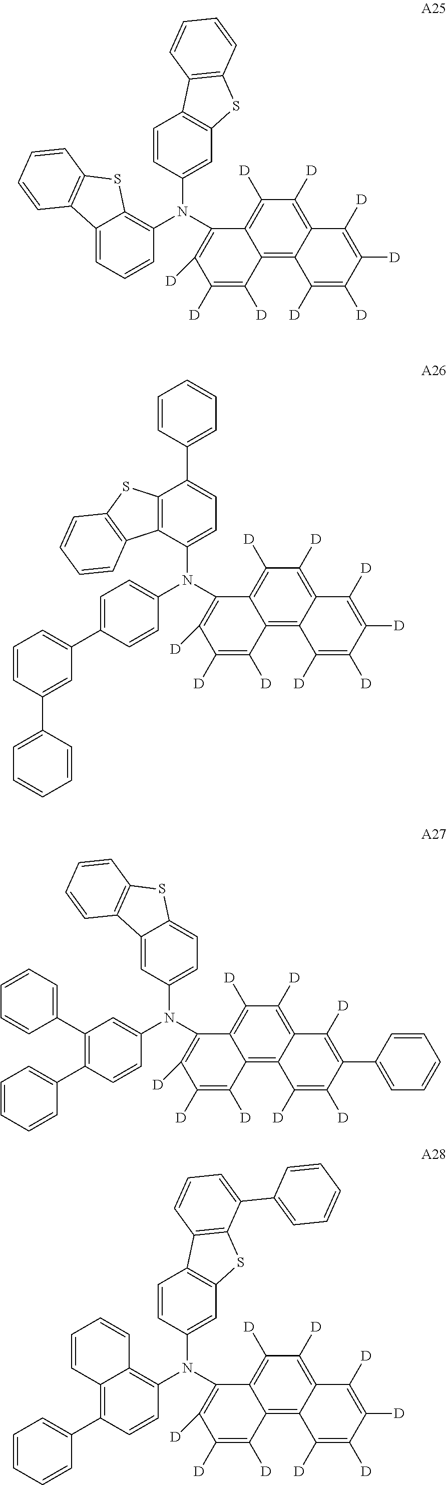

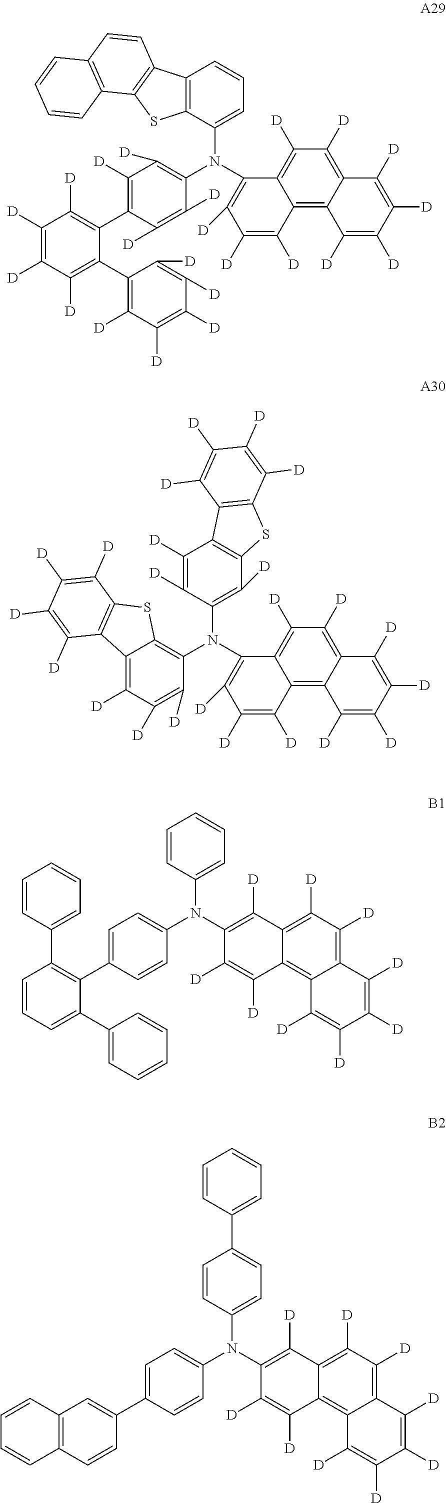

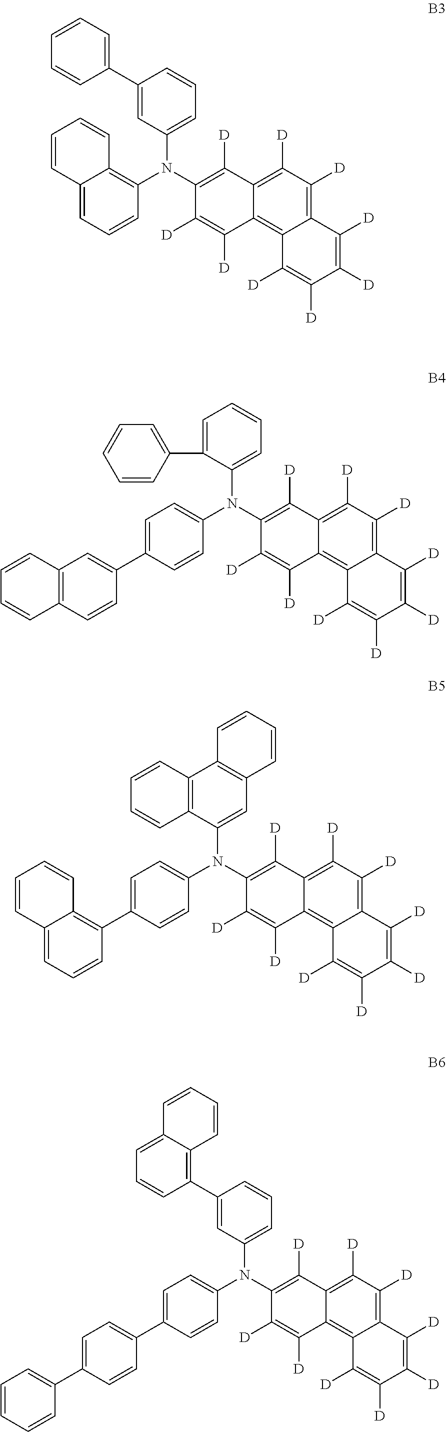 [SMILES: [2H]c1c([2H])c([2H])c2c(c1[2H])c([2H])c([2H])c1c([2H])c(N(c3cccc(-c4ccccc4)c3)c3cccc4ccccc34)c([2H])c([2H])c12 ; [2H]c1c([2H])c([2H])c2c(c1[2H])c([2H])c([2H])c1c([2H])c(N(c3ccc(-c4ccc5ccccc5c4)cc3)c3ccccc3-c3ccccc3)c([2H])c([2H])c12 ; [2H]c1c([2H])c([2H])c2c(c1[2H])c([2H])c([2H])c1c([2H])c(N(c3ccc(-c4cccc5ccccc45)cc3)c3cc4ccccc4c4ccccc34)c([2H])c([2H])c12 ; [2H]c1c([2H])c([2H])c2c(c1[2H])c([2H])c([2H])c1c([2H])c(N(c3ccc(-c4ccc(-c5ccccc5)cc4)cc3)c3cccc(-c4cccc5ccccc45)c3)c([2H])c([2H])c12]

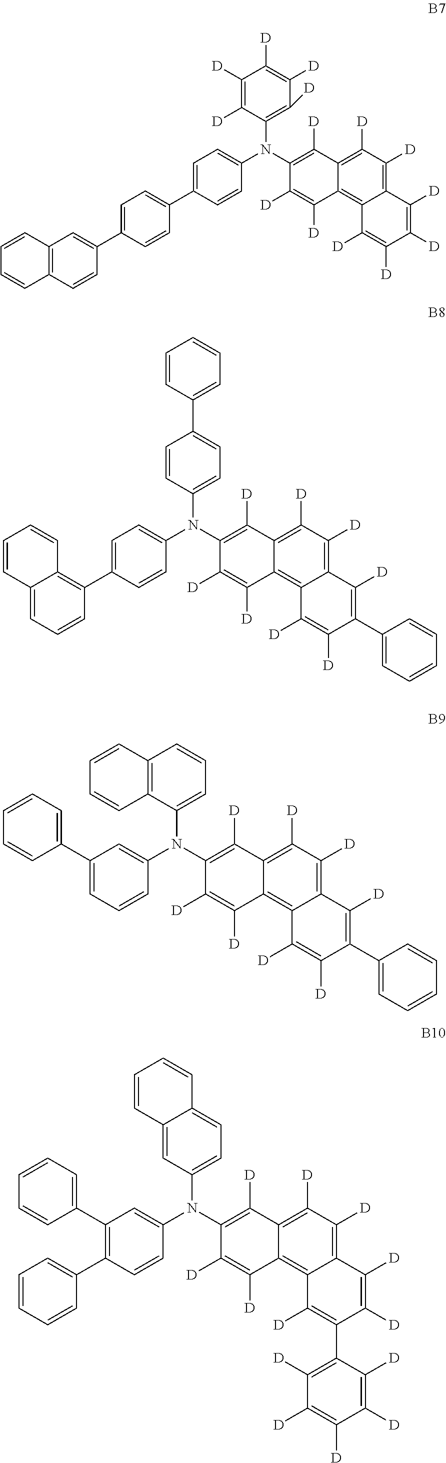

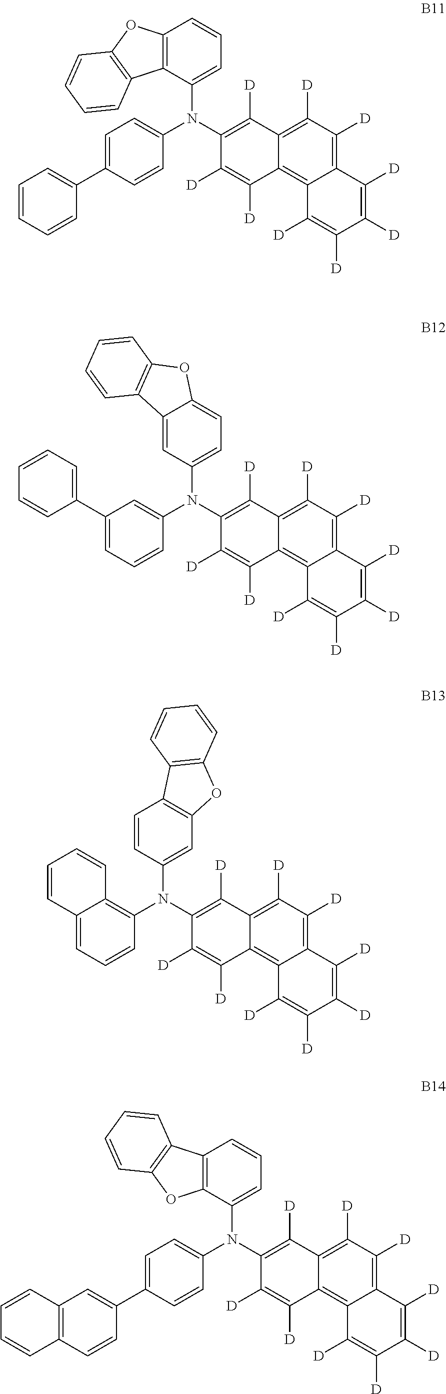 [SMILES: [2H]c1c([2H])c([2H])c2c(c1[2H])c([2H])c([2H])c1c([2H])c(N(c3ccc(-c4ccccc4)cc3)c3cccc4oc5ccccc5c34)c([2H])c([2H])c12 ; [2H]c1c([2H])c([2H])c2c(c1[2H])c([2H])c([2H])c1c([2H])c(N(c3cccc(-c4ccccc4)c3)c3ccc4oc5ccccc5c4c3)c([2H])c([2H])c12 ; [2H]c1c([2H])c([2H])c2c(c1[2H])c([2H])c([2H])c1c([2H])c(N(c3ccc4c(c3)oc3ccccc34)c3cccc4ccccc34)c([2H])c([2H])c12 ; [2H]c1c([2H])c([2H])c2c(c1[2H])c([2H])c([2H])c1c([2H])c(N(c3ccc(-c4ccc5ccccc5c4)cc3)c3cccc4c3oc3ccccc34)c([2H])c([2H])c12]

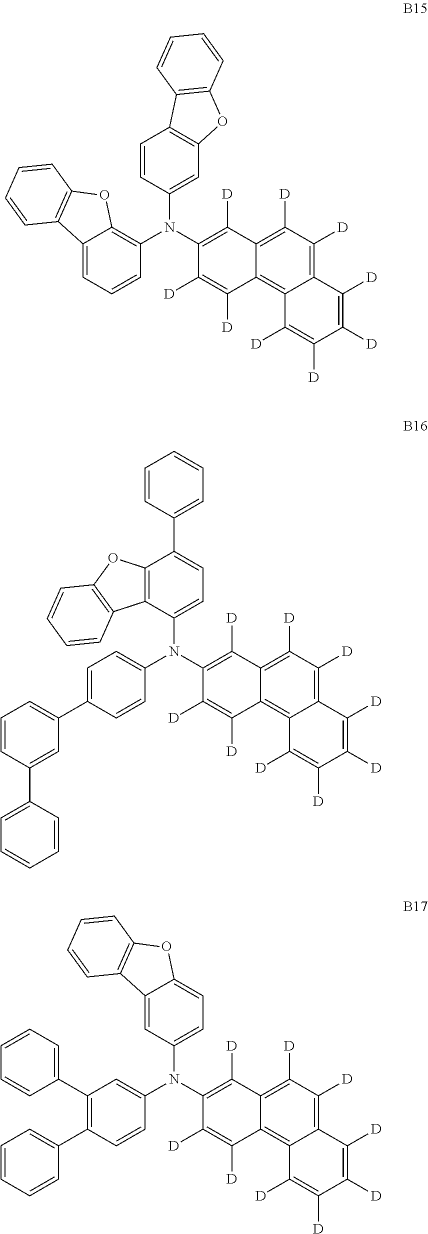 [SMILES: [2H]c1c([2H])c([2H])c2c(c1[2H])c([2H])c([2H])c1c([2H])c(N(c3ccc4c(c3)oc3ccccc34)c3cccc4c3oc3ccccc34)c([2H])c([2H])c12 ; [2H]c1c([2H])c([2H])c2c(c1[2H])c([2H])c([2H])c1c([2H])c(N(c3ccc(-c4cccc(-c5ccccc5)c4)cc3)c3ccc(-c4ccccc4)c4oc5ccccc5c34)c([2H])c([2H])c12 ; [2H]c1c([2H])c([2H])c2c(c1[2H])c([2H])c([2H])c1c([2H])c(N(c3ccc(-c4ccccc4)c(-c4ccccc4)c3)c3ccc4oc5ccccc5c4c3)c([2H])c([2H])c12]

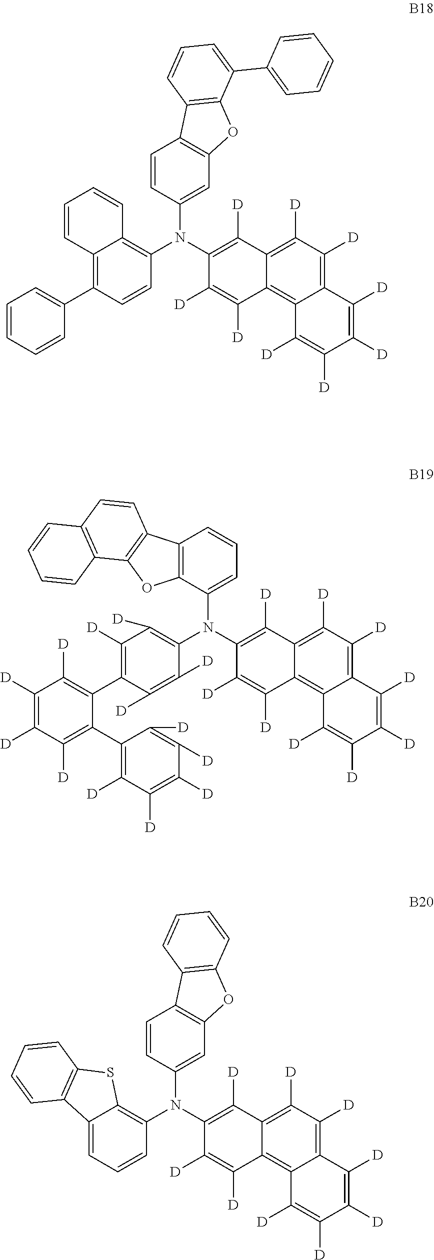 [SMILES: [2H]c1c([2H])c([2H])c2c(c1[2H])c([2H])c([2H])c1c([2H])c(N(c3ccc4c(c3)oc3c(-c5ccccc5)cccc34)c3ccc(-c4ccccc4)c4ccccc34)c([2H])c([2H])c12 ; [2H]c1c([2H])c([2H])c(-c2c([2H])c([2H])c([2H])c([2H])c2-c2c([2H])c([2H])c(N(c3c([2H])c([2H])c4c(c3[2H])c([2H])c([2H])c3c([2H])c([2H])c([2H])c([2H])c34)c3cccc4c3oc3c5ccccc5ccc43)c([2H])c2[2H])c([2H])c1[2H] ; [2H]c1c([2H])c([2H])c2c(c1[2H])c([2H])c([2H])c1c([2H])c(N(c3ccc4c(c3)oc3ccccc34)c3cccc4c3sc3ccccc34)c([2H])c([2H])c12]

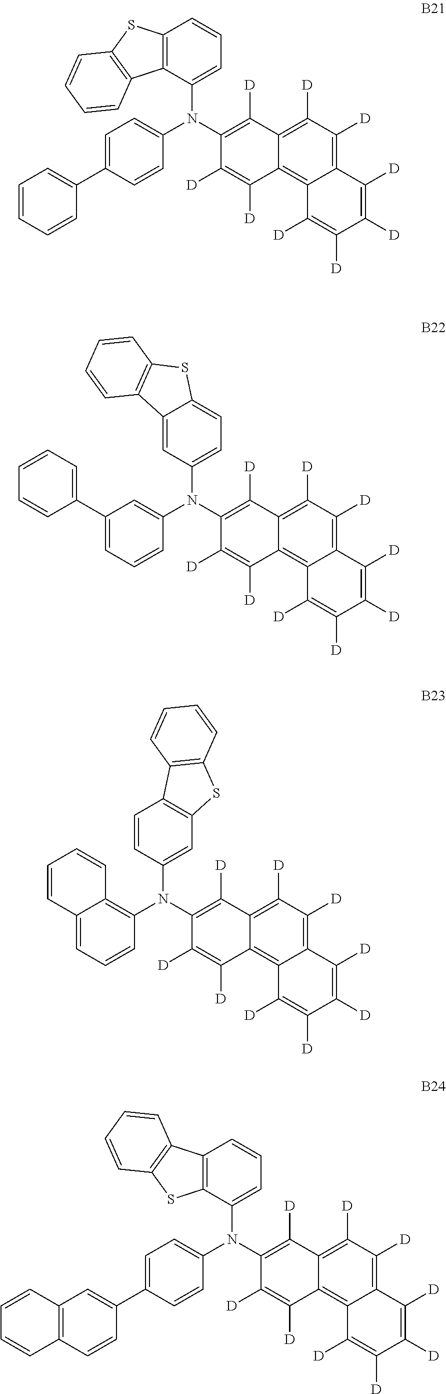 [SMILES: [2H]c1c([2H])c([2H])c2c(c1[2H])c([2H])c([2H])c1c([2H])c(N(c3ccc(-c4ccccc4)cc3)c3cccc4sc5ccccc5c34)c([2H])c([2H])c12 ; [2H]c1c([2H])c([2H])c2c(c1[2H])c([2H])c([2H])c1c([2H])c(N(c3cccc(-c4ccccc4)c3)c3ccc4sc5ccccc5c4c3)c([2H])c([2H])c12 ; [2H]c1c([2H])c([2H])c2c(c1[2H])c([2H])c([2H])c1c([2H])c(N(c3ccc4c(c3)sc3ccccc34)c3cccc4ccccc34)c([2H])c([2H])c12 ; [2H]c1c([2H])c([2H])c2c(c1[2H])c([2H])c([2H])c1c([2H])c(N(c3ccc(-c4ccc5ccccc5c4)cc3)c3cccc4c3sc3ccccc34)c([2H])c([2H])c12]

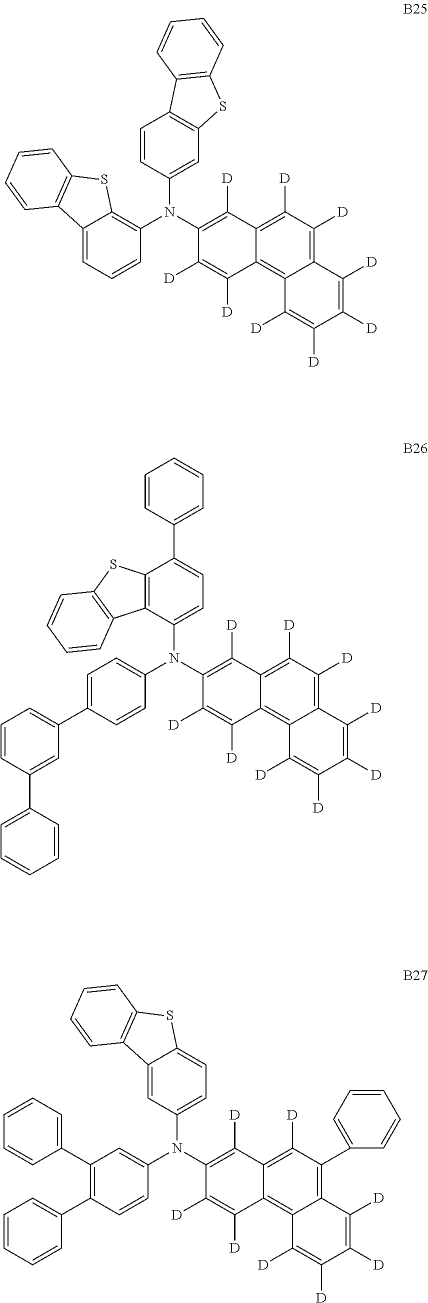 [SMILES: [2H]c1c([2H])c([2H])c2c(c1[2H])c([2H])c([2H])c1c([2H])c(N(c3ccc4c(c3)sc3ccccc34)c3cccc4c3sc3ccccc34)c([2H])c([2H])c12 ; [2H]c1c([2H])c([2H])c2c(c1[2H])c([2H])c([2H])c1c([2H])c(N(c3ccc(-c4cccc(-c5ccccc5)c4)cc3)c3ccc(-c4ccccc4)c4sc5ccccc5c34)c([2H])c([2H])c12 ; [2H]c1c([2H])c([2H])c2c(c1[2H])c(-c1ccccc1)c([2H])c1c([2H])c(N(c3ccc(-c4ccccc4)c(-c4ccccc4)c3)c3ccc4sc5ccccc5c4c3)c([2H])c([2H])c12]

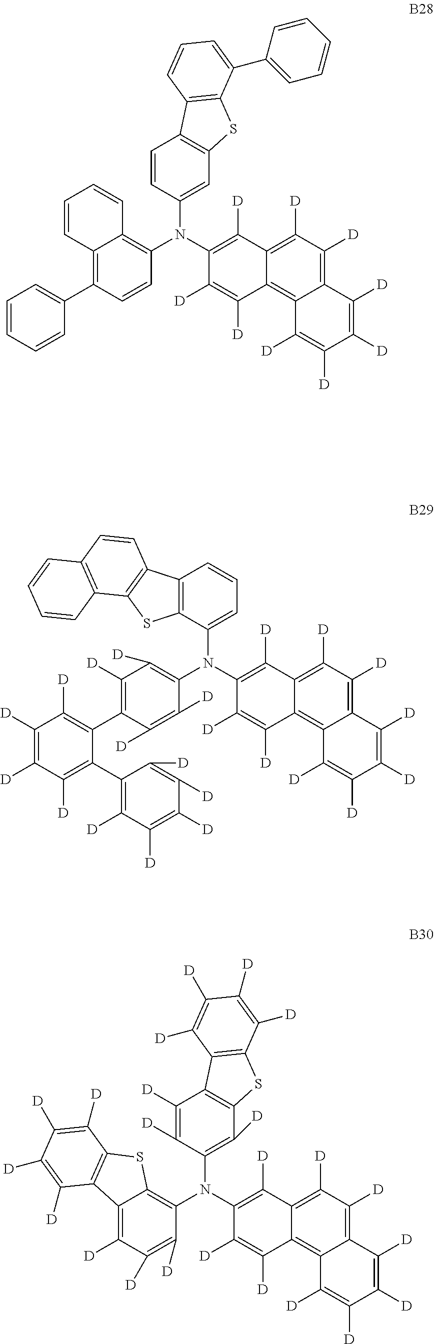

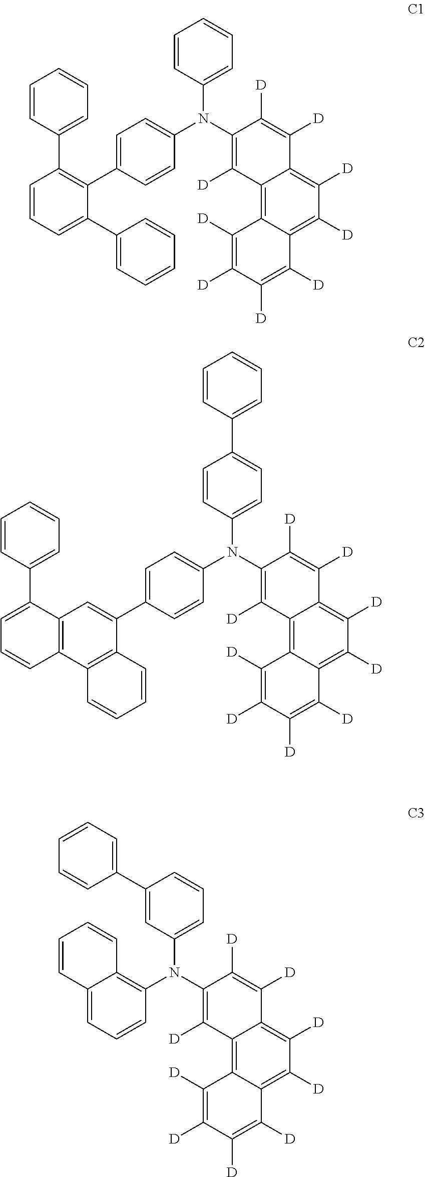

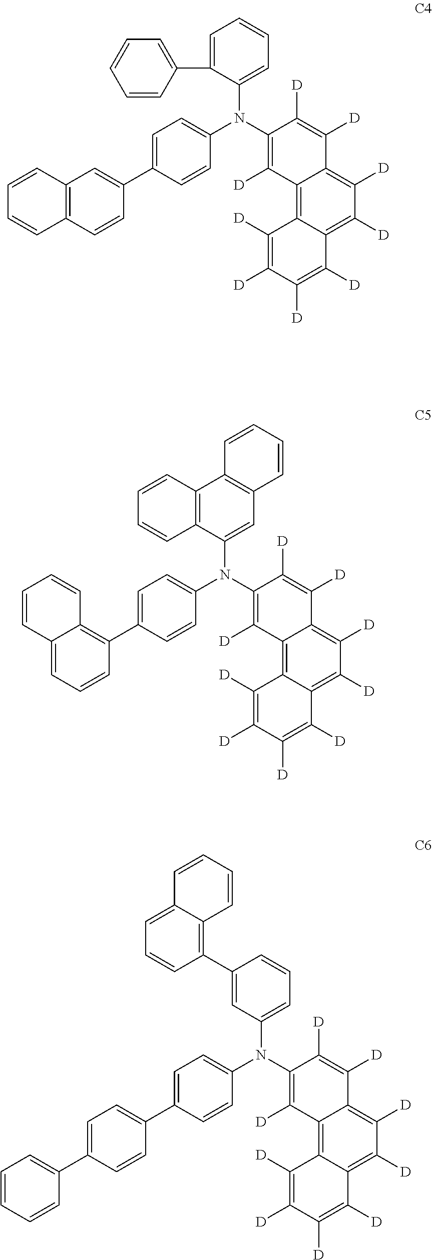 [SMILES: [2H]c1c([2H])c([2H])c2c(c1[2H])c([2H])c([2H])c1c([2H])c([2H])c(N(c3ccc(-c4ccc5ccccc5c4)cc3)c3ccccc3-c3ccccc3)c([2H])c12 ; [2H]c1c([2H])c([2H])c2c(c1[2H])c([2H])c([2H])c1c([2H])c([2H])c(N(c3ccc(-c4cccc5ccccc45)cc3)c3cc4ccccc4c4ccccc34)c([2H])c12 ; [2H]c1c([2H])c([2H])c2c(c1[2H])c([2H])c([2H])c1c([2H])c([2H])c(N(c3ccc(-c4ccc(-c5ccccc5)cc4)cc3)c3cccc(-c4cccc5ccccc45)c3)c([2H])c12]

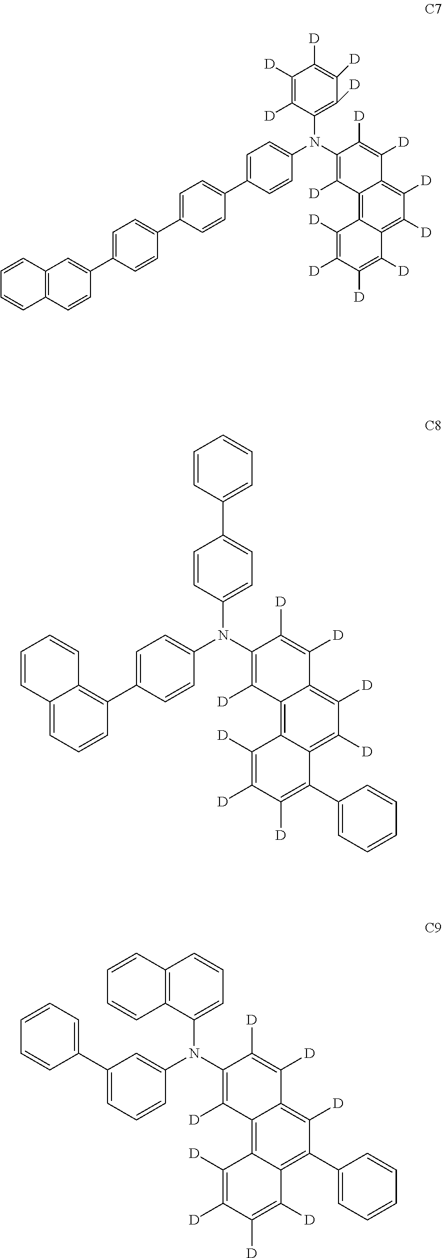 [SMILES: [2H]c1c([2H])c([2H])c(N(c2ccc(-c3ccc(-c4ccc(-c5ccc6ccccc6c5)cc4)cc3)cc2)c2c([2H])c([2H])c3c([2H])c([2H])c4c([2H])c([2H])c([2H])c([2H])c4c3c2[2H])c([2H])c1[2H] ; [2H]c1c(N(c2ccc(-c3ccccc3)cc2)c2ccc(-c3cccc4ccccc34)cc2)c([2H])c2c(c1[2H])c([2H])c([2H])c1c(-c3ccccc3)c([2H])c([2H])c([2H])c12 ; [2H]c1c(N(c2cccc(-c3ccccc3)c2)c2cccc3ccccc23)c([2H])c2c(c1[2H])c([2H])c(-c1ccccc1)c1c([2H])c([2H])c([2H])c([2H])c12]

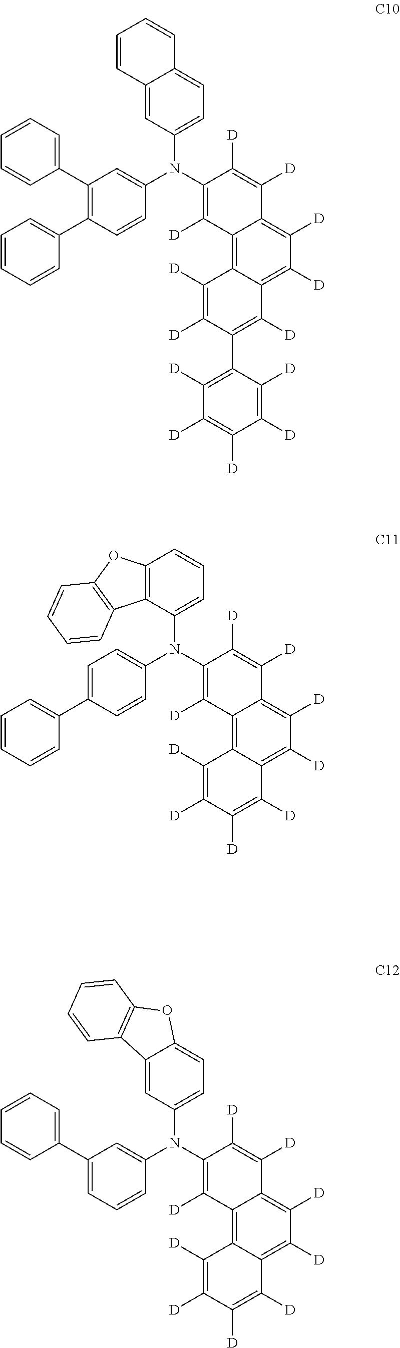 [SMILES: [2H]c1c([2H])c([2H])c(-c2c([2H])c([2H])c3c(c2[2H])c([2H])c([2H])c2c([2H])c([2H])c(N(c4ccc(-c5ccccc5)c(-c5ccccc5)c4)c4ccc5ccccc5c4)c([2H])c23)c([2H])c1[2H] ; [2H]c1c([2H])c([2H])c2c(c1[2H])c([2H])c([2H])c1c([2H])c([2H])c(N(c3ccc(-c4ccccc4)cc3)c3cccc4oc5ccccc5c34)c([2H])c12 ; [2H]c1c([2H])c([2H])c2c(c1[2H])c([2H])c([2H])c1c([2H])c([2H])c(N(c3cccc(-c4ccccc4)c3)c3ccc4oc5ccccc5c4c3)c([2H])c12]

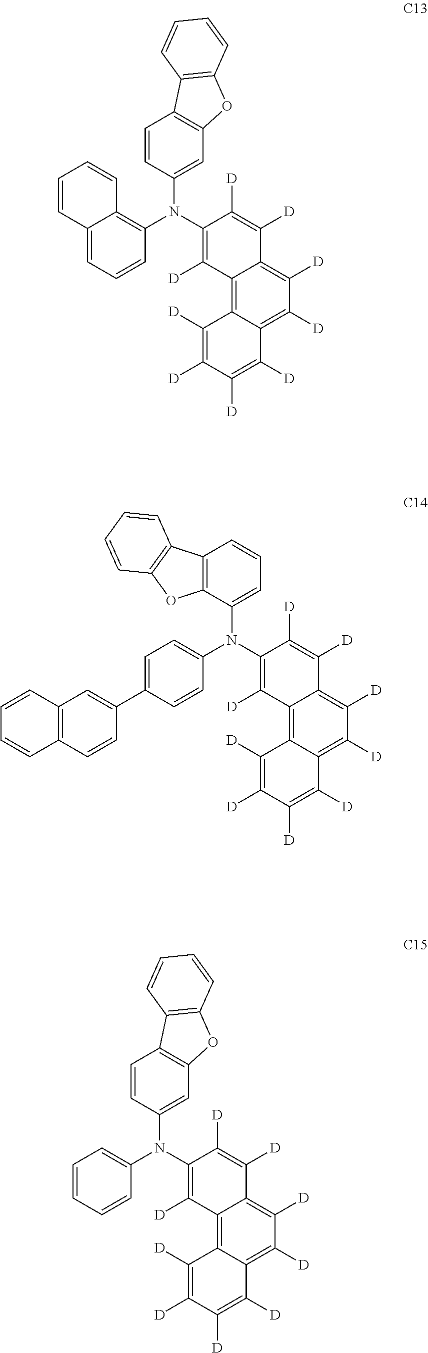 [SMILES: [2H]c1c([2H])c([2H])c2c(c1[2H])c([2H])c([2H])c1c([2H])c([2H])c(N(c3ccc4c(c3)oc3ccccc34)c3cccc4ccccc34)c([2H])c12 ; [2H]c1c([2H])c([2H])c2c(c1[2H])c([2H])c([2H])c1c([2H])c([2H])c(N(c3ccc(-c4ccc5ccccc5c4)cc3)c3cccc4c3oc3ccccc34)c([2H])c12 ; [2H]c1c([2H])c([2H])c2c(c1[2H])c([2H])c([2H])c1c([2H])c([2H])c(N(c3ccccc3)c3ccc4c(c3)oc3ccccc34)c([2H])c12]

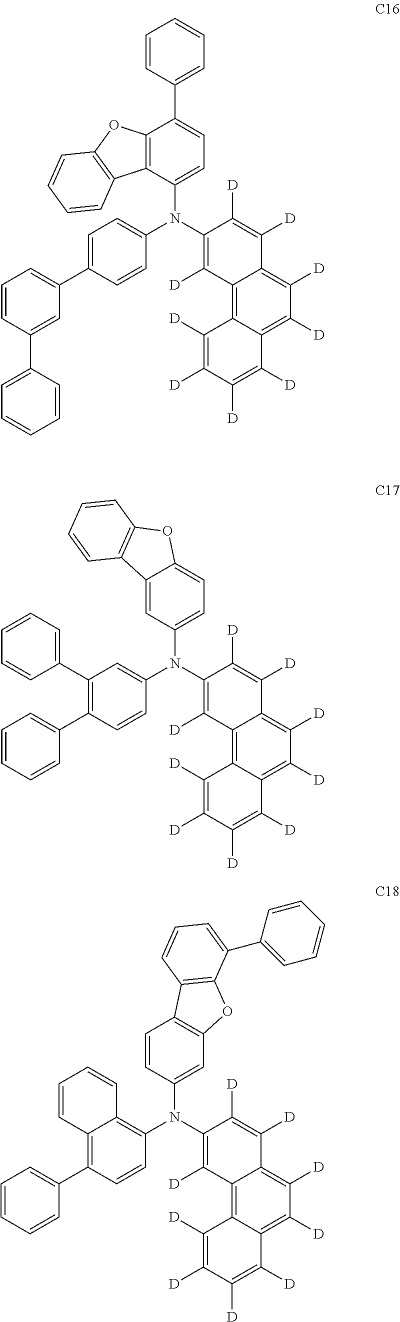 [SMILES: [2H]c1c([2H])c([2H])c2c(c1[2H])c([2H])c([2H])c1c([2H])c([2H])c(N(c3ccc(-c4cccc(-c5ccccc5)c4)cc3)c3ccc(-c4ccccc4)c4oc5ccccc5c34)c([2H])c12 ; [2H]c1c([2H])c([2H])c2c(c1[2H])c([2H])c([2H])c1c([2H])c([2H])c(N(c3ccc(-c4ccccc4)c(-c4ccccc4)c3)c3ccc4oc5ccccc5c4c3)c([2H])c12 ; [2H]c1c([2H])c([2H])c2c(c1[2H])c([2H])c([2H])c1c([2H])c([2H])c(N(c3ccc4c(c3)oc3c(-c5ccccc5)cccc34)c3ccc(-c4ccccc4)c4ccccc34)c([2H])c12]

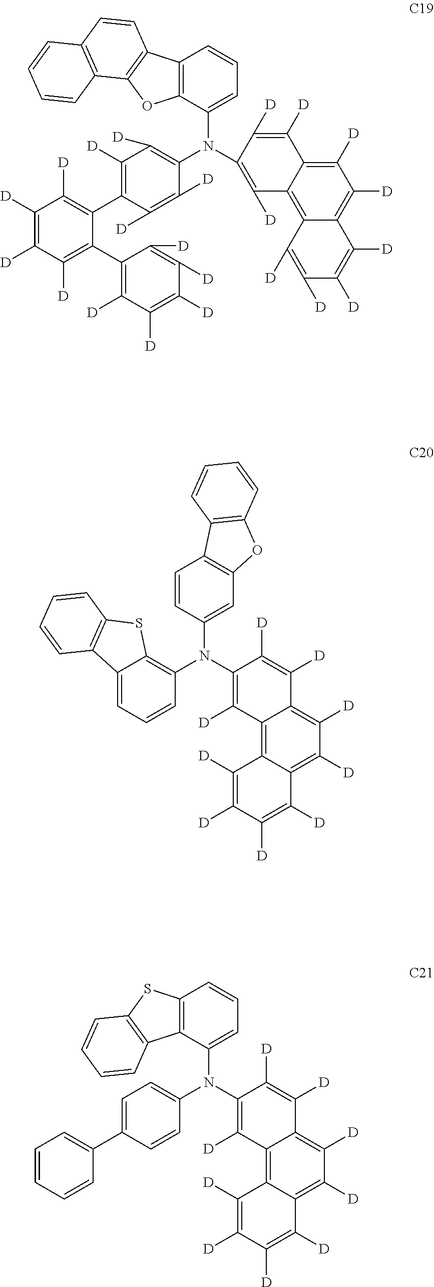 [SMILES: [2H]c1c([2H])c([2H])c(-c2c([2H])c([2H])c([2H])c([2H])c2-c2c([2H])c([2H])c(N(c3c([2H])c([2H])c4c([2H])c([2H])c5c([2H])c([2H])c([2H])c([2H])c5c4c3[2H])c3cccc4c3oc3c5ccccc5ccc43)c([2H])c2[2H])c([2H])c1[2H] ; [2H]c1c([2H])c([2H])c2c(c1[2H])c([2H])c([2H])c1c([2H])c([2H])c(N(c3ccc4c(c3)oc3ccccc34)c3cccc4c3sc3ccccc34)c([2H])c12 ; [2H]c1c([2H])c([2H])c2c(c1[2H])c([2H])c([2H])c1c([2H])c([2H])c(N(c3ccc(-c4ccccc4)cc3)c3cccc4sc5ccccc5c34)c([2H])c12]

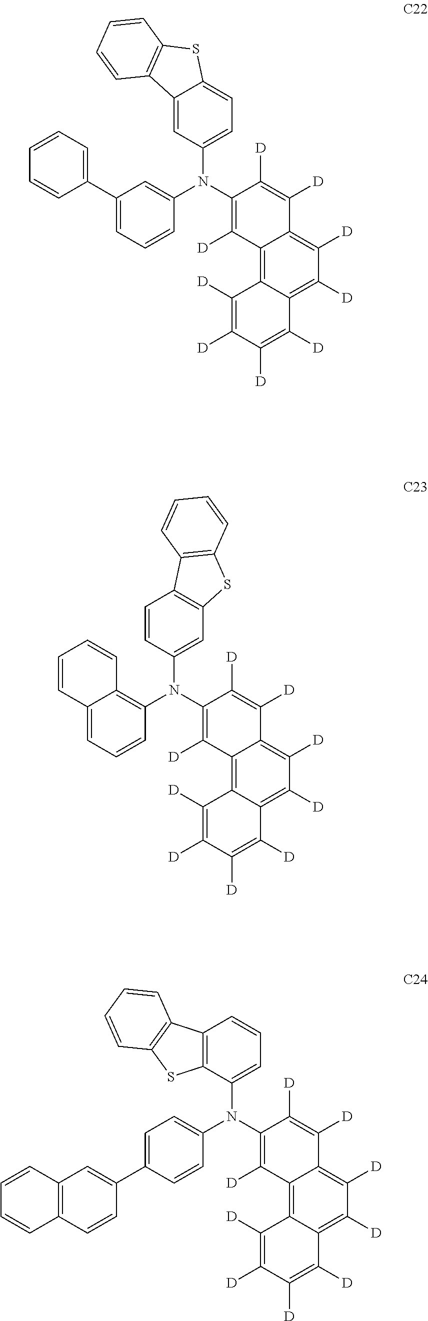 [SMILES: [2H]c1c([2H])c([2H])c2c(c1[2H])c([2H])c([2H])c1c([2H])c([2H])c(N(c3cccc(-c4ccccc4)c3)c3ccc4sc5ccccc5c4c3)c([2H])c12 ; [2H]c1c([2H])c([2H])c2c(c1[2H])c([2H])c([2H])c1c([2H])c([2H])c(N(c3ccc4c(c3)sc3ccccc34)c3cccc4ccccc34)c([2H])c12 ; [2H]c1c([2H])c([2H])c2c(c1[2H])c([2H])c([2H])c1c([2H])c([2H])c(N(c3ccc(-c4ccc5ccccc5c4)cc3)c3cccc4c3sc3ccccc34)c([2H])c12]

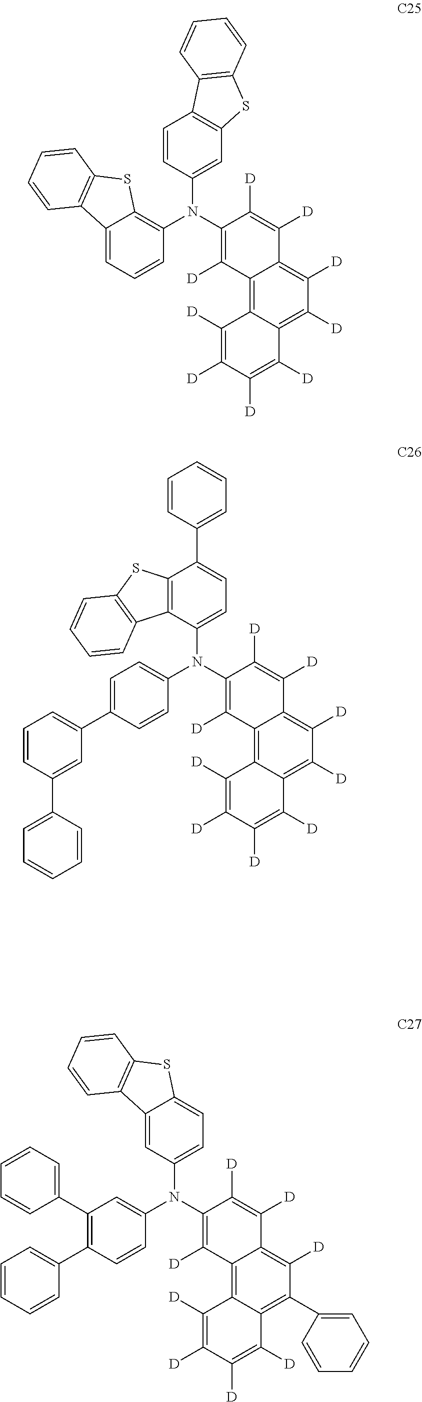 [SMILES: [2H]c1c([2H])c([2H])c2c(c1[2H])c([2H])c([2H])c1c([2H])c([2H])c(N(c3ccc4c(c3)sc3ccccc34)c3cccc4c3sc3ccccc34)c([2H])c12 ; [2H]c1c([2H])c([2H])c2c(c1[2H])c([2H])c([2H])c1c([2H])c([2H])c(N(c3ccc(-c4cccc(-c5ccccc5)c4)cc3)c3ccc(-c4ccccc4)c4sc5ccccc5c34)c([2H])c12 ; [2H]c1c(N(c2ccc(-c3ccccc3)c(-c3ccccc3)c2)c2ccc3sc4ccccc4c3c2)c([2H])c2c(c1[2H])c([2H])c(-c1ccccc1)c1c([2H])c([2H])c([2H])c([2H])c12]

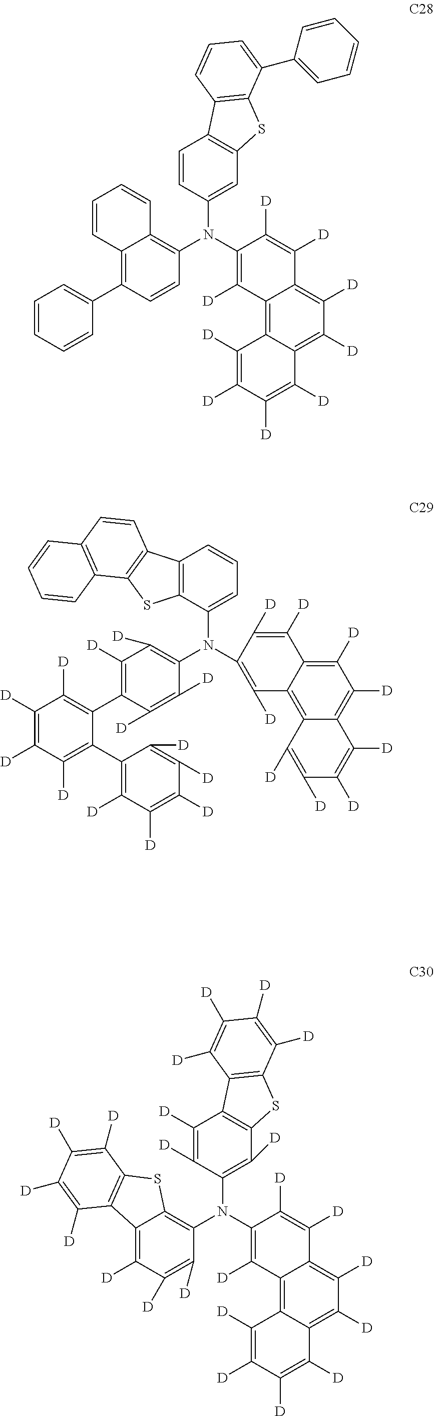

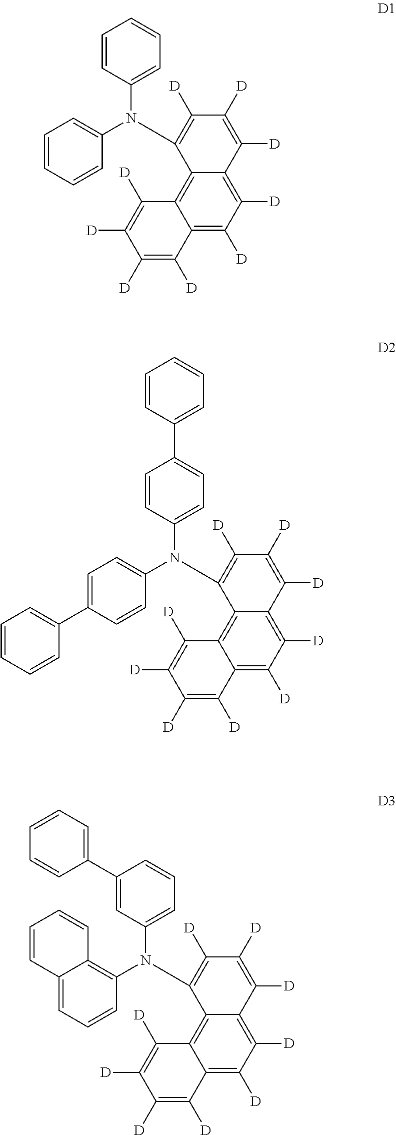 [SMILES: [2H]c1c([2H])c([2H])c2c(c1[2H])c([2H])c([2H])c1c([2H])c([2H])c([2H])c(N(c3ccccc3)c3ccccc3)c12 ; [2H]c1c([2H])c([2H])c2c(c1[2H])c([2H])c([2H])c1c([2H])c([2H])c([2H])c(N(c3ccc(-c4ccccc4)cc3)c3ccc(-c4ccccc4)cc3)c12 ; [2H]c1c([2H])c([2H])c2c(c1[2H])c([2H])c([2H])c1c([2H])c([2H])c([2H])c(N(c3cccc(-c4ccccc4)c3)c3cccc4ccccc34)c12]

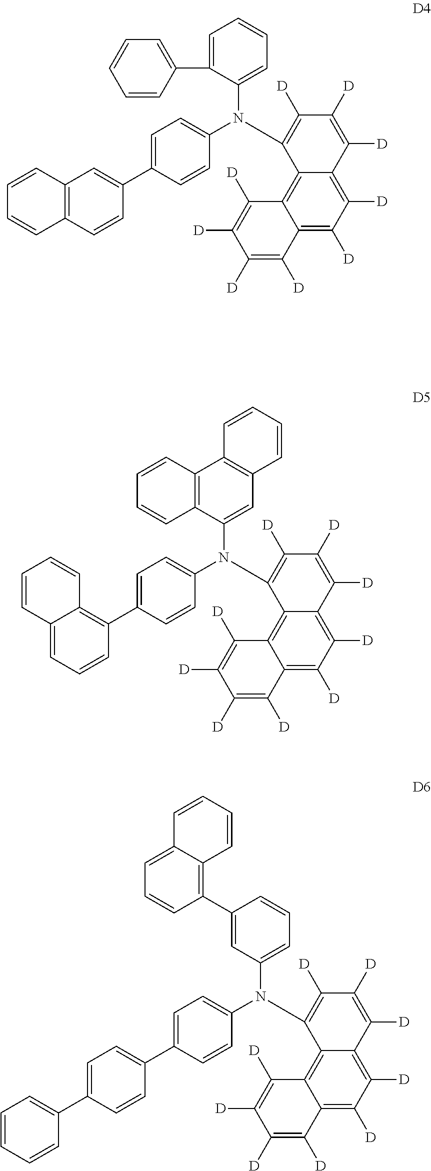 [SMILES: [2H]c1c([2H])c([2H])c2c(c1[2H])c([2H])c([2H])c1c([2H])c([2H])c([2H])c(N(c3ccc(-c4ccc5ccccc5c4)cc3)c3ccccc3-c3ccccc3)c12 ; [2H]c1c([2H])c([2H])c2c(c1[2H])c([2H])c([2H])c1c([2H])c([2H])c([2H])c(N(c3ccc(-c4cccc5ccccc45)cc3)c3cc4ccccc4c4ccccc34)c12 ; [2H]c1c([2H])c([2H])c2c(c1[2H])c([2H])c([2H])c1c([2H])c([2H])c([2H])c(N(c3ccc(-c4ccc(-c5ccccc5)cc4)cc3)c3cccc(-c4cccc5ccccc45)c3)c12]

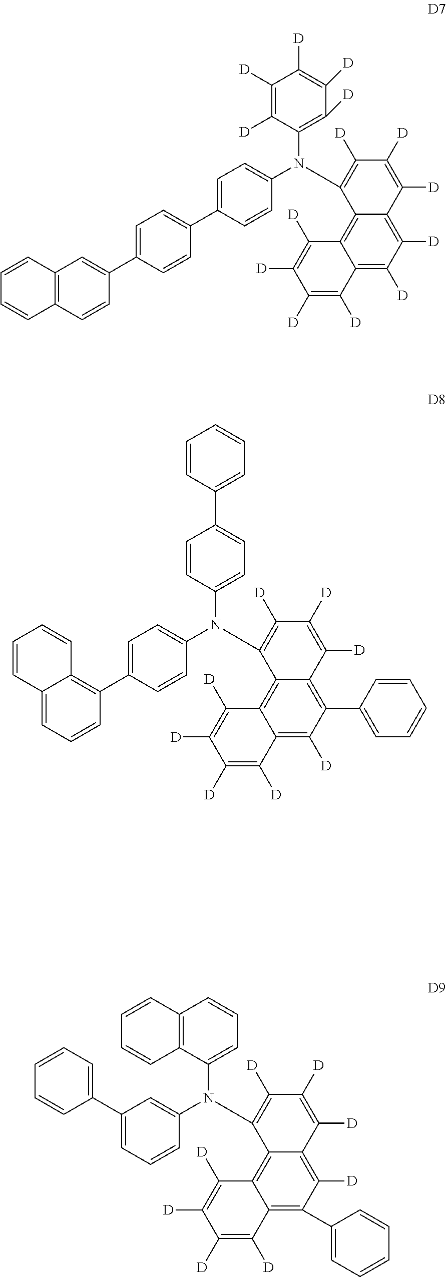 [SMILES: [2H]c1c([2H])c([2H])c(N(c2ccc(-c3ccc(-c4ccc5ccccc5c4)cc3)cc2)c2c([2H])c([2H])c([2H])c3c([2H])c([2H])c4c([2H])c([2H])c([2H])c([2H])c4c23)c([2H])c1[2H] ; [2H]c1c([2H])c([2H])c2c(c1[2H])c([2H])c(-c1ccccc1)c1c([2H])c([2H])c([2H])c(N(c3ccc(-c4ccccc4)cc3)c3ccc(-c4cccc5ccccc45)cc3)c12 ; [2H]c1c([2H])c([2H])c2c(c1[2H])c(-c1ccccc1)c([2H])c1c([2H])c([2H])c([2H])c(N(c3cccc(-c4ccccc4)c3)c3cccc4ccccc34)c12]

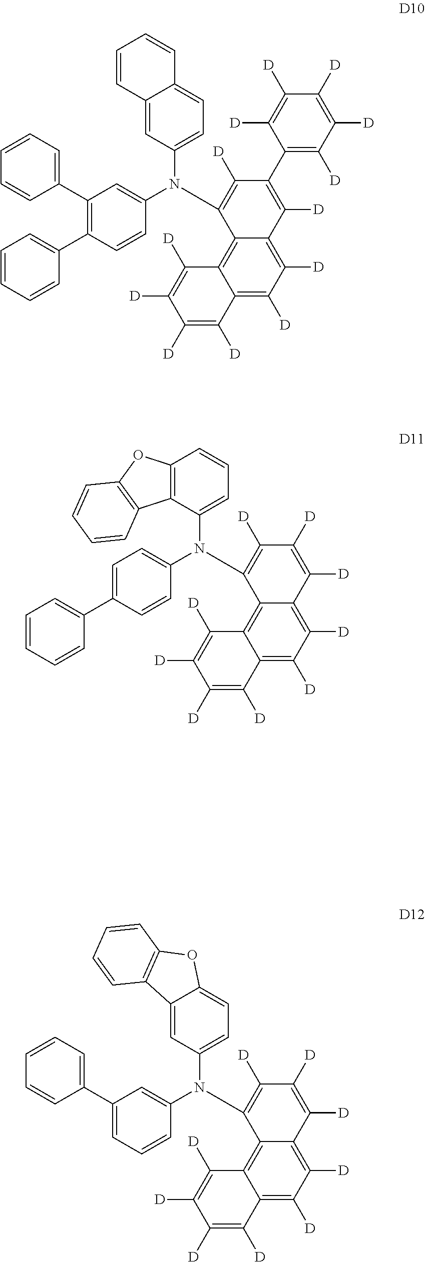 [SMILES: [2H]c1c([2H])c([2H])c(-c2c([2H])c(N(c3ccc(-c4ccccc4)c(-c4ccccc4)c3)c3ccc4ccccc4c3)c3c(c2[2H])c([2H])c([2H])c2c([2H])c([2H])c([2H])c([2H])c23)c([2H])c1[2H] ; [2H]c1c([2H])c([2H])c2c(c1[2H])c([2H])c([2H])c1c([2H])c([2H])c([2H])c(N(c3ccc(-c4ccccc4)cc3)c3cccc4oc5ccccc5c34)c12 ; [2H]c1c([2H])c([2H])c2c(c1[2H])c([2H])c([2H])c1c([2H])c([2H])c([2H])c(N(c3cccc(-c4ccccc4)c3)c3ccc4oc5ccccc5c4c3)c12]

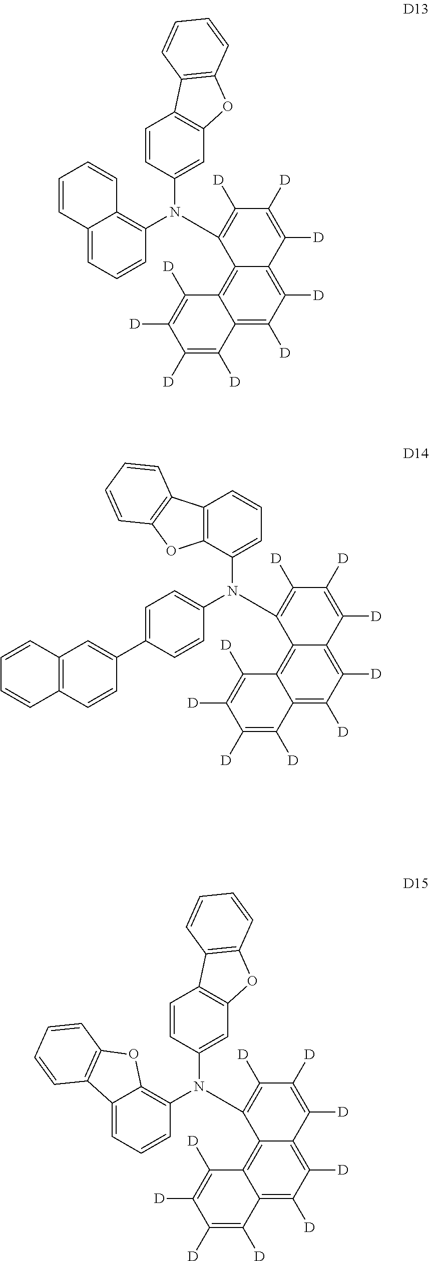 [SMILES: [2H]c1c([2H])c([2H])c2c(c1[2H])c([2H])c([2H])c1c([2H])c([2H])c([2H])c(N(c3ccc4c(c3)oc3ccccc34)c3cccc4ccccc34)c12 ; [2H]c1c([2H])c([2H])c2c(c1[2H])c([2H])c([2H])c1c([2H])c([2H])c([2H])c(N(c3ccc(-c4ccc5ccccc5c4)cc3)c3cccc4c3oc3ccccc34)c12 ; [2H]c1c([2H])c([2H])c2c(c1[2H])c([2H])c([2H])c1c([2H])c([2H])c([2H])c(N(c3ccc4c(c3)oc3ccccc34)c3cccc4c3oc3ccccc34)c12]

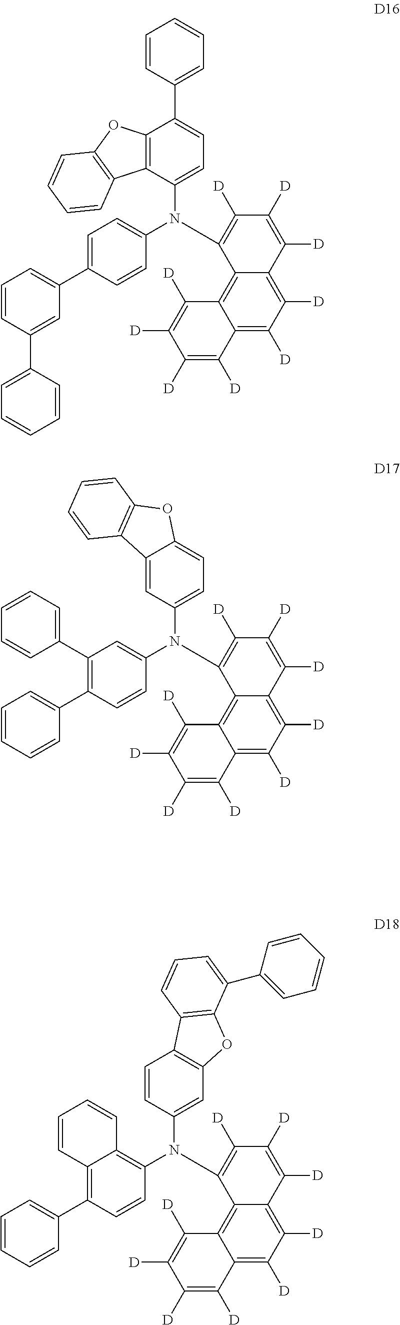 [SMILES: [2H]c1c([2H])c([2H])c2c(c1[2H])c([2H])c([2H])c1c([2H])c([2H])c([2H])c(N(c3ccc(-c4cccc(-c5ccccc5)c4)cc3)c3ccc(-c4ccccc4)c4oc5ccccc5c34)c12 ; [2H]c1c([2H])c([2H])c2c(c1[2H])c([2H])c([2H])c1c([2H])c([2H])c([2H])c(N(c3ccc(-c4ccccc4)c(-c4ccccc4)c3)c3ccc4oc5ccccc5c4c3)c12 ; [2H]c1c([2H])c([2H])c2c(c1[2H])c([2H])c([2H])c1c([2H])c([2H])c([2H])c(N(c3ccc4c(c3)oc3c(-c5ccccc5)cccc34)c3ccc(-c4ccccc4)c4ccccc34)c12]

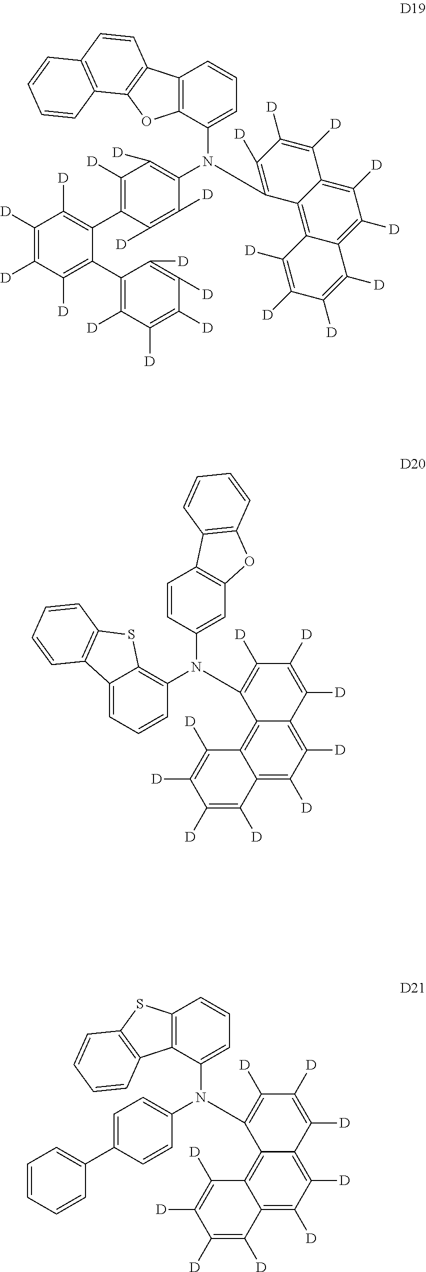 [SMILES: [2H]c1c([2H])c([2H])c(-c2c([2H])c([2H])c([2H])c([2H])c2-c2c([2H])c([2H])c(N(c3cccc4c3oc3c5ccccc5ccc43)c3c([2H])c([2H])c([2H])c4c([2H])c([2H])c5c([2H])c([2H])c([2H])c([2H])c5c34)c([2H])c2[2H])c([2H])c1[2H] ; [2H]c1c([2H])c([2H])c2c(c1[2H])c([2H])c([2H])c1c([2H])c([2H])c([2H])c(N(c3ccc4c(c3)oc3ccccc34)c3cccc4c3sc3ccccc34)c12 ; [2H]c1c([2H])c([2H])c2c(c1[2H])c([2H])c([2H])c1c([2H])c([2H])c([2H])c(N(c3ccc(-c4ccccc4)cc3)c3cccc4sc5ccccc5c34)c12]

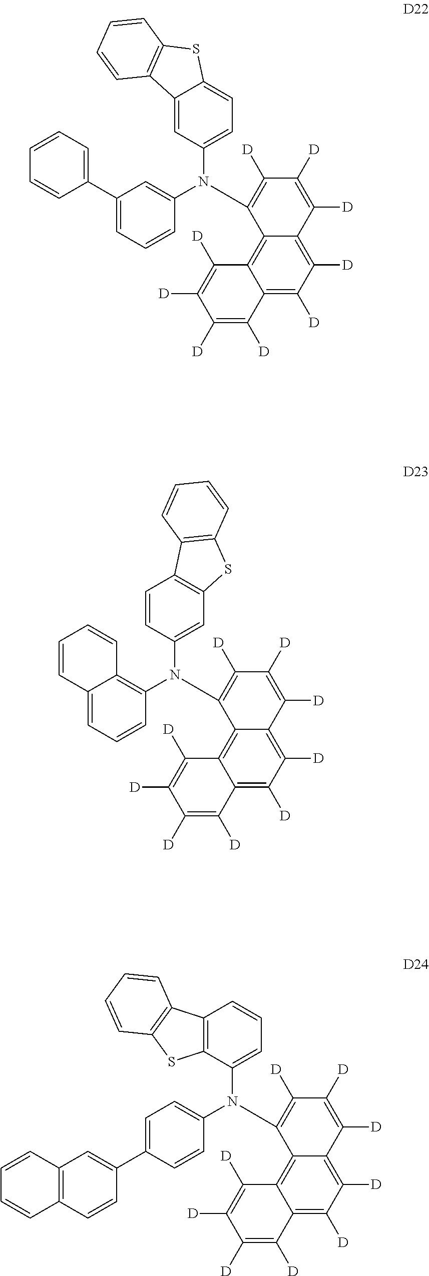 [SMILES: [2H]c1c([2H])c([2H])c2c(c1[2H])c([2H])c([2H])c1c([2H])c([2H])c([2H])c(N(c3cccc(-c4ccccc4)c3)c3ccc4sc5ccccc5c4c3)c12 ; [2H]c1c([2H])c([2H])c2c(c1[2H])c([2H])c([2H])c1c([2H])c([2H])c([2H])c(N(c3ccc4c(c3)sc3ccccc34)c3cccc4ccccc34)c12 ; [2H]c1c([2H])c([2H])c2c(c1[2H])c([2H])c([2H])c1c([2H])c([2H])c([2H])c(N(c3ccc(-c4ccc5ccccc5c4)cc3)c3cccc4c3sc3ccccc34)c12]

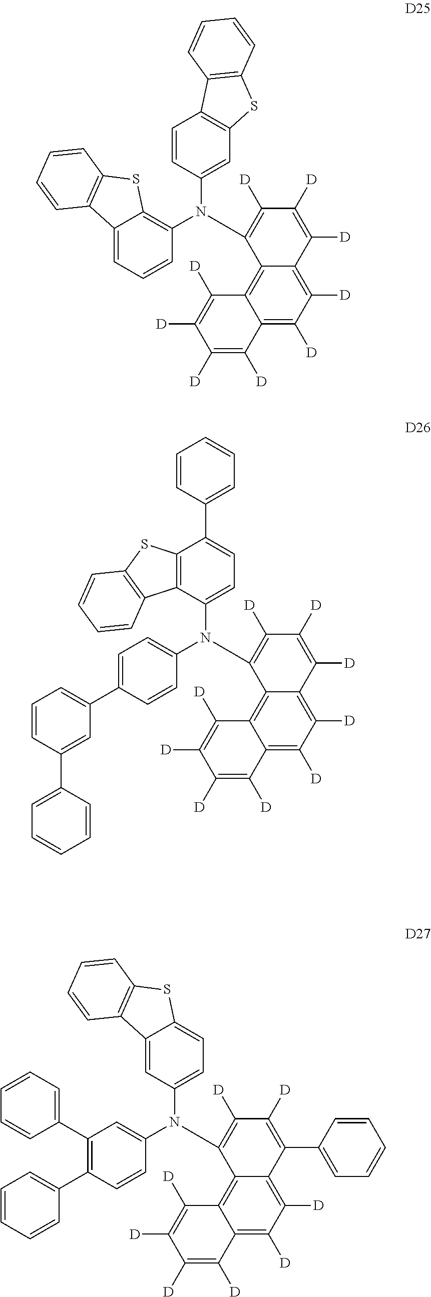 [SMILES: [2H]c1c([2H])c([2H])c2c(c1[2H])c([2H])c([2H])c1c([2H])c([2H])c([2H])c(N(c3ccc4c(c3)sc3ccccc34)c3cccc4c3sc3ccccc34)c12 ; [2H]c1c([2H])c([2H])c2c(c1[2H])c([2H])c([2H])c1c([2H])c([2H])c([2H])c(N(c3ccc(-c4cccc(-c5ccccc5)c4)cc3)c3ccc(-c4ccccc4)c4sc5ccccc5c34)c12 ; [2H]c1c([2H])c([2H])c2c(c1[2H])c([2H])c([2H])c1c(-c3ccccc3)c([2H])c([2H])c(N(c3ccc(-c4ccccc4)c(-c4ccccc4)c3)c3ccc4sc5ccccc5c4c3)c12]

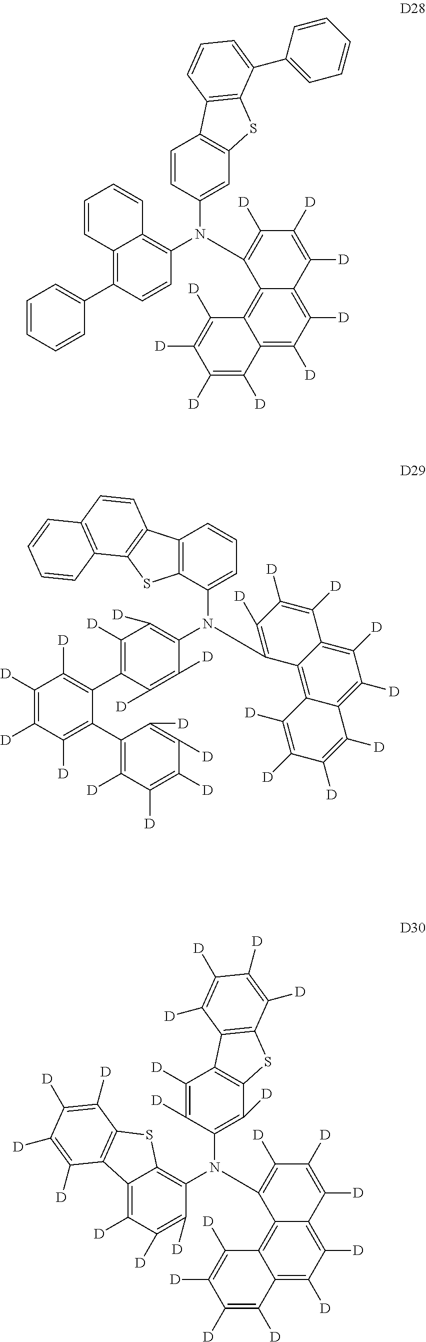

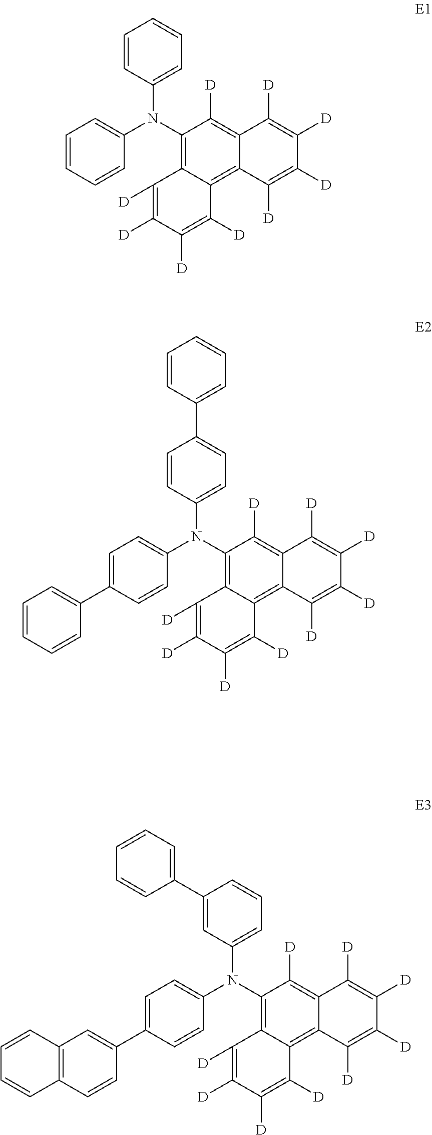 [SMILES: [2H]c1c([2H])c([2H])c2c(c1[2H])c([2H])c(N(c1ccccc1)c1ccccc1)c1c([2H])c([2H])c([2H])c([2H])c12 ; [2H]c1c([2H])c([2H])c2c(c1[2H])c([2H])c(N(c1ccc(-c3ccccc3)cc1)c1ccc(-c3ccccc3)cc1)c1c([2H])c([2H])c([2H])c([2H])c12 ; [2H]c1c([2H])c([2H])c2c(c1[2H])c([2H])c(N(c1ccc(-c3ccc4ccccc4c3)cc1)c1cccc(-c3ccccc3)c1)c1c([2H])c([2H])c([2H])c([2H])c12]

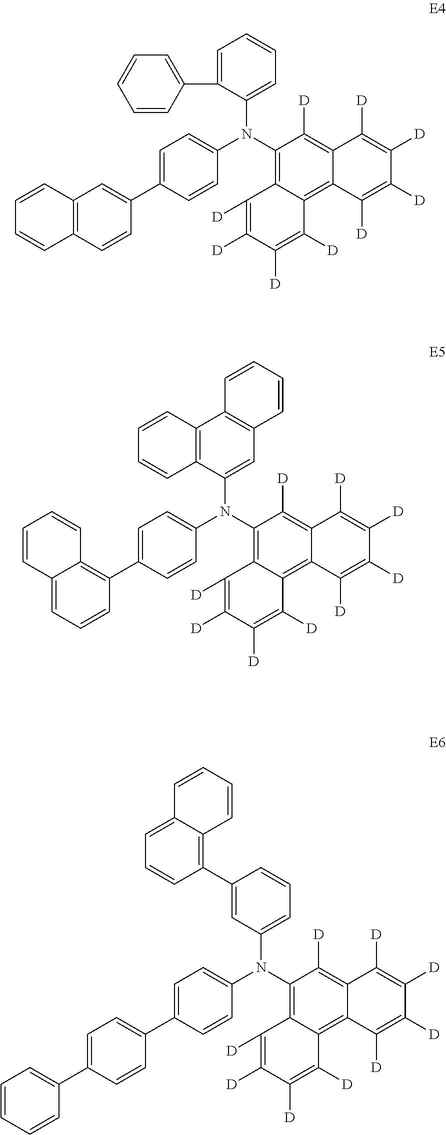 [SMILES: [2H]c1c([2H])c([2H])c2c(c1[2H])c([2H])c(N(c1ccc(-c3ccc4ccccc4c3)cc1)c1ccccc1-c1ccccc1)c1c([2H])c([2H])c([2H])c([2H])c12 ; [2H]c1c([2H])c([2H])c2c(c1[2H])c([2H])c(N(c1ccc(-c3cccc4ccccc34)cc1)c1cc3ccccc3c3ccccc13)c1c([2H])c([2H])c([2H])c([2H])c12 ; [2H]c1c([2H])c([2H])c2c(c1[2H])c([2H])c(N(c1ccc(-c3ccc(-c4ccccc4)cc3)cc1)c1cccc(-c3cccc4ccccc34)c1)c1c([2H])c([2H])c([2H])c([2H])c12]

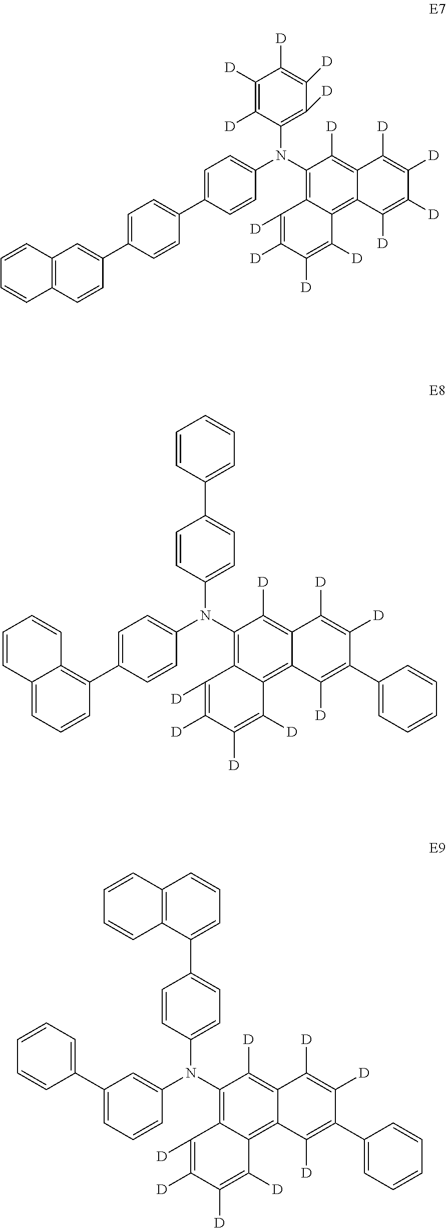 [SMILES: [2H]c1c([2H])c([2H])c(N(c2ccc(-c3ccc(-c4ccc5ccccc5c4)cc3)cc2)c2c([2H])c3c([2H])c([2H])c([2H])c([2H])c3c3c([2H])c([2H])c([2H])c([2H])c23)c([2H])c1[2H] ; [2H]c1c(-c2ccccc2)c([2H])c2c(c1[2H])c([2H])c(N(c1ccc(-c3ccccc3)cc1)c1ccc(-c3cccc4ccccc34)cc1)c1c([2H])c([2H])c([2H])c([2H])c12 ; [2H]c1c(-c2ccccc2)c([2H])c2c(c1[2H])c([2H])c(N(c1ccc(-c3cccc4ccccc34)cc1)c1cccc(-c3ccccc3)c1)c1c([2H])c([2H])c([2H])c([2H])c12]

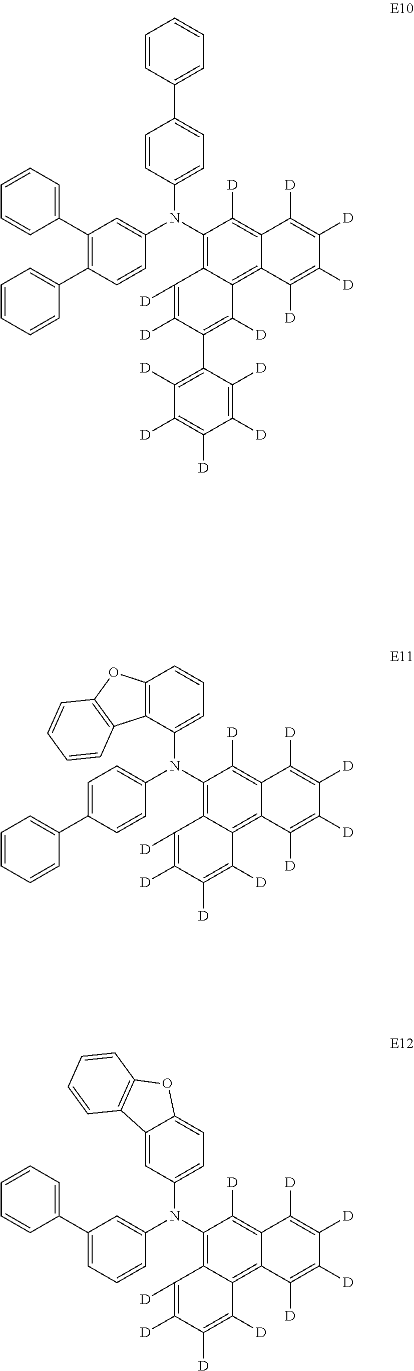 [SMILES: [2H]c1c([2H])c([2H])c(-c2c([2H])c([2H])c3c(N(c4ccc(-c5ccccc5)cc4)c4ccc(-c5ccccc5)c(-c5ccccc5)c4)c([2H])c4c([2H])c([2H])c([2H])c([2H])c4c3c2[2H])c([2H])c1[2H] ; [2H]c1c([2H])c([2H])c2c(c1[2H])c([2H])c(N(c1ccc(-c3ccccc3)cc1)c1cccc3oc4ccccc4c13)c1c([2H])c([2H])c([2H])c([2H])c12 ; [2H]c1c([2H])c([2H])c2c(c1[2H])c([2H])c(N(c1cccc(-c3ccccc3)c1)c1ccc3oc4ccccc4c3c1)c1c([2H])c([2H])c([2H])c([2H])c12]

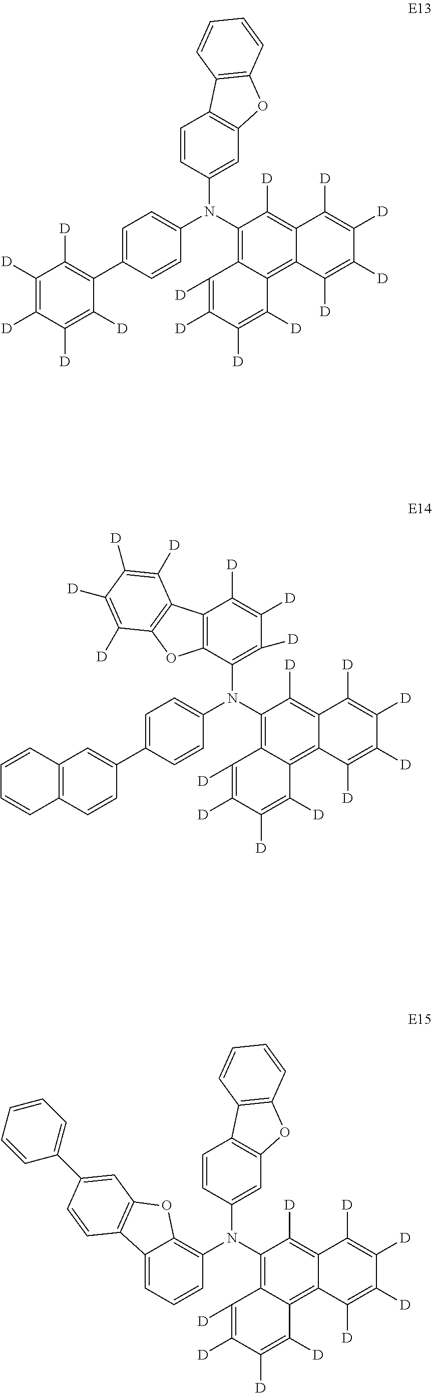 [SMILES: [2H]c1c([2H])c([2H])c(-c2ccc(N(c3ccc4c(c3)oc3ccccc34)c3c([2H])c4c([2H])c([2H])c([2H])c([2H])c4c4c([2H])c([2H])c([2H])c([2H])c34)cc2)c([2H])c1[2H] ; [2H]c1c([2H])c([2H])c2c(oc3c(N(c4ccc(-c5ccc6ccccc6c5)cc4)c4c([2H])c5c([2H])c([2H])c([2H])c([2H])c5c5c([2H])c([2H])c([2H])c([2H])c45)c([2H])c([2H])c([2H])c32)c1[2H] ; [2H]c1c([2H])c([2H])c2c(c1[2H])c([2H])c(N(c1ccc3c(c1)oc1ccccc13)c1cccc3c1oc1cc(-c4ccccc4)ccc13)c1c([2H])c([2H])c([2H])c([2H])c12]

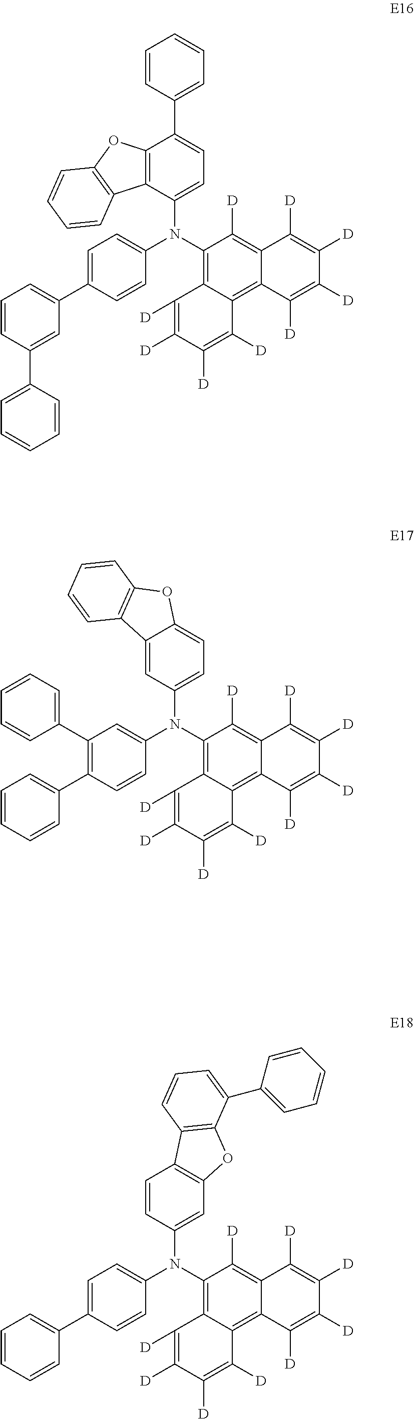 [SMILES: [2H]c1c([2H])c([2H])c2c(c1[2H])c([2H])c(N(c1ccc(-c3cccc(-c4ccccc4)c3)cc1)c1ccc(-c3ccccc3)c3oc4ccccc4c13)c1c([2H])c([2H])c([2H])c([2H])c12 ; [2H]c1c([2H])c([2H])c2c(c1[2H])c([2H])c(N(c1ccc(-c3ccccc3)c(-c3ccccc3)c1)c1ccc3oc4ccccc4c3c1)c1c([2H])c([2H])c([2H])c([2H])c12 ; [2H]c1c([2H])c([2H])c2c(c1[2H])c([2H])c(N(c1ccc(-c3ccccc3)cc1)c1ccc3c(c1)oc1c(-c4ccccc4)cccc13)c1c([2H])c([2H])c([2H])c([2H])c12]

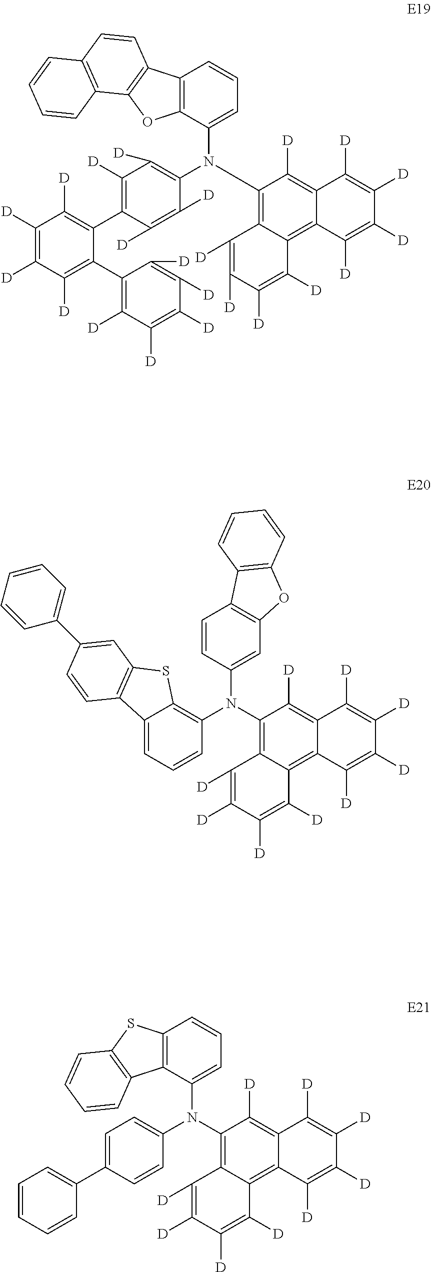 [SMILES: [2H]c1c([2H])c([2H])c(-c2c([2H])c([2H])c([2H])c([2H])c2-c2c([2H])c([2H])c(N(c3cccc4c3oc3c5ccccc5ccc43)c3c([2H])c4c([2H])c([2H])c([2H])c([2H])c4c4c([2H])c([2H])c([2H])c([2H])c34)c([2H])c2[2H])c([2H])c1[2H] ; [2H]c1c([2H])c([2H])c2c(c1[2H])c([2H])c(N(c1ccc3c(c1)oc1ccccc13)c1cccc3c1sc1cc(-c4ccccc4)ccc13)c1c([2H])c([2H])c([2H])c([2H])c12 ; [2H]c1c([2H])c([2H])c2c(c1[2H])c([2H])c(N(c1ccc(-c3ccccc3)cc1)c1cccc3sc4ccccc4c13)c1c([2H])c([2H])c([2H])c([2H])c12]

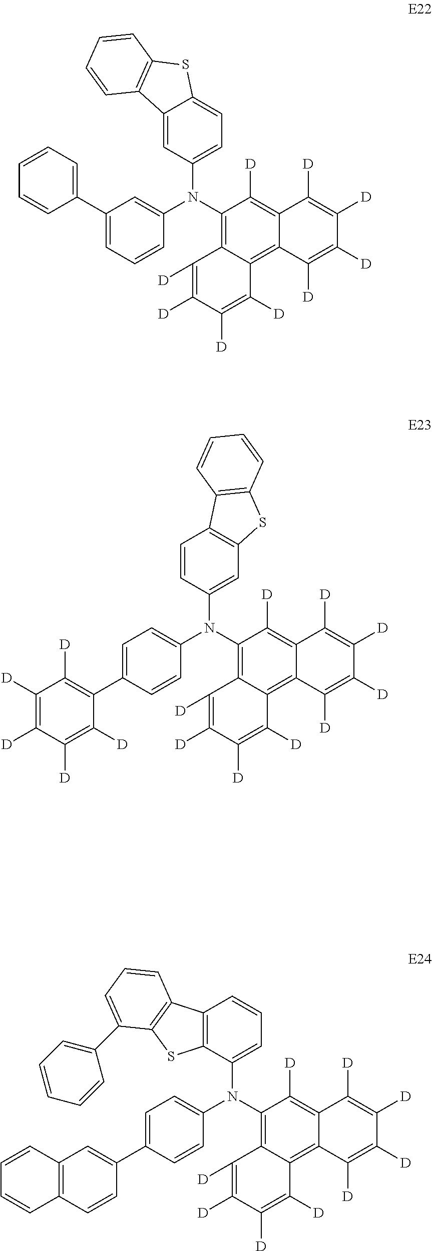 [SMILES: [2H]c1c([2H])c([2H])c2c(c1[2H])c([2H])c(N(c1cccc(-c3ccccc3)c1)c1ccc3sc4ccccc4c3c1)c1c([2H])c([2H])c([2H])c([2H])c12 ; [2H]c1c([2H])c([2H])c(-c2ccc(N(c3ccc4c(c3)sc3ccccc34)c3c([2H])c4c([2H])c([2H])c([2H])c([2H])c4c4c([2H])c([2H])c([2H])c([2H])c34)cc2)c([2H])c1[2H] ; [2H]c1c([2H])c([2H])c2c(c1[2H])c([2H])c(N(c1ccc(-c3ccc4ccccc4c3)cc1)c1cccc3c1sc1c(-c4ccccc4)cccc13)c1c([2H])c([2H])c([2H])c([2H])c12]

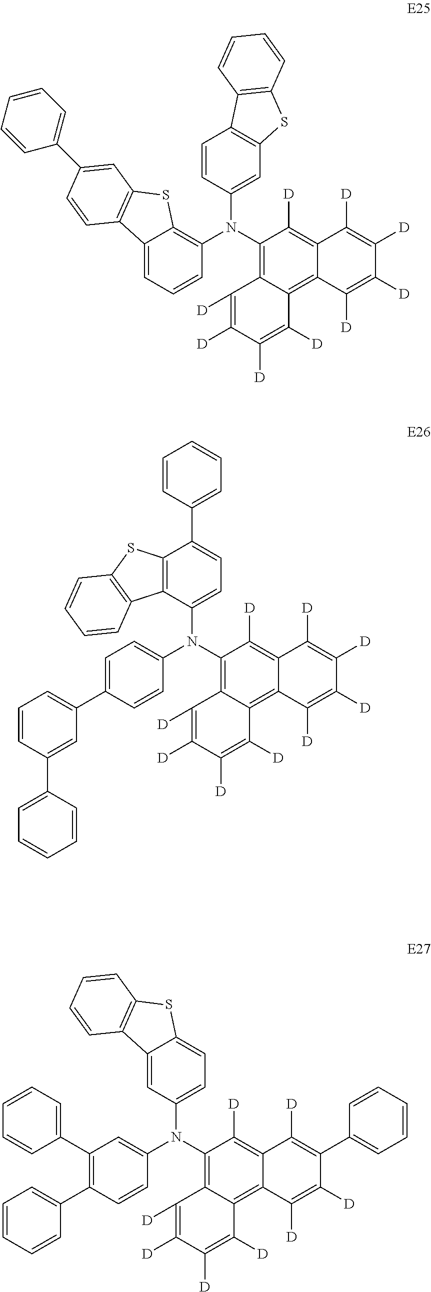 [SMILES: [2H]c1c([2H])c([2H])c2c(c1[2H])c([2H])c(N(c1ccc3c(c1)sc1ccccc13)c1cccc3c1sc1cc(-c4ccccc4)ccc13)c1c([2H])c([2H])c([2H])c([2H])c12 ; [2H]c1c([2H])c([2H])c2c(c1[2H])c([2H])c(N(c1ccc(-c3cccc(-c4ccccc4)c3)cc1)c1ccc(-c3ccccc3)c3sc4ccccc4c13)c1c([2H])c([2H])c([2H])c([2H])c12 ; [2H]c1c([2H])c([2H])c2c(c1[2H])c(N(c1ccc(-c3ccccc3)c(-c3ccccc3)c1)c1ccc3sc4ccccc4c3c1)c([2H])c1c([2H])c(-c3ccccc3)c([2H])c([2H])c12]

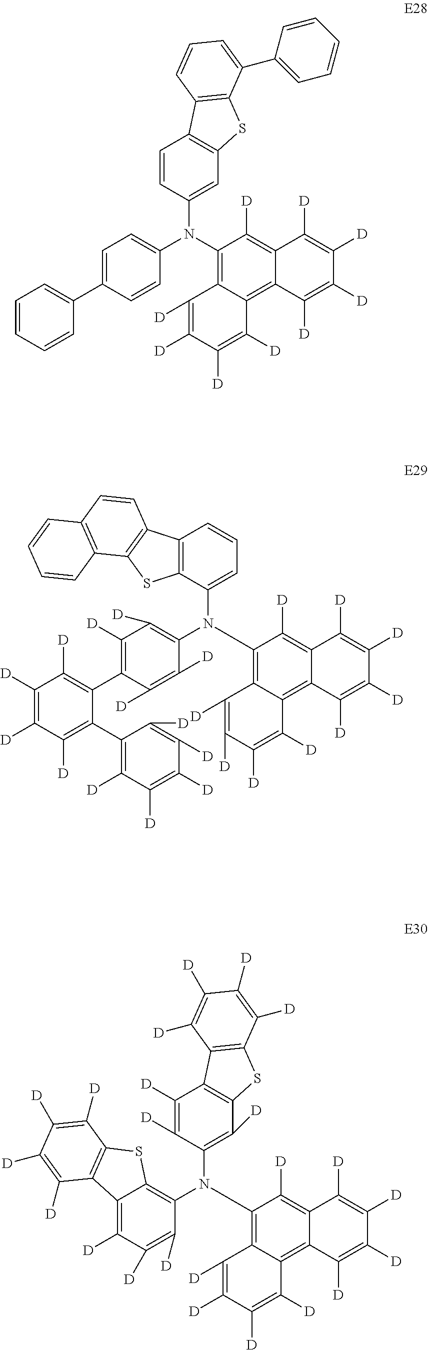

The amine compound according to one or more embodiments may include a phenanthrene group represented by Formula 1 and deuterated, and an aromatic amine group represented by Formula 2 and directly bonded to the phenanthrene group. In addition, in the amine compound according to one or more embodiments, the types (kinds) of aromatic substituents constituting the amine group may be specified depending on a bonding location of the aromatic amine group bonded to the deuterated phenanthrene group. The amine compound according to one or more embodiments has high planarity, which may lead to excellent or suitable electric stability and high charge transport ability due to a combination of the deuterated phenanthrene group and the amine group directly bonded to the deuterated phenanthrene group. The amine compound according to one or more embodiments has high planarity due to a combination of the deuterated phenanthrene group and the aromatic amine group directly bonded to the deuterated phenanthrene group, and thus may have excellent or suitable electrical stability and high charge transport ability. In addition, due to deuteration, in which a carbon-protium bond is replaced with a carbon-deuterium bond, the amine compound according to one or more embodiments may exhibit characteristics of improved chemical stability. Therefore, the amine compound according to one or more embodiments may have improved hole transport ability and lifespan. Consequently, the light-emitting element according to one or more embodiments including the amine compound according to one or more embodiments may have improved luminous efficiency and lifespan.

In the light-emitting element ED according to one or more embodiments, the hole transport region HTR may further include a compound represented by Formula H-1. For example, the light-emitting element ED according to one or more embodiments may include the compound represented by Formula H-1 in another layer of the hole transport region HTR, which does not include the amine compound according to one or more embodiments of Formula 1. However, embodiments of the present disclosure are not limited thereto.

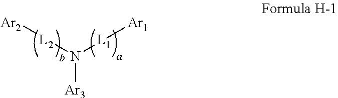

In Formula H-1, L₁ and L₂ may each independently be a direct linkage, a substituted or unsubstituted arylene group having ring-forming carbons of 6 to 30, or a substituted or unsubstituted heteroarylene group having ring-forming carbons of 2 to 30. a and b may each independently be an integer of 0 to 10. In one or more embodiments, if (e.g., when) a or b is an integer of 2 or greater, a plurality of L₁'s and a plurality of L₂'s may each independently be a substituted or unsubstantiated arylene group having ring-forming carbons of 6 to 30 or a substituted or unsubstantiated heteroarylene group having ring-forming carbons of 2 to 30.

In Formula H-1, Ar₁ and Ar₂ may each independently be a substituted or unsubstituted aryl group having ring-forming carbons of 6 to 30 or a substituted or unsubstituted heteroaryl group having ring-forming carbons of 2 to 30. In addition, in Formula H-1, Ar₃ may be a substituted or unsubstituted aryl group having ring-forming carbons of 6 to 30 or a substituted or unsubstituted heteroaryl group having ring-forming carbons of 2 to 30.

In one or more embodiments, the compound represented by Formula H-1 may be a monoamine compound. In one or more embodiments, the compound represented by Formula H-1 may be a diamine compound in which at least one selected from among Ar₁ to Ar₃ includes an amine group as a substituent. In one or more embodiments, the compound represented by Formula H-1 may be a carbazole-based compound in which at least one selected from among Ar₁ and Ar₂ includes a substituted or unsubstituted carbazole group or a fluorene-based compound in which at least one selected from among Ar₁ and Ar₂ includes a substituted or unsubstituted fluorene group.

The compound represented by Formula H-1 may be any one selected from among compounds in Compound Group H. However, the compounds present in Compound Group H are suggested as mere examples, the compound represented by Formula H-1 is not limited to the compounds present in Compound Group H.

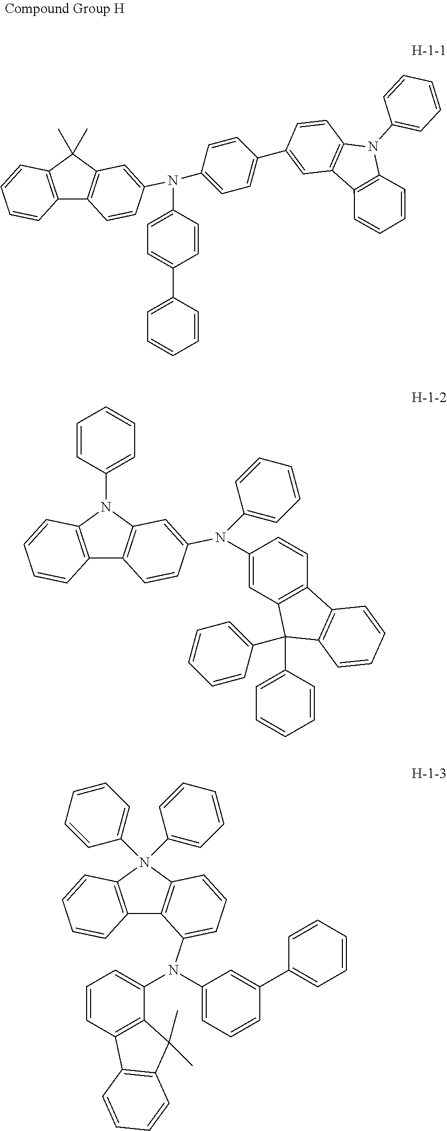

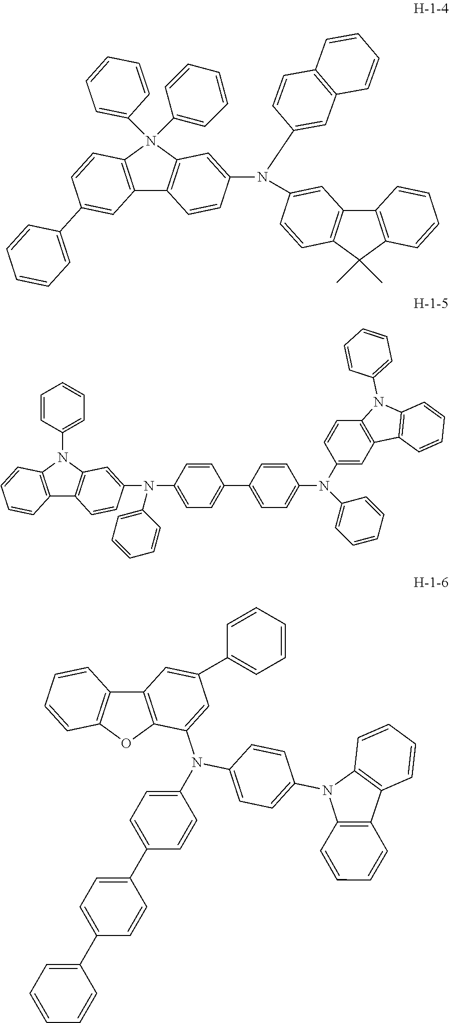

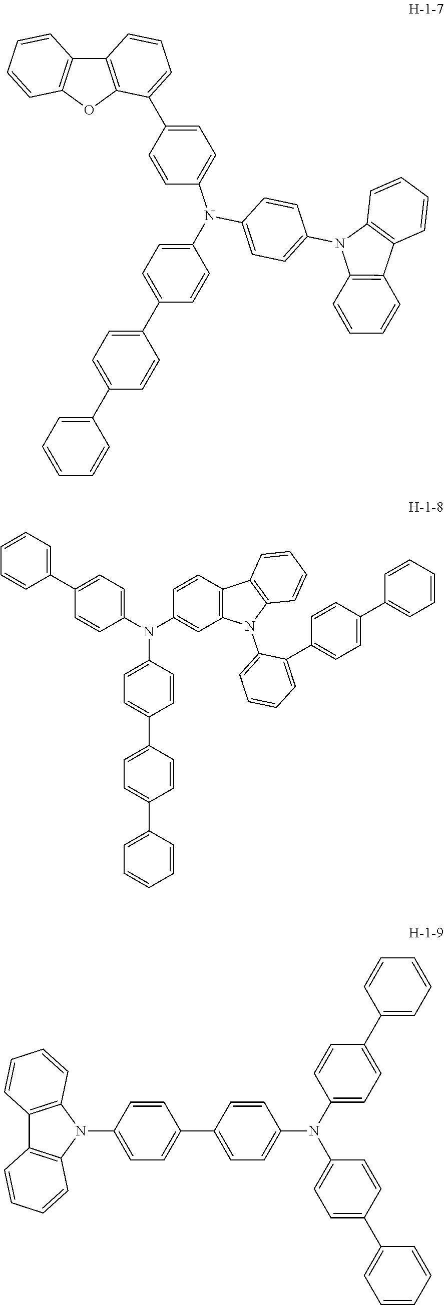 [SMILES: c1ccc(-c2ccc(-c3ccc(N(c4ccc(-c5cccc6c5oc5ccccc56)cc4)c4ccc(-n5c6ccccc6c6ccccc65)cc4)cc3)cc2)cc1 ; c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1]

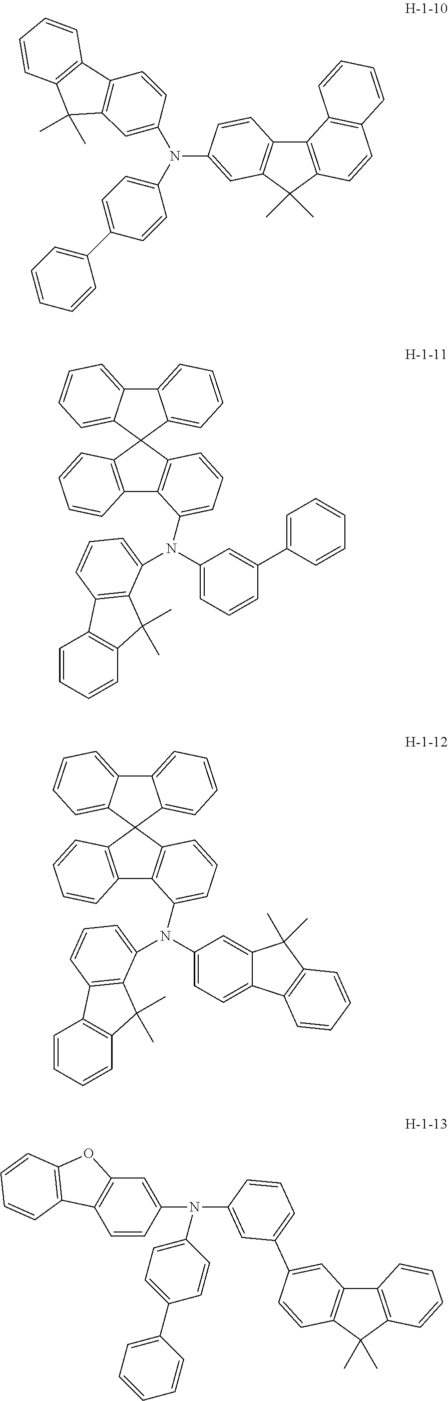 [SMILES: CC1(C)c2ccccc2-c2ccc(N(c3ccc(-c4ccccc4)cc3)c3ccc4c(c3)C(C)(C)c3ccc5ccccc5c3-4)cc21 ; CC1(C)c2ccccc2-c2cccc(N(c3cccc(-c4ccccc4)c3)c3cccc4c3-c3ccccc3C43c4ccccc4-c4ccccc43)c21 ; CC1(C)c2ccccc2-c2ccc(N(c3cccc4c3-c3ccccc3C43c4ccccc4-c4ccccc43)c3cccc4c3C(C)(C)c3ccccc3-4)cc21 ; CC1(C)c2ccccc2-c2cc(-c3cccc(N(c4ccc(-c5ccccc5)cc4)c4ccc5c(c4)oc4ccccc45)c3)ccc21]

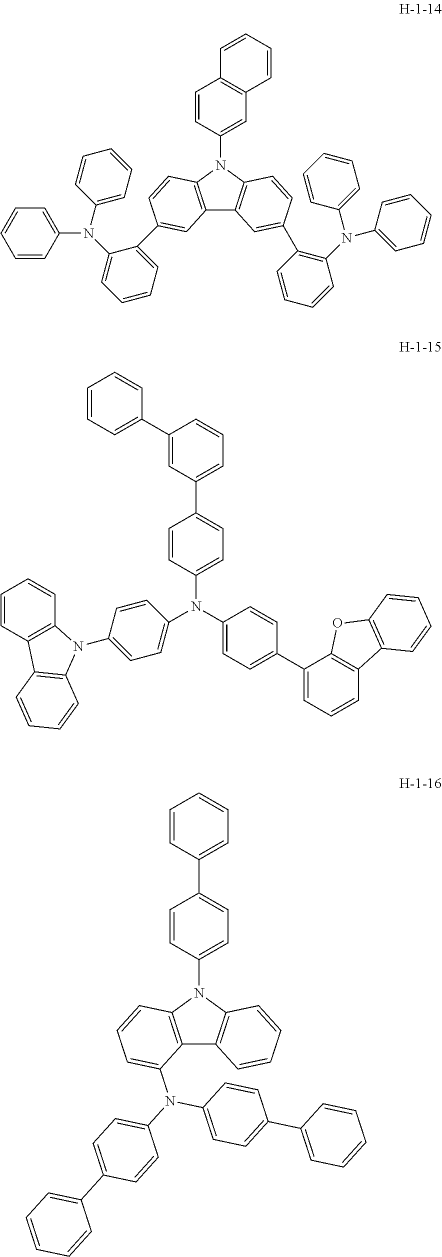 [SMILES: c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1 ; c1ccc(-c2cccc(-c3ccc(N(c4ccc(-c5cccc6c5oc5ccccc56)cc4)c4ccc(-n5c6ccccc6c6ccccc65)cc4)cc3)c2)cc1 ; c1ccc(-c2ccc(N(c3ccc(-c4ccccc4)cc3)c3cccc4c3c3ccccc3n4-c3ccc(-c4ccccc4)cc3)cc2)cc1]

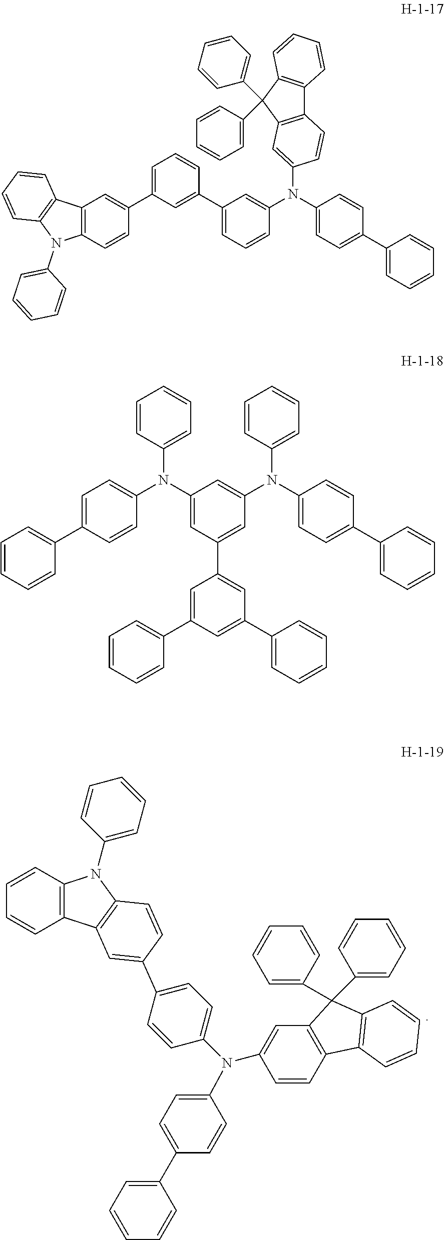 [SMILES: c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1 ; c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

In one or more embodiments, the hole transport region HTR may further include one or more selected from among a phthalocyanine compound such as copper phthalocyanine, N¹, N^{1′}-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino]triphenylamine (m-MTDATA), 4,4′4″-tris(N,N-diphenylamino)triphenylamine (TDATA), 4,4′,4″-tris[N-(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(naphthalen-1-yl)-N,N′-diphenyl-benzidine (NPB), polyetherketone including triphenylamine (TPAPEK), 4-isopropyl-4′-methyldiphenyliodonium [Tetrakis(pentafluorophenyl)borate], dipyrazino[2,3-f: 2′,3′-h]quinoxaline-2,3,6,7,10,11-hexacarbonitrile (HATCN), and/or the like.

In one or more embodiments, the hole transport region HTR may further include one or more selected from among a carbazole-based derivative such as N-phenyl carbazole and/or polyvinyl carbazole, a fluorene-based derivative, a triphenylamine-based derivative such as N,N′-bis(3-methylphenyl)-N,N′-diphenyl-[1,1′-biphenyl]-4,4′-diamine (TPD) and/or 4,4′,4″-tris(N-carbazolyl)triphenylamine (TCTA), N,N′-di(naphthalen-1-yl)-N,N′-diphenyl-benzidine (NPB), 4,4′-cyclohexylidene bis[N,N-bis(4-methylphenyl)benzenamine](TAPC), 4,4′-bis[N,N′-(3-tolyl)amino]-3,3′-dimethylbiphenyl (HMTPD), 1,3-bis(N-carbazolyl)benzene (mCP), and/or the like.

In one or more embodiments, the hole transport region HTR may further include one or more selected from among 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), and/or the like.

The hole transport region HTR may include one or more of the above-described compounds of the hole transport region in at least one selected from among the hole injection layer HIL, the hole transport layer HTL, the emission auxiliary layer EAL, and the electron-blocking layer EBL.

The hole transport region HTR may have a thickness of about 100 Å to about 10000 Å or may have, for example, about 100 Å to about 5000 Å. If (e.g., when) the hole transport region HTR includes the hole injection layer HIL, the hole injection layer HIL may have a thickness of, for example, about 30 Å to about 1000 Å. If (e.g., when) the hole transport region HTR includes the hole transport layer HTL, the hole transport layer HTL may have a thickness of about 30 Å to about 1000 Å. For example, if (e.g., when) the hole transport region HTR includes the electron-blocking layer EBL, the electron-blocking layer EBL may have a thickness of about 10 Å to about 1000 Å. When the thicknesses of the hole transport region HTR, the hole injection layer HIL, the hole transport layer HTL, and the electron-blocking layer EBL fall within the above-described ranges, hole transport characteristics at a satisfactory level may be obtained without a substantial increase in driving voltage.

In one or more embodiments, the hole transport region HTR may further include a charge generation material for improving conductivity in addition to one or more of the above-mentioned materials. The charge generation material may be uniformly (e.g., substantially uniformly) or ununiformly distributed in the hole transport region HTR. The charge generation material may be, for example, a p-dopant. The p-dopant may include at least one selected from among a halogenated metal compound (e.g., a metal halide), a quinone derivative, a metal oxide, and a cyano group-containing compound, but embodiments of the present disclosure are not limited thereto. For example, examples of the p-dopant may include a halogenated metal compound such as CuI and/or RbI, a quinone derivative such as tetracyanoquinodimethane (TCNQ) and/or 2,3,5,6-tetrafluoro-7,7′8,8-tetracyanoquinodimethane (F4-TCNQ), a metal oxide such as tungsten oxide and/or molybdenum oxide, a cyano group-containing compound such as dipyrazino[2,3-f: 2′,3′-h]quinoxaline-2,3,6,7,10,11-hexacarbonitrile (HATCN) and/or 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9), and/or the like. However, embodiments of the disclosure are not limited thereto.

As described above, the hole transport region HTR may further include, in addition to the hole injection layer HIL and the hole transport layer HTL, at least one selected from among an emission auxiliary layer EAL and an electron-blocking layer EBL. The emission auxiliary layer EAL may compensate for a resonance distance depending on a wavelength of light emitted from the light-emitting layer EML and may control a hole charge balance, and thus may increase light luminous efficiency. In addition, the emission auxiliary layer EAL may serve to prevent or reduce electrons from being injected into the hole transport region HTR. The emission auxiliary layer EAL may include materials that may be included in the hole transport region HTR. The electron-blocking layer EBL serves to prevent or reduce electrons from being injected into the hole transport region HTR from the electron transport region ETR.

In the light-emitting element ED, the light-emitting layer EML may be provided on the hole transport region HTR. The light-emitting layer EML may have a thickness of, for example, about 100 Å to about 1000 Å, or about 100 Å to about 300 Å. The light-emitting layer EML may have a structure of a single layer including (e.g., consisting of) a single material, a single layer composed of a plurality of different materials, or a multilayer having a plurality of layers composed of a plurality of different materials.

In the light-emitting element ED according to one or more embodiments, the light-emitting layer EML may be to emit blue light. The light-emitting element ED according to one or more embodiments includes the amine compound according to one or more embodiments in the hole transport region HTR, and thus may exhibit characteristics of high efficiency and long lifespan in a blue light-emitting region. The light-emitting element ED according to one or more embodiments includes the amine compound according to one or more embodiments in the hole transport region HTR, and thus the light-emitting layer EML may be to emit blue fluorescence.

In addition, in the light-emitting element ED according to one or more embodiments, the light-emitting layer EML may be to emit light in a different wavelength region that is not blue light. The light-emitting element ED according to one or more embodiments includes the amine compound according to one or more embodiments in the hole transport region HTR and may exhibit characteristics of high efficiency and long lifespan in a light-emitting region with different wavelengths that is not blue light region. The light-emitting element ED according to one or more embodiments includes the amine compound according to one or more embodiments in the hole transport region HTR, and the light-emitting layer EML may be to emit fluorescence. However, embodiments of the present disclosure are not limited thereto.

In the light-emitting element ED according to one or more embodiments, the light-emitting layer EML may include an anthracene derivative, a pyrene derivative, a fluoranthene derivative, a chrysene derivative, a dihydrobenzanthracene derivative, or a triphenylene derivative. For example, in one or more embodiments, the light-emitting layer EML may include an anthracene derivative or a pyrene derivative. In one or more embodiments, the light-emitting layer EML may include one or more selected from among Compounds E-2-1 to E-2-24.

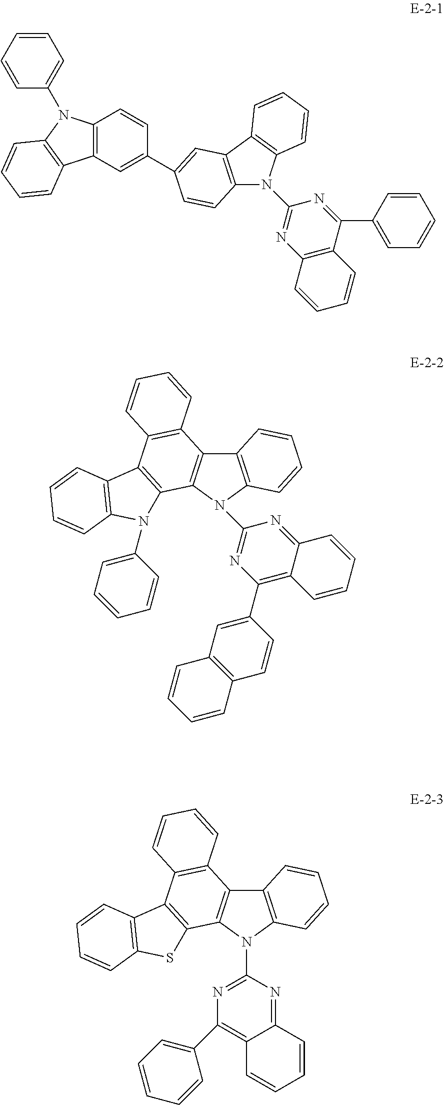 [SMILES: c1ccc(-c2nc(-n3c4ccccc4c4cc(-c5ccc6c(c5)c5ccccc5n6-c5ccccc5)ccc43)nc3ccccc23)cc1 ; c1ccc(-n2c3ccccc3c3c4ccccc4c4c5ccccc5n(-c5nc(-c6ccc7ccccc7c6)c6ccccc6n5)c4c32)cc1 ; c1ccc(-c2nc(-n3c4ccccc4c4c5ccccc5c5c6ccccc6sc5c43)nc3ccccc23)cc1]

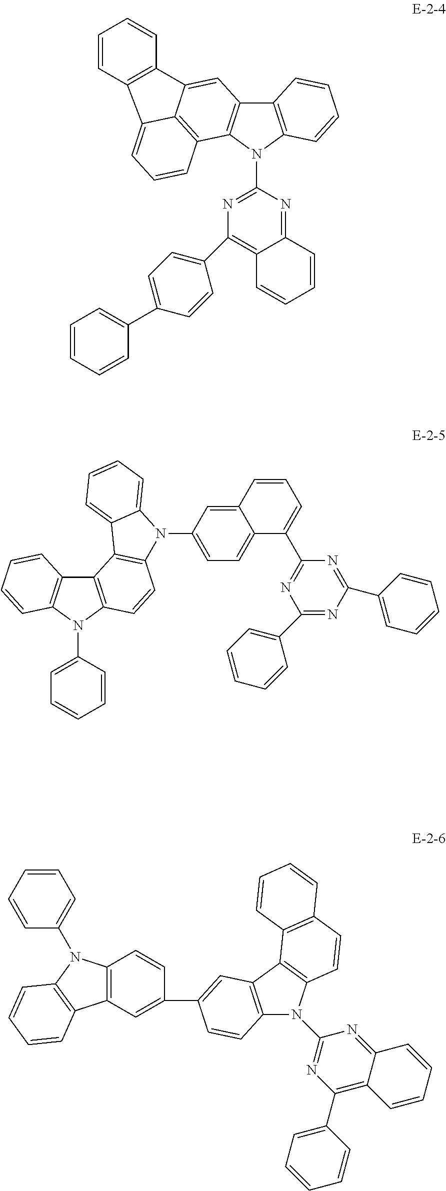 [SMILES: c1ccc(-c2ccc(-c3nc(-n4c5ccccc5c5cc6c7c(cccc7c54)-c4ccccc4-6)nc4ccccc34)cc2)cc1 ; c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1]

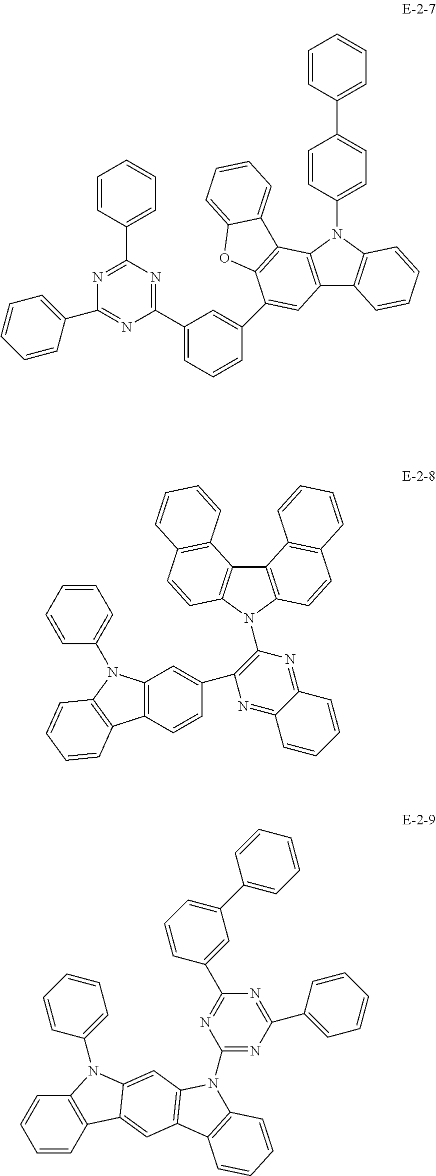 [SMILES: c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-n2c3ccccc3c3ccc(-c4nc5ccccc5nc4-n4c5ccc6ccccc6c5c5c6ccccc6ccc54)cc32)cc1 ; c1ccc(-c2cccc(-c3nc(-c4ccccc4)nc(-n4c5ccccc5c5cc6c7ccccc7n(-c7ccccc7)c6cc54)n3)c2)cc1]

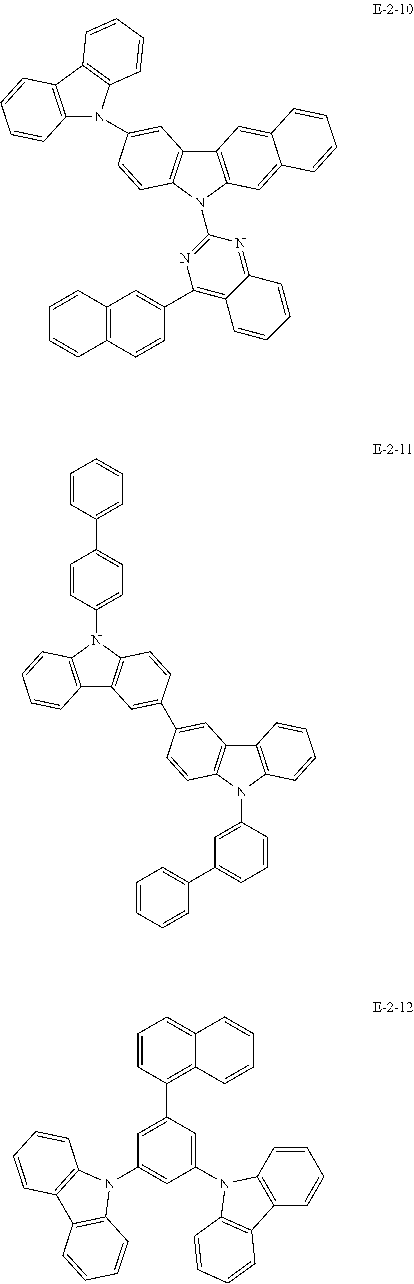 [SMILES: c1ccc2cc(-c3nc(-n4c5ccc(-n6c7ccccc7c7ccccc76)cc5c5cc6ccccc6cc54)nc4ccccc34)ccc2c1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc2c(-c3cc(-n4c5ccccc5c5ccccc54)cc(-n4c5ccccc5c5ccccc54)c3)cccc2c1]

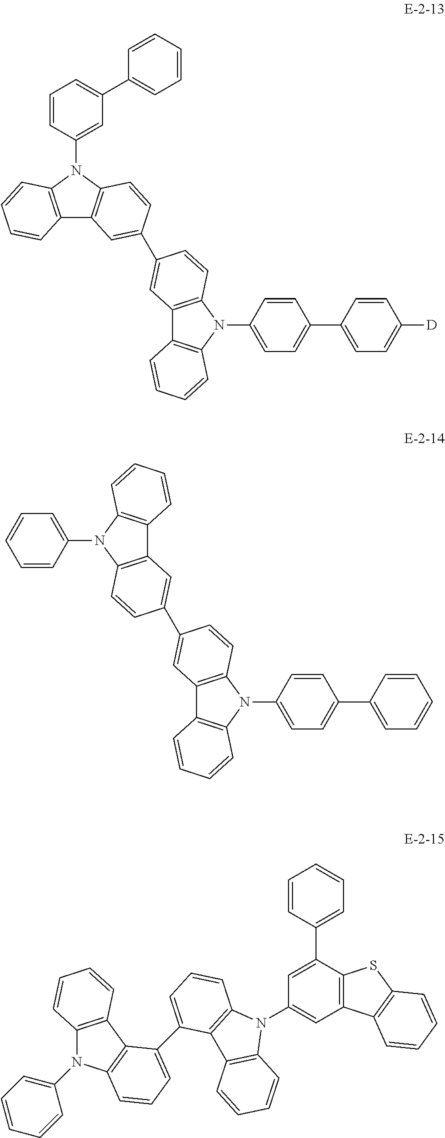 [SMILES: [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

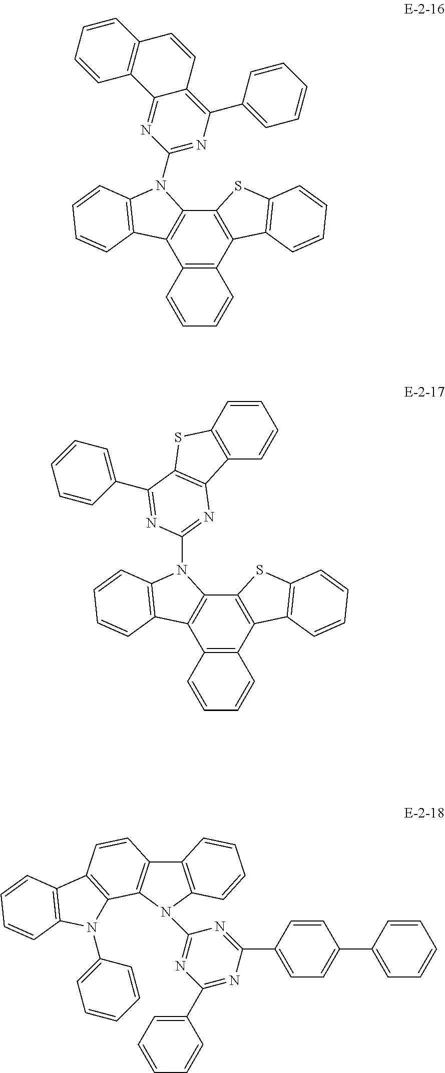 [SMILES: c1ccc(-c2nc(-n3c4ccccc4c4c5ccccc5c5c6ccccc6sc5c43)nc3c2ccc2ccccc23)cc1 ; c1ccc(-c2nc(-n3c4ccccc4c4c5ccccc5c5c6ccccc6sc5c43)nc3c2sc2ccccc23)cc1 ; c1ccc(-c2ccc(-c3nc(-c4ccccc4)nc(-n4c5ccccc5c5ccc6c7ccccc7n(-c7ccccc7)c6c54)n3)cc2)cc1]

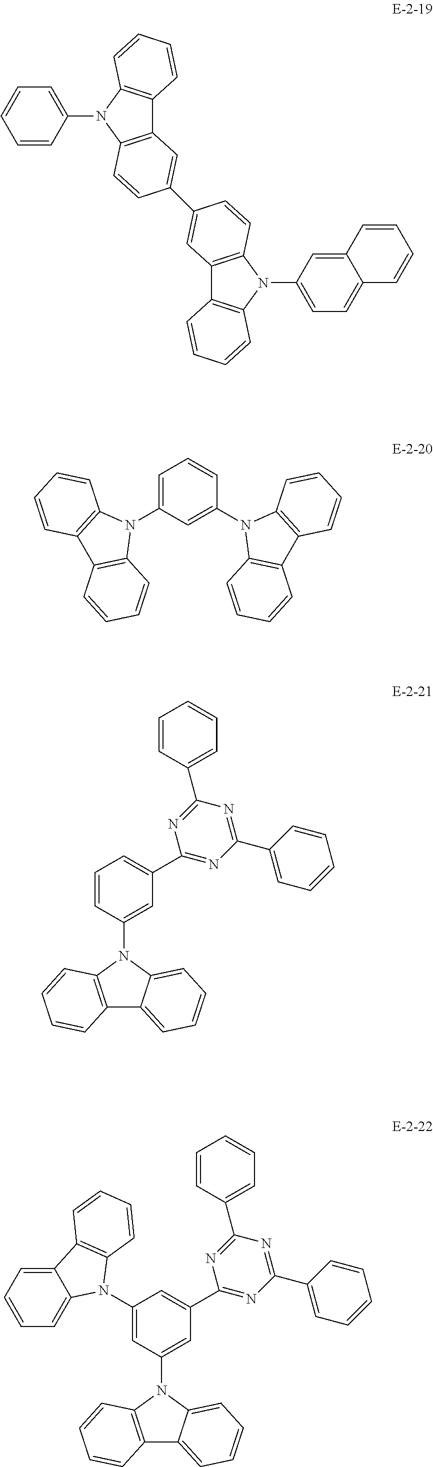 [SMILES: c1ccc(-n2c3ccccc3c3cc(-c4ccc5c(c4)c4ccccc4n5-c4ccc5ccccc5c4)ccc32)cc1 ; c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1]

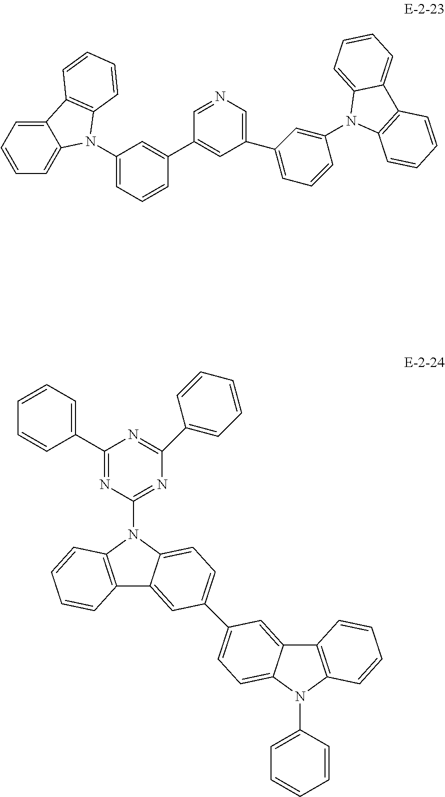 [SMILES: c1cc(-c2cncc(-c3cccc(-n4c5ccccc5c5ccccc54)c3)c2)cc(-n2c3ccccc3c3ccccc32)c1 ; c1ccc(-c2nc(-c3ccccc3)nc(-n3c4ccccc4c4cc(-c5ccc6c(c5)c5ccccc5n6-c5ccccc5)ccc43)n2)cc1]

In The light-emitting element ED according to one or more embodiments, illustrated in FIGS. 3 to 7, The light-emitting layer EML may include a host and a dopant, and the light-emitting layer EML may include a compound represented by Formula E-1. The compound represented by Formula E-1 may be used as a fluorescent host material.

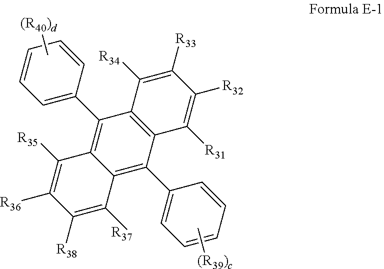

In Formula E-1, R₃₁ to R₄₀ may each independently be hydrogen, deuterium, a halogen, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, and/or independently bonded to an adjacent group to form a ring. In one or more embodiments, one or more selected from among R₃₁ to R₄₀ may be bonded to an adjacent group to form a saturated hydrocarbon ring, an unsaturated hydrocarbon ring, a saturated hetero ring, or an unsaturated hetero ring.

In Formula E-1, c and d may each independently be an integer of 0 to 5.

The compound represented by Formula E-1 may be any one selected from among Compounds E1 to E19.

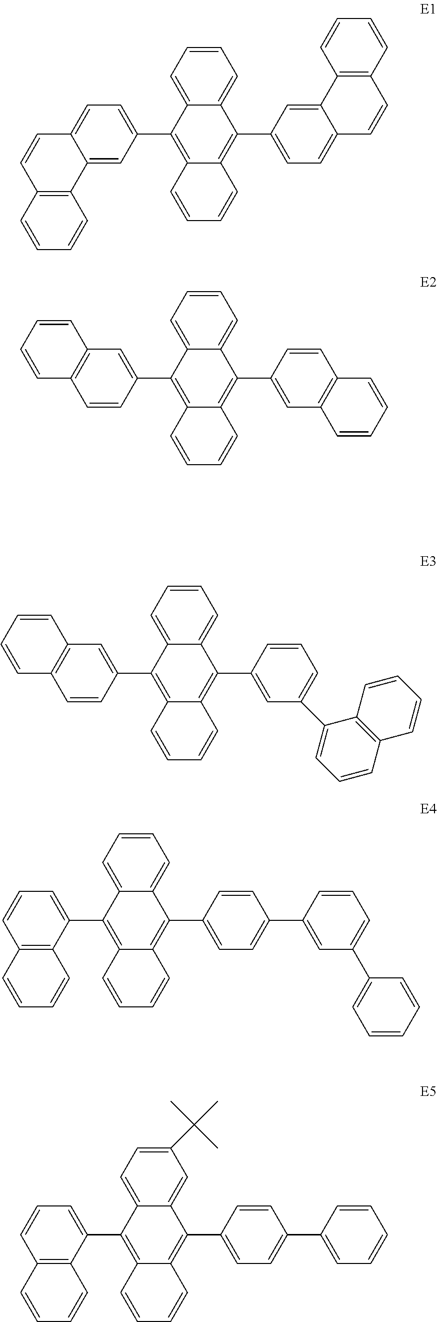

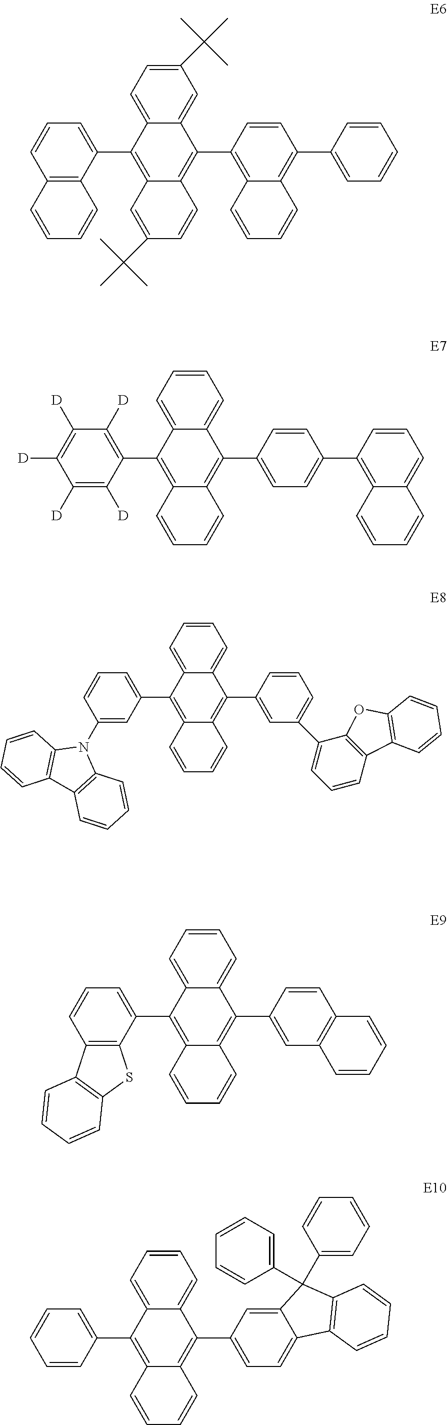

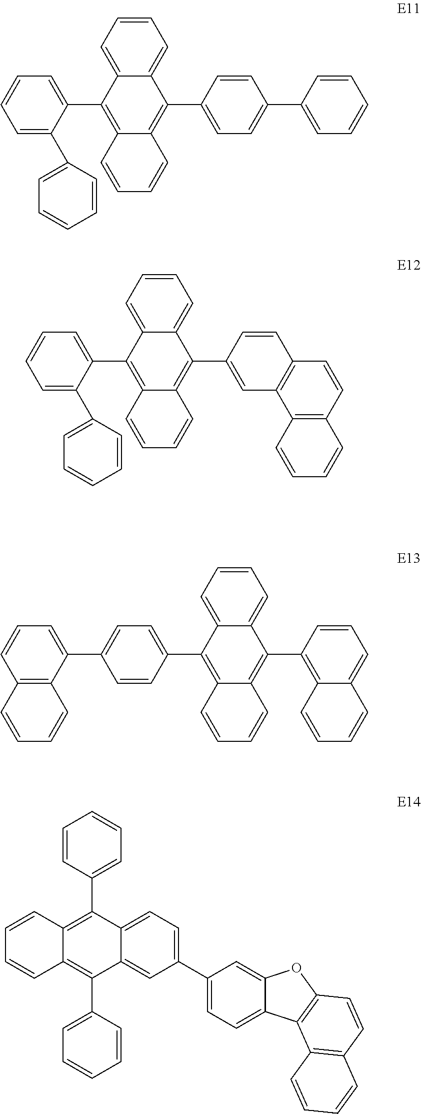

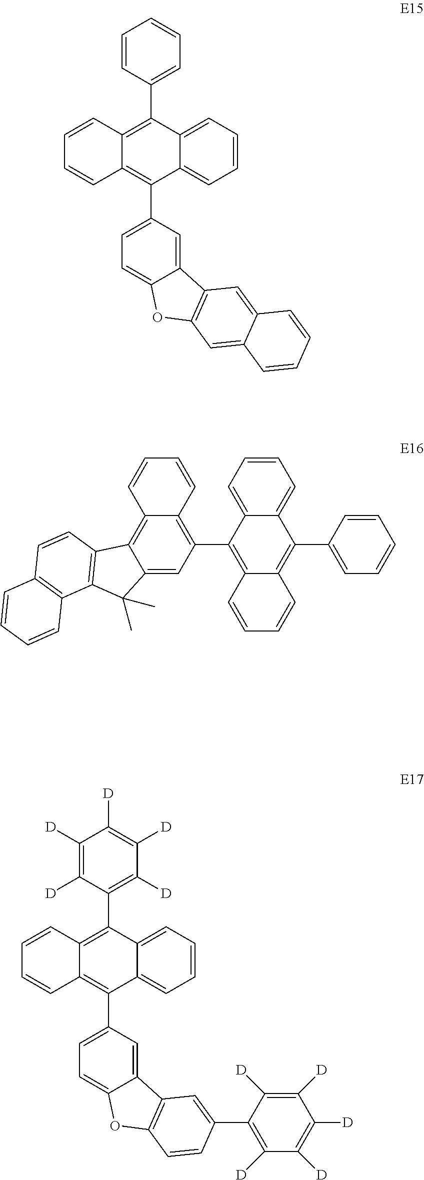

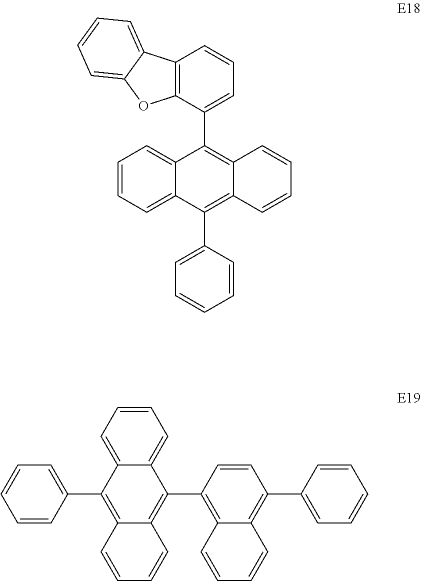

In one or more embodiments, the light-emitting layer may include a compound represented by Formula E-2a or Formula E-2b. The compound represented by Formula E-2a or Formula E-2b may be used as a host material for phosphorescent element (e.g., as a phosphorescence host material).

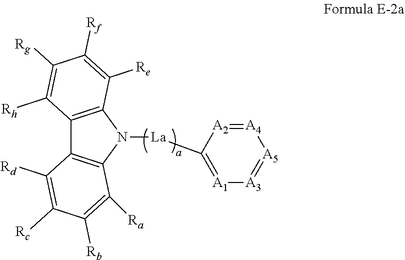

In Formula E-2a, a may be an integer of 0 to 10, La may be a direct linkage, a substituted or unsubstituted arylene group having 6 to 30 ring-forming carbon atoms, or a substituted or unsubstituted heteroarylene group having 2 to 30 ring-forming carbon atoms. In one or more embodiments, if (e.g., when) a is an integer of 2 or greater, a plurality of La's may each independently be a substituted or unsubstituted arylene group having 6 to 30 ring-forming carbon atoms or a substituted or unsubstituted heteroarylene group having 2 to 30 ring-forming carbon atoms.

In addition, in Formula E-2a, A₁ to A₅ may each independently be N or CRᵢ. Rₐ to Rᵢ may each independently be hydrogen, deuterium, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, and/or independently bonded to an adjacent group to form a ring. In one or more embodiments, one or more selected from among Rₐ to Rᵢ may be bonded to an adjacent group to form a hydrocarbon ring or a hetero ring containing N, O, S, and/or the like, as a ring-forming atom.

In one or more embodiments, in Formula E-2a, two or three selected from among A₁ to A₅ may be N, and the rest may be CRᵢ.

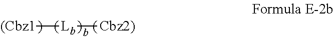

In Formula E-2b, Cbz1 and Cbz2 may each independently be an unsubstituted carbazole group or a carbazole group substituted with an aryl group having 6 to 30 ring-forming carbon atoms. L_{b} may be a direct linkage, a substituted or unsubstituted arylene group having 6 to 30 ring-forming carbon atoms, or a substituted or unsubstituted heteroarylene group having 2 to 30 ring-forming carbon atoms. b may be an integer of 0 to 10, and if (e.g., when) b is an integer of 2 or greater, a plurality of L_{b}'s may each independently be a substituted or unsubstituted arylene group having 6 to 30 ring-forming carbon atoms or a substituted or unsubstituted heteroarylene group having 2 to 30 ring-forming carbon atoms.

The compound represented by Formula E-2a or Formula E-2b may be any one selected from among compounds in Compound Group E-2. However, the compounds listed in Compound Group E-2 are mere example, the compound represented by Formula E-2a or Formula E-2b is not limited to the compounds listed in Compound Group E-2.

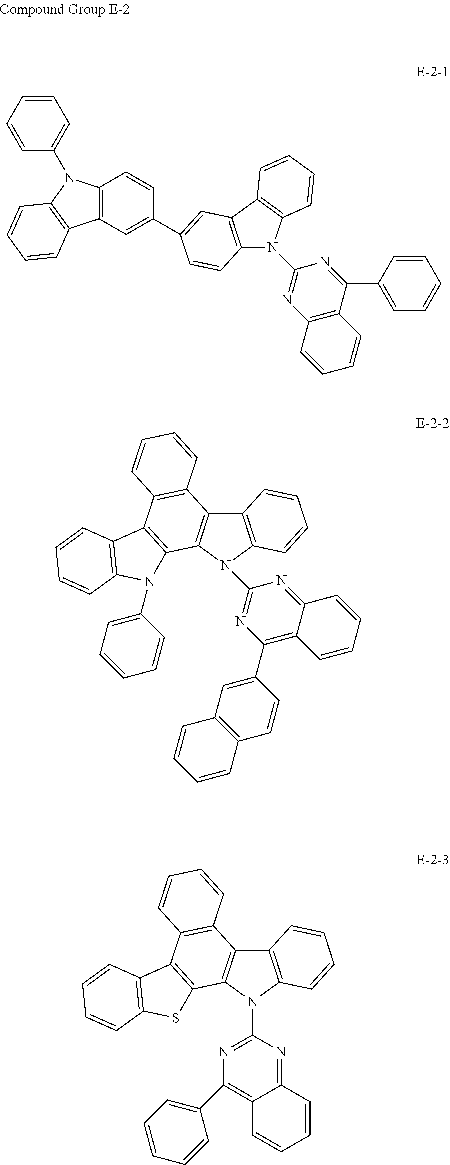 [SMILES: c1ccc(-c2nc(-n3c4ccccc4c4cc(-c5ccc6c(c5)c5ccccc5n6-c5ccccc5)ccc43)nc3ccccc23)cc1 ; c1ccc(-n2c3ccccc3c3c4ccccc4c4c5ccccc5n(-c5nc(-c6ccc7ccccc7c6)c6ccccc6n5)c4c32)cc1 ; c1ccc(-c2nc(-n3c4ccccc4c4c5ccccc5c5c6ccccc6sc5c43)nc3ccccc23)cc1]

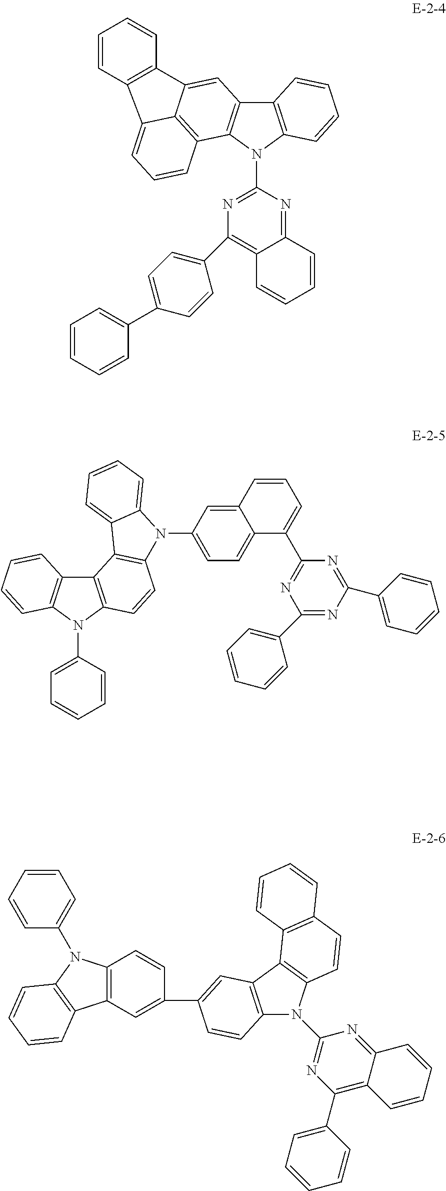 [SMILES: c1ccc(-c2ccc(-c3nc(-n4c5ccccc5c5cc6c7c(cccc7c54)-c4ccccc4-6)nc4ccccc34)cc2)cc1 ; c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1]

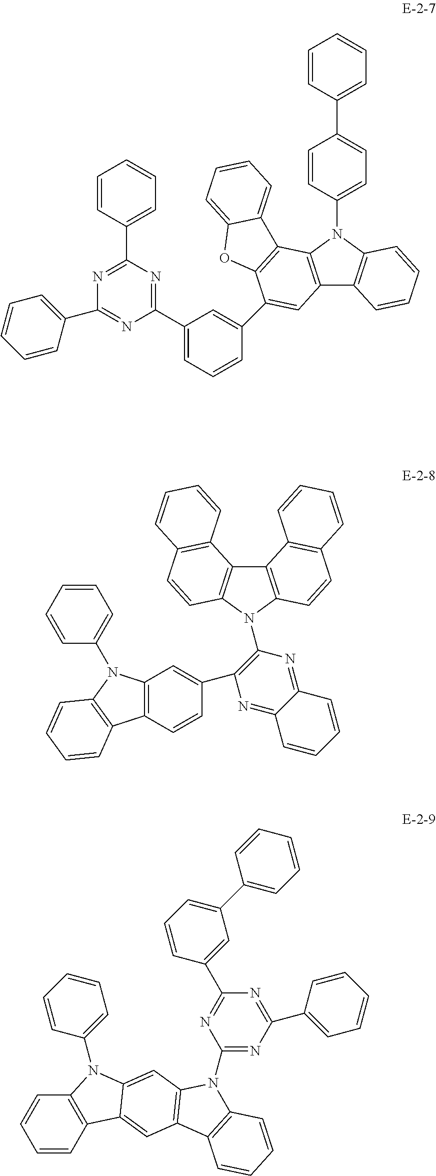 [SMILES: c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-n2c3ccccc3c3ccc(-c4nc5ccccc5nc4-n4c5ccc6ccccc6c5c5c6ccccc6ccc54)cc32)cc1 ; c1ccc(-c2cccc(-c3nc(-c4ccccc4)nc(-n4c5ccccc5c5cc6c7ccccc7n(-c7ccccc7)c6cc54)n3)c2)cc1]

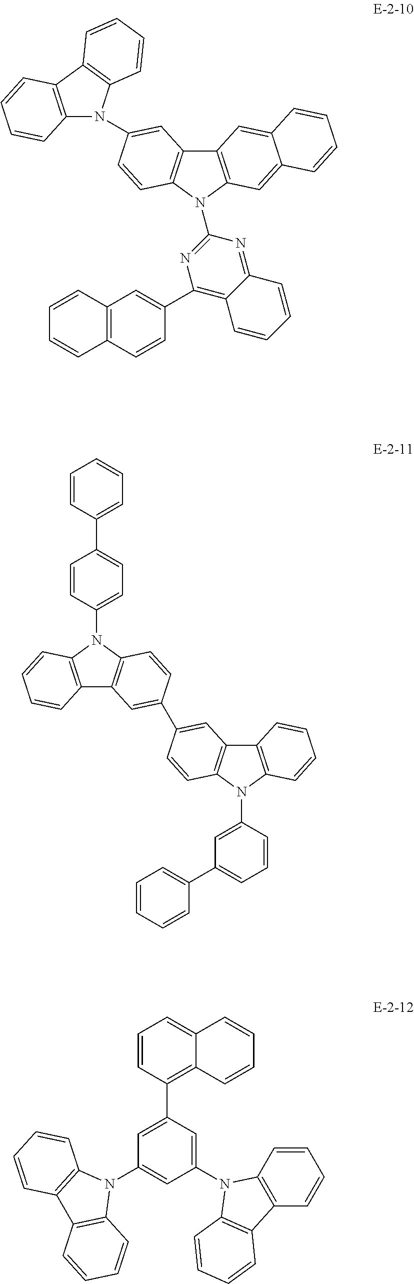 [SMILES: c1ccc2cc(-c3nc(-n4c5ccc(-n6c7ccccc7c7ccccc76)cc5c5cc6ccccc6cc54)nc4ccccc34)ccc2c1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc2c(-c3cc(-n4c5ccccc5c5ccccc54)cc(-n4c5ccccc5c5ccccc54)c3)cccc2c1]

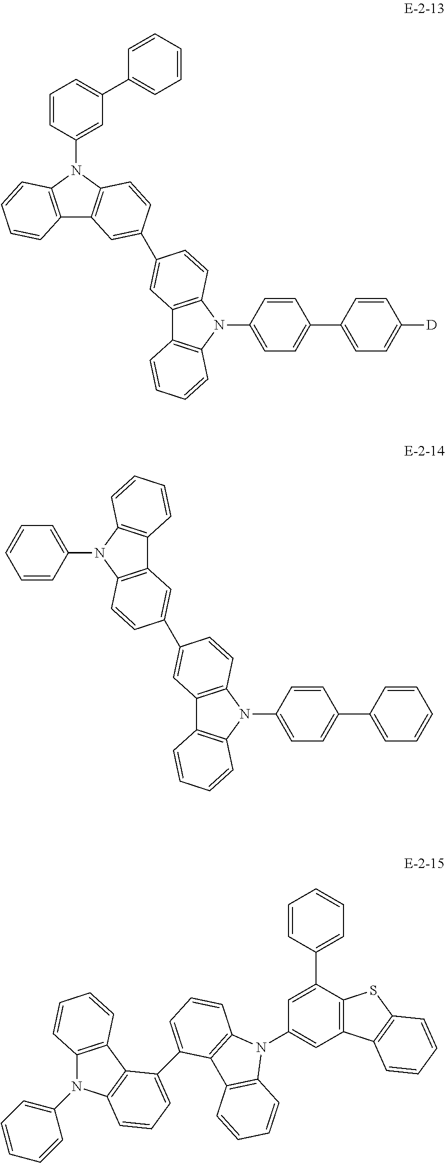 [SMILES: [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

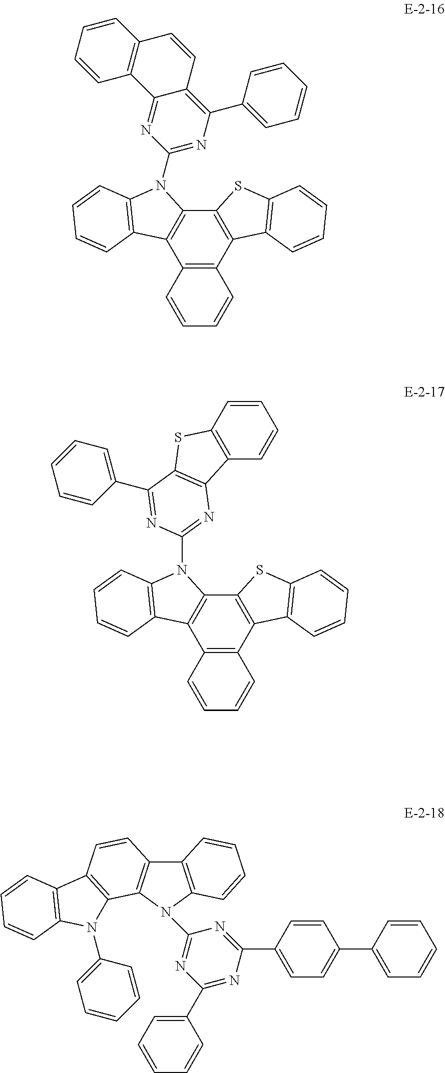 [SMILES: c1ccc(-c2nc(-n3c4ccccc4c4c5ccccc5c5c6ccccc6sc5c43)nc3c2ccc2ccccc23)cc1 ; c1ccc(-c2nc(-n3c4ccccc4c4c5ccccc5c5c6ccccc6sc5c43)nc3c2sc2ccccc23)cc1 ; c1ccc(-c2ccc(-c3nc(-c4ccccc4)nc(-n4c5ccccc5c5ccc6c7ccccc7n(-c7ccccc7)c6c54)n3)cc2)cc1]

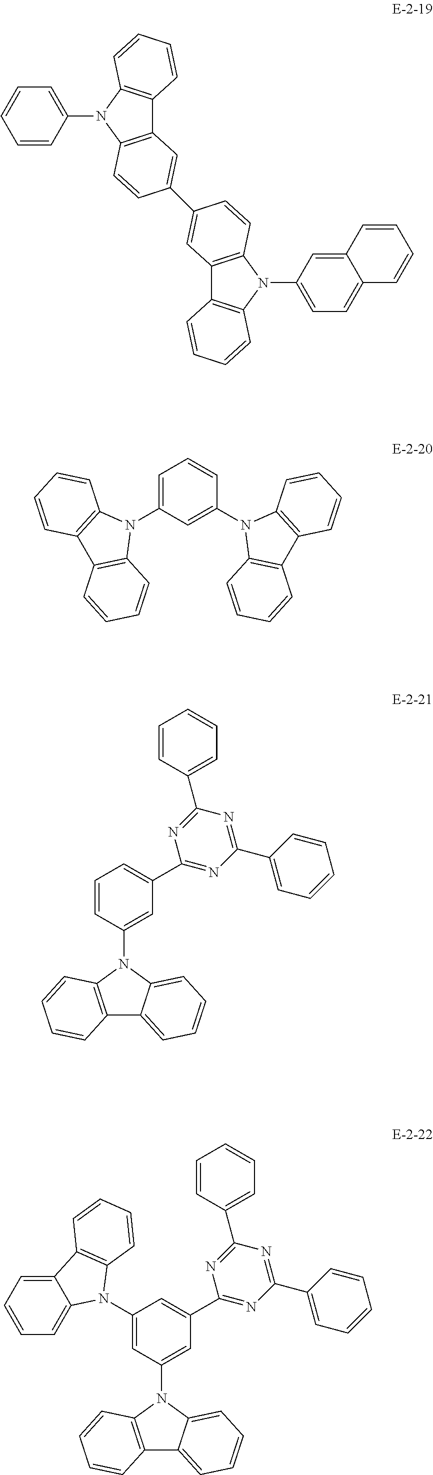 [SMILES: c1ccc(-n2c3ccccc3c3cc(-c4ccc5c(c4)c4ccccc4n5-c4ccc5ccccc5c4)ccc32)cc1 ; c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1]

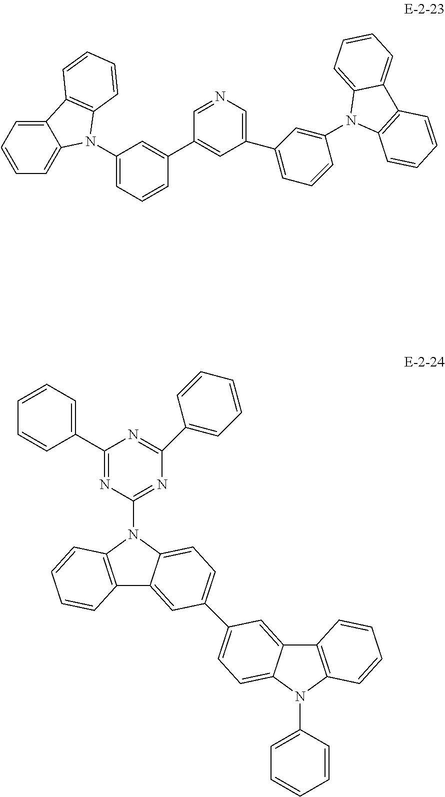 [SMILES: c1cc(-c2cncc(-c3cccc(-n4c5ccccc5c5ccccc54)c3)c2)cc(-n2c3ccccc3c3ccccc32)c1 ; c1ccc(-c2nc(-c3ccccc3)nc(-n3c4ccccc4c4cc(-c5ccc6c(c5)c5ccccc5n6-c5ccccc5)ccc43)n2)cc1]

In one or more embodiments, the light-emitting layer EML may further include materials suitable in the art as a host material. For example, the light-emitting layer EML may include, as a host material, at least one of bis(4-(9H-carbazol-9-yl) phenyl) diphenylsilane (BCPDS), (4-(1-(4-(diphenylamino) phenyl) cyclohexyl) phenyl) diphenyl-phosphine oxide (POPCPA), bis[2-(diphenylphosphino)phenyl]ether oxide (DPEPO), 4,4′-bis(N-carbazolyl)-1,1′-biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), or 1,3,5-tris(1-phenyl-1H-benzo[d]imidazol-2-yl)benzene (TPBi). However, embodiments of the present disclosure are not limited thereto, and, for example, examples of the host material may include tris(8-hydroxyquinolinato)aluminum (Alq3), 9,10-di(naphthalen-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene(MADN), hexaphenyl cyclotriphosphazene (CP1), 1,4-bis(triphenylsilyl)benzene (UGH2), hexaphenylcyclotrisiloxane (DPSiO3), octaphenylcyclotetra siloxane (DPSiO4), and/or the like.

In one or more embodiments, the light-emitting layer EML may include a compound represented by Formula M-a or Formula M-b. The compound represented by Formula M-a or Formula M-b may be used as a phosphorescent dopant material.

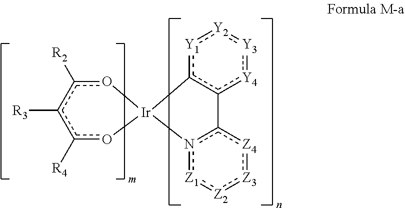

In Formula M-a, Y₁ to Y₄ and Z₁ to Z₄ may each independently CR₁ or N, R₁ to R₄ may each independently be hydrogen, deuterium, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, and/or independently bonded to an adjacent group to form a ring. In Formula M-a, m is 0 or 1, and n is 2 or 3. In Formula M-a, if (e.g., when) m is 0, n is 3, and if (e.g., when) m is 1, and n is 2.

The compound represented by Formula M-a may be any one selected from among Compounds M-a1 to M-a25. However, Compounds M-a1 to M-a25 are for explanatory purposes, and the compound represented by Formula M-a is not limited to the compounds listed in Compounds M-a1 to M-a25.

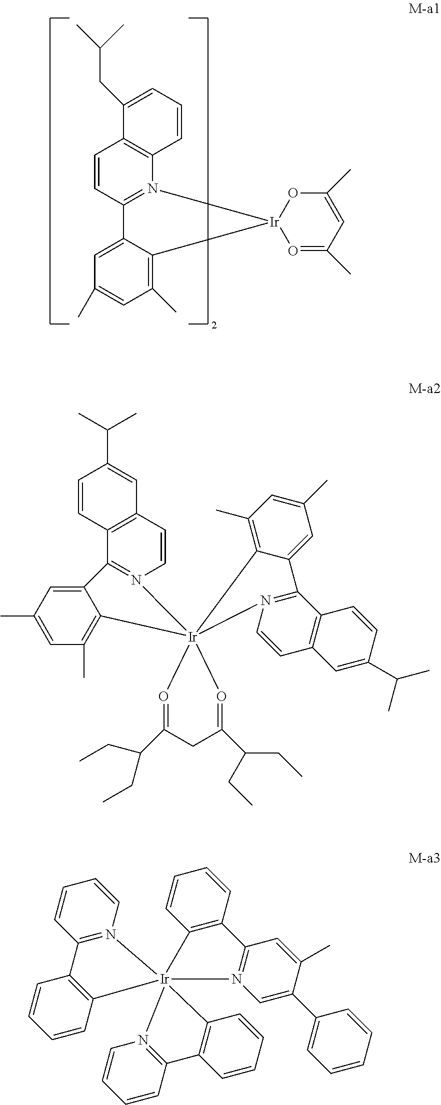

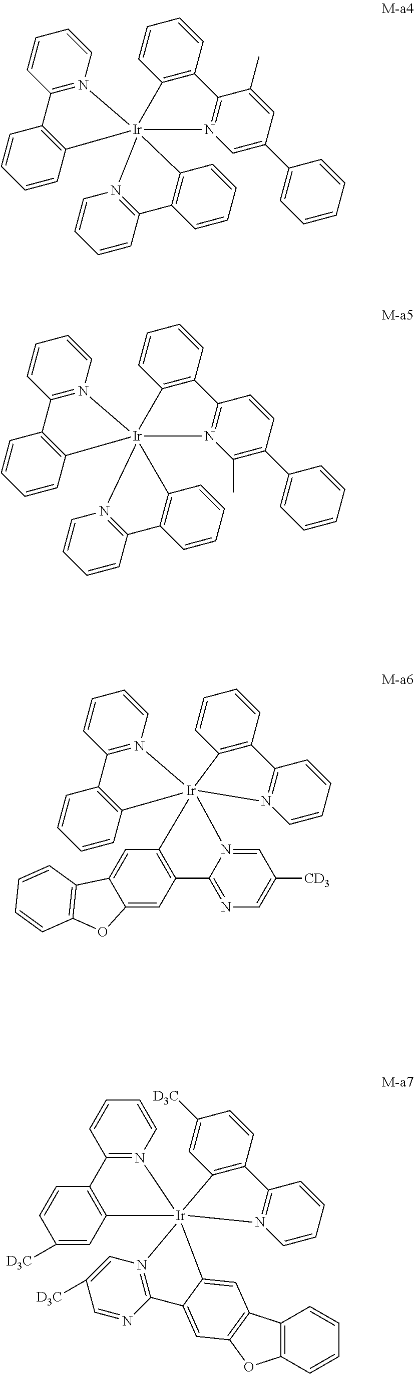

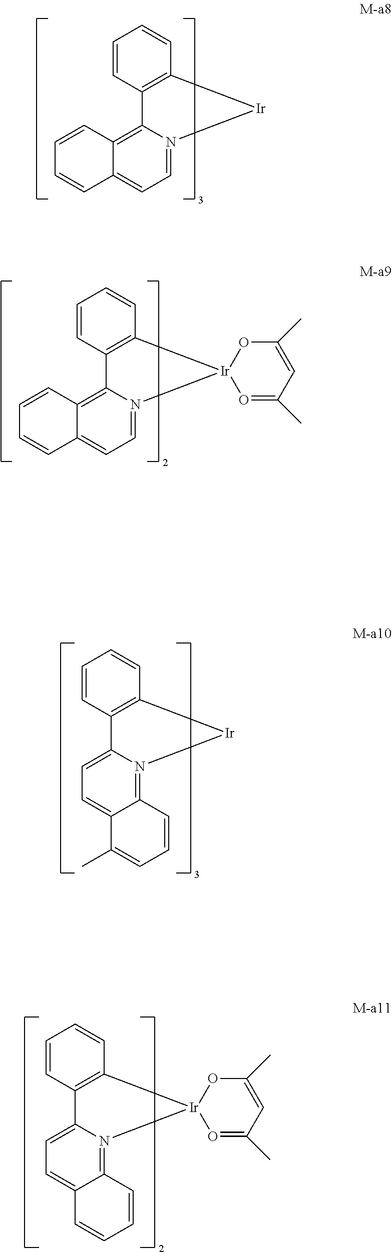

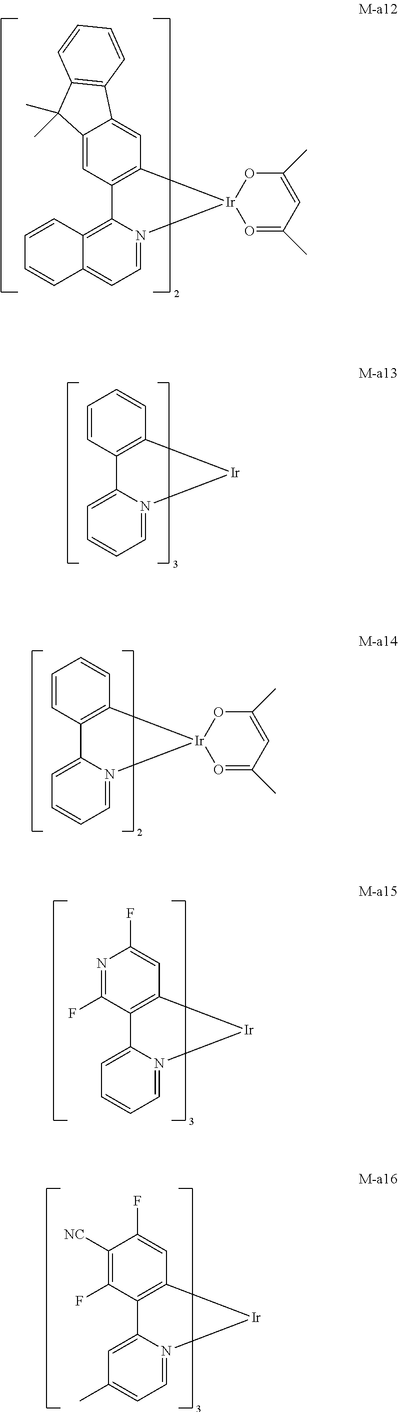

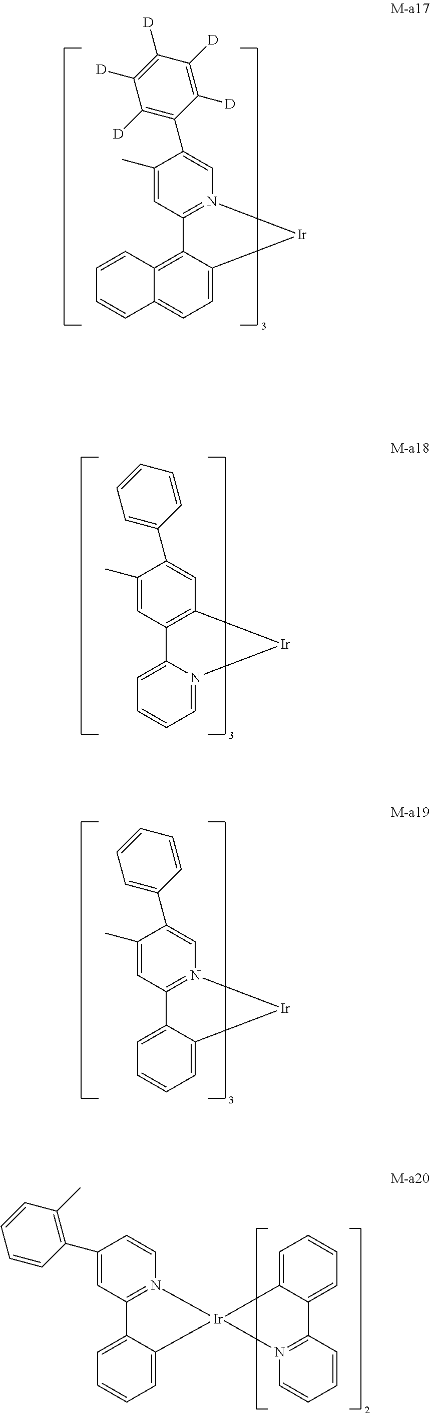

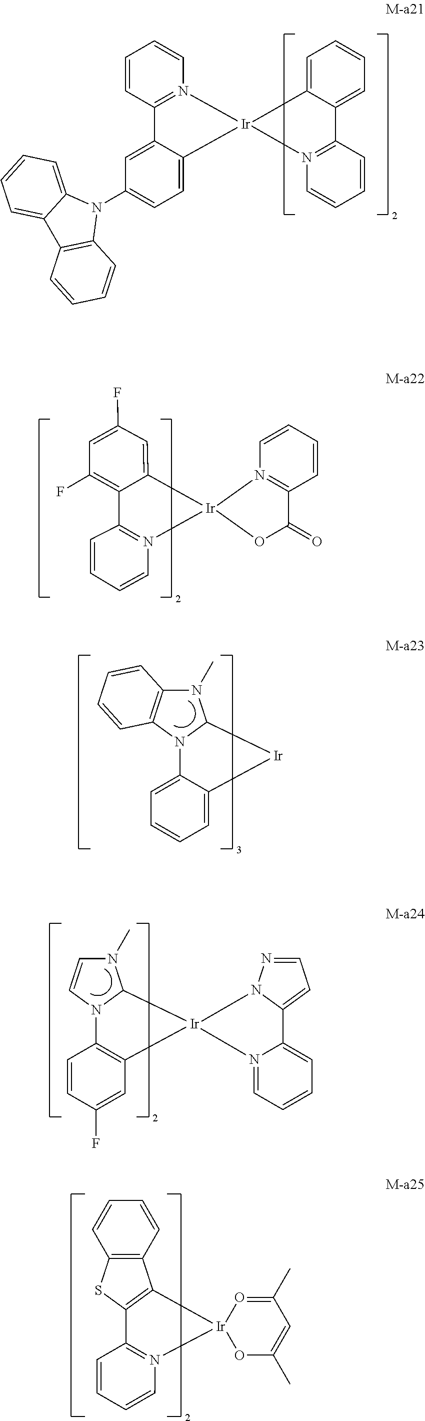

Compounds M-a1 and M-a2 may be used as a red dopant material, and Compounds M-a3 to M-a5 may be used as a green dopant material.

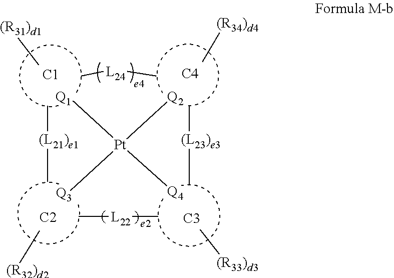

In Formula M-b, Q₁ to Q₄ may each independently be C or N, C₁ to C₄ may each independently be a substituted or unsubstituted hydrocarbon ring of 5 to 30 ring-forming carbon atoms or a substituted or unsubstituted heterocycle of 2 to 30 ring-forming carbon atoms. L₂₁ to L₂₄ may each independently be a direct linkage, *—O—*, *—S—*,

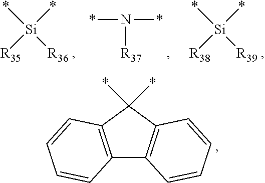

a substituted or unsubstituted alkylene group of 1 to 20 carbon atoms, a substituted or unsubstituted arylene group of 6 to 30 ring-forming carbon atoms, or a substituted or unsubstituted heteroarylene group of 2 to 30 ring-forming carbon atoms, and e1 to e4 may each independently be 0 or 1. R₃₁ to R₃₉ may each independently be hydrogen, deuterium, a halogen, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and/or independently combined with an adjacent group to form a ring, and d1 to d4 may each independently be an integer of 0 to 4.

The compound represented by Formula M-b may be used as a blue phosphorescence dopant or a green phosphorescence dopant.

The compound represented by Formula M-b may be any one selected from among the compounds listed below. However, those compounds are mere examples, and the compound represented by Formula M-b is not limited to the compounds represented below.

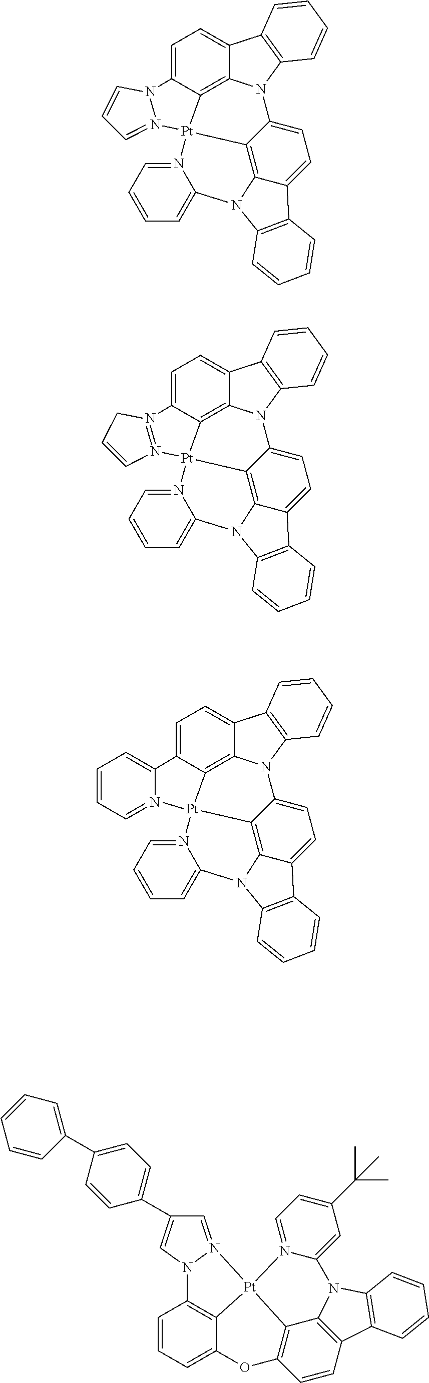

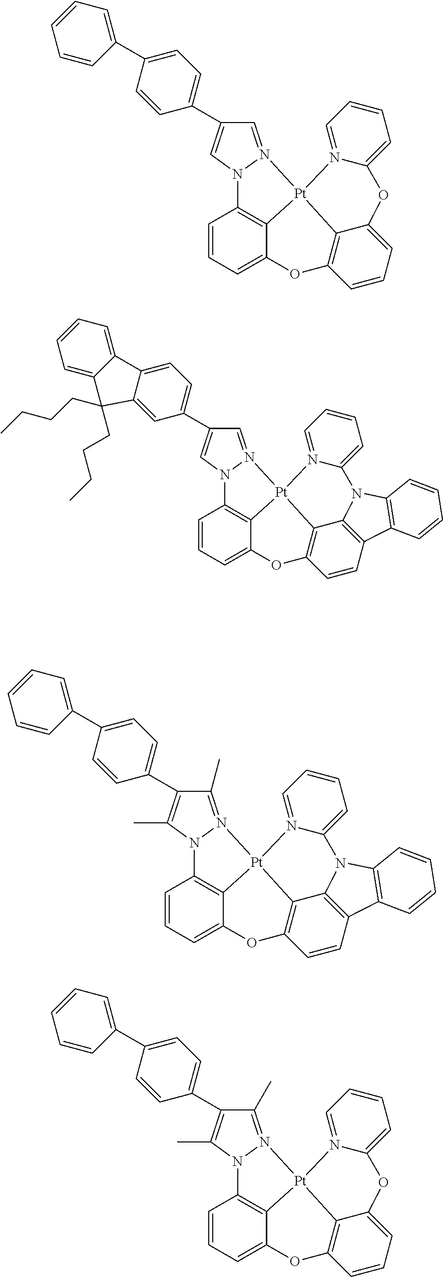

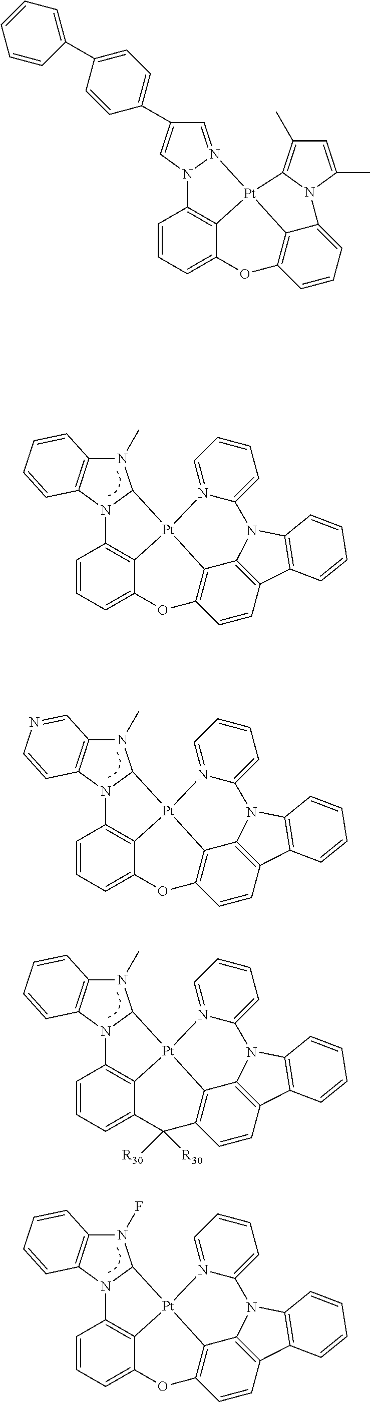

In the compounds above, R, R₃₈, and R₃₉ may each independently be hydrogen, deuterium, a halogen, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In one or more embodiments, the light-emitting layer EML may include one or more compounds represented by any one selected from among Formula F-a to Formula F-c. The compounds represented by Formula F-a to Formula F-c may be used as fluorescence dopant materials.

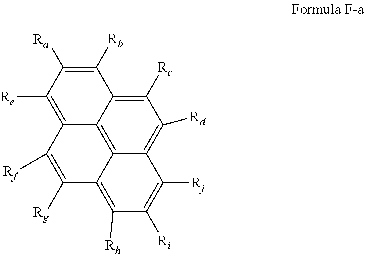

In Formula F-a, two selected from among Rₐ to Rⱼ may each independently be substituted with *—NAr₁Ar₂. The rest among Rₐ to Rⱼ, unsubstituted with *—NAr₁Ar₂ may each independently be hydrogen, deuterium, a halogen, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In *—NAr₁Ar₂, Ar₁ and Ar₂ may each independently be a substituted or unsubstituted aryl group having 6 to 30 ring-forming carbon atoms or a substituted or unsubstituted heteroaryl group having 2 to 30 ring-forming carbon atoms. For example, in one or more embodiments, at least one selected from among Ar₁ and Ar₂ may be a heteroaryl group containing O or S as a ring-forming atom.

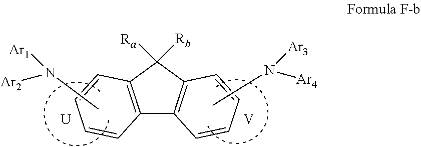

In Formula F-b, Rₐ and Rb may each independently be hydrogen, deuterium, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, and/or independently bonded to an adjacent group to form a ring. Ar₁ to Ar₄ may each independently be a substituted or unsubstituted aryl group having 6 to 30 ring-forming carbon atoms or a substituted or unsubstituted heteroaryl group having 2 to 30 ring-forming carbon atoms.

In Formula F-b, U and V may each independently be a substituted or unsubstituted hydrocarbon ring having 5 to 30 ring-forming carbon atoms or a substituted or unsubstituted hetero ring having 2 to 30 ring-forming carbon atoms.

In Formula F-b, the numbers of rings represented by U and V may each independently be 0 or 1. For example, in Formula F-b, if (e.g., when) the number of U or V is 1, one ring forms a part of a fused ring at the designated part by U or V, and if (e.g., when) the number of U or V is 0, no ring is present at the designated part by U or V. For example, if (e.g., when) the number of U is 0 and the number of V is 1, or if (e.g., when) the number of U is 1 and the number of V is 0, a fused ring having a fluorene core in Formula F-b may be a ring compound with four rings. In one or more embodiments, if (e.g., when) the number of both (e.g., simultaneously) U and V is 0, the fused ring in Formula F-b may be a ring compound with three rings. In one or more embodiments, if (e.g., when) the number of both (e.g., simultaneously) U and V is 1, a fused ring having the fluorene core of Formula F-b may be a ring compound with five rings.

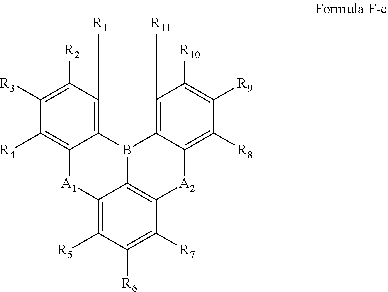

In Formula F-c, A₁ and A₂ may each independently be O, S, Se, or NRₘ, and Rₘ may be hydrogen, deuterium, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. R₁ to R₁₁ may each independently be hydrogen, deuterium, a halogen, a cyano group, a substituted or unsubstituted amine group, a substituted or unsubstituted boryl group, a substituted or unsubstituted oxy group, a substituted or unsubstituted thio group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and/or independently combined with an adjacent group to form a ring.

In Formula F-c, A₁ and A₂ may each independently be combined with the substituents of an adjacent ring to form a fused ring. For example, if A₁ and A₂ may each independently be NRₘ, A₁ may be combined with R₄ or R₅ to form a ring. In addition, A₂ may be combined with R₇ or R₈ to form a ring.

In one or more embodiments, the light-emitting layer EML may include, as a suitable dopant material, one or more selected from among a styryl derivative (e.g. 1,4-bis[2-(3-N-ethylcarbazolyl)vinyl]benzene (BCzVB), 4-(di-p-tolylamino)-4′-[(di-p-tolylamino)styryl]stilbene (DPAVB), N-(4-((E)-2-(6-((E)-4-(diphenylamino)styryl)naphthalen-2-yl)vinyl)phenyl)-N-phenylbenzenamine (N-BDAVBi)), 4,4′-bis[2-(4-(N,N-diphenylamino)phenyl)vinyl]biphenyl (DPAVBi), perylene and a derivative thereof (e.g., 2, 5, 8, 11-tetra-t-butylperylene (TBP)), pyrene and a derivative thereof (e.g. 1, 1-dipyrene, 1, 4-dipyrenylbenzene, 1, 4-bis(N, N-diphenylamino)pyrene), and/or the like.

In one or more embodiments, the light-emitting layer EML may include a suitable phosphorescent dopant material. For example, examples of the phosphorescent dopant may include a metal complex containing iridium (Ir), platinum (Pt), osmium (Os), gold (Au), titanium (Ti), zirconium (Zr), hafnium (Hf), europium (Eu), terbium (Tb), or thulium (Tm), and/or the like. For example, iridium(III) bis(4,6-difluorophenylpyridinato-N,C2′)picolinate (Flrpic), bis(2,4-difluorophenylpyridinato)-tetrakis(1-pyrazolyl)borate iridium (Ill) (Fir6), or platinum octaethyl porphyrin (PtOEP) may be used as a phosphorescent dopant. However, embodiments of the present disclosure are not limited thereto.

In the light-emitting element ED according to one or more embodiments, the light-emitting layer EML may be a delayed fluorescence light-emitting layer including a host and a dopant. For example, the light-emitting layer EML may be to emit thermally activated delayed fluorescence (TADF). In the light-emitting element ED according to one or more embodiments, the light-emitting layer EML may include a suitable thermally active delayed fluorescence dopant.

In one or more embodiments, the light-emitting layer EML of the light-emitting element ED may include a plurality of host materials, a thermally activated delayed fluorescent dopant, and a phosphorescent sensitizer.

In one or more embodiments, the light-emitting layer EML may include a quantum dot material. The quantum dot may have a core/shell structure. The core of the quantum dots may be selected from among a Group II-VI compound, a Group I-II-VI compound, a Group II-IV-VI compound, a Group I-II-IV-VI compound, a Group II-IV-V compound, a Group Ill-VI compound, a Group I-III-VI compound, a Group Ill-V compound, a Group Ill-II-V compound, a Group IV-VI compound, a Group IV element, a Group IV compound, and/or a (e.g., any suitable) combination thereof.

The Group II-VI compound may be selected from the group consisting of a binary compound selected from the group consisting of CdSe, CdTe, CdS, ZnS, ZnSe, ZnTe, ZnO, HgS, HgSe, HgTe, MgSe, MgS, and a (e.g., any suitable) mixture thereof, a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and a (e.g., any suitable) mixture thereof, and a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, and a (e.g., any suitable) mixture thereof.

The Group III-VI compound may include a binary compound such as In₂S₃ and/or In₂Se₃, a ternary compound such as InGaS₃ and/or InGaSes, or any combination thereof.

The Group I-III-VI compound may be selected from among a ternary compound selected from the group consisting of AgInS, AgInS₂, CuInS, CuInS₂, AgGaS₂, CuGaS₂ CuGaO₂, AgGaO₂, AgAlO₂, and a (e.g., any suitable) mixture thereof, and/or a quaternary compound such as AgInGaS₂ and/or CulnGaS₂.

The Group III-V compound may be selected from the group consisting of a binary compound selected from the group consisting of GaN, GaP, GaAs, GaSb, AlN, AIP, AIAs, AISb, InN, InP, InAs, InSb, and a (e.g., any suitable) mixture thereof, a ternary compound selected from the group consisting of GaNP, GaNAs, GaNSb, GaPAs, GaPSb, AlNP, AlNAs, AlNSb, AIPAs, AIPSb, InGaP, InAIP, InNP, InNAs, InNSb, InPAs, InPSb, and a (e.g., any suitable) mixture thereof, and a quaternary compound selected from the group consisting of GaAlNP, GaAlNAs, GaAlNSb, GaAlPAs, GaAlPSb, GaInNP, GaInNAs, GalnNSb, GaInPAs, GalnPSb, InAINP, InAINAs, InAINSb, InAIPAs, InAIPSb, and a (e.g., any suitable) mixture thereof. In one or more embodiments, the Group Ill-V compound may further include a Group II metal. For example, InZnP, and/or the like, may be selected as a Group III—II-V compound.

The Group IV-VI compound may be selected from the group consisting of a binary compound selected from the group consisting of SnS, SnSe, SnTe, PbS, PbSe, PbTe, and/or a (e.g., any suitable) mixture thereof, a ternary compound selected from the group consisting of SnSeS, SnSeTe, SnSTe, PbSeS, PbSeTe, PbSTe, SnPbS, SnPbSe, SnPbTe, and a (e.g., any suitable) mixture thereof, and a quaternary compound selected from the group consisting of SnPbSSe, SnPbSeTe, SnPbSTe, and a (e.g., any suitable) mixture thereof. The Group IV element may be selected from the group consisting of Si, Ge, and a (e.g., any suitable) mixture thereof. The Group IV compound may be a binary compound selected from the group consisting of SiC, SiGe, and a (e.g., any suitable) mixture thereof.

Each element included in a polynary compound such as the binary compound, the ternary compound, or the quaternary compound may be present in a particle with a substantially uniform or non-uniform concentration distribution. For example, the formulae refer to the types (kinds) of elements included in the compounds, and elemental ratios in the compound may be different. For example, AgInGaS₂ may refer to AgInₓGa₁₋ₓS₂ (where x is a real number of 0 to 1).

In one or more embodiments, the quantum dot may have a single structure in which the concentration of each element included in the quantum dot is substantially uniform, or a double structure of core-shell. For example, the material included in the core may be different from the material included in the shell.

The shell of the quantum dot may serve as a protection layer to prevent or reduce the chemical deformation of the core to maintain semiconductor properties, and/or a charging layer to impart electrophoresis properties to the quantum dot. The shell may be a single layer or a multilayer. An interface between the core and the shell may have a concentration gradient in which the concentration of an element present in the shell becomes lower towards the center.

An example of the shell of the quantum dots may include a metal or non-metal oxide, a semiconductor compound, and/or a (e.g., any suitable) combination thereof. For example, the metal or non-metal oxide for the shell may be a binary compound such as SiO₂, Al₂O₃, TiO₂, ZnO, MnO, Mn₂₀₃, Mn₃₀₄, CuO, FeO, Fe₂O₃, Fe₃O₄, CoO, Co₃O₄, or NiO, or a ternary compound such as MgA₁₂O₄, CoFe₂O₄, NiFe₂O₄, or CoMn₂O₄, but embodiments of the present disclosure are not limited thereto.

Also, examples of the semiconductor compound suitable as a shell may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AIAs, AIP, AISb, and/or the like, but embodiments of the disclosure are not limited thereto.

Each element included in a polynary compound such as the binary compound, or the ternary compound may be present in a particle with a substantially uniform or non-uniform concentration distribution. For example, the formulae refer to the types (kinds) of elements included in the compounds, and elemental ratios in the compound may be different.

The quantum dot may have a full width at half maximum (FWHM) of an emission spectrum of about 45 nm or less, about 40 nm or less, or about 30 nm or less, and color purity or color reproducibility of the quantum dot may be improved in the above ranges. In addition, light emitted through such a quantum dot is emitted in all directions, and thus a wide viewing angle may be improved.

In addition, although the form of the quantum dot is not particularly limited as long as it is a form generally used in the art, for example, the quantum dot in the form of spherical nanoparticles, pyramidal nanoparticles, multi-arm nanoparticles, cubic nanoparticles, nanotubes, nanowires, nanofibers, nanoplate particles, and/or the like, may be used.

As the size of the quantum dot is adjusted or the elemental ratio in the quantum dot compound is adjusted, it may control the energy band gap of the quantum dot, and thus light in one or more suitable wavelength ranges may be obtained in a quantum dot light-emitting layer. Therefore, when the quantum dots described above are used (e.g., using different sizes of quantum dots or different elemental ratios in the quantum dot compound), and the light-emitting element, which emits light in one or more suitable wavelengths, may be implemented. For example, the size of the quantum dots or the elemental ratio in the quantum dot compound may be adjusted to enable the quantum dots to emit red, green, and/or blue light. In one or more embodiments, the quantum dots may be configured to emit white light by combining one or more suitable colors of light.

In each of the light-emitting elements ED of embodiments illustrated in FIGS. 3 to 7, the electron transport region ETR is provided on the light-emitting layer EML. The electron transport region ETR may include at least one of a hole blocking layer HBL, an electron transport layer ETL, or an electron injection layer EIL, but embodiments of the present disclosure are not limited thereto.

The electron transport region ETR may have a single layer formed of a single material, a single layer formed of a plurality of different materials, or a multilayer structure including a plurality of layers formed of a plurality of different materials.

For example, in one or more embodiments, the electron transport region ETR may have a single layer structure of the electron injection layer EIL or the electron transport layer ETL, or may have a single layer structure formed of an electron injection material and/or an electron transport material. In one or more embodiments, the electron transport region ETR may have a single layer structure formed of a plurality of different materials, or may have a structure in which an electron transport layer ETL/electron injection layer EIL, a hole blocking layer HBL/electron transport layer ETL/electron injection layer EIL are stacked in order (e.g., in the stated order) from the light-emitting layer EML, but embodiments of the present disclosure are not limited thereto. The electron transport region ETR may have a thickness, for example, from about 1,000 Å to about 1,500 Å.

The electron transport region ETR may be formed using one or more suitable methods such as a vacuum deposition method, a spin coating method, a casting method, a Langmuir-Blodgett (LB) method, an inkjet printing method, a laser printing method, and/or a laser induced thermal imaging (LITI) method.

In one or more embodiments, the electron transport region ETR may include a compound represented by Formula ET-2.

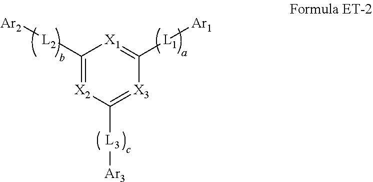

In Formula ET-2, at least one selected from among X₁ to X₃ is N, and the rest are CRₐ. Rₐ may be hydrogen, deuterium, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. Ar₁ to Ar₃ may each independently be hydrogen, deuterium, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In Formula ET-2, a to c may each independently be an integer of 0 to 10. In Formula ET-2, L₁ to L₃ may each independently be a direct linkage, a substituted or unsubstituted arylene group having 6 to 30 ring-forming carbon atoms, or a substituted or unsubstituted heteroarylene group having 2 to 30 ring-forming carbon atoms. In one or more embodiments, if (e.g., when) a to c may each independently be an integer of 2 or greater, L₁'s to L₃'s may each independently be a substituted or unsubstituted arylene group having 6 to 30 ring-forming carbon atoms or a substituted or unsubstituted heteroarylene group having 2 to 30 ring-forming carbon atoms.

In one or more embodiments, the electron transport region ETR may include an anthracene-based compound. However, embodiments of the disclosure are not limited thereto, for example, the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzimidazol-1-yl)phenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(biphenyl-4-yl)-4-phenyl-5-tert-butylphenyl-1,2,4-triazole (TAZ), 4-(naphthalen-1-yl)-3,5-diphenyl-4H-1,2,4-triazole (NTAZ), 2-(biphenyl-4-yl)-5-(4-tert-butylphenyl)-1,3,4-oxadiazole (tBu-PBD), bis(2-methyl-8-quinolinolato-N1,O8)-(1,1′-biphenyl-4-olato)aluminum (BAIq), beryllium bis(benzoquinolin-10-olate) (Bebq₂), 9,10-di(naphthalen-2-yl)anthracene (ADN), 1,3-bis[3,5-di(pyridin-3-yl)phenyl]benzene (BmPyPhB), and/or a (e.g., any suitable) mixture thereof.

In one or more embodiments, the electron transport region ETR may include at least one selected from among Compound ET1 to Compound ET36.

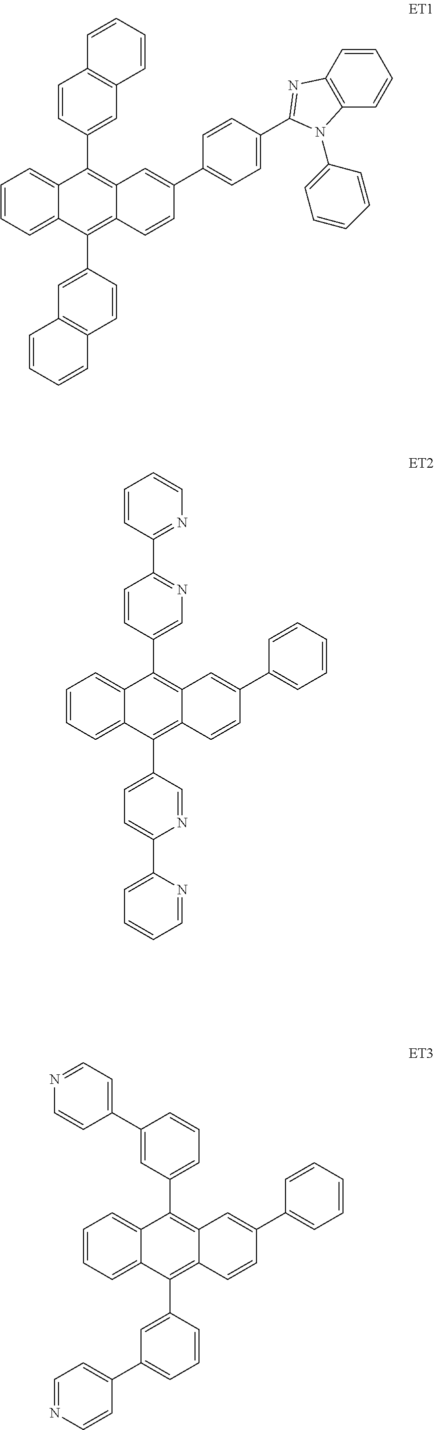 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

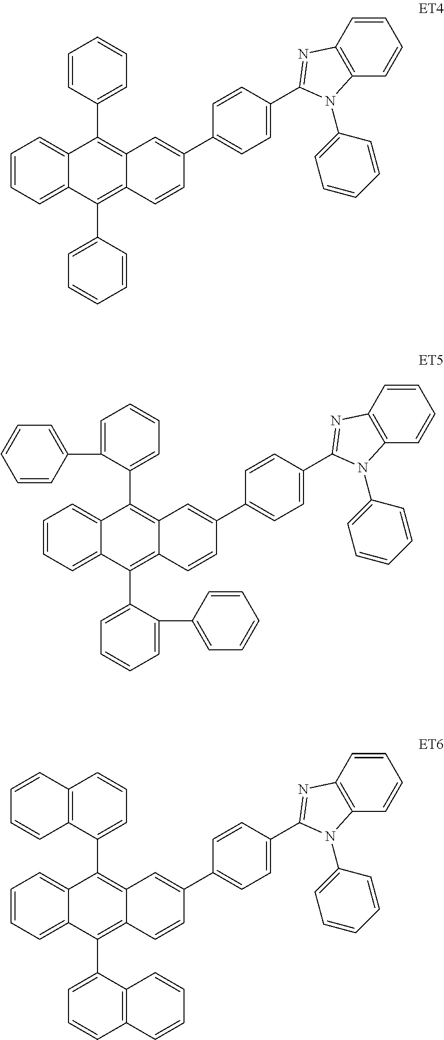 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

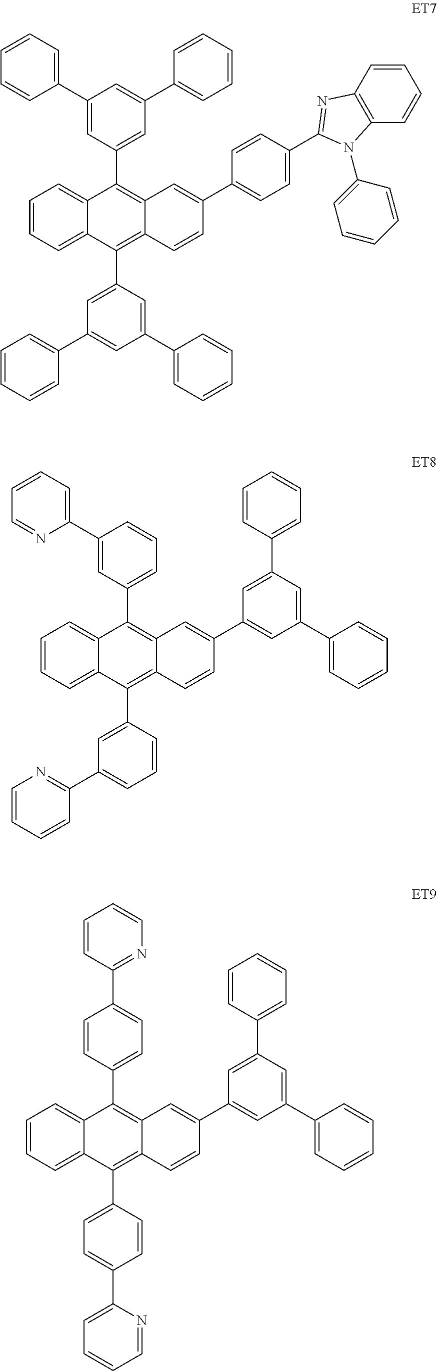 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

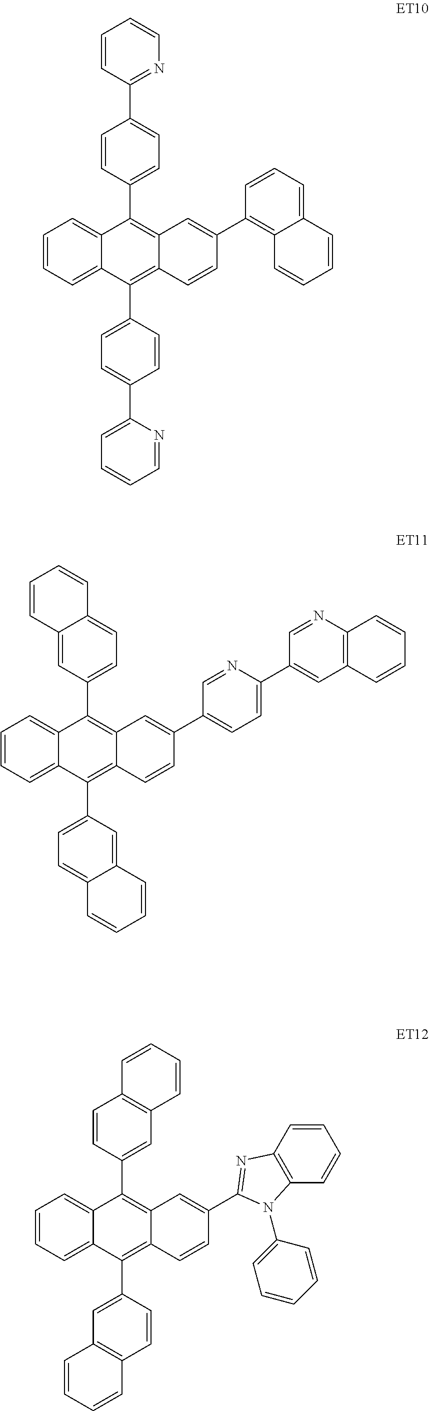 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

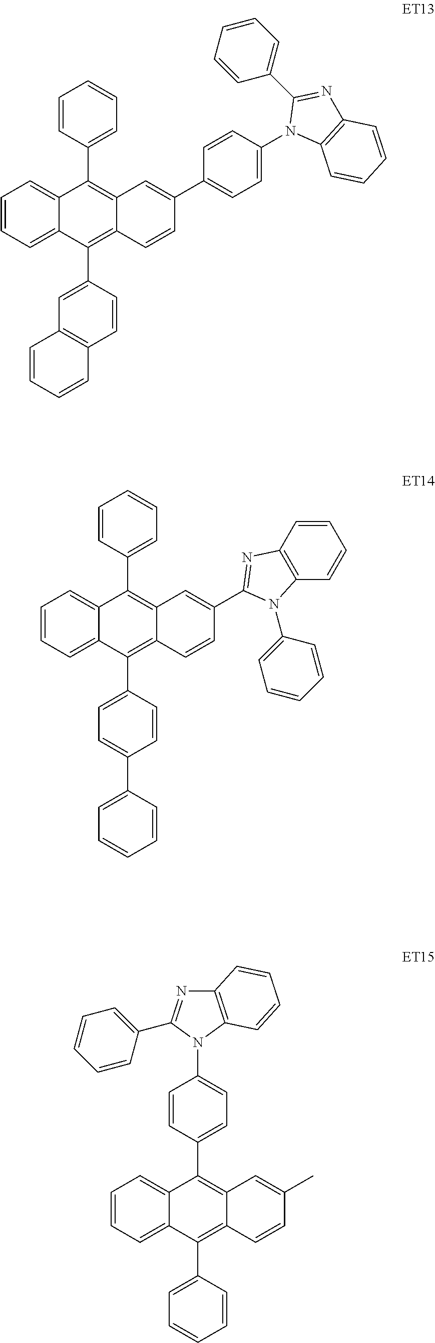 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

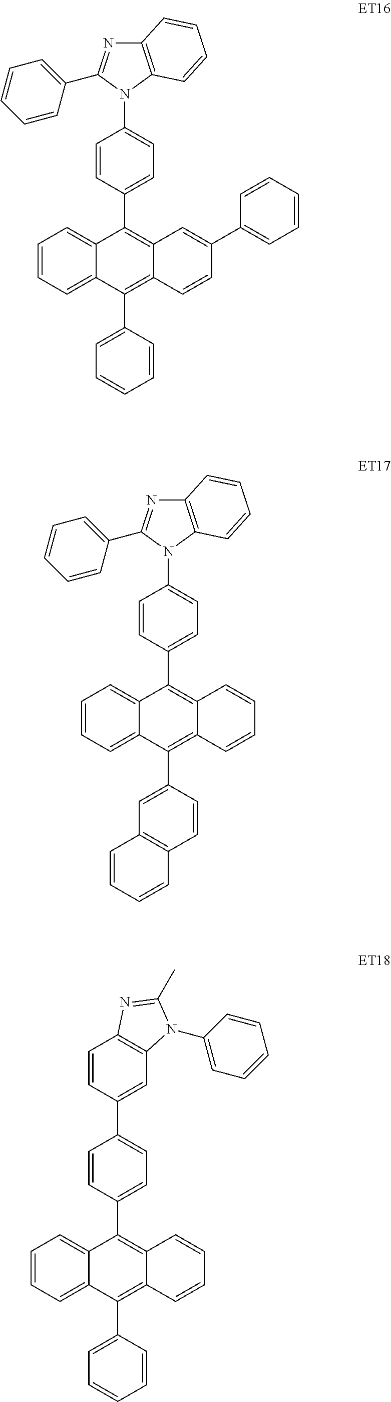 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

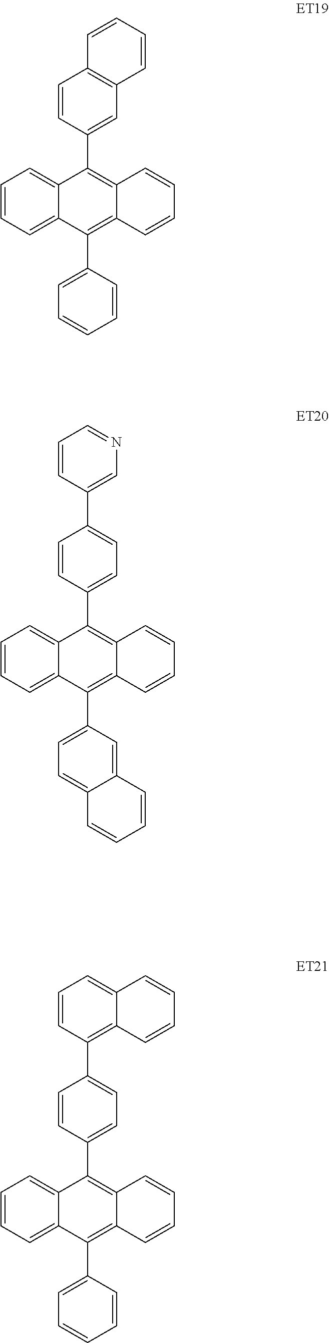

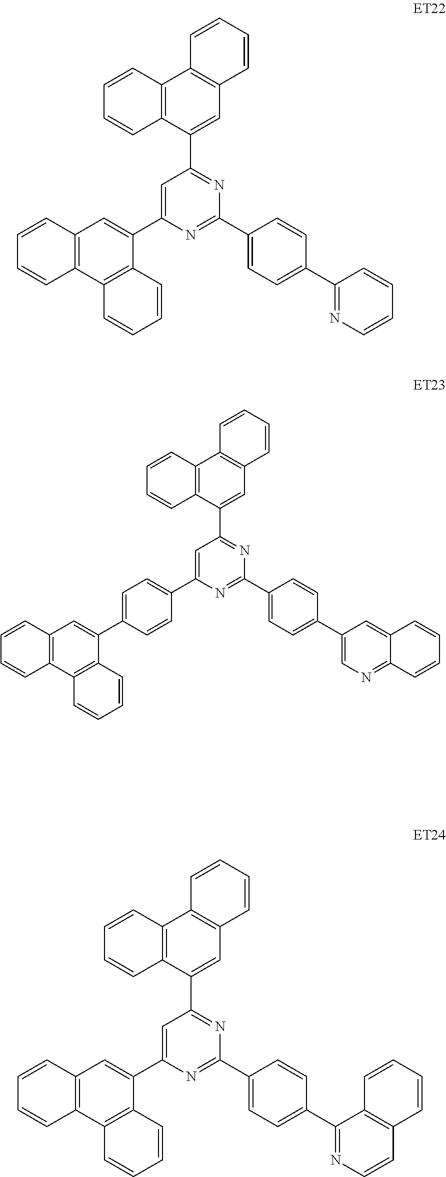 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1]

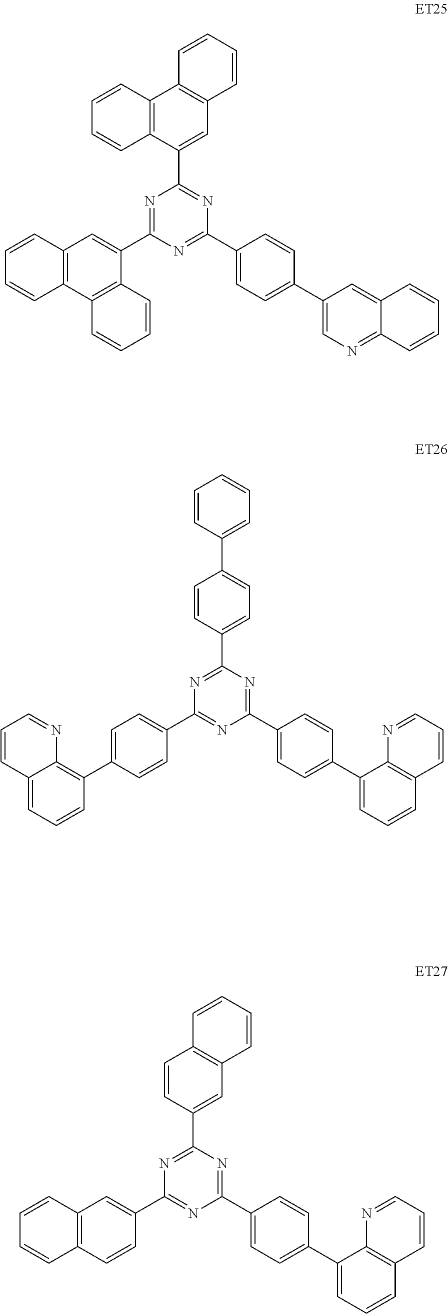 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

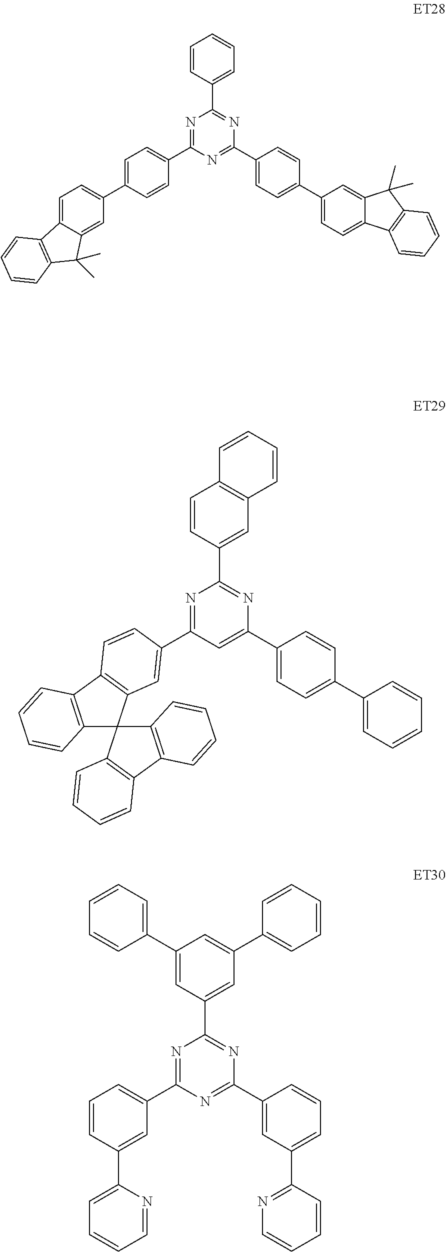 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

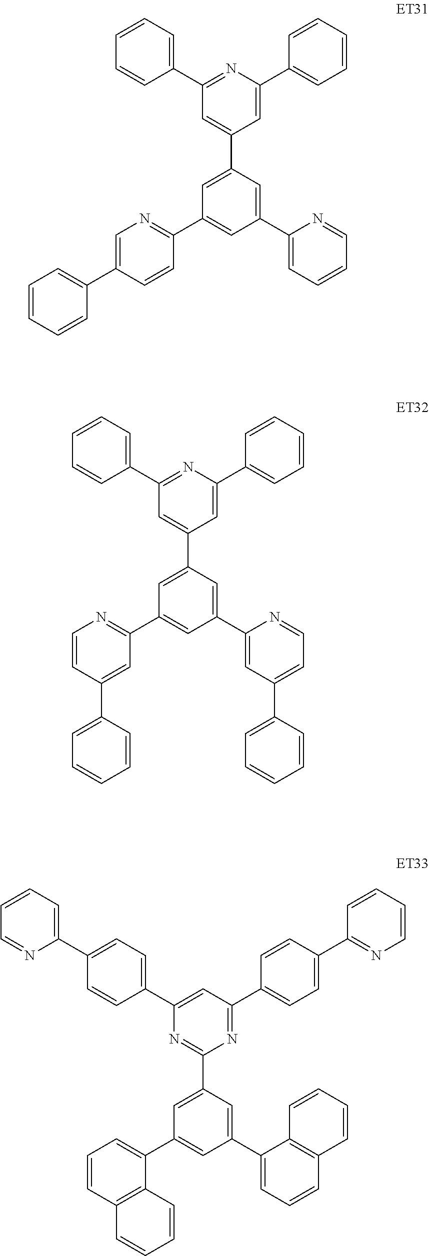 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

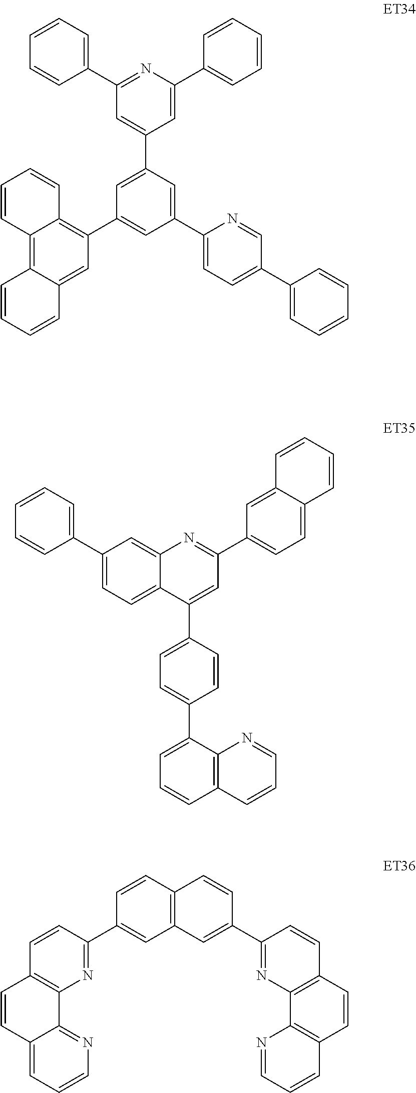 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

In one or more embodiments, the electron transport region ETR may include a metal halide such as LiF, NaCl, CsF, RbCI, RbI, CuI, and/or KI, a lanthanide metal such as Yb, or a co-deposited material of the metal halide and the lanthanide metal.

For example, in one or more embodiments, the electron transport region ETR may include KI:Yb, RbI:Yb, LiF:Yb, and/or the like, as the co-deposited material. In one or more embodiments, the electron transport region ETR may be formed using a metal oxide such as Li₂O and/or BaO, or 8-hydroxyl-lithium quinolate (Liq), and/or the like, but embodiments of the present disclosure are not limited thereto. The electron transport region ETR may also be formed of a mixture material of an electron transport material and an insulating organometallic salt. The insulating organometallic salt may be a material having an energy band gap of about 4 eV or more. For example, the insulating organometallic salt may include, for example, a metal acetate, a metal benzoate, a metal acetoacetate, a metal acetylacetonate, or a metal stearate.

In one or more embodiments, the electron transport region ETR may further include at least one of 2,9-dimethyl-4,7-diphenyl-1,10-phenanthroline (BCP), diphenyl(4-(triphenylsilyl)phenyl)phosphine oxide (TSPO1), or 4,7-diphenyl-1,10-phenanthroline (Bphen) in addition to one or more of the above-described materials, but embodiments of the present disclosure are not limited thereto.

The electron transport region ETR may include one or more of the above-described compounds of the hole transport region in at least one of the electron injection layer EIL, the electron transport layer ETL, or the hole blocking layer HBL.

If (e.g., when) the electron transport region ETR includes the electron transport layer ETL, the electron transport layer ETL may have a thickness of about 100 Å to about 1,000 Å, for example, about 150 Å to about 500 Å. If (e.g., when) the thickness of the electron transport layer ETL satisfies the aforementioned ranges, satisfactory electron transport characteristics may be obtained without a substantial increase in driving voltage. If (e.g., when) the electron transport region ETR includes the electron injection layer EIL, the electron injection layer EIL may have a thickness of about 1 Å to about 100 Å, for example, about 3 Å to about 90 Å. If (e.g., when) the thickness of the electron injection layer EIL satisfies the above-described ranges, satisfactory electron injection characteristics may be obtained without a substantial increase in driving voltage.

The second electrode EL2 may be provided on the electron transport region ETR. The second electrode EL2 may be a common electrode. The second electrode EL2 may be a cathode or an anode, but embodiments of the present disclosure are not limited thereto. For example, if (e.g., when) the first electrode EL1 is an anode, the second electrode EL2 may be a cathode, and if (e.g., when) the first electrode EL1 is a cathode, the second electrode EL2 may be an anode.

The second electrode EL2 may be a transmissive electrode, a transflective electrode, or a reflective electrode. When the second electrode EL2 is a transmissive electrode, the second electrode EL2 may be formed of a transparent metal oxide, for example, indium tin oxide (ITO), indium zinc oxide (IZO), zinc oxide (ZnO), indium tin zinc oxide (ITZO), and/or the like.

When the second electrode EL2 is a transflective electrode or a reflective electrode, the second electrode EL2 may include Ag, Mg, Cu, A₁, Pt, Pd, Au, Ni, Nd, Ir, Cr, Li, Ca, LiF/Ca, LiF/Al, Mo, Ti, Yb, W, a compound thereof, or a mixture thereof (e.g., AgMg, AgYb, or MgAg). In one or more embodiments, the second electrode EL2 may have a multilayer structure including a reflective film or a transflective film formed of one or more of the above-described materials, and a transparent conductive film formed of ITO, IZO, ZnO, ITZO, and/or the like. For example, in one or more embodiments, the second electrode EL2 may include one of the above-described metal materials, one or more combinations of at least two metal materials of the above-described metal materials, an oxide of the above-described metal materials, and/or the like.

In one or more embodiments the second electrode EL2 may be connected with an auxiliary electrode. If (e.g., when) the second electrode EL2 is connected with the auxiliary electrode, the resistance of the second electrode EL2 may be decreased.

In one or more embodiments, a capping layer CPL may further be arranged on the second electrode EL2 of the light-emitting element ED of one or more embodiments. The capping layer CPL may include a multilayer or a single layer.

In one or more embodiments, the capping layer CPL may be an organic layer or an inorganic layer. For example, if (e.g., when) the capping layer CPL contains an inorganic material, the inorganic material may include an alkaline metal compound (e.g., LiF), an alkaline earth metal compound (e.g., MgF2), SiON, SiNx, SiOy, and/or the like.

In one or more embodiments, if (e.g., when) the capping layer CPL includes an organic material, the organic material may include 2,2′-dimethyl-N,N′-di-[(1-naphthyl)-N,N′-diphenyl]-1,1′-biphenyl-4,4′-diamine(α-NPD), NPB, TPD, m-MTDATA, Alq₃, CuPc, N4,N4,N4′,N4′-tetra(biphenyl-4-yl)biphenyl-4,4′-diamine (TPD15), 4,4′,4″-tris(carbazol-9-yl)triphenylamine (TCTA), and/or the like, or an epoxy resin, or an acrylate such as methacrylate. However, embodiments of the present disclosure are not limited thereto, for example, the capping layer CPL may include at least one selected from among Compounds P1 to P5.

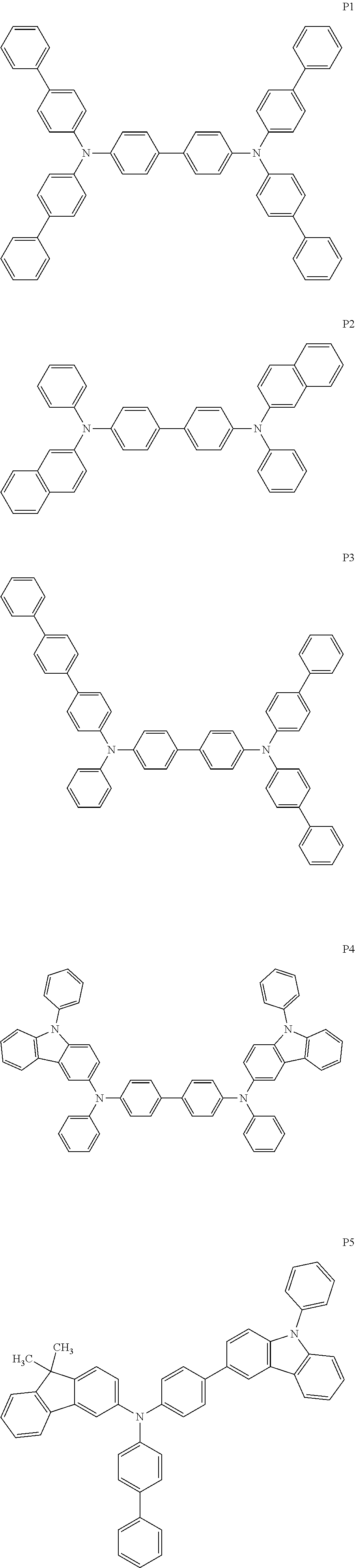 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; CC1(C)c2ccccc2-c2cc(N(c3ccc(-c4ccccc4)cc3)c3ccc(-c4ccc5c(c4)c4ccccc4n5-c4ccccc4)cc3)ccc21]

In one or more embodiments, a refractive index of the capping layer CPL may be about 1.6 or more. For example, in one or more embodiments, the refractive index of the capping layer CPL may be about 1.6 or more with respect to light in a wavelength range of about 550 nm to about 660 nm.

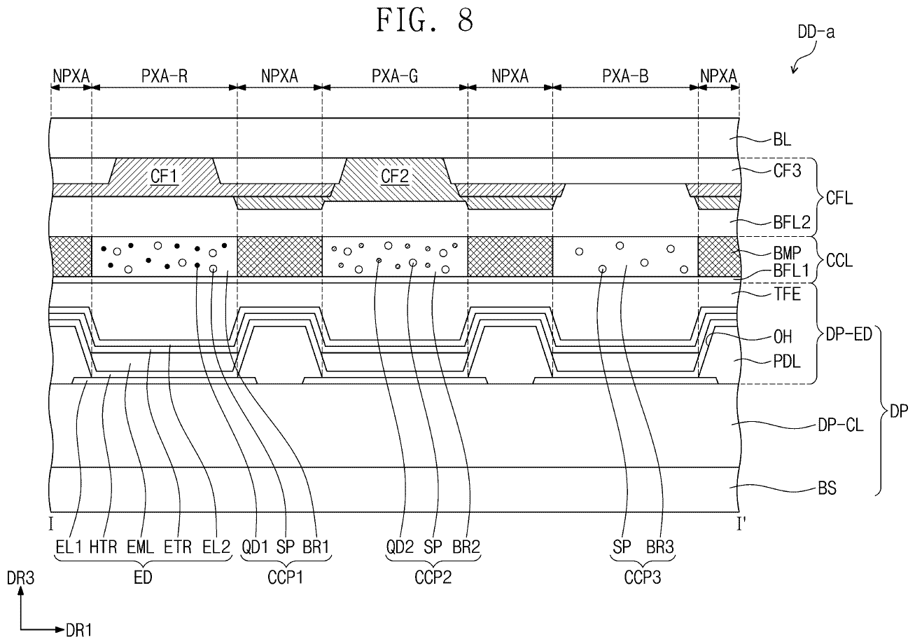

Each of FIGS. 8 to 11 is a cross-sectional view of a display device according to one or more embodiments of the present disclosure. Hereinafter, in describing the display devices of embodiments with reference to FIGS. 8 to 11, the duplicated features which have been described in FIGS. 1 to 7 will not be described again, only their differences will be mainly described.

Referring to FIG. 8, the display device DD-a according to one or more embodiments may include a display panel DP including a display element layer DP-ED, a light control layer CCL arranged on the display panel DP, and a color filter layer CFL. In one or more embodiments, as illustrated in FIG. 8, the display panel DP may include a base layer BS, a circuit layer DP-CL provided on the base layer BS, and the display element layer DP-ED, and the display element layer DP-ED may include a light-emitting element ED.

The light-emitting element ED may include a first electrode EL1, a hole transport region HTR arranged on the first electrode EL1, a light-emitting layer EML arranged on the hole transport region HTR, an electron transport region ETR arranged on the light-emitting layer EML, and a second electrode EL2 arranged on the electron transport region ETR. In one or more embodiments, the structures of the light-emitting elements of FIG. 3 to FIG. 7 described above may be equally applied to the structure of the light-emitting element ED illustrated in FIG. 8.

The light-emitting element ED illustrated in FIG. 8 may include the amine compound according to one or more embodiments in the hole transport region HTR. Therefore, the light-emitting element ED may exhibit characteristics of high efficiency and long lifespan. In addition, the light-emitting element ED according to one or more embodiments may exhibit characteristics of high light efficiency and improved lifespan in a blue light-emitting region. The light-emitting element ED according to one or more embodiments includes the amine compound according to one or more embodiments in the hole transport region HTR, thereby exhibiting characteristics of high efficiency and long lifespan, and thus the display device DD-a according to one or more embodiments may exhibit excellent or suitable display quality.

Referring to FIG. 8, the light-emitting layer EML may be arranged in an opening OH defined in a pixel defining layer PDL. For example, the light-emitting layer EML which is divided by the pixel defining layer PDL and provided corresponding to each of light emitting regions PXA-R, PXA-G, and PXA-B may be to emit light in substantially the same wavelength range. In the display device DD-a of one or more embodiments, the light-emitting layer EML may be to emit blue light. In one or more embodiments, the light-emitting layer EML may be provided as a common layer in the entire light emitting regions PXA-R, PXA-G, and PXA-B.

The light control layer CCL may be arranged on the display panel DP. Although the light control layer CCL is shown as being arranged above (e.g., on) the display element layer DP-ED, embodiments of the present disclosure are not limited to this, for example, in one or more embodiments, the light control layer CCL may be arranged below the display element layer DP-ED. The light control layer CCL may include a light conversion body. The light conversion body may be a quantum dot, a phosphor, and/or the like. The light conversion body may be to emit provided light by converting the wavelength thereof. For example, the light control layer CCL may a layer containing a quantum dot or a layer containing a phosphor.

The light control layer CCL may include a plurality of light control parts CCP1, CCP2, and CCP3. The light control parts CCP1, CCP2, and CCP3 may be spaced and/or apart (e.g., spaced apart or separated) from one another.

Referring to FIG. 8, divided patterns BMP may be arranged between the light control parts CCP1, CCP2, and CCP3 which are spaced and/or apart (e.g., spaced apart or separated) from one another, but embodiments of the present disclosure are not limited thereto. FIG. 8 illustrates that the divided patterns BMP do not overlap the light control parts CCP1, CCP2, and CCP3, but, in one or more embodiments, at least a portion of the edges of the light control parts CCP1, CCP2, and CCP3 may overlap the divided patterns BMP.

The light control layer CCL may include a first light control part CCP1 containing a first quantum dot QD1 which converts first color light provided from the light-emitting element ED into second color light, a second light control part CCP2 containing a second quantum dot QD2 which converts the first color light into third color light, and a third light control part CCP3 which transmits the first color light.

In one or more embodiments, the first light control part CCP1 may provide red light that is the second color light, and the second light control part CCP2 may provide green light that is the third color light. The third light control part CCP3 may provide blue light by transmitting the blue light that is the first color light provided from the light-emitting element ED. For example, in one or more embodiments, the first quantum dot QD1 may be a red quantum dot to emit red light, and the second quantum dot QD2 may be a green quantum dot to emit green light. The same as described above on quantum dots may be applied with respect to the quantum dots QD1 and QD2.

In one or more embodiments, the light control layer CCL may further include a scatterer SP. The first light control part CCP1 may include the first quantum dot QD1 and the scatterer SP, the second light control part CCP2 may include the second quantum dot QD2 and the scatterer SP, and the third light control part CCP3 may not include (e.g., may exclude) any quantum dot but include the scatterer SP.

The scatterer SP may be inorganic particles. For example, the scatterer SP may include at least one of TiO₂, ZnO, Al₂O₃, SiO₂, or hollow sphere silica. In one or more embodiments, the scatterer SP may include any one selected from among TiO₂, ZnO, Al₂O₃, SiO₂, and hollow sphere silica, or may be a mixture of at least two materials selected from among TiO₂, ZnO, Al₂O₃, SiO₂, and hollow sphere silica.

The first light control part CCP1, the second light control part CCP2, and the third light control part CCP3 may respectively include base resins BR1, BR2, and BR3 in which the quantum dots QD1 and QD2 and the scatterer SP are dispersed accordingly. In one or more embodiments, the first light control part CCP1 may include the first quantum dot QD1 and the scatterer SP dispersed in a first base resin BR1, the second light control part CCP2 may include the second quantum dot QD2 and the scatterer SP dispersed in a second base resin BR2, and the third light control part CCP3 may include the scatterer SP dispersed in a third base resin BR3.

The base resins BR1, BR2, and BR3 are media in which the quantum dots QD1 and QD2 and the scatterer SP are dispersed accordingly, and may each be formed of one or more suitable resin compositions, which may be generally referred to as a binder. For example, the base resins BR1, BR2, and BR3 may each independently be an acrylic-based resin, a urethane-based resin, a silicone-based resin, an epoxy-based resin, and/or the like. The base resins BR1, BR2, and BR3 may be transparent resins. In one or more embodiments, the first base resin BR1, the second base resin BR2, and the third base resin BR3 may be the same as or different from one another.

In one or more embodiments, the light control layer CCL may include a barrier layer BFL1. The barrier layer BFL1 may serve to prevent or reduce the penetration of moisture and/or oxygen (hereinafter, referred to as ‘moisture/oxygen’). The barrier layer BFL1 may block the light control parts CCP1, CCP2, and CCP3 from being exposed to moisture/oxygen. In one or more embodiments, the barrier layer BFL1 may cover the light control parts CCP1, CCP2, and CCP3. In one or more embodiments, a barrier layer BFL2 may be provided between the light control parts CCP1, CCP2, and CCP3 and the color filter layer CFL.

The barrier layers BFL1 and BFL2 may each include at least one inorganic layer. For example, in one or more embodiments, the barrier layers BFL1 and BFL2 may each include an inorganic material. For example, the barrier layers BFL1 and BFL2 may each independently include a silicon nitride, an aluminum nitride, a zirconium nitride, a titanium nitride, a hafnium nitride, a tantalum nitride, a silicon oxide, an aluminum oxide, a titanium oxide, a tin oxide, a cerium oxide, a silicon oxynitride, a metal thin film which secures a transmittance, and/or the like. In one or more embodiments, the barrier layers BFL1 and BFL2 may each independently further include an organic film. The barrier layers BFL1 and BFL2 may each independently be formed of a single layer or a plurality of layers.

In the display device DD-a of one or more embodiments, the color filter layer CFL may be arranged on the light control layer CCL. For example, in one or more embodiments, the color filter layer CFL may be directly arranged on the light control layer CCL. In these embodiments, the barrier layer BFL2 may not be provided.

The color filter layer CFL may include color filters CF1, CF2, and CF3. In one or more embodiments, the color filter layer CFL may include a first filter CF1 configured to transmit the second color light, a second filter CF2 configured to transmit the third color light, and a third filter CF3 configured to transmit the first color light. For example, in one or more embodiments, the first filter CF1 may be a red filter, the second filter CF2 may be a green filter, and the third filter CF3 may be a blue filter. The filters CF1, CF2, and CF3 each may include a polymeric photosensitive resin and a pigment and/or a dye. For example, the first filter CF1 may include a red pigment and/or a red dye, the second filter CF2 may include a green pigment and/or a green dye, and the third filter CF3 may include a blue pigment and/or a blue dye.

Embodiments of the present disclosure are not limited thereto, for example, the third filter CF3 may not include (e.g., may exclude) any pigment or any dye. The third filter CF3 may include a polymeric photosensitive resin and may not include (e.g., may exclude) a pigment or a dye. The third filter CF3 may be transparent. The third filter CF3 may be formed of a transparent photosensitive resin.

In one or more embodiments, the first filter CF1 and the second filter CF2 may be a yellow filter. The first filter CF1 and the second filter CF2 may not be separated but be provided as one filter.

In one or more embodiments, the color filter layer CFL may further include a light shielding part. The light shielding part may be a black matrix. The light shielding part may include an organic light shielding material and/or an inorganic light shielding material each containing a black pigment and/or a black dye. The light shielding part may prevent or reduce light leakage, and may separate boundaries between adjacent filters CF1, CF2, and CF3. In one or more embodiments, the light shielding part may be formed of a blue filter.

The first to third filters CF1, CF2, and CF3 may be arranged corresponding to the red light emitting region PXA-R, the green light emitting region PXA-G, and the blue light emitting region PXA-B, respectively.

A base substrate BL may be arranged on the color filter layer CFL. The base substrate BL may be a member which provides a base surface on which the color filter layer CFL, the light control layer CCL, and/or the like are arranged. The base substrate BL may be a glass substrate, a metal substrate, a plastic substrate, and/or the like. However, embodiments of the present disclosure are not limited thereto, and the base substrate BL may be an inorganic layer, an organic layer, or a composite material layer. In one or more embodiments, the base substrate BL may not be provided.

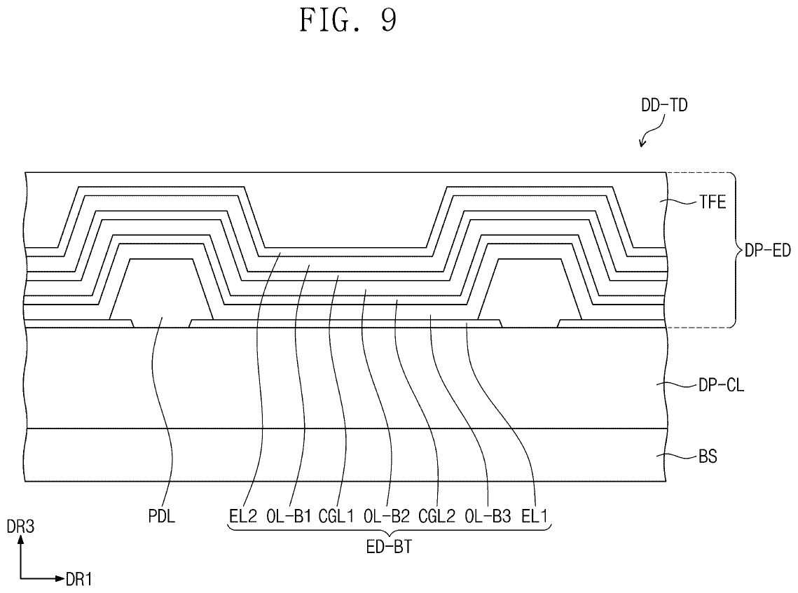

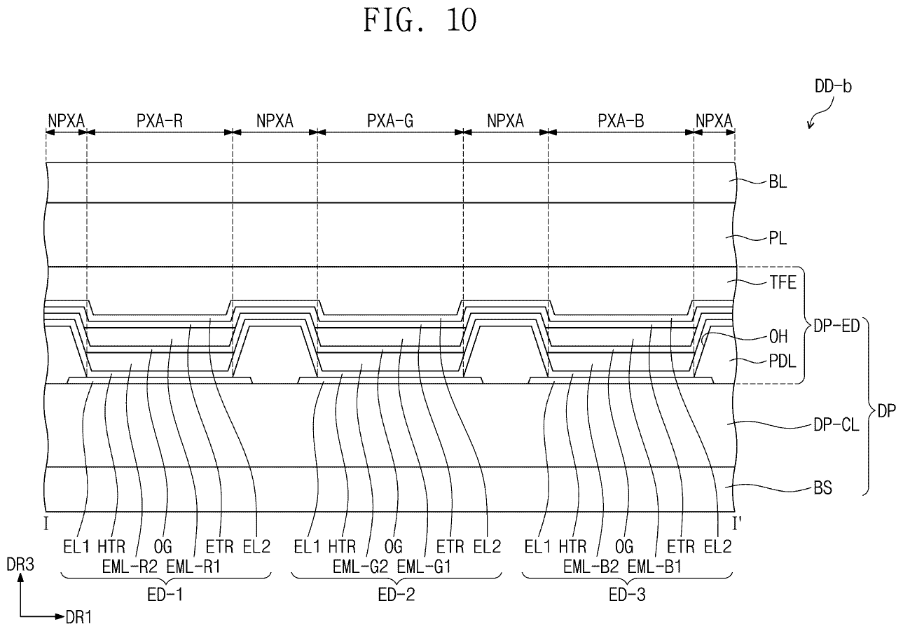

FIG. 9 is a cross-sectional view illustrating a portion of a display device according to one or more embodiments of the present disclosure. In a display device DD-TD according to one or more embodiments, a light-emitting element ED-BT may include a plurality of light-emitting structures OL-B1, OL-B2, and OL-B3.

At least one selected from among the plurality of emission structures OL-B1, OL-B2, and OL-B3 may include the amine compound according to one or more embodiments. Therefore, the light-emitting element ED-BT may exhibit characteristics of high efficiency and long lifespan.

The light-emitting element ED-BT may include a first electrode EL1 and a second electrode EL2 which face each other, and the plurality of light emitting structures OL-B1, OL-B2, and OL-B3 sequentially stacked in a thickness direction between the first electrode EL1 and the second electrode EL2. The light emitting structures OL-B1, OL-B2, and OL-B3 each may include a light-emitting layer EML (FIG. 8) and a hole transport region HTR and an electron transport region ETR arranged with the light-emitting layer EML (FIG. 8) located therebetween. For example, the light-emitting element ED-BT included in the display device DD-TD of one or more embodiments may be a light-emitting element having a tandem structure and including a plurality of light-emitting layers.

In one or more embodiments illustrated in FIG. 9, all light beams respectively emitted from the light emitting structures OL-B1, OL-B2, and OL-B3 may be blue light. However, embodiments of the present disclosure are not limited thereto, for example, in one or more embodiments, the light beams respectively emitted from the light emitting structures OL-B1, OL-B2, and OL-B3 may have wavelength ranges different from one another. For example, in one or more embodiments, the light-emitting element ED-BT including the plurality of light emitting structures OL-B1, OL-B2, and OL-B3 which emit light beams having wavelength ranges different from one another may be to emit white light.

Charge generation layers CGL1 and CGL2 may be respectively arranged between neighboring light-emitting structures OL-B1, OL-B2 and OL-B3. The charge generation layers CGL1 and CGL2 may include a p-type (kind) charge (e.g., P-charge) generation layer and/or an n-type (kind) charge (e.g., N-charge) generation layer.

In one or more embodiments illustrated in FIG. 9, at least one selected from among the emission structures OL-B1, OL-B2, and OL-B3 may include the amine compound according to one or more embodiments in the hole transport region HTR (FIG. 8). Therefore, the at least one selected from among the emission structures OL-B1, OL-B2, and OL-B3 may exhibit characteristics of high efficiency and long lifespan.

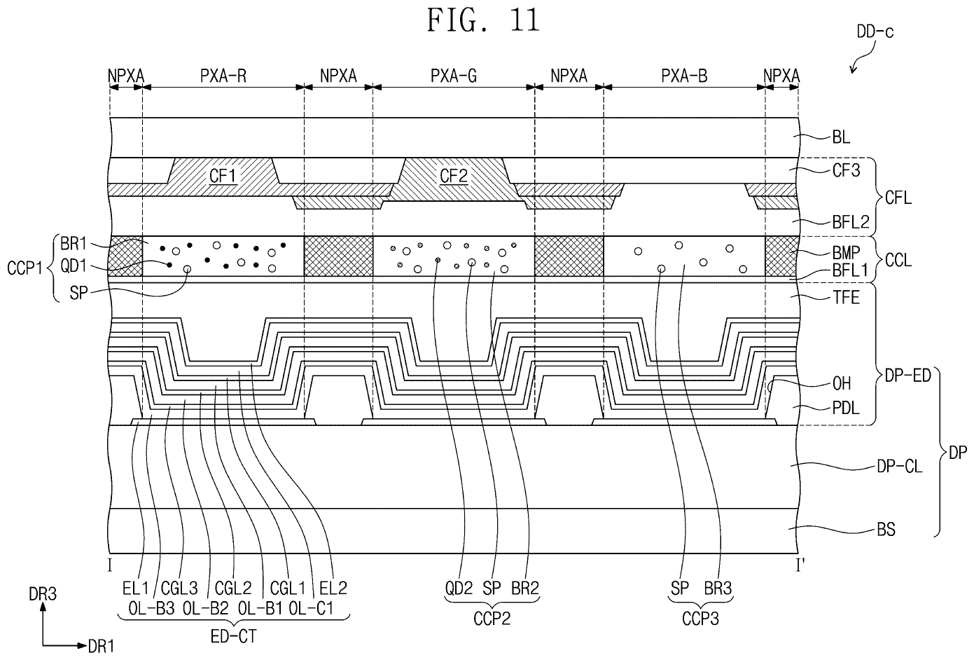

Referring to FIG. 10, a display device DD-b according to one or more embodiments may include the light-emitting elements ED-1, ED-2, and ED-3, in each of which two light-emitting layers are stacked. At least one selected from among the light-emitting elements ED-1, ED-2, and ED-3 may include the amine compound according to one or more embodiments. Therefore, the light-emitting elements ED-1, ED-2, and ED-3 may exhibit characteristics of high efficiency and long lifespan. For example, the light-emitting element ED-3 according to one or more embodiments may exhibit characteristics of high efficiency and improved lifespan in a blue light-emitting region, and the light-emitting element ED-2 may exhibit characteristics of high efficiency and improved lifespan in a green light-emitting region.

Compared to the display device DD of one or more embodiments illustrated in FIG. 2, the display devise DD-b illustrated in FIG. 10 has a difference in that the first to third light-emitting elements ED-1, ED-2, and ED-3 each include two light-emitting layers stacked in a thickness direction. In each of the first to third light-emitting elements ED-1, ED-2, and ED-3, the two light-emitting layers may be to emit light in substantially the same wavelength region.

In one or more embodiments, the first light-emitting element ED-1 may include a first red light-emitting layer EML-R1 and a second red light-emitting layer EML-R2. The second light-emitting element ED-2 may include a first green light-emitting layer EML-G1 and a second green light-emitting layer EML-G2. In addition, the third light-emitting element ED-3 may include a first blue light-emitting layer EML-B1 and a second blue light-emitting layer EML-B2. An emission auxiliary part OG may be arranged between the first red light-emitting layer EML-R1 and the second red light-emitting layer EML-R2, between the first green light-emitting layer EML-G1 and the second green light-emitting layer EML-G2, and between the first blue light-emitting layer EML-B1 and the second blue light-emitting layer EML-B2.

The emission auxiliary part OG may include a single layer or a multilayer. The emission auxiliary part OG may include a charge generation layer. For example, in one or more embodiments, the emission auxiliary part OG may include an electron transport region, a charge generation layer, and a hole transport region that are sequentially stacked (e.g., in the stated order). The emission auxiliary part OG may be provided as a common layer across the whole of the first to third light-emitting elements ED-1, ED-2, and ED-3. However, embodiments of the present disclosure are not limited thereto, and the emission auxiliary part OG may be provided by being patterned within the openings OH defined in the pixel defining layer PDL.

The first red light-emitting layer EML-R1, the first green light-emitting layer EML-G1, and the first blue light-emitting layer EML-B1 may each be arranged between the electron transport region ETR and the emission auxiliary part OG. The second red light-emitting layer EML-R2, the second green light-emitting layer EML-G2, and the second blue light-emitting layer EML-B2 may each be arranged between the emission auxiliary part OG and the hole transport region HTR.

For example, in one or more embodiments, the first light-emitting element ED-1 may include a first electrode EL1, a hole transport region HTR, a second red light-emitting layer EML-R2, an emission auxiliary part OG, a first red light-emitting layer EML-R1, an electron transport region ETR, and a second electrode EL2 that are sequentially stacked (e.g., in the stated order). The second light-emitting element ED-2 may include a first electrode EL1, a hole transport region HTR, a second green light-emitting layer EML-G2, an emission auxiliary part OG, a first green light-emitting layer EML-G1, an electron transport region ETR, and a second electrode EL2 that are sequentially stacked (e.g., in the stated order). The third light-emitting element ED-3 may include a first electrode EL1, a hole transport region HTR, a second blue light-emitting layer EML-B2, an emission auxiliary part OG, a first blue light-emitting layer EML-B1, an electron transport region ETR, and a second electrode EL2 that are sequentially stacked (e.g., in the stated order).

The light-emitting elements ED-1, ED-2, and ED-3 include the amine compound according to one or more embodiments in the hole transport region HTR, thereby exhibiting characteristics of high efficiency and long lifespan, and thus the display device DD-b according to one or more embodiments may exhibit excellent or suitable display quality.

In one or more embodiments, an optical auxiliary layer PL may be arranged on the display element layer DP-ED. The optical auxiliary layer PL may include a polarizing layer. The optical auxiliary layer PL may be arranged on the display panel DP and control reflected light in the display panel DP due to external light. In one or more embodiments, the optical auxiliary layer PL in the display device may not be provided.

Different from FIG. 9 and FIG. 10, a display device DD-c of FIG. 11 is illustrated to include four light emitting structures OL-B1, OL-B2, OL-B3, and OL-C1. A light-emitting element ED-CT may include a first electrode EL1 and a second electrode EL2 opposite to (e.g., facing) each other, and the first to fourth light emitting structures OL-B1, OL-B2, OL-B3, and OL-C1 stacked in a thickness direction between the first electrode EL1 and the second electrode EL2.

At least selected from among first to fourth emission structures OL-B1, OL-B2, OL-B3, and OL-C1 may include the amine compound according to one or more embodiments. Therefore, the light-emitting element ED-CT may exhibit characteristics of high efficiency and long lifespan. In addition, the display device DD-c according to one or more embodiments may exhibit excellent or suitable display quality.

Charge generation layers CGL1, CGL2, and CGL3 may be separately arranged between the first to fourth light emitting structures OL-B1, OL-B2, OL-B3, and OL-C1. In one or more embodiments, among the four light emitting structures, the first to third light emitting structures OL-B1, OL-B2, and OL-B3 may be to emit blue light, and the fourth light emitting structure OL-C1 may be to emit green light. However, embodiments of the present disclosure are not limited thereto, for example, the first to fourth light emitting structures OL-B1, OL-B2, OL-B3, and OL-C1 may each be to emit light beams in different wavelength regions. The charge generation layers CGL1, CGL2, and CGL3 arranged between adjacent light emitting structures OL-B1, OL-B2, OL-B3, and OL-C1 may include a p-type (kind) charge (e.g., P-charge) generation layer and/or an n-type (kind) charge (e.g., N-charge) generation layer.

In one or more embodiments, an electronic apparatus may include the display device including a plurality of light-emitting elements, and a control part which controls the display device. The electronic apparatus of one or more embodiments may be a device that is activated according to an electrical signal. The electronic apparatus may include display device of one or more suitable embodiments. For example, the electronic apparatus may include not only large-sized electronic apparatuses such as a television set, a monitor, or an outdoor billboard but also include small- and medium-sized electronic apparatuses such as a personal computer, a laptop computer, a personal digital terminal, a display device for a vehicle, a game console, a portable electronic device, or a camera. In one or more embodiments, the electronic apparatus may include at least of a flat panel display, a curved display, a computer monitor, a medical monitor, a television, a billboard, an indoor light, an outdoor light, a light for signaling, a head-up display, a fully transparent display, a partially transparent display, a flexible display, a rollable display, a foldable display, a stretchable display, a laser printer, a telephone, a portable phone, a tablet personal computer, a phablet, a personal digital assistant, a wearable device, a laptop computer, a digital camera, a camcorder, a viewfinder, a micro display, a three-dimension (3D) display, a virtual reality display, an augmented reality display, a vehicle, a video wall with multiple displays tiled together, a theater screen, a stadium screen, a phototherapy device, or a signboard.

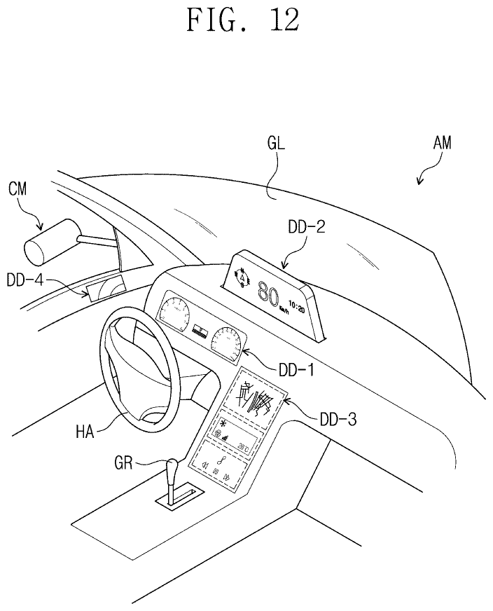

FIG. 12 is a view illustrating a vehicle AM in which first to fourth display devices DD-1, DD-2, DD-3, and DD-4 are arranged. According to one or more embodiments, at least one selected from among the first to fourth display devices DD-1, DD-2, DD-3, and DD-4 may include the same configuration as one of display devices DD, DD-TD, DD-a, DD-b, and DD-c described with reference to FIGS. 1, 2, and 8 to 11.

FIG. 12 illustrates a vehicle AM, but this is a mere example, and the first to fourth display devices DD-1, DD-2, DD-3, and DD-4 may be arranged in other transportation apparatuses such as bicycles, motorcycles, trains, ships, and/or airplanes. In addition, at least one selected from among the first to fourth display devices DD-1, DD-2, DD-3, and DD-4 including the same configuration as one of the display devices DD, DD-TD, DD-a, DD-b, and DD-c of one or more embodiments may be employed in a personal computer, a laptop computer, a personal digital terminal, a game console, a portable electronic device, a television, a monitor, an outdoor billboard, and/or the like. These are merely provided as embodiments, and these display devices may be employed in other electronic apparatuses unless departing from the disclosure.

In one or more embodiments, at least one selected from among first to fourth display devices DD-1, DD-2, DD-3, and DD-4 may include the light-emitting element ED described with reference to FIG. 3 to FIG. 7. The at least one selected from among the first to fourth display devices DD-1, DD-2, DD-3, and DD-4 may include the amine compound according to one or more embodiments. Therefore, the first to fourth display devices DD-1, DD-2, DD-3, and DD-4 which includes the amine compound according to one or more embodiments may have improved display efficiency and display lifespan. In addition, the first to fourth display devices DD-1, DD-2, DD-3, and DD-4 that includes the amine compound according to one or more embodiments may exhibit excellent or suitable display quality.

Referring to FIG. 12, the vehicle AM may include a steering wheel HA and a gear GR for driving the vehicle AM. In addition, the vehicle AM may include a front window GL arranged so as to face a driver.

A first display device DD-1 may be arranged in a first region overlapping with the steering wheel HA. For example, the first display device DD-1 may be a digital cluster which displays first information of the vehicle AM. The first information may include a first scale which indicates a driving speed of the vehicle AM, a second scale which indicates an engine speed (that is, revolutions per minute (RPM)), an image which indicates a fuel state, and/or the like. The first scale and the second scale may each be indicated as a digital image.

A second display device DD-2 may be arranged in a second region opposite to (e.g., facing) a driver seat and overlapping with the front window GL. The driver seat may be a seat in which the steering wheel HA faces. For example, the second display device DD-2 may be a head up display (HUD) which displays second information of the vehicle AM. The second display device DD-2 may be optically transparent. The second information may include digital numbers which indicate a driving speed, and may further include information such as the current time. In one or more embodiments, the second information of the second display device DD-2 may be projected to and displayed on the front window GL.

A third display device DD-3 may be arranged in a third region adjacent to the gear GR. For example, the third display device DD-3 may be arranged between the driver seat and a passenger seat and may be a center information display (CID) for the vehicle for displaying third information. The passenger seat may be a seat spaced and/or apart (e.g., spaced apart or separated) from the driver seat with the gear GR arranged therebetween. The third information may include information about traffic (e.g., navigation information), playing music or radio or a video (or an image), temperatures inside the vehicle AM, and/or the like.

A fourth display device DD-4 may be spaced and/or apart (e.g., spaced apart or separated) from the steering wheel HA and the gear GR, and may be arranged in a fourth region adjacent to a side of the vehicle AM. For example, the fourth display device DD-4 may be a digital side-view mirror which displays fourth information. The fourth display device DD-4 may display an image outside the vehicle AM taken by a camera module CM arranged outside the vehicle AM. The fourth information may include an image outside the vehicle AM.

The above-described first to fourth information may be examples, and the first to fourth display devices DD-1, DD-2, DD-3, and DD-4 may further display information about the inside and outside of the vehicle AM. The first to fourth information may include different information. However, embodiments of the present disclosure are not limited thereto, for example, a part of the first to fourth information may include the same information as one another.

Hereinafter, referring to Examples and Comparative Examples, the amine compound according to one or more embodiments of the present disclosure and the light-emitting element of one or more embodiments will be described in more detail. In addition, Examples shown herein are for explanatory purposes only to facilitate the understanding of the present disclosure, and thus, the scope of the disclosure is not limited thereto.

### Examples

### 1. Synthesis of Amine Compound According to One or More Embodiments

Synthetic methods of the amine compounds according to one or more embodiments will be explained by exemplifying synthetic methods of Compounds A24, B2, B3, B11, C1, C7, C15, C29, D2, and E14. In addition, the synthetic methods of the amine compounds which will be explained hereinafter are examples, and the synthetic method of the amine compound according to one or more embodiments of the present disclosure is not limited to Examples described herein.

### (1) Synthesis of Compound A24

Amine Compound A24 according to one or more embodiments may be synthesized according to, for example, Reaction Scheme 1.

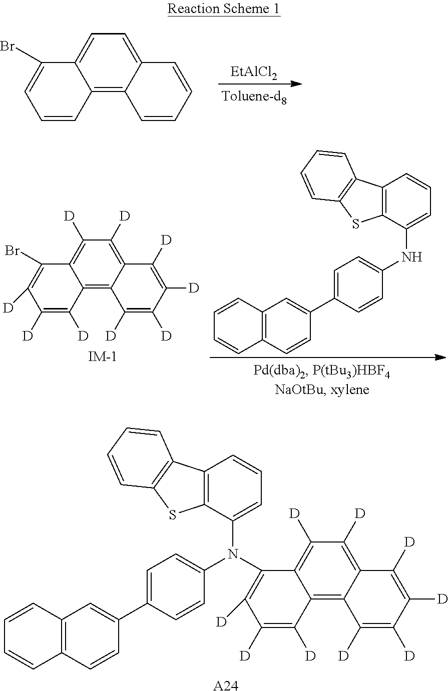

### Synthesis of Intermediate IM-1

Under an argon (Ar) atmosphere, 1-bromophenanthrene 20.00 g (77.78 mmol) and toluene-d₈ 156 mL were added to a 500 mL three-neck flask, the flask was cooled in an ice-bath, ethylaluminum dichloride (EtAlCl₂) in hexane (ca. 1 M) of 7.78 mL (0.1 equiv., 7.78 mmol) was added dropwise thereto and the mixture was stirred. The mixture was stirred for 1 hour while cooling with ice, then the reaction solution was quenched with 3 N hydrochloric acid, and water was added to separate an organic layer. The organic layer was sequentially washed with a saturated aqueous sodium hydrogen carbonate and a saturated saline solution, and then was dried over anhydrous MgSO₄. A crude product obtained by performing filtration of MgSO₄ and concentration of the organic layer was purified to obtain Intermediate IM-1 (15.7 g, yield of 76%, deuteration rate of 90.5%).

By Fast atom bombardment-Mass spectroscopy (FAB-MS) measurement of the obtained product, a molecular ion peak was observed at the mass number of m/z=265, and thus the obtained product was identified as Intermediate IM-1.

### Synthesis of Compound A24

Under an argon atmosphere, Intermediate IM-1 8.00 g (30.1 mmol), bis(dibenzylideneacetone)palladium(0) (Pd(dba)₂) 0.86 g (0.05 equiv., 1.5 mmol), NaO^{t}Bu 3.47 g (1.2 equiv., 36.1 mmol), xylene 300 mL, N-(4-(naphthalen-2-yl)phenyl)dibenzo[b,d]thiophen-4-amine 12.07 g (1.0 equiv., 30.05 mmol), and P(^{t}Bu₃)HBF₄ 1.74 g (0.2 equiv., 6.01 mmol) were sequentially added to a 1000 mL three-neck flask, and were heated, refluxed, and stirred. After air cooling to a room temperature, the reaction solution was filtered through Florisil, and the filtrate was concentrated. The obtained crude product was purified to obtain Compound A24 (11.8 g, yield of 67%, deuteration rate of 25%).

By FAB-MS measurement of the obtained product, a molecular ion peak was observed at the mass number of m/z=586, and thus the obtained product was identified as Compound A-24.

### (2) Synthesis of Compound B2

Amine Compound B2 according to one or more embodiments may be synthesized according to, for example, Reaction Scheme 2.

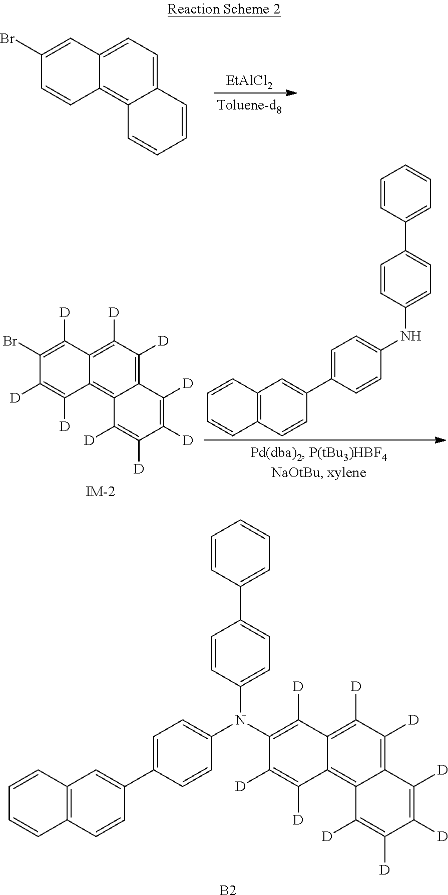

### Synthesis of Intermediate IM-2

Under an argon atmosphere, 2-bromophenanthrene 30.00 g (116.7 mmol) and toluene-d₈ 233 mL were added to a 500 mL three-neck flask, the flask was cooled in an ice-bath, EtAlCl₂ in hexane (ca. 1 M) of 11.67 mL (0.1 equiv., 11.67 mmol) was added dropwise thereto and the mixture was stirred. The mixture was stirred for 1 hour while cooling with ice, then the reaction solution was quenched with 3 N hydrochloric acid, and water was added to separate an organic layer. The organic layer was sequentially washed with a saturated aqueous sodium hydrogen carbonate and a saturated saline solution, and then was dried over anhydrous MgSO₄. A crude product obtained by performing filtration of MgSO₄ and concentration of the organic layer was purified to obtain Intermediate IM-2 (27.2 g, yield of 88%, deuteration rate of 90.1%).

By FAB-MS measurement of the obtained product, a molecular ion peak was observed at the mass number of m/z=265, and thus the obtained product was identified as Intermediate IM-2.

### Synthesis of Compound B2

Under an argon atmosphere, Intermediate IM-2 5.00 g (18.8 mmol), Pd(dba)₂ 0.54 g (0.05 equiv., 0.94 mmol), NaO^{t}Bu 2.17 g (1.2 equiv., 22.5 mmol), xylene 188 mL, N-(4-(naphthalen-2-yl)phenyl)-[1,1′-biphenyl]-4-amine 6.98 g (1.0 equiv., 18.8 mmol), and P(^{t}Bu₃)HBF₄ 1.09 g (0.2 equiv., 3.76 mmol) were sequentially added to a 500 mL three-neck flask, and were heated, refluxed, and stirred. After air cooling to a room temperature, the reaction solution was filtered through Florisil, and the filtrate was concentrated. An obtained crude product was purified to obtain Compound B2 (8.7 g, yield of 83%, deuteration rate of 24%).

By FAB-MS measurement of the obtained product, a molecular ion peak was observed at the mass number of m/z=556, and thus the obtained product was identified as Compound B2.

### (3) Synthesis of Compound B3

Amine Compound B3 according to one or more embodiments may be synthesized according to, for example, Reaction Scheme 3.

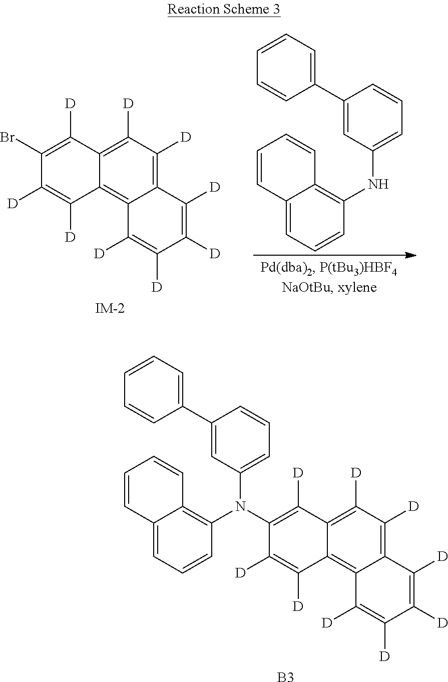

Under an argon atmosphere, Intermediate IM-2 5.00 g (18.8 mmol), Pd(dba)₂ 0.54 g (0.05 equiv., 0.94 mmol), NaOtBu 2.17 g (1.2 equiv., 22.5 mmol), xylene 188 mL, N-([1,1′-biphenyl]-3-yl)naphthalen-1-amine 5.55 g (1.0 equiv., 18.8 mmol), and P(^{t}Bu₃)HBF₄ 1.09 g (0.2 equiv., 3.76 mmol) were sequentially added to a 500 mL three-neck flask, and were heated, refluxed, and stirred. After air cooling to a room temperature, the reaction solution was filtered through Florisil, and the filtrate was concentrated. An obtained crude product was purified to obtain Compound B3 (6.7 g, yield of 74%, deuteration rate of 28%).

By FAB-MS measurement of the obtained product, a molecular ion peak was observed at the mass number of m/z=480, and thus the obtained product was identified as Compound B3.

### (4) Synthesis of Compound B11

Amine Compound B11 according to one or more embodiments may be synthesized according to, for example, Reaction Scheme 4.

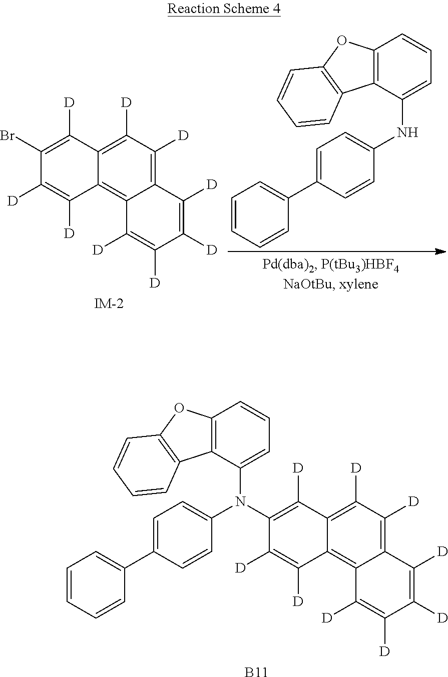

Under an argon atmosphere, Intermediate IM-2 6.00 g (22.5 mmol), Pd(dba)₂ 0.65 g (0.05 equiv., 1.13 mmol), NaOtBu 2.60 g (1.2 equiv., 27.1 mmol), xylene 225 mL, N-([1,1′-biphenyl]-4-yl)dibenzo[b,d]furan-1-amine 7.56 g (1.0 equiv., 22.5 mmol), and P(^{t}Bu₃)HBF₄ 1.31 g (0.2 equiv., 4.51 mmol) were sequentially added to a 500 mL three-neck flask, and were heated, refluxed, and stirred. After air cooling to a room temperature, the reaction solution was filtered through Florisil, and the filtrate was concentrated. An obtained crude product was purified to obtain Compound B11 (9.0 g, yield of 77%, deuteration rate of 27%).

By FAB-MS measurement of the obtained product, a molecular ion peak was observed at the mass number of m/z=520, and thus the obtained product was identified as Compound B11.

### (5) Synthesis of Compound C1

Amine Compound C1 according to one or more embodiments may be synthesized according to, for example, Reaction Scheme 5.

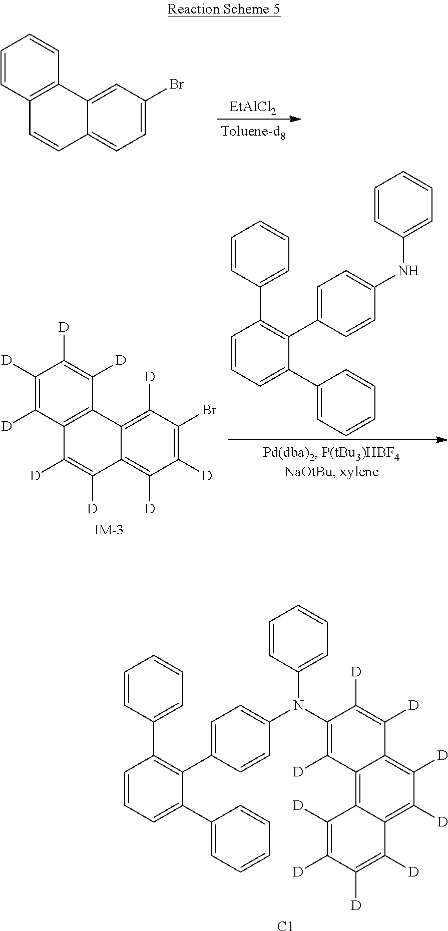

### Synthesis of Intermediate IM-3

Under an argon atmosphere, 3-bromophenanthrene 32.00 g (124.5 mmol) toluene-d₈ 249 mL were added to a 1000 mL three-neck flask, the flask was cooled in an ice-bath, EtAlCl₂ in hexane (ca. 1 M) of 12.45 mL (0.1 equiv., 12.45 mmol) was added dropwise thereto and the mixture was stirred. The mixture was stirred for 1 hour while cooling with ice, then the reaction solution was quenched with 3 N hydrochloric acid, and water was added to separate an organic layer. The organic layer was sequentially washed with a saturated aqueous sodium hydrogen carbonate and a saturated saline solution, and then was dried over anhydrous MgSO₄. The crude product obtained by performing filtration of MgSO₄ and concentration of the organic layer was purified to obtain Intermediate IM-3 (17.8 g, yield of 54%, deuteration rate of 90.9%).

By FAB-MS measurement of the obtained product, a molecular ion peak was observed at the mass number of m/z=265, and thus the obtained product was identified as Intermediate IM-3.

### Synthesis of Compound C1

Under an argon atmosphere, Intermediate IM-3 5.00 g (18.8 mmol), Pd(dba)₂ 0.54 g (0.05 equiv., 0.94 mmol), NaOtBu 2.17 g (1.2 equiv., 22.5 mmol), xylene 188 mL, N,6′-diphenyl-[1,1′:2′,1″-terphenyl]-4-amine 7.47 g (1.0 equiv., 18.8 mmol), and P(^{t}Bu₃)HBF₄ 1.09 g (0.2 equiv., 3.76 mmol) were sequentially added to a 500 mL three-neck flask, and were heated, refluxed, and stirred. After air cooling to a room temperature, the reaction solution was filtered through Florisil, and the filtrate was concentrated. The obtained crude product was purified to obtain Compound C1 (7.5 g, yield of 69%, deuteration rate of 22%).

By FAB-MS measurement of the obtained product, a molecular ion peak was observed at the mass number of m/z=582, and thus the obtained product was identified as Compound C1.

### (6) Synthesis of Compound C7

Amine Compound C7 according to one or more embodiments may be synthesized according to, for example, Reaction Scheme 6.

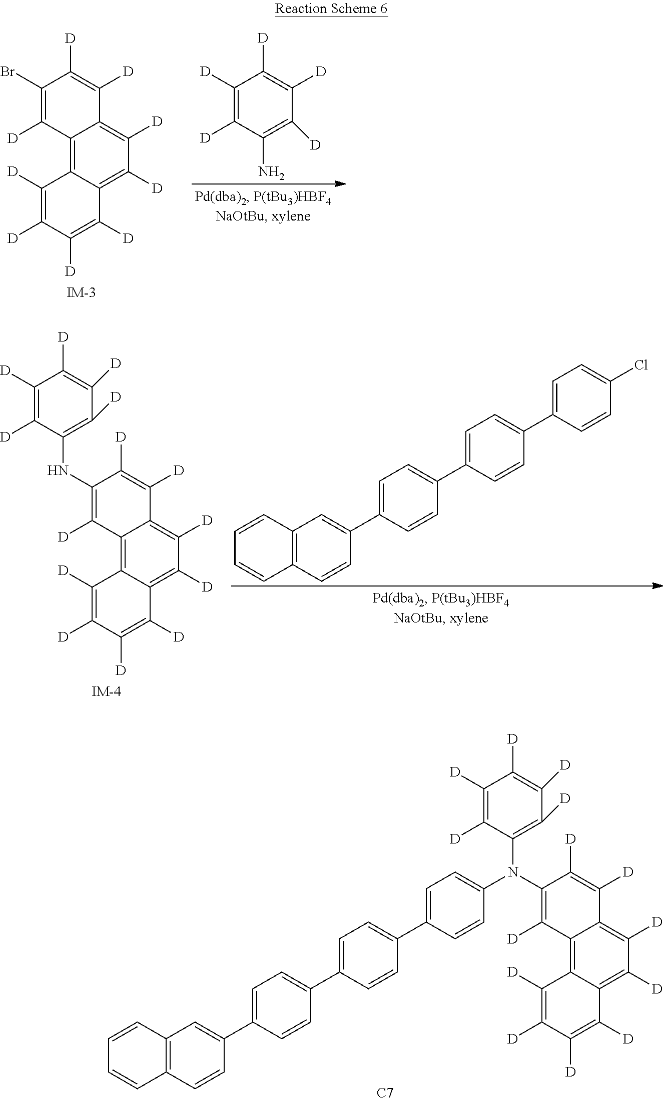

### Synthesis of Intermediate IM-4

Under an argon atmosphere, Intermediate IM-3 6.00 g (22.5 mmol), Pd(dba)₂ 0.65 g (0.05 equiv., 1.13 mmol), NaO^{t}Bu 2.60 g (1.2 equiv., 27.1 mmol), xylene 225 mL, benzen-d₅-amine 2.21 g (1.0 equiv., 22.5 mmol), and P(^{t}Bu₃)HBF₄ 1.31 g (0.2 equiv., 4.51 mmol) were added to a 500 mL three-neck flask, and were heated, refluxed, and stirred. After air cooling to a room temperature, the reaction solution was filtered through Florisil, and the filtrate was concentrated. The obtained crude product was purified to obtain Intermediate IM-4 (5.6 g, yield of 88%).

By FAB-MS measurement of the obtained product, a molecular ion peak was observed at the mass number of m/z=283, and thus the obtained product was identified as Intermediate IM-4.

### Synthesis of Compound C7

Under an argon atmosphere, Intermediate IM-4 5.6 g (20 mmol), Pd(dba)₂ 0.57 g (0.05 equiv., 0.99 mmol), NaOtBu 2.28 g (1.2 equiv., 23.7 mmol), xylene 198 mL, 2-(4″-chloro-[1,1′:4′,1″-terphenyl]-4-yl)naphthalene 7.72 g (1.0 equiv., 19.8 mmol), and P(^{t}Bu₃)HBF₄ 1.15 g (0.2 equiv., 3.95 mmol) were added to a 500 mL three-neck flask, and were heated, refluxed, and stirred. After air cooling to a room temperature, the reaction solution was filtered through Florisil, and the filtrate was concentrated. The obtained crude product was purified to obtain Compound C7 (9.1 g, yield of 72%, deuteration rate of 35%).

### (7) Synthesis of Compound C15

Amine Compound C15 according to one or more embodiments may be synthesized according to, for example, Reaction Scheme 7.

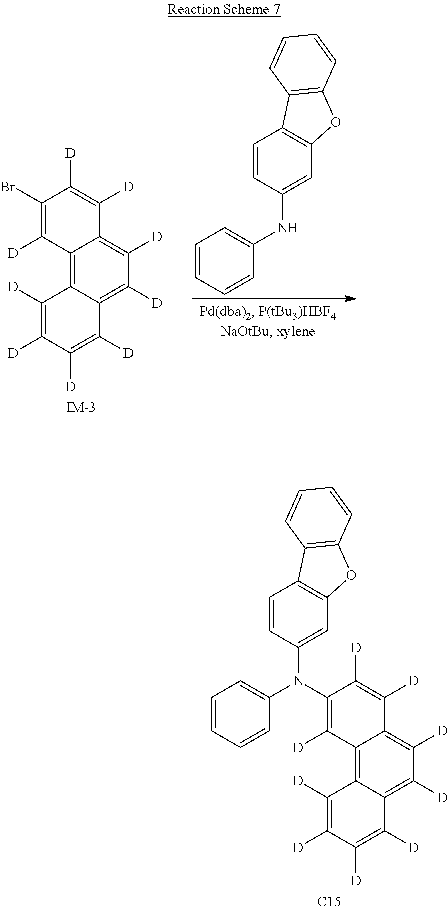

Under an argon atmosphere, Intermediate IM-3 5.00 g (18.8 mmol), Pd(dba)₂ 0.54 g (0.05 equiv., 0.94 mmol), NaOtBu 2.17 g (1.2 equiv., 22.5 mmol), xylene 188 mL, N-phenyldibenzo[b,d]furan-3-amine 4.87 g (1.0 equiv., 18.8 mmol), and P(^{t}Bu₃)HBF₄ 1.09 g (0.2 equiv., 3.76 mmol) were sequentially added to a 500 mL three-neck flask, and were heated, refluxed, and stirred. After air cooling to room temperature, the reaction solution was filtered through Florisil, and the filtrate was concentrated. The obtained crude product was purified to obtain Compound C15 (6.91 g, yield of 83%, deuteration rate of 35%).

By FAB-MS measurement of the obtained product, a molecular ion peak was observed at the mass number of m/z=444, and thus the obtained product was identified as Compound C15.

### (8) Synthesis of Compound C29

Amine Compound C29 according to one or more embodiments may be synthesized according to, for example, Reaction Scheme 8.

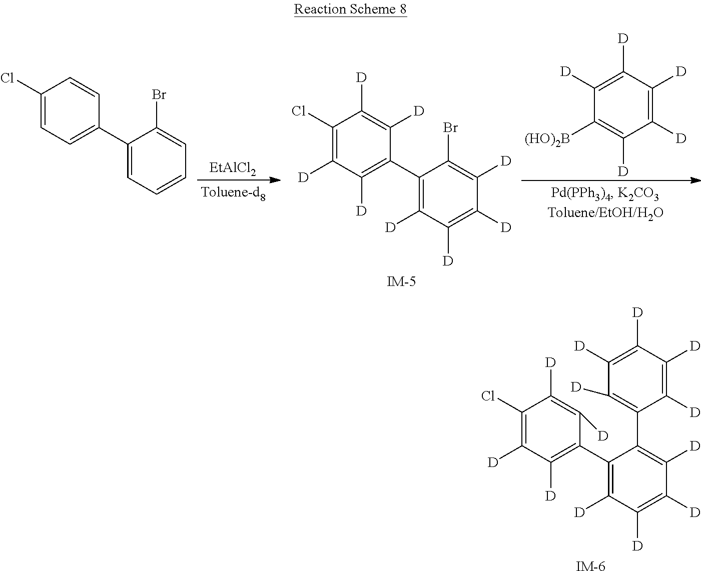

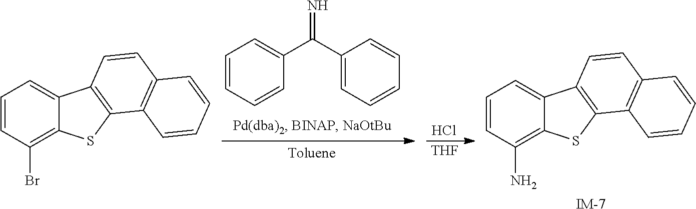

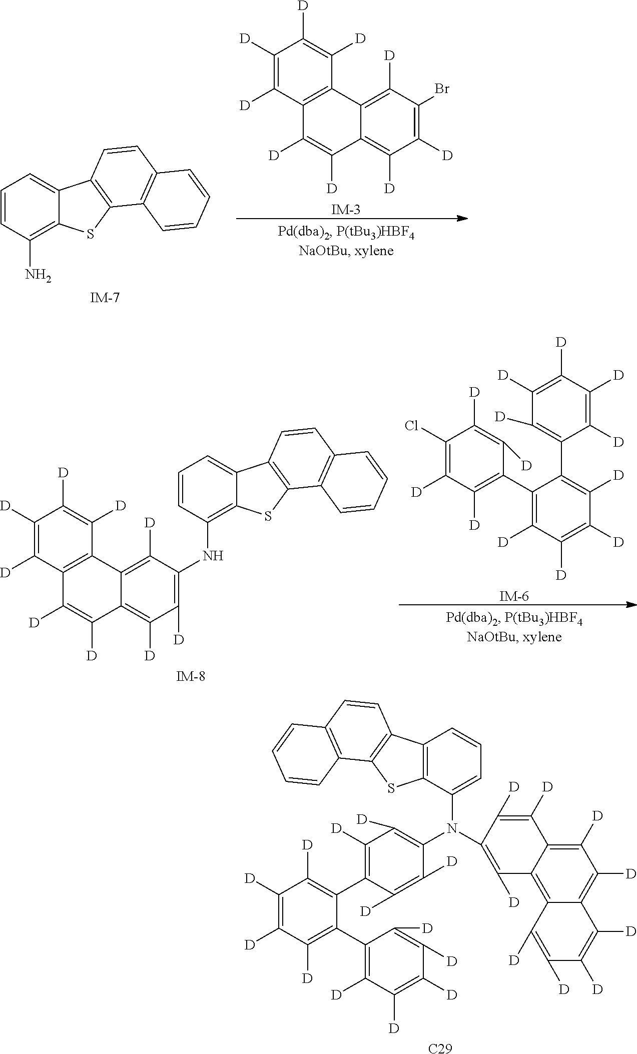

### Synthesis of Intermediate IM-5

Under an argon atmosphere, 2-bromo-4′-chloro-1,1′-biphenyl 11.00 g (41.11 mmol) and toluene-d₈ 82 mL were added to a 300 mL three-neck flask, the flask was cooled in ice, then EtAlCl₂ in hexane (ca. 1 M) of 4.11 mL (0.1 equiv., 4.11 mmol) was added dropwise thereto, and the mixture was additionally stirred. The mixture was checked for 1 hour while cooling with ice, then the reaction solution was quenched with 3 N hydrochloric acid, and water was added to separate an organic layer. The organic layer was sequentially washed with a saturated aqueous sodium hydrogen carbonate and a saturated saline solution, and then was dried over anhydrous MgSO₄. The filtration of MgSO₄ and concentration of the organic layer were performed, and the obtained crude product was purified to obtain Intermediate IM-5 of 12 g, which is a mixture with purities.

### Synthesis of Intermediate IM-6

Under an argon atmosphere, Intermediate IM-5 12 g, phenyl-d₅-boronic acid 13.58 g (107.0 mmol), Pd(PPh₃)₄ 9.51 g (8.23 mmol), K₂CO₃ 34.12 g (246.9 mmol), toluene 165 mL, ethanol (EtOH) 82 mL, H₂O 41 mL were sequentially added to a 1000 mL three-neck flask, and were heated, refluxed, and stirred. Then, after cooling to a room temperature, water and toluene were added to the reaction solvent to separate an organic layer. The organic layer was washed with a saline solution, and then was dried over anhydrous MgSO₄. The crude product obtained by performing filtration of MgSO₄ and concentration of the organic layer was purified to obtain Intermediate IM-6 (7.6 g, yield of 67%, deuteration rate of 94.9%).

By FAB-MS measurement of the obtained product, a molecular ion peak was observed at the mass number of m/z=277, and thus the obtained product was identified as Intermediate IM-6.

### Synthesis of Intermediate IM-7

Under an argon atmosphere, 10-bromobenzo[b]naphtho[2,1-d]thiophene 40.00 g (127.7 mmol), Pd(dba)₂ 0.73 g (0.01 equiv., 1.3 mmol), 2,2′-bis(diphenylphosphino)-1,1′-binaphthyl (BINAP) 1.59 g (0.02 equiv., 2.55 mmol), NaOtBu 18.41 g (1.5 equiv., 191.6 mmol), toluene 255 mL, and benzophenone imine 27.78 g (1.2 equiv., 153.3 mmol) were sequentially added to a 1000 mL three-neck flask, and were heated, refluxed, and stirred. Then, after cooling to a room temperature, the crude product obtained by filtering the reaction solution through silica gel was dissolved in 300 mL of THE. Concentrated hydrochloric acid of 25 mL was added dropwise while stirring the reaction solution at a room temperature, and the generated hydrochloride salt was filtered to obtain 31 g of the filtrate. The hydrochloride salt was neutralized with NaOtBu while suspended in THE and EtOH to concentrate the solution, and then water and toluene were added to separate an organic layer. The organic layer was washed with a saline solution, and then dried over anhydrous MgSO₄. Intermediate IM-7 (25.31 g, yield of 79%) was obtained by performing filtration of MgSO₄ and concentration of the organic layer.

By FAB-MS measurement of the obtained product, a molecular ion peak was observed at the mass number of m/z=249, and thus the obtained product was identified as Intermediate IM-7.

### Synthesis of Intermediate IM-8

Under an argon atmosphere, Intermediate IM-7 6.6 g (26 mmol), Pd(dba)₂ 0.3 g (0.02 equiv., 0.5 mmol), NaOtBu 2.7 g (1.1 equiv., 28 mmol), xylene 500 mL, Intermediate IM-3 6.8 g (1.0 equiv., 26 mmol), and P(^{t}Bu₃)HBF₄ 0.6 g (0.08 equiv., 2 mmol) were sequentially added to a 1000 mL three-neck flask, and were heated, refluxed, and stirred. After cooling to a room temperature, the crude product obtained by performing the filtration of the reaction solution through silica gel, and the concentration of the filtrate was purified to obtain Intermediate IM-8. (5.86 g, yield of 53%)

By FAB-MS measurement of the obtained product, a molecular ion peak was observed at the mass number of m/z=434, and thus the obtained product was identified as Intermediate IM-8.

### Synthesis of Compound C29

Under an argon atmosphere, Intermediate IM-8 5.86 g (13.5 mmol), Pd(dba)₂ 0.23 g (0.03 equiv., 0.41 mmol), NaOtBu 1.62 g (1.25 equiv., 16.9 mmol), xylene 70 mL, Intermediate IM-6 4.13 g (1.1 equiv., 14.9 mmol), and P(tBu₃)HBF₄ 0.47 g (0.12 equiv., 1.6 mmol) were sequentially added to a 200 mL three-neck flask, and were heated, refluxed, and stirred. After cooling to room temperature, the reaction solution was filtered through Florisil, and the filtrate was concentrated. The obtained crude product was purified to obtain Compound C29 (7.92 g, yield of 87%, deuteration rate of 63.3%).

By FAB-MS measurement of the obtained product, a molecular ion peak was observed at the mass number of m/z=675, and thus the obtained product was identified as Compound C29.

### (9) Synthesis of Compound D2

Amine Compound D2 according to one or more embodiments may be synthesized according to, for example, Reaction Scheme 9.

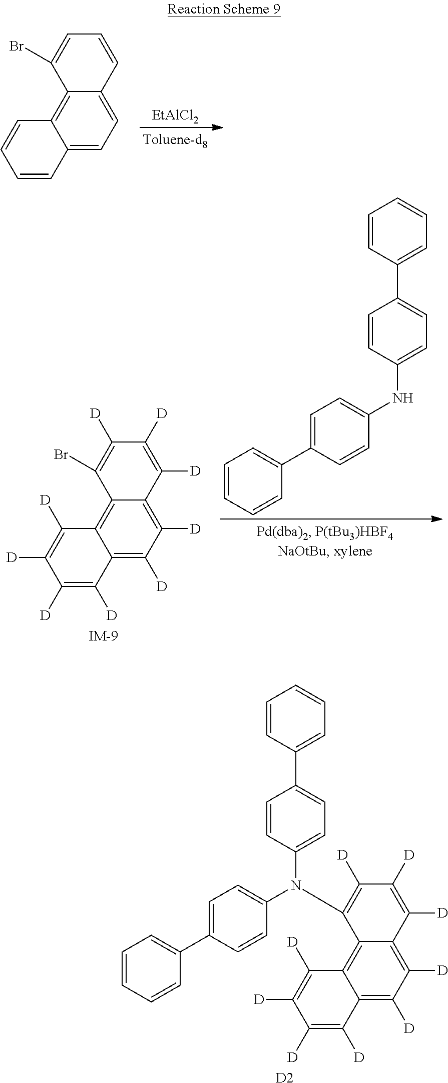

### Synthesis of Intermediate IM-9

Under an argon atmosphere, 4-bromophenanthrene 5.00 g (19.5 mmol) and toluene-d₈ 39 mL were added to a 200 mL three-neck flask, the flask was cooled in an ice-bath, EtAlCl₂ in hexane (ca. 1 M) of 1.95 mL (0.1 equiv., 1.95 mmol) was added dropwise thereto and the mixture was stirred. The mixture was stirred for 1 hour while cooling with ice, then the reaction solution was quenched with 3 N hydrochloric acid, and water was added to separate an organic layer. The organic layer was sequentially cleansed with a saturated aqueous sodium hydrogen carbonate, and a saturated saline solution, and then was dried over anhydrous MgSO₄. The crude product obtained by performing filtration of MgSO₄ and concentration of the organic layer was purified to obtain Intermediate IM-9 (3.8 g, yield of 73%, deuteration rate of 89.8%).

By FAB-MS measurement of the obtained product, a molecular ion peak was observed at the mass number of m/z=265, and thus the obtained product was identified as Intermediate IM-9.

### Synthesis of Compound D2

Under an argon (Ar) atmosphere, Intermediate IM-9 3.8 g (14 mmol), Pd(dba)₂ 0.41 g (0.05 equiv., 0.71 mmol), NaOtBu 1.65 g (1.2 equiv., 17.1 mmol), xylene 142 mL, di([1,1′-biphenyl]-4-yl)amine 4.59 g (1.0 equiv., 14.3 mmol), and P(^{t}Bu₃)HBF₄ 0.83 g (0.2 equiv., 2.86 mmol) were sequentially added to a 300 mL three-neck flask, and were heated, refluxed, and stirred. After air cooling to a room temperature, the reaction solution was filtered through Florisil, and the filtrate was concentrated. The obtained crude product was purified to obtain Compound D2 (4.48 g, yield of 60%, deuteration rate of 23%).

By FAB-MS measurement of the obtained product, a molecular ion peak was observed at the mass number of m/z=520, and thus the obtained product was identified as Compound D2.

### (10) Synthesis of Compound E14

Amine Compound E14 according to one or more embodiments may be synthesized according to, for example, Reaction Scheme 10.

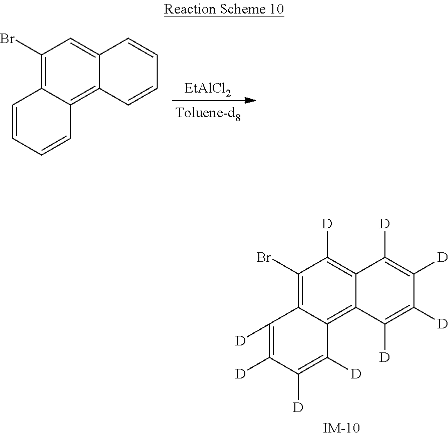

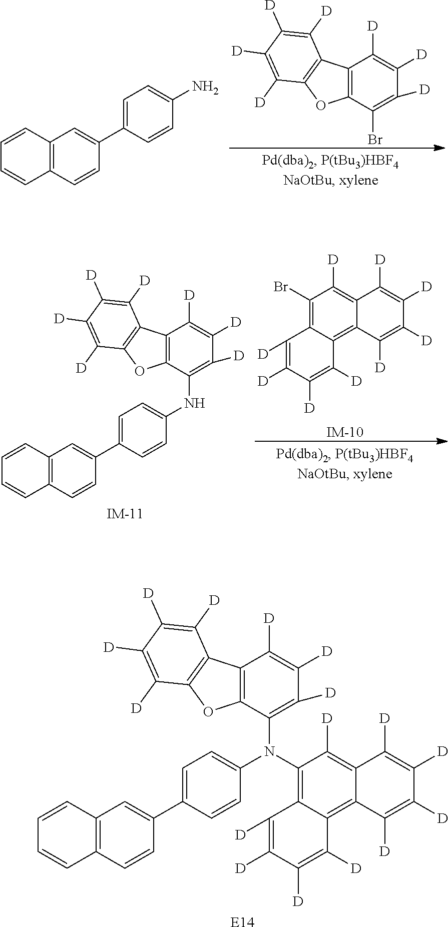

### Synthesis of Intermediate IM-10

Under an argon atmosphere, 9-bromophenanthrene 25.00 g (97.23 mmol) and toluene-d₈ 194 mL were added to a 500 mL three-neck flask, the flask was cooled in an ice-bath, EtAlCl₂ in hexane (ca. 1 M) of 9.72 mL (0.1 equiv., 9.72 mmol) was added dropwise thereto and the mixture was stirred. The mixture was stirred for 1 hour while cooling with ice, then the reaction solution was quenched with 3 N hydrochloric acid, and water was added to separate an organic layer. The organic layer was sequentially washed with a saturated aqueous sodium hydrogen carbonate, and a saturated saline solution, and then was dried over anhydrous MgSO₄. The crude product obtained by performing filtration of MgSO₄ and concentration of the organic layer was purified to obtain Intermediate IM-10 (20.4 g, yield of 79%, deuteration rate of 90.7%).

By FAB-MS measurement of the obtained product, a molecular ion peak was observed at the mass number of m/z=265, and thus the obtained product was identified as Intermediate IM-10.

### Synthesis of Intermediate IM-11

Under an argon atmosphere, 4-(naphthalen-2-yl)aniline 10.00 g (45.60 mmol), Pd(dba)₂ 1.31 g (0.05 equiv., 2.28 mmol), NaO^{t}Bu 5.26 g (1.2 equiv., 54.7 mmol), xylene 456 mL, 4-bromodibenzo[b,d]furan-1,2,3,6,7,8,9-d₇ 11.59 g (1.0 equiv., 45.60 mmol), and P(^{t}Bu₃)HBF₄ 2.65 g (0.2 equiv., 9.12 mmol) were sequentially added to a 1000 mL three-neck flask, and were heated, refluxed, and stirred. After air cooling to a room temperature, the reaction solution was filtered through Florisil, and the filtrate was concentrated. The obtained crude product was purified to obtain Intermediate IM-11 (14.36 g, yield of 80%).

By FAB-MS measurement of the obtained product, a molecular ion peak was observed at the mass number of m/z=392, and thus the obtained product was identified as Intermediate IM-11.

### Synthesis of Compound E14

Under an argon atmosphere Intermediate IM-11 8.00 g (20.4 mmol), Pd(dba)₂ 0.59 g (0.05 equiv., 1.0 mmol), NaOtBu 2.35 g (1.2 equiv., 24.5 mmol), xylene 204 mL, Intermediate IM-10 5.43 g (1.0 equiv., 20.4 mmol), and P(^{t}Bu₃)HBF₄ 1.18 g (0.2 equiv., 4.08 mmol) were sequentially added to a 500 mL three-neck flask, and were heated, refluxed, and stirred. After air cooling to room temperature, the reaction solution was filtered through Florisil, and the filtrate was concentrated. The obtained crude product was purified to obtain Compound E14 (9.92 g, yield of 82%, deuteration rate of 44%).

By FAB-MS measurement of the obtained product, a molecular ion peak was observed at the mass number of m/z=591, and thus the obtained product was identified as Compound E14.

### 2. Manufacture and Evaluation of Light-Emitting Element

### (1) Manufacture of Light-Emitting Element

A light-emitting element including an amine compound according to one or more embodiments in a hole transport layer was manufactured by a method described herein. Light-emitting elements according to Example 1 to Example 10 were each manufactured using the respective amine compounds according to one or more embodiments as a material for the hole transport layer. Light-emitting elements according to Comparative Example 1 to Comparative Example 21 were each manufactured using Comparative Example Compounds R₁ to R₂₁ as a material for the hole transport layer, respectively. Example Compounds used in Examples 1 to 10 and Comparative Example Compounds used in Comparative Examples 1 to 21 are as follows.

### Example Compounds

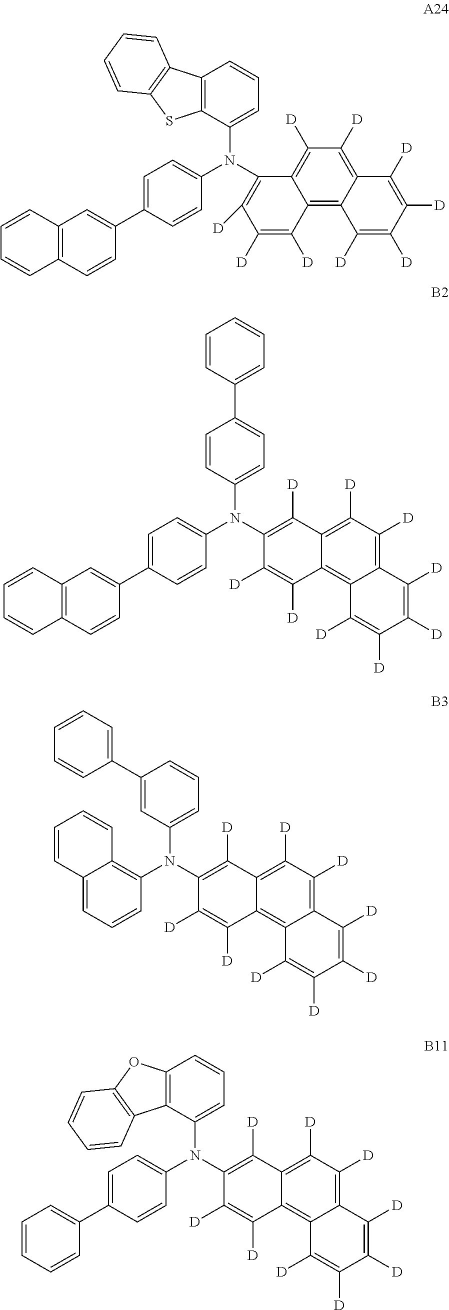 [SMILES: [2H]c1c([2H])c([2H])c2c(c1[2H])c([2H])c([2H])c1c(N(c3ccc(-c4ccc5ccccc5c4)cc3)c3cccc4c3sc3ccccc34)c([2H])c([2H])c([2H])c12 ; [2H]c1c([2H])c([2H])c2c(c1[2H])c([2H])c([2H])c1c([2H])c(N(c3ccc(-c4ccccc4)cc3)c3ccc(-c4ccc5ccccc5c4)cc3)c([2H])c([2H])c12 ; [2H]c1c([2H])c([2H])c2c(c1[2H])c([2H])c([2H])c1c([2H])c(N(c3cccc(-c4ccccc4)c3)c3cccc4ccccc34)c([2H])c([2H])c12 ; [2H]c1c([2H])c([2H])c2c(c1[2H])c([2H])c([2H])c1c([2H])c(N(c3ccc(-c4ccccc4)cc3)c3cccc4oc5ccccc5c34)c([2H])c([2H])c12]

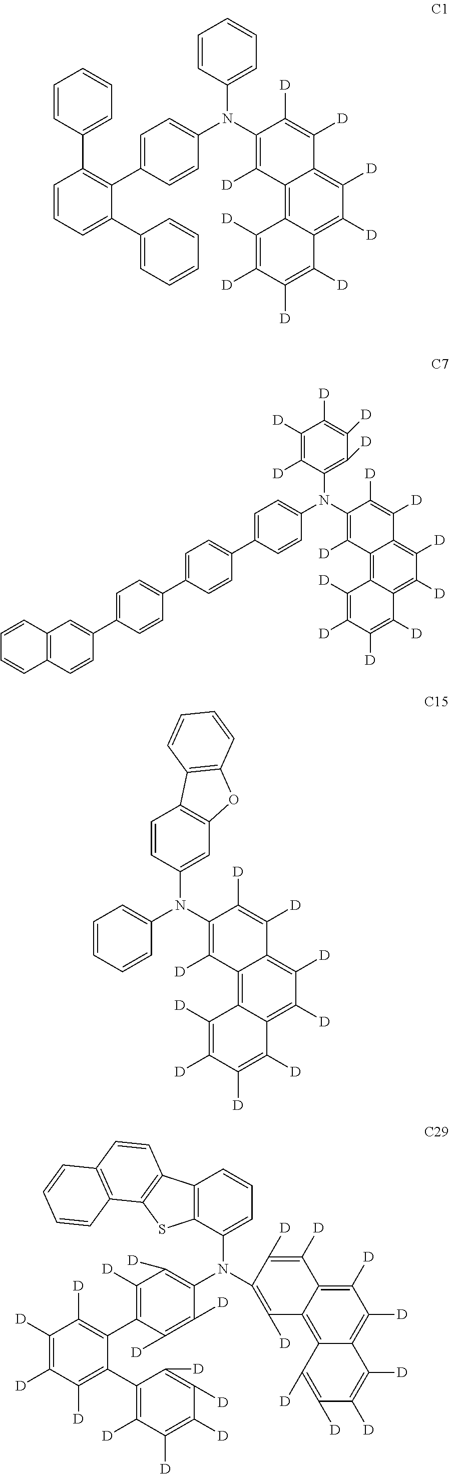

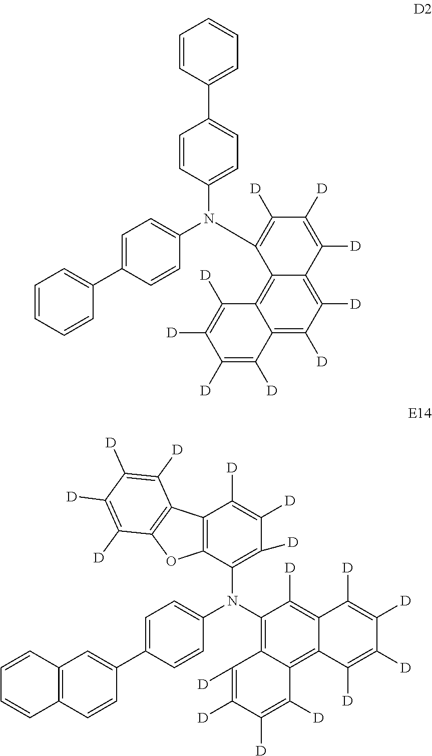 [SMILES: [2H]c1c([2H])c([2H])c2c(c1[2H])c([2H])c([2H])c1c([2H])c([2H])c([2H])c(N(c3ccc(-c4ccccc4)cc3)c3ccc(-c4ccccc4)cc3)c12 ; [2H]c1c([2H])c([2H])c2c(oc3c(N(c4ccc(-c5ccc6ccccc6c5)cc4)c4c([2H])c5c([2H])c([2H])c([2H])c([2H])c5c5c([2H])c([2H])c([2H])c([2H])c45)c([2H])c([2H])c([2H])c32)c1[2H]]

### Comparative Example Compounds

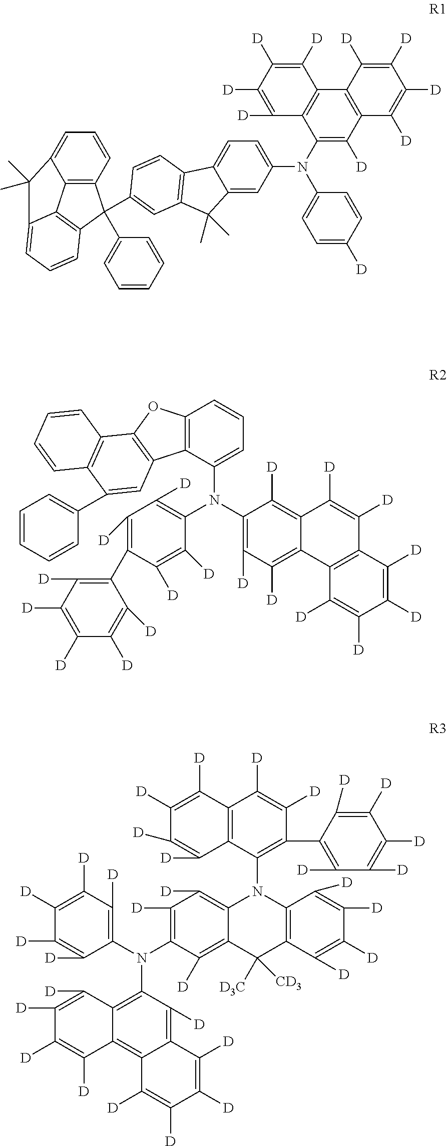

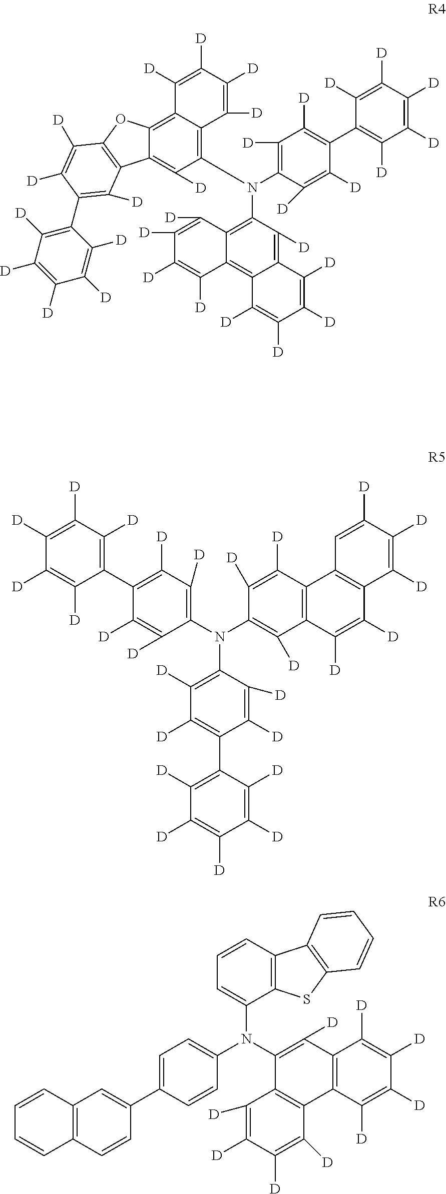

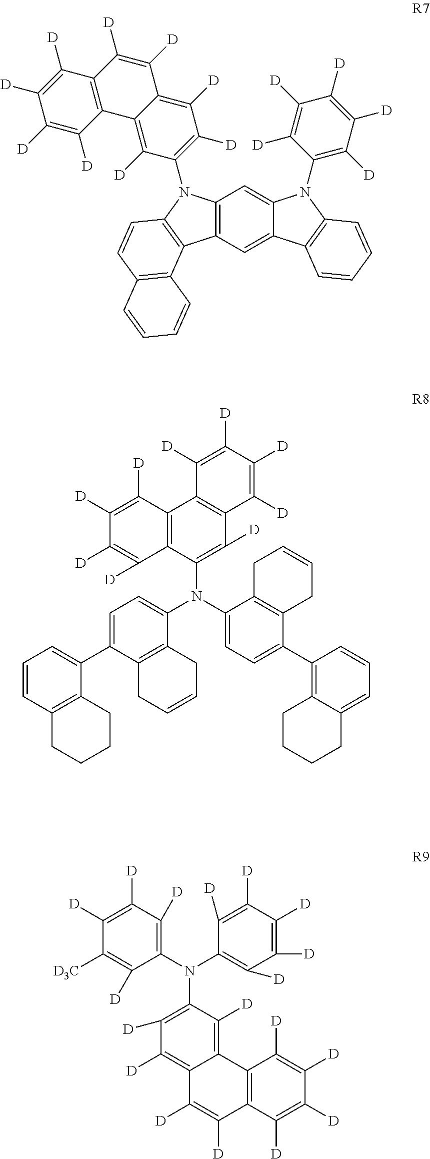 [SMILES: [2H]c1c([2H])c([2H])c(-n2c3ccccc3c3cc4c5c6ccccc6ccc5n(-c5c([2H])c([2H])c6c([2H])c([2H])c7c([2H])c([2H])c([2H])c([2H])c7c6c5[2H])c4cc32)c([2H])c1[2H] ; [2H]c1c([2H])c([2H])c2c(c1[2H])c([2H])c(N(c1ccc(-c3cccc4c3CCCC4)c3c1CC=CC3)c1ccc(-c3cccc4c3CCCC4)c3c1CC=CC3)c1c([2H])c([2H])c([2H])c([2H])c12 ; [2H]c1c([2H])c([2H])c(N(c2c([2H])c([2H])c([2H])c(C([2H])([2H])[2H])c2[2H])c2c([2H])c([2H])c3c([2H])c([2H])c4c([2H])c([2H])c([2H])c([2H])c4c3c2[2H])c([2H])c1[2H]]

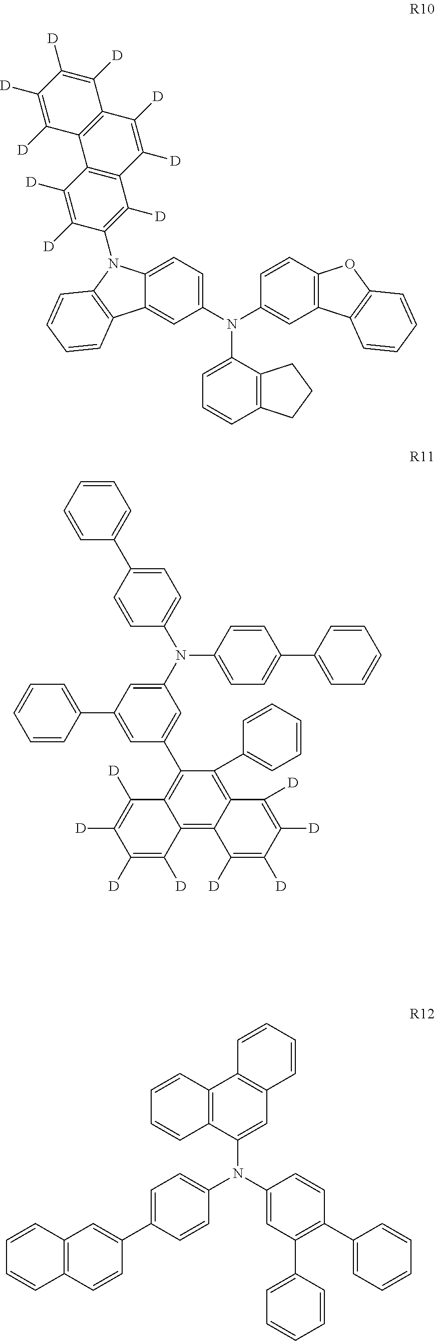 [SMILES: [2H]c1c([2H])c([2H])c2c(c1[2H])c([2H])c([2H])c1c([2H])c(-n3c4ccccc4c4cc(N(c5ccc6oc7ccccc7c6c5)c5cccc6c5CCC6)ccc43)c([2H])c([2H])c12 ; [2H]c1c([2H])c([2H])c2c(c1[2H])c(-c1ccccc1)c(-c1cc(-c3ccccc3)cc(N(c3ccc(-c4ccccc4)cc3)c3ccc(-c4ccccc4)cc3)c1)c1c([2H])c([2H])c([2H])c([2H])c12 ; c1ccc(-c2ccc(N(c3ccc(-c4ccc5ccccc5c4)cc3)c3cc4ccccc4c4ccccc34)cc2-c2ccccc2)cc1]

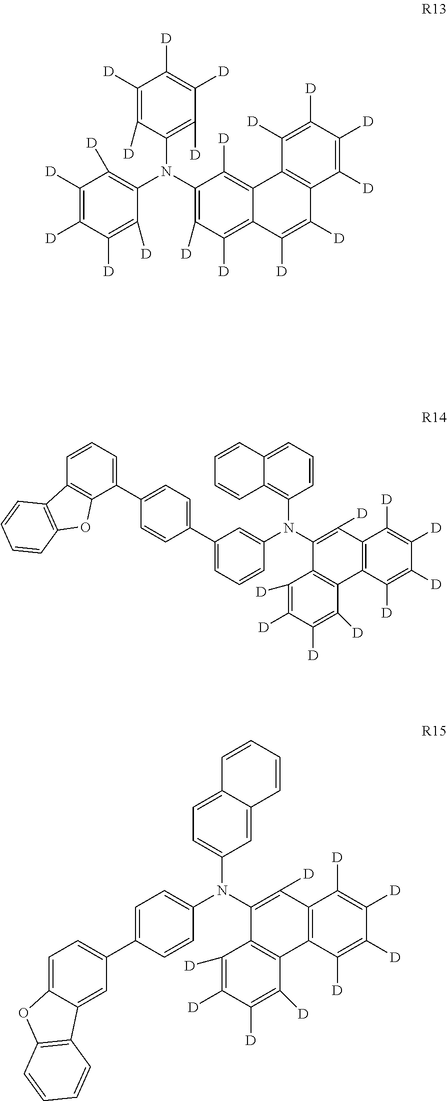 [SMILES: [2H]c1c([2H])c([2H])c(N(c2c([2H])c([2H])c([2H])c([2H])c2[2H])c2c([2H])c([2H])c3c([2H])c([2H])c4c([2H])c([2H])c([2H])c([2H])c4c3c2[2H])c([2H])c1[2H] ; [2H]c1c([2H])c([2H])c2c(c1[2H])c([2H])c(N(c1cccc(-c3ccc(-c4cccc5c4oc4ccccc45)cc3)c1)c1cccc3ccccc13)c1c([2H])c([2H])c([2H])c([2H])c12 ; [2H]c1c([2H])c([2H])c2c(c1[2H])c([2H])c(N(c1ccc(-c3ccc4oc5ccccc5c4c3)cc1)c1ccc3ccccc3c1)c1c([2H])c([2H])c([2H])c([2H])c12]

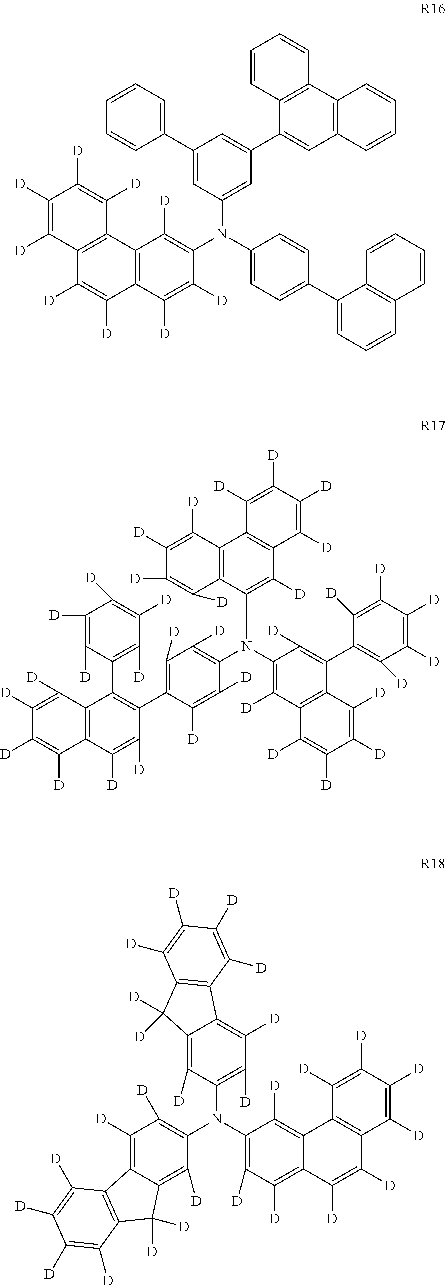

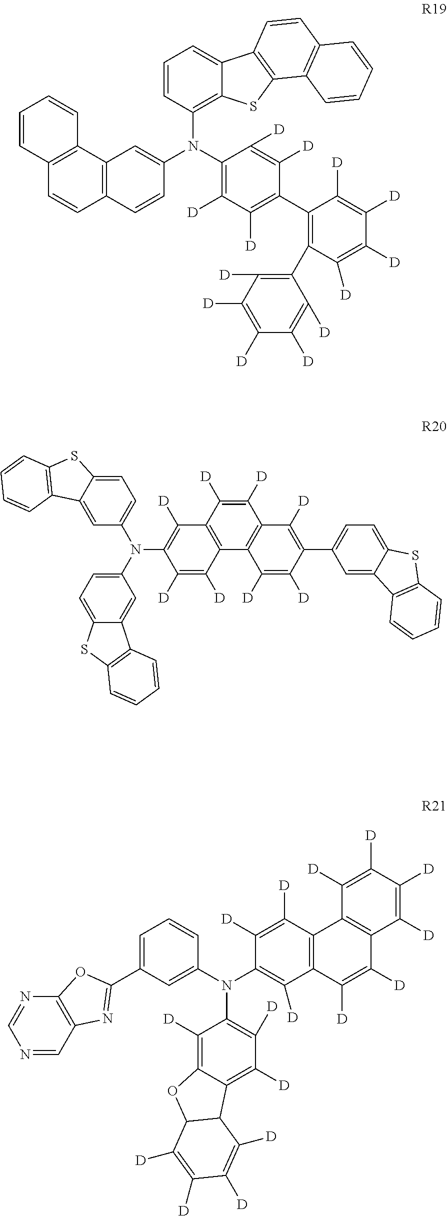 [SMILES: [2H]c1c([2H])c([2H])c(-c2c([2H])c([2H])c([2H])c([2H])c2-c2c([2H])c([2H])c(N(c3ccc4ccc5ccccc5c4c3)c3cccc4c3sc3c5ccccc5ccc43)c([2H])c2[2H])c([2H])c1[2H] ; [2H]c1c(-c2ccc3sc4ccccc4c3c2)c([2H])c2c([2H])c([2H])c3c([2H])c(N(c4ccc5sc6ccccc6c5c4)c4ccc5sc6ccccc6c5c4)c([2H])c([2H])c3c2c1[2H] ; [2H]C1=C([2H])C2Oc3c([2H])c(N(c4cccc(-c5nc6cncnc6o5)c4)c4c([2H])c([2H])c5c(c4[2H])c([2H])c([2H])c4c([2H])c([2H])c([2H])c([2H])c45)c([2H])c([2H])c3C2C([2H])=C1[2H]]

A glass substrate, on which ITO of about 150 nm was patterned as a first electrode, was cleansed using ultrasonic waves with isopropyl alcohol and ultrapure water, for about 5 minutes each. After ultrasonic wave cleansing, the ITO glass substrate was exposed to UV for about 30 minutes and treated with ozone. Then, 2-TNATA was deposited to a thickness of about 60 nm to form a hole injection layer. The example compound or comparative example compound was deposited to a thickness of about 30 nm to form a hole transport layer.

On the hole transport layer, TBP and ADN were co-deposited to form a light-emitting layer with a thickness of about 25 nm. TBP and ADN were co-deposited at a ratio of about 3:97. Thereafter, Alq₃ to a thickness of about 25 nm and LiF to a thickness of about 1 nm were sequentially deposited to form an electron transport region.

Then Al was deposited to a thickness of about 100 nm to form a second electrode.

In the manufacture of the light-emitting elements, the hole transport region, the light-emitting layer, the electron transport region, and the second electrode were formed using a vacuum evaporator.

The compounds used for the manufacture of the light-emitting elements are as follows.

### Materials Used for the Manufacture of Light-Emitting Element

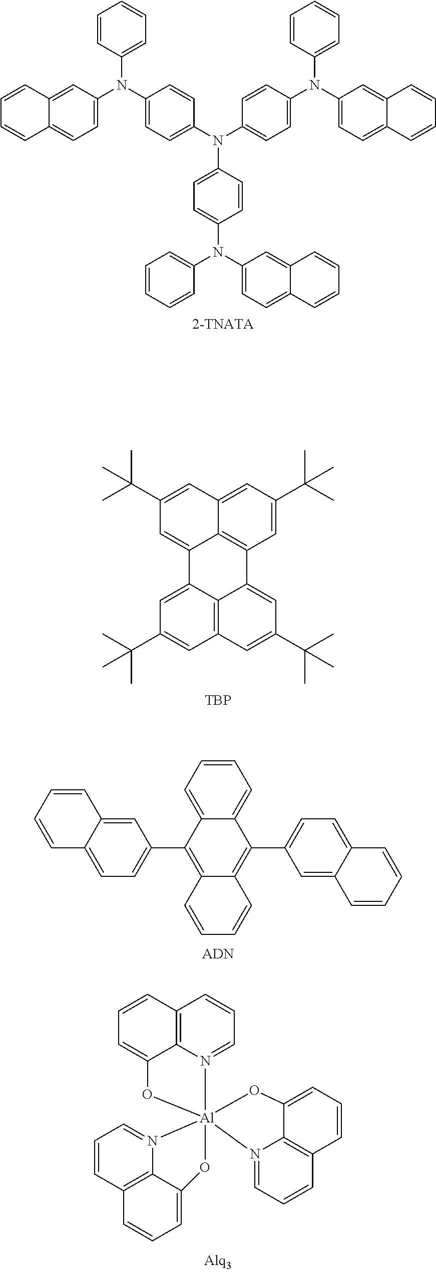

### (2) Evaluation of Light-Emitting Element

The light-emitting elements according to Examples and Comparative Examples were each evaluated and the results are listed in Table 1. In Table 1, luminous efficiency and element lifespan of the light emitting element according to each of Examples and Comparative Examples are shown. The evaluation of the current density, voltage, and luminous efficiency of each of the light emitting elements was conducted using a Source Meter of 2400 series, made by Keithley Instruments Co., a luminance and color meter of CS-200, made by Konica Minolta Co., Ltd., and PC Program LabVIEW 8.2 for measurement, made by Japan National Instruments Co., Ltd., in a dark room.

The luminous efficiency was measured at a current density of 10 mA/cm² and the value was relatively expressed with respect to the luminous efficiency of the light-emitting element according to Comparative Example 19 as 100%. Time taken for initial luminance of 100 cd/m² to decrease to its 50% was measured, and the relative element lifespan of each light-emitting element was calculated with respect to a value of the light-emitting element according to Comparative Example 19 as 100% and the relative values are listed in Table 1.

TABLE 1

| No. of light- | Material for hole | Luminous | Element lifespan |
|---|---|---|---|
| emitting element | transport layer | efficiency (%) | (LT50)(%) |
| Example 1 | Compound A24 | 110% | 122% |
| Example 2 | Compound B2 | 111% | 118% |
| Example 3 | Compound B3 | 113% | 116% |
| Example 4 | Compound B11 | 115% | 125% |
| Example 5 | Compound C1 | 113% | 121% |
| Example 6 | Compound C7 | 113% | 130% |
| Example 7 | Compound C15 | 112% | 127% |
| Example 8 | Compound C29 | 113% | 132% |
| Example 9 | Compound D2 | 111% | 124% |
| Example 10 | Compound E14 | 114% | 130% |
| Comparative | Comparative | 97% | 61% |
| Example 1 | Example | | |
| | Compound R1 | | |
| Comparative | Comparative | 102% | 84% |
| Example 2 | Example | | |
| | Compound R2 | | |
| Comparative | Comparative | 95% | 65% |
| Example 3 | Example | | |
| | Compound R3 | | |
| Comparative | Comparative | 101% | 92% |
| Example 4 | Example | | |
| | Compound R4 | | |
| Comparative | Comparative | 103% | 93% |
| Example 5 | Example | | |
| | Compound R5 | | |
| Comparative | Comparative | 103% | 88% |
| Example 6 | Example | | |
| | Compound R6 | | |
| Comparative | Comparative | 92% | 68% |
| Example 7 | Example | | |
| | Compound R7 | | |
| Comparative | Comparative | 98% | 59% |
| Example 8 | Example | | |
| | Compound R8 | | |
| Comparative | Comparative | 99% | 38% |
| Example 9 | Example | | |
| | Compound R9 | | |
| Comparative | Comparative | 97% | 70% |
| Example 10 | Example | | |
| | Compound R10 | | |
| Comparative | Comparative | 102% | 81% |
| Example 11 | Example | | |
| | Compound R11 | | |
| Comparative | Comparative | 99% | 91% |
| Example 12 | Example | | |
| | Compound R12 | | |
| Comparative | Comparative | 98% | 57% |
| Example 13 | Example | | |
| | Compound R13 | | |
| Comparative | Comparative | 103% | 88% |
| Example 14 | Example | | |
| | Compound R14 | | |
| Comparative | Comparative | 102% | 91% |
| Example 15 | Example | | |
| | Compound R15 | | |
| Comparative | Comparative | 103% | 76% |
| Example 16 | Example | | |
| | Compound R16 | | |
| Comparative | Comparative | 105% | 93% |
| Example 17 | Example | | |
| | Compound R17 | | |
| Comparative | Comparative | 98% | 88% |
| Example 18 | Example | | |
| | Compound R18 | | |
| Comparative | Comparative | 100% | 100% |
| Example 19 | Example | | |
| | Compound R19 | | |
| Comparative | Comparative | 98% | 80% |
| Example 20 | Example | | |
| | Compound R20 | | |
| Comparative | Comparative | 85% | 55% |
| Example 21 | Example | | |
| | Compound R21 | | |

Referring to Table. 1, compared to light-emitting elements according to Comparative Example 1 to Comparative Example 21, each of light-emitting elements according to Example 1 to Example 10 exhibits element characteristics of high efficiency and long lifespan. Example compounds include a deuterated phenanthrene group and an aromatic amine group directly bonded to the phenanthrene group at a specific bonding position, thereby having, compared to Comparative Example compounds, sufficiently high electron density and high planarity according to the specified substituent group and the substitution position, and thus may exhibit excellent or suitable hole transport ability. For example, because the Comparative Example compounds have substituent characteristics distinct from Example compounds such as the types (kinds) of aromatic substituents, a bonding position, or a bonding manner, electron density may become excessively (or substantially) high, which may lead to reduced stability or characteristics of reduced stability of the compounds. Therefore, Comparative Example compounds may have lower stability and reduced charge mobility than Example compounds.

In addition, Example compounds may exhibit beneficial characteristics of improved stability due to kinetic isotope effects by the deuterated phenanthrene group in addition to the aromatic amine group.

In the descriptions of Examples and Comparative Examples, carbon atoms in phenanthrene are numbered as follows.

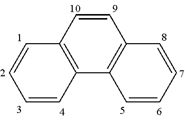 [SMILES: c1ccc2c(c1)ccc1ccccc12]

The light-emitting elements according to Comparative Example 1 and Comparative Example 18 each exhibits reduced luminous efficiency and element lifespan, compared to the light-emitting elements according to Example 1 to Example 10. Comparative Example Compound R1 and Comparative Example Compound R18 each includes a fluorene moiety in the amine group, the fluorene moiety contains a quaternary carbon, and a bond at a location thereof may easily be dissociated by heat.

Therefore, in the Comparative Example compounds including the fluorene moiety, decomposition may be caused during deposition. Therefore, it is considered that the light-emitting element according to Comparative Example 1 including Comparative Example Compound R1 and the light-emitting element according to Comparative Example 18 including Comparative Example Compound R18 exhibit reduced luminous efficiency and element lifespan, compared to the light-emitting elements according to Example 1 to Example 10.

The light-emitting elements according to Comparative Example 2 and Comparative Example 4 each exhibits reduced luminous efficiency and element lifespan, compared to the light-emitting elements according to Example 1 to Example 10. Comparative Example Compound R2 and Comparative Example Compound R4 each includes a benzonaphthofuran moiety substituted with an aryl group, and Comparative Example Compound R2 and Comparative Example Compound R4 have relatively high deposition temperatures. In the case of Comparative Example Compound R2, a nitrogen atom of the amine group is bonded at a second position of phenanthrene, thus planarity thereof becomes greater, and thus the deposition temperature increases. Therefore, it can be thought that the Comparative Example compound decomposes during deposition, and thus the light-emitting elements have deteriorated performance. In the case of Comparative Example Compound R4, a phenanthrene moiety is bonded to nitrogen at a ninth position thereof, which makes reactivity at a tenth position of the phenanthrene moiety relatively high. It can be considered that decomposition of the Comparative Example compound proceeds during deposition due to the described characteristics of the reactivity in the compound and high deposition temperature, and thus element performance is reduced.

Each of the light-emitting elements according to Comparative Example 3, Comparative Example 8, and Comparative Example 10 exhibits reduced luminous efficiency and element lifespan, compared to the light-emitting elements according to Example 1 to Example 2. An alkyl group in Comparative Example Compound R3 and an aliphatic ring in each of Comparative Example Compound R8 and Comparative Example Compound R10 are not stabilized through conjugation compared to the aromatic ring, and thus Comparative Example Compounds R3, R8, and R10 have deteriorated heat resistance compared to Example compounds. Therefore, in the cases of Comparative Examples R3, R8, and R10, the performance after deposition is reduced, and the light-emitting elements according to Comparative Example 3, Comparative Example 8, and Comparative Example 10 each exhibit deteriorated element characteristics.

The light-emitting element according to Comparative Example 5 has reduced luminance efficiency and element lifespan. In Comparative Example Compound R5, a nitrogen atom of the amine group is bonded at a location of R2 in Formula 1 of Example compounds according to the disclosure, and the amine group at the same time includes two aryl groups each having ring-forming carbons of smaller than 16, which is distinct from a structure of the Example compounds. A skeleton of the aryl group having ring-forming carbons of smaller than 16 has a relatively narrow conjugation and may not sufficiently delocalize holes and electrons, thereby exhibiting characteristics of reduced stability. Therefore, Example compounds including the deuterated phenanthrene skeleton, and the amine group including at least one among an aryl group having ring-forming carbons of at least 16, an aryl group having a fused structure, and/or a heteroaryl group, may have excellent or suitable stability compared to Comparative Example Compound R5. Therefore, it can be considered that each of the light-emitting elements according to Examples has improved element characteristics compared to the light-emitting element according to Comparative Example 5.

The light-emitting element according to Comparative Example 6 has reduced luminous efficiency and element lifespan compared to the light-emitting elements according to Example 1 to Example 10. Comparative Example Compound R6 includes a dibenzothiophene moiety bonded to a nitrogen atom at a fourth position of the dibenzothiophene moiety, and a heteroaromatic substituent such as dibenzofuran and dibenzothiophene has a twisted skeleton that does not conjugate with other substituents bonded to the nitrogen atom due to a large volume. Accordingly, in the case of Comparative Example Compound R6, electrons are localized, resulting in reduced stability. In addition, in the case of Comparative Example Compound R6, nitrogen of the amine group is bonded at a ninth position of phenanthrene, electrons are sufficient near nitrogen, resulting in more reduced stability. Therefore, it can be considered that Comparative Example Compound 6 has deteriorated element performance.

The light-emitting element according to Comparative Example 7 has reduced luminous efficiency and element lifespan compared to the light-emitting elements according to Example 1 to Example 10. Comparative Example Compound 7 includes, compared to Example compounds, a carbazole moiety instead of an amine moiety. The carbazole moiety has lower hole transport ability than the amine moiety, and thus a carrier balance is broken. It can be considered that the light-emitting element according to Comparative Example 7 has reduced element performance due to characteristics of Comparative Example Compound R7.

The light-emitting elements according to Comparative Example 9 and Comparative Example 13 have reduced luminous efficiency and element lifespan compared to the light-emitting elements according to Example 1 to Example 10. Comparative Example Compounds R9 and R13 each include a phenyl group as a substituent bonded to a nitrogen atom of the amine group, the phenyl group has narrow conjugation characteristics, and thus may not make holes and electrons sufficiently delocalized. Therefore, Comparative Example Compounds R9 and R13 exhibit characteristics of reduced stability. Example compounds including a deuterated phenanthrene skeleton and an amine group including at least one among an aryl group or heteroaryl group, each having ring-forming carbons of 10 or more, thus, may have excellent or suitable stability compared to Comparative Example Compounds R9 and R13. As a result, the light-emitting elements according to Examples may have improved element characteristics compared to the light-emitting elements according to Comparative Examples 9 and 13.

The light-emitting element according to Comparative Example 11 has reduced luminous efficiency and element lifespan compared to the light-emitting elements according to Example 1 to Example 10. In Comparative Example Compound R11, a phenanthrene moiety and an amine group are not directly bonded, but are bonded via a linker, which is distinct from the Example compounds. In the case of Comparative Example Compound R11, it is considered that, because the phenanthrene moiety having high planarity is bonded to the amine group via a linker, decomposition easily occurs during deposition, and thus the light-emitting element according to Comparative Example 11 has characteristics of reduced lifespan.

The light-emitting elements according to Comparative Example 12 and Comparative Example 19 have reduced luminous efficiency and element lifespan compared to the light-emitting elements according to Example 1 to Example 10. In each of Comparative Example Compounds R12 and R19, a phenanthrene moiety is not deuterated, which is distinct from Example compounds. In each of Example compounds, its phenanthrene moiety is deuterated, and thus stability may be improved due to kinetic isotope effects. Therefore, it is considered that the light-emitting elements according to Example compounds have element characteristics of long lifespan and high efficiency, compared to the light-emitting elements according to Comparative Example Compound 12 and Comparative Example Compound 19.

The light-emitting elements according to Comparative Example 14, Comparative Example 15, and Comparative Example 17 have reduced luminous efficiency and element lifespan compared to the light-emitting elements according to Example 1 to Example 10. A naphthalene skeleton is highly reactive, and if (e.g., when) a nitrogen atom of an amine group including such a naphthyl group is bonded at a ninth position of a phenanthrene moiety, stability of the compound decreases. Because Comparative Example Compounds R14, R15, and R17 have the described characteristics, the light-emitting elements according to Comparative Examples 14, 15, and 17 have deteriorated element characteristics, compared to the light-emitting elements according to Examples.

The light-emitting element according to Comparative Example 16 has reduced luminous efficiency and element lifespan compared to the light-emitting elements according to Example 1 to Example 10. When an amine group is bonded at a third position of a phenanthrene moiety and the amine group includes a cyclic group having carbons of 26 or greater as a substituent, as Comparative Example Compound 16, deposition temperature increases. Therefore, the compound may be decomposed during the deposition, and thus the light-emitting element may have a reduced lifespan. In Example compounds, compared to Comparative Example Compound 16, if (e.g., when) the amine group is bonded at a third position of the phenanthrene moiety, a case where two substituents substituted for a nitrogen atom of the amine group are at the same time a cyclic compound having 26 or more carbons is excluded, and if (e.g., when) one substituent is a cyclic compound having 26 or more carbons, the rest one is one selected from among a cyclic compound and a heteroaryl group each having 15 or less carbons, and thus decomposition of the compound may be suppressed or reduced.

The light-emitting element according to Comparative Example 20 has reduced luminous efficiency and element lifespan compared to the light-emitting elements according to Example 1 to Example 10. In a phenanthrene moiety, if (e.g., when) a heteroaryl group is substituted at the remaining location thereof where an amine group is not bonded, a charge balance may become poor, and thus thermal stability may decrease. In cases of Example compounds, compared to Comparative Example Compound R20, no heteroaryl group is included and substituted for the phenanthrene moiety as a substituent, and thus deterioration of charge balance and material decomposition may be suppressed or reduced. Therefore, it can be seen that the light-emitting elements according to Examples each have excellent or suitable element characteristics compared to the light-emitting element according to Comparative Example 20.

The light-emitting element according to Comparative Example 21 has reduced luminous efficiency and element lifespan compared to the light-emitting elements according to Example 1 to Example 10. Comparative Example Compound 21 includes a heteroaryl group having a plurality of hetero atoms such as an oxazolopyrimidine group in the molecule, and in this case, a balance of the compound may be significantly deteriorated. Therefore, when a heteroaryl group including 4 or more hetero atoms is included such as Comparative Example Compound R21, a charge balance may become poor, and thus a hole transport ability may decrease when used as a material for a hole transport region. Therefore, it can be seen that the light-emitting elements according to Examples using Example compounds including no heteroaryl group that contains 4 or more hetero atoms, as materials for a hole transport layer, have excellent or suitable element characteristics compared to the light-emitting element according to Comparative Example 21.

The amine compound according to one or more embodiments includes a deuterated phenanthrene group and an aromatic amine group including a substituent of aryl or heteroaryl, and has a structure in which the amine group and the phenanthrene group are directly bonded. Thus, the amine compound according to one or more embodiments may have excellent or suitable stability and hole transport ability.

The light-emitting element according to one or more embodiments including the amine compound according to one or more embodiments in the hole transport region may exhibit characteristics of excellent or suitable luminous efficiency and long lifespan. In addition, the light-emitting element according to one or more embodiments that is to emit blue light or green light includes the amine compound according to one or more embodiments in the hole transport region, and thus may exhibit characteristics of high efficiency and long lifespan.

The light-emitting element according to one or more embodiments includes the amine compound according to one or more embodiments in the hole transport region, and thus may exhibit characteristics of high efficiency and long lifespan.

The amine compound according to one or more embodiments may be used as a material for achieving light-emitting element characteristics of improved high efficiency and long lifespan.

The display device according to one or more embodiments may exhibit excellent or suitable display quality by including the light-emitting element according to one or more embodiments.

In other words, the amine compound described in one or more embodiments includes a deuterated phenanthrene group and an aromatic amine group with an aryl or heteroaryl substituent, directly bonded. This structure provides excellent stability and hole transport ability. Light-emitting elements incorporating this amine compound in the hole transport region exhibit high luminous efficiency and long lifespan, particularly for elements emitting blue or green light. These light-emitting elements demonstrate high efficiency and long lifespan, making the amine compound a valuable material for achieving improved light-emitting element characteristics. Consequently, display devices using these light-emitting elements show excellent display quality. For example, the amine compound's unique structure enhances stability and hole transport ability, leading to light-emitting elements with superior efficiency and lifespan. This makes it an ideal material for high-performance display devices, ensuring excellent display quality.

As utilized herein, the terms “substantially,” “about,” or similar terms are used as terms of approximation and not as terms of degree, and are intended to account for the inherent deviations in measured or calculated values that would be recognized by those of ordinary skill in the art. “About” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, or 5% of the stated value.

In the context of the present application and unless otherwise defined, the terms “use,” “using,” and “used” may be considered synonymous with the terms “utilize,” “utilizing,” and “utilized,” respectively.

Any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

The light-emitting element, the display apparatus/device, the electronic apparatus/device, the manufacturing apparatuses thereof, or any other relevant apparatuses/devices or components according to embodiments of the present disclosure described herein may be implemented utilizing any suitable hardware, firmware (e.g., an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

In the present disclosure, each suitable feature of the various embodiments of the disclosure may be combined or combined with each other, partially or entirely, and may be technically interlocked and operated in various suitable ways, and each embodiment may be implemented independently of each other or in conjunction with each other in any suitable manner unless otherwise stated or implied.

Hither to, although one or more embodiments of disclosure have been described with reference to certain example embodiments, it is understood that one or more suitable changes and modifications can be made by one ordinary skilled in the art within the spirit and scope of present disclosure as hereinafter claimed.

Accordingly, the technical scope of the disclosure is not limited to the contents set forth in the detailed description of the disclosure, but is defined by the appended claims and equivalents thereof.

What is claimed is:

1. A light-emitting element comprising:
a first electrode;
a second electrode on the first electrode;
an light-emitting layer between the first electrode and the second electrode; and
a hole transport region between the first electrode and the second electrode and comprising an amine compound represented by Formula 1 and Formula 2: 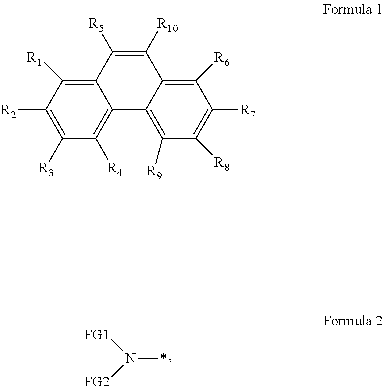
wherein,
in Formula 1, at least one selected from among R₁ to R₅ is an amine group represented by Formula 2, and at least one selected from among the remaining R₁ to R₅ and R₆ to R₁₀ is deuterium, and the remaining that is not deuterium is a substituted or unsubstituted aryl group having ring-forming carbons of 6 to 40,
in Formula 2, FG1 and FG2 are each independently a substituted or unsubstituted aryl group having ring-forming carbons of 6 to 40 except for a fluorenyl group or a substituted or unsubstituted heteroaryl group comprising 1 to 3 hetero atoms and having ring-forming carbons of 5 to 40, and FG1 and FG2 comprise no alkyl group as a substituent,
when R₂ is the amine group represented by Formula 2:
a case where FG1 and FG2 are each a substituted or unsubstituted aryl group having ring-forming carbons of 6 to 15, at the same time, is excluded; and
a case where FG1 or FG2 is a benzonaphthofuranyl group substituted with an aryl group is excluded,
when R₃ is the amine group represented by Formula 2:
a case where FG1 and FG2 are each a substituted or unsubstituted aryl group having ring-forming carbons of 6 to 9, at the same time, is excluded; and
when one of FG1 and FG2 is an aryl group having a total ring-forming carbons of 26 or more, the other is an aryl group having a total ring-forming carbons of 6 to 15 or a substituted or unsubstituted heteroaryl group having ring-forming carbons of 5 to 40, and
when R₅ is the amine group represented by Formula 2:
when FG1 or FG2 is a 4-dibenzofuranyl group or a 4-dibenzothiophenyl group, at least one hydrogen of the 4-dibenzofuranyl group or the 4-dibenzothiophenyl group is substituted with a substituent; and
cases where FG1 or FG2 is a naphthyl group or a benzonaphthofuranyl group substituted with an aryl group are excluded.

2. The light-emitting element of claim 1, wherein:
the hole transport region comprises at least one selected from among a hole injection layer, a hole transport layer, an electron-blocking layer, and an emission auxiliary layer; and
at least one selected from among the hole injection layer, the hole transport layer, the electron-blocking layer, and the emission auxiliary layer comprises the amine compound.

3. The light-emitting element of claim 1, wherein:
the hole transport region comprises a hole injection layer on the first electrode and a hole transport layer on the hole injection layer, the hole transport layer comprising the amine compound.

4. The light-emitting element of claim 1, wherein the amine compound is represented by any one selected from among Formula 1A to Formula 1E: 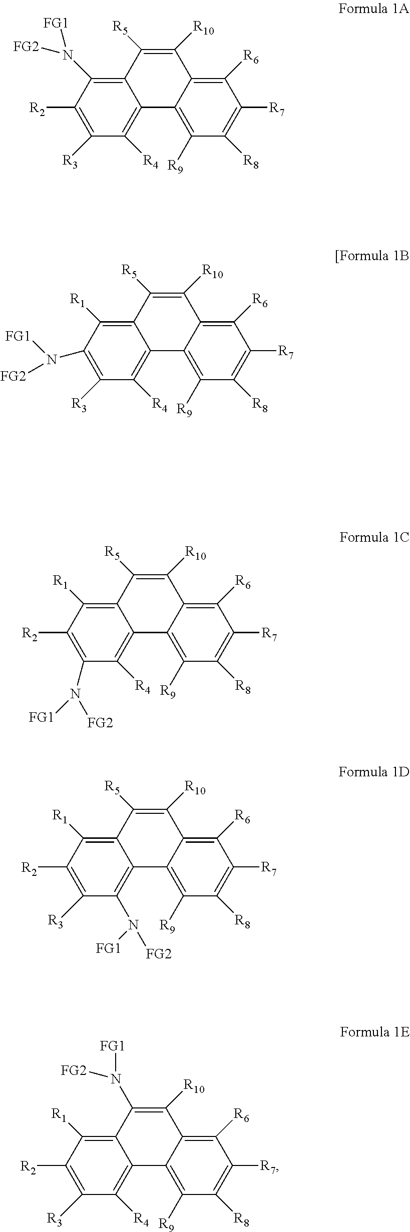
in Formula 1A to Formula 1E,
R₁ to R₁₀, FG1, and FG2 each being the same as defined in Formula 1 and Formula 2.

5. The light-emitting element of claim 1,
wherein, in Formula 1,
the remaining of R₁ to R₅ that is not the amine group represented by Formula 2, and R₆ to R₁₀ are all deuterium(s), or
any one selected from among the remaining of R₁ to R₅ that is not the amine group represented by Formula 2, and R₆ to R₁₀ is a substituted or unsubstituted phenyl group, and the remaining are deuterium(s).

6. The light-emitting element of claim 1, wherein at least one hydrogen of FG1 and FG2, except those in R₁ to R₅ and R₆ to R₁₀, is substituted with deuterium.

7. The light-emitting element of claim 1, wherein the amine compound is a monoamine compound comprising no additional amino group as a substituent.

8. The light-emitting element of claim 1, wherein the light-emitting layer comprises a compound represented by Formula E-1: 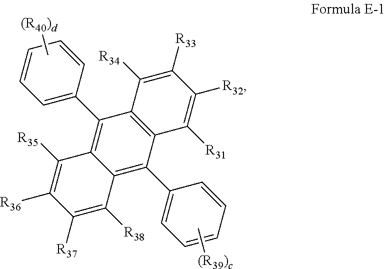
in Formula E-1,
c and d being each independently an integer of 0 to 5, and
R₃₁ to R₄₀ being each independently hydrogen, deuterium, a halogen, a substituted or unsubstituted silyl group, a substituted or unsubstituted thio group, a substituted or unsubstituted oxy group, a substituted or unsubstituted alkyl group having carbons of 1 to 10, a substituted or unsubstituted alkenyl group having carbons of 2 to 10, a substituted or unsubstituted aryl group having ring-forming carbons of 6 to 30, or a substituted or unsubstituted heteroaryl group having ring-forming carbons of 2 to 30, and/or being bonded to an adjacent group to form a ring.

9. The light-emitting element of claim 1, wherein the light-emitting layer is configured to emit blue light or green light.

10. The light-emitting element of claim 1, wherein the amine compound is any one selected from among compounds in Compound Group 1: 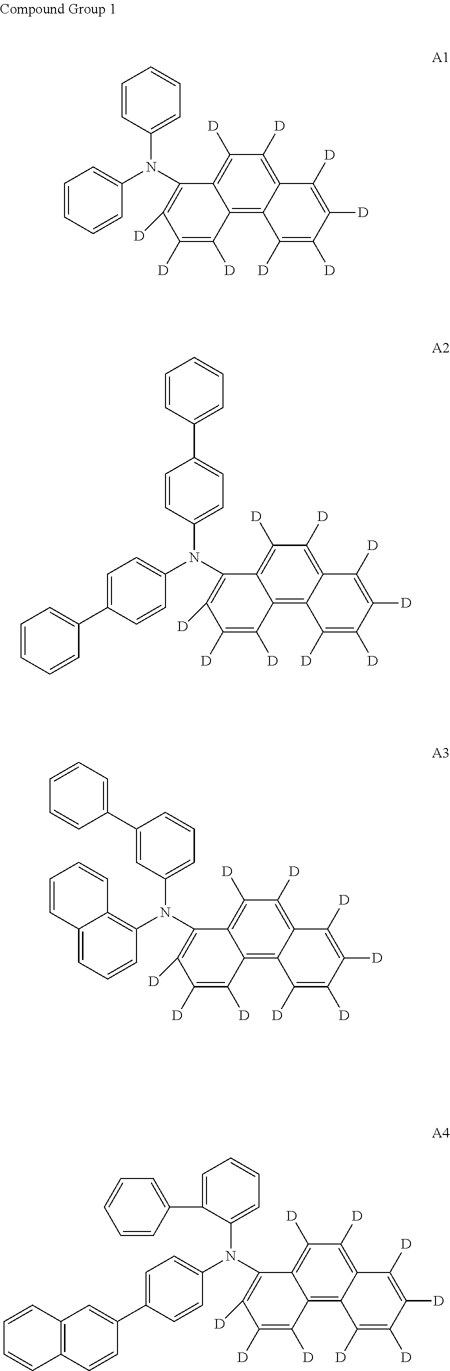 [SMILES: [2H]c1c([2H])c([2H])c2c(c1[2H])c([2H])c([2H])c1c(N(c3ccccc3)c3ccccc3)c([2H])c([2H])c([2H])c12 ; [2H]c1c([2H])c([2H])c2c(c1[2H])c([2H])c([2H])c1c(N(c3ccc(-c4ccccc4)cc3)c3ccc(-c4ccccc4)cc3)c([2H])c([2H])c([2H])c12 ; [2H]c1c([2H])c([2H])c2c(c1[2H])c([2H])c([2H])c1c(N(c3cccc(-c4ccccc4)c3)c3cccc4ccccc34)c([2H])c([2H])c([2H])c12 ; [2H]c1c([2H])c([2H])c2c(c1[2H])c([2H])c([2H])c1c(N(c3ccc(-c4ccc5ccccc5c4)cc3)c3ccccc3-c3ccccc3)c([2H])c([2H])c([2H])c12] 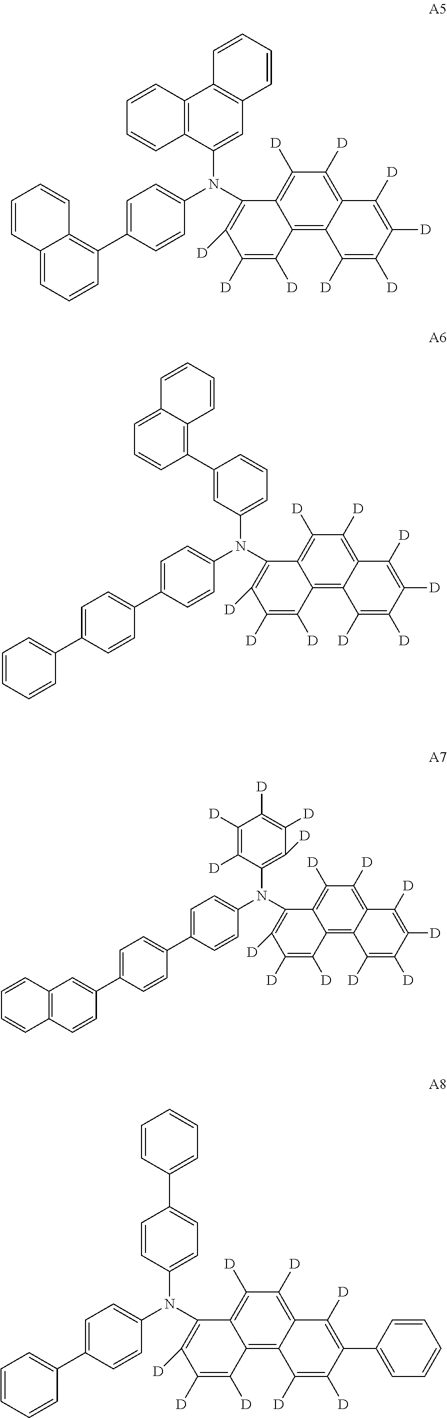 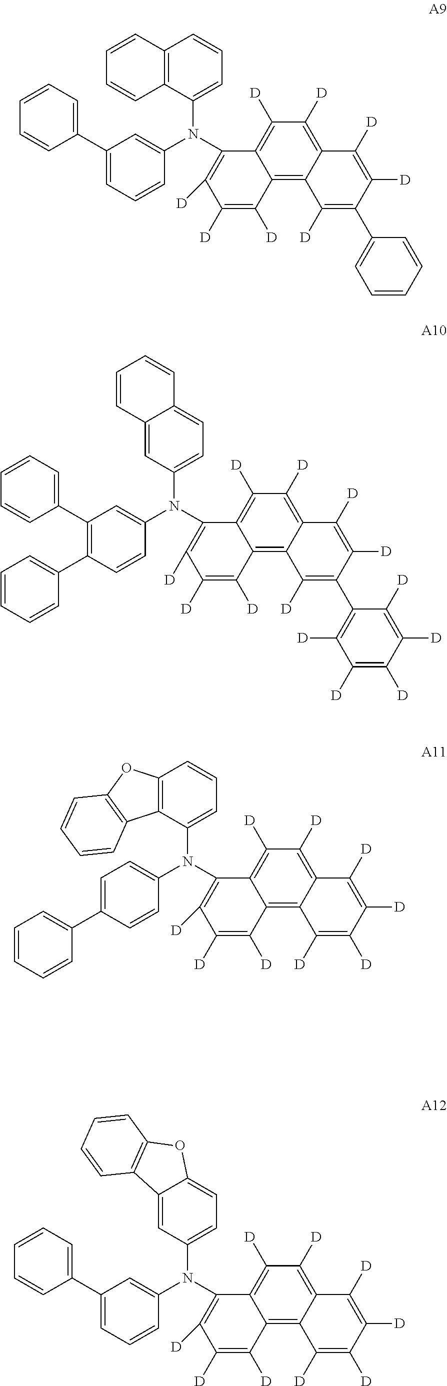 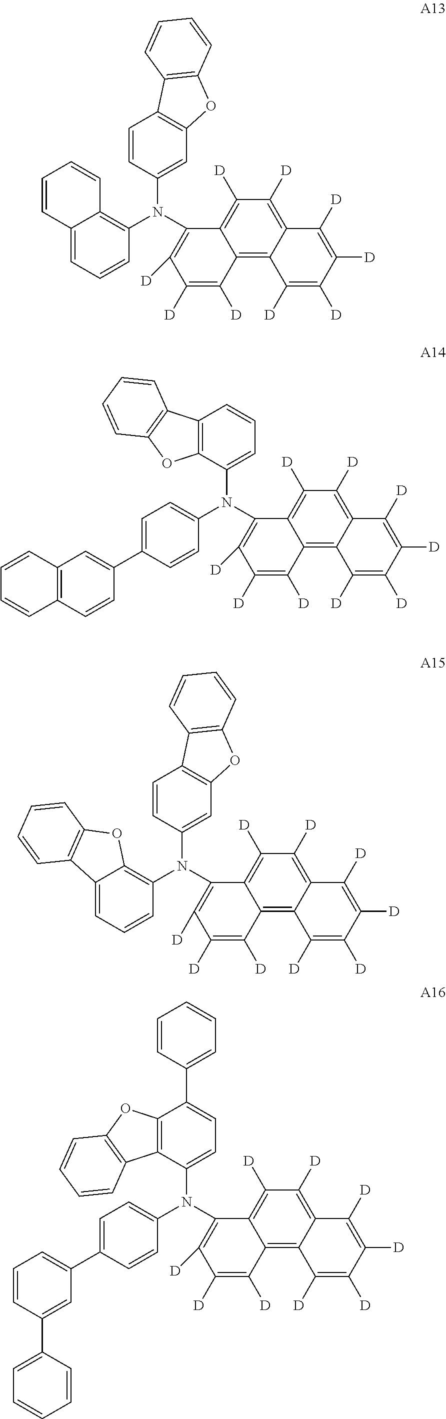 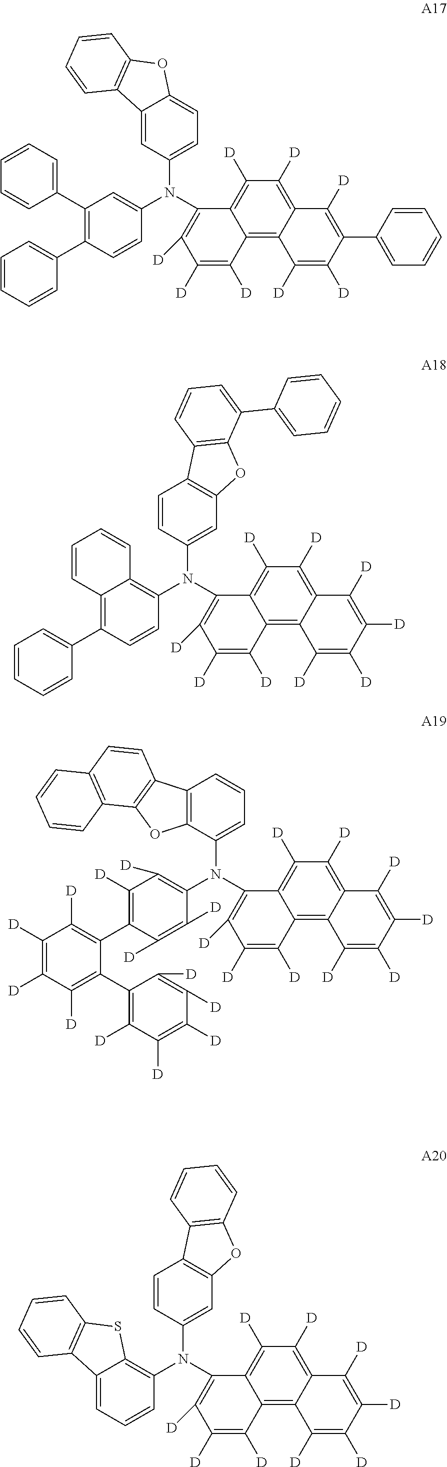 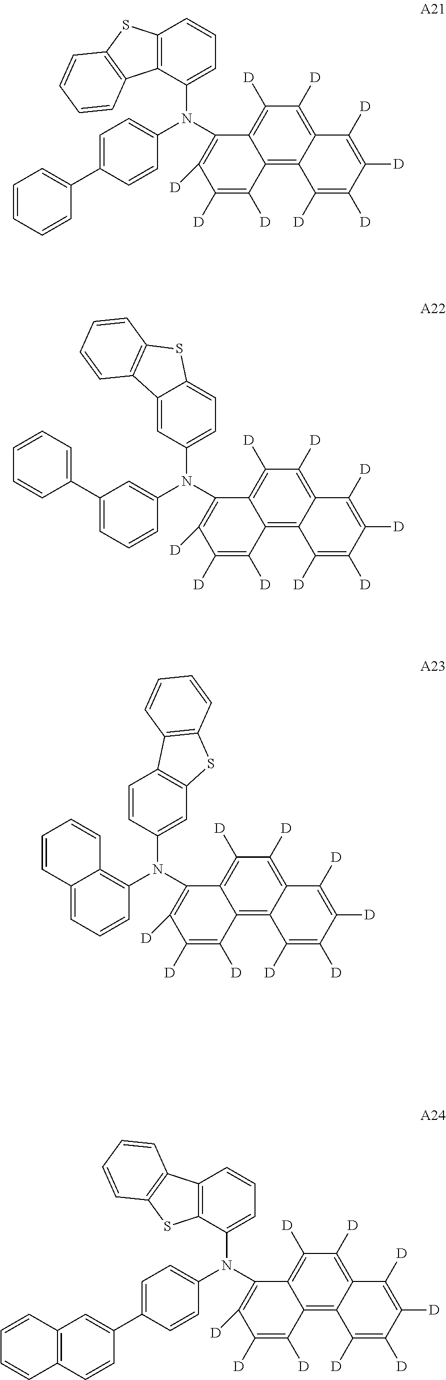 [SMILES: [2H]c1c([2H])c([2H])c2c(c1[2H])c([2H])c([2H])c1c(N(c3ccc(-c4ccccc4)cc3)c3cccc4sc5ccccc5c34)c([2H])c([2H])c([2H])c12 ; [2H]c1c([2H])c([2H])c2c(c1[2H])c([2H])c([2H])c1c(N(c3cccc(-c4ccccc4)c3)c3ccc4sc5ccccc5c4c3)c([2H])c([2H])c([2H])c12 ; [2H]c1c([2H])c([2H])c2c(c1[2H])c([2H])c([2H])c1c(N(c3ccc4c(c3)sc3ccccc34)c3cccc4ccccc34)c([2H])c([2H])c([2H])c12 ; [2H]c1c([2H])c([2H])c2c(c1[2H])c([2H])c([2H])c1c(N(c3ccc(-c4ccc5ccccc5c4)cc3)c3cccc4c3sc3ccccc34)c([2H])c([2H])c([2H])c12] 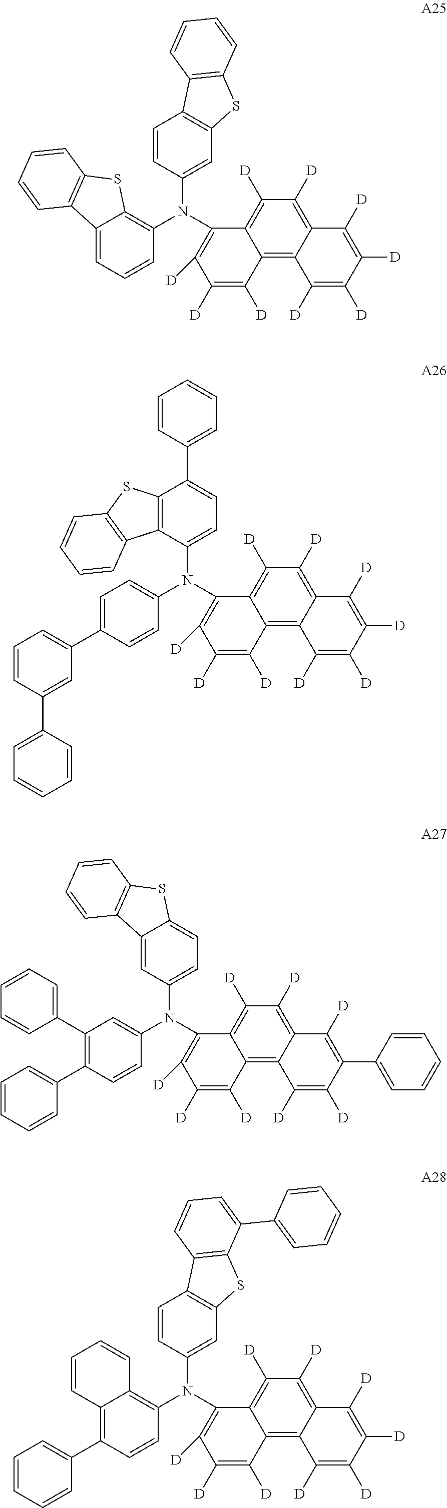 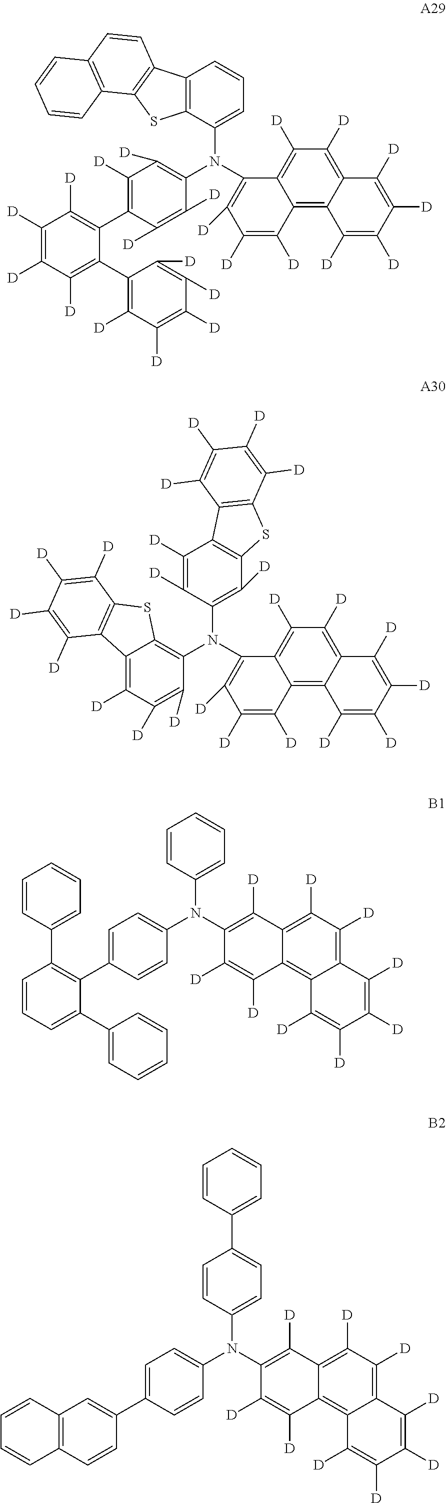 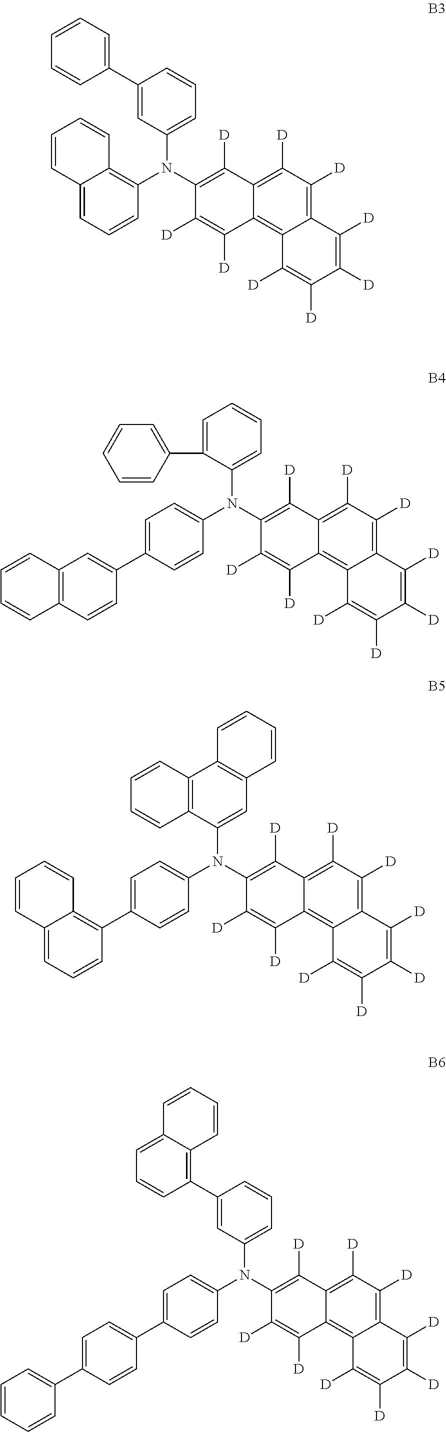 [SMILES: [2H]c1c([2H])c([2H])c2c(c1[2H])c([2H])c([2H])c1c([2H])c(N(c3cccc(-c4ccccc4)c3)c3cccc4ccccc34)c([2H])c([2H])c12 ; [2H]c1c([2H])c([2H])c2c(c1[2H])c([2H])c([2H])c1c([2H])c(N(c3ccc(-c4ccc5ccccc5c4)cc3)c3ccccc3-c3ccccc3)c([2H])c([2H])c12 ; [2H]c1c([2H])c([2H])c2c(c1[2H])c([2H])c([2H])c1c([2H])c(N(c3ccc(-c4cccc5ccccc45)cc3)c3cc4ccccc4c4ccccc34)c([2H])c([2H])c12 ; [2H]c1c([2H])c([2H])c2c(c1[2H])c([2H])c([2H])c1c([2H])c(N(c3ccc(-c4ccc(-c5ccccc5)cc4)cc3)c3cccc(-c4cccc5ccccc45)c3)c([2H])c([2H])c12] 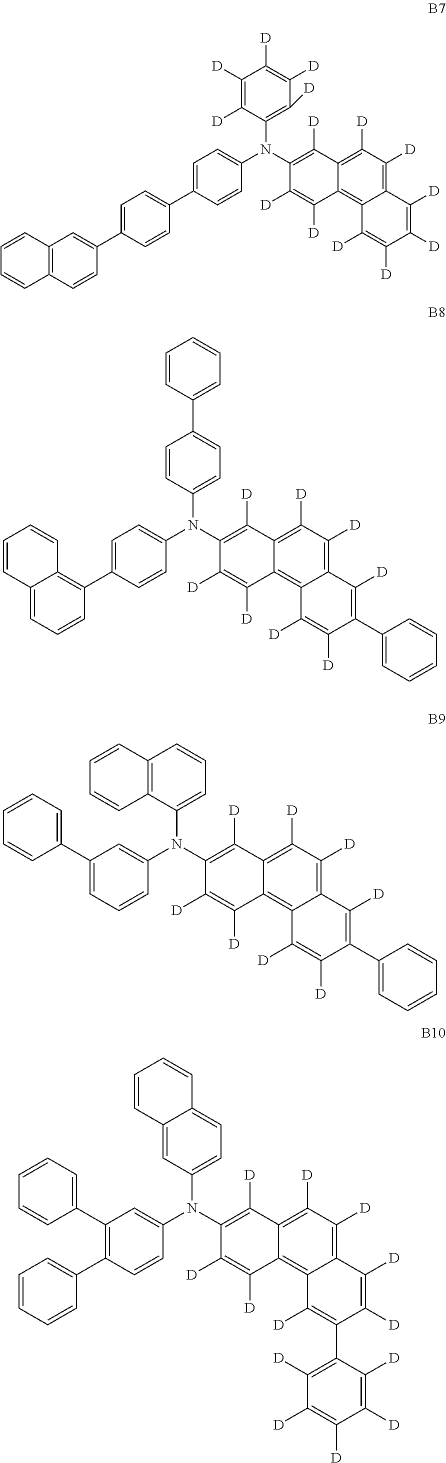 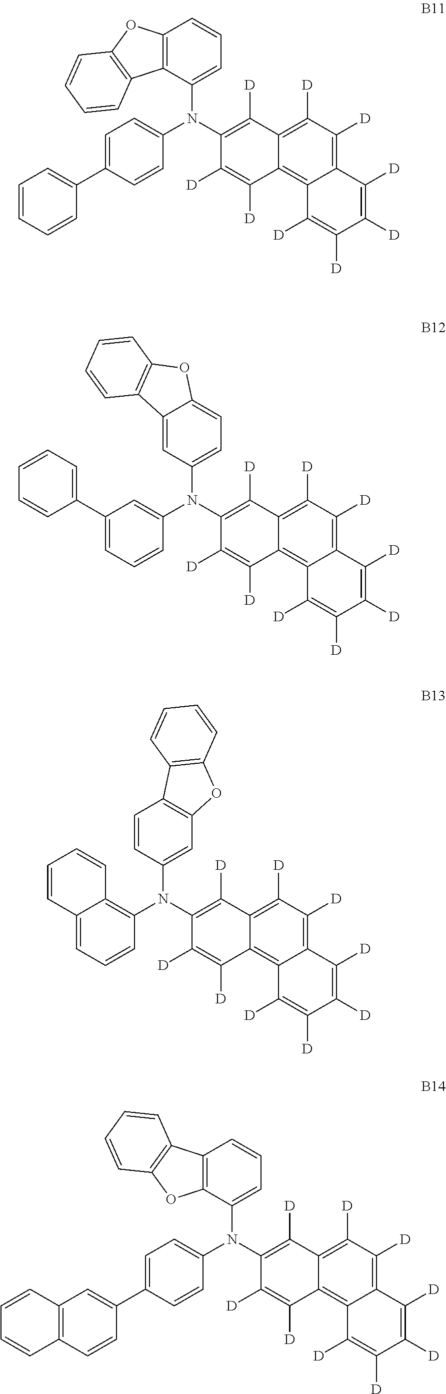 [SMILES: [2H]c1c([2H])c([2H])c2c(c1[2H])c([2H])c([2H])c1c([2H])c(N(c3ccc(-c4ccccc4)cc3)c3cccc4oc5ccccc5c34)c([2H])c([2H])c12 ; [2H]c1c([2H])c([2H])c2c(c1[2H])c([2H])c([2H])c1c([2H])c(N(c3cccc(-c4ccccc4)c3)c3ccc4oc5ccccc5c4c3)c([2H])c([2H])c12 ; [2H]c1c([2H])c([2H])c2c(c1[2H])c([2H])c([2H])c1c([2H])c(N(c3ccc4c(c3)oc3ccccc34)c3cccc4ccccc34)c([2H])c([2H])c12 ; [2H]c1c([2H])c([2H])c2c(c1[2H])c([2H])c([2H])c1c([2H])c(N(c3ccc(-c4ccc5ccccc5c4)cc3)c3cccc4c3oc3ccccc34)c([2H])c([2H])c12] 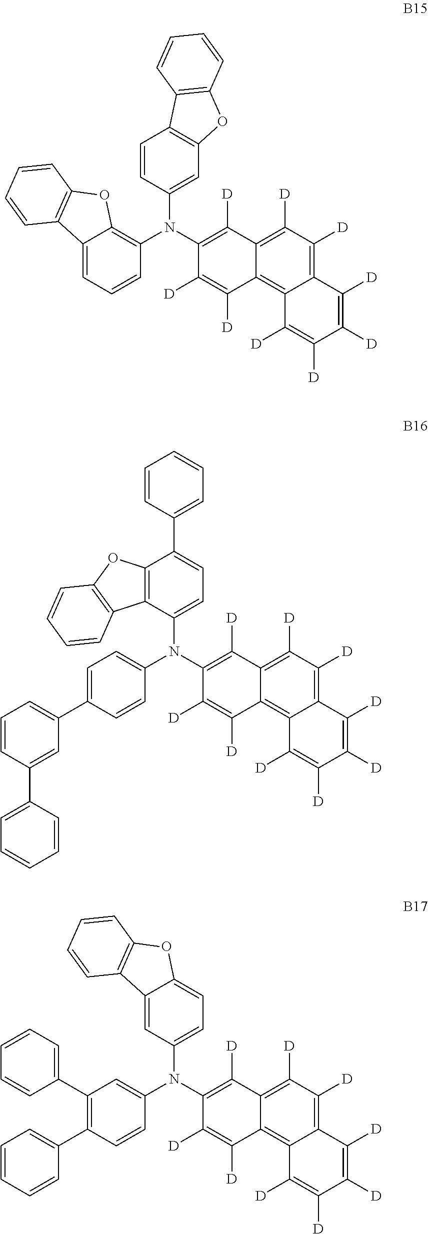 [SMILES: [2H]c1c([2H])c([2H])c2c(c1[2H])c([2H])c([2H])c1c([2H])c(N(c3ccc4c(c3)oc3ccccc34)c3cccc4c3oc3ccccc34)c([2H])c([2H])c12 ; [2H]c1c([2H])c([2H])c2c(c1[2H])c([2H])c([2H])c1c([2H])c(N(c3ccc(-c4cccc(-c5ccccc5)c4)cc3)c3ccc(-c4ccccc4)c4oc5ccccc5c34)c([2H])c([2H])c12 ; [2H]c1c([2H])c([2H])c2c(c1[2H])c([2H])c([2H])c1c([2H])c(N(c3ccc(-c4ccccc4)c(-c4ccccc4)c3)c3ccc4oc5ccccc5c4c3)c([2H])c([2H])c12] 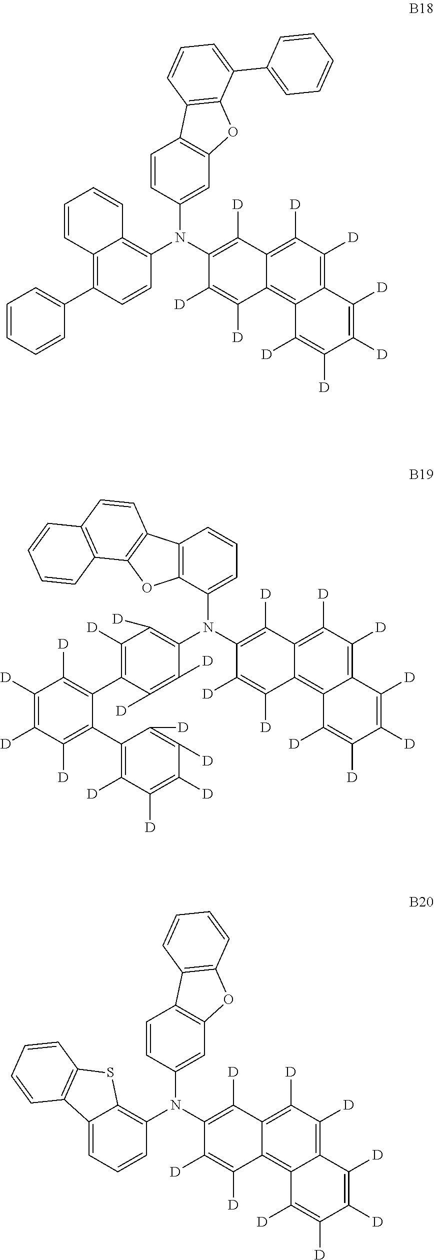 [SMILES: [2H]c1c([2H])c([2H])c2c(c1[2H])c([2H])c([2H])c1c([2H])c(N(c3ccc4c(c3)oc3c(-c5ccccc5)cccc34)c3ccc(-c4ccccc4)c4ccccc34)c([2H])c([2H])c12 ; [2H]c1c([2H])c([2H])c(-c2c([2H])c([2H])c([2H])c([2H])c2-c2c([2H])c([2H])c(N(c3c([2H])c([2H])c4c(c3[2H])c([2H])c([2H])c3c([2H])c([2H])c([2H])c([2H])c34)c3cccc4c3oc3c5ccccc5ccc43)c([2H])c2[2H])c([2H])c1[2H] ; [2H]c1c([2H])c([2H])c2c(c1[2H])c([2H])c([2H])c1c([2H])c(N(c3ccc4c(c3)oc3ccccc34)c3cccc4c3sc3ccccc34)c([2H])c([2H])c12] 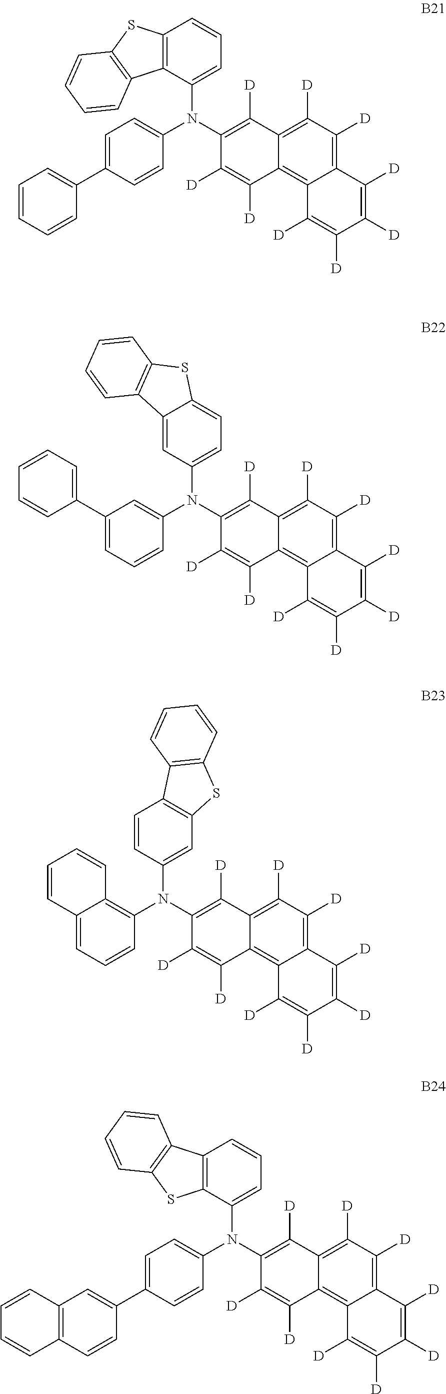 [SMILES: [2H]c1c([2H])c([2H])c2c(c1[2H])c([2H])c([2H])c1c([2H])c(N(c3ccc(-c4ccccc4)cc3)c3cccc4sc5ccccc5c34)c([2H])c([2H])c12 ; [2H]c1c([2H])c([2H])c2c(c1[2H])c([2H])c([2H])c1c([2H])c(N(c3cccc(-c4ccccc4)c3)c3ccc4sc5ccccc5c4c3)c([2H])c([2H])c12 ; [2H]c1c([2H])c([2H])c2c(c1[2H])c([2H])c([2H])c1c([2H])c(N(c3ccc4c(c3)sc3ccccc34)c3cccc4ccccc34)c([2H])c([2H])c12 ; [2H]c1c([2H])c([2H])c2c(c1[2H])c([2H])c([2H])c1c([2H])c(N(c3ccc(-c4ccc5ccccc5c4)cc3)c3cccc4c3sc3ccccc34)c([2H])c([2H])c12] 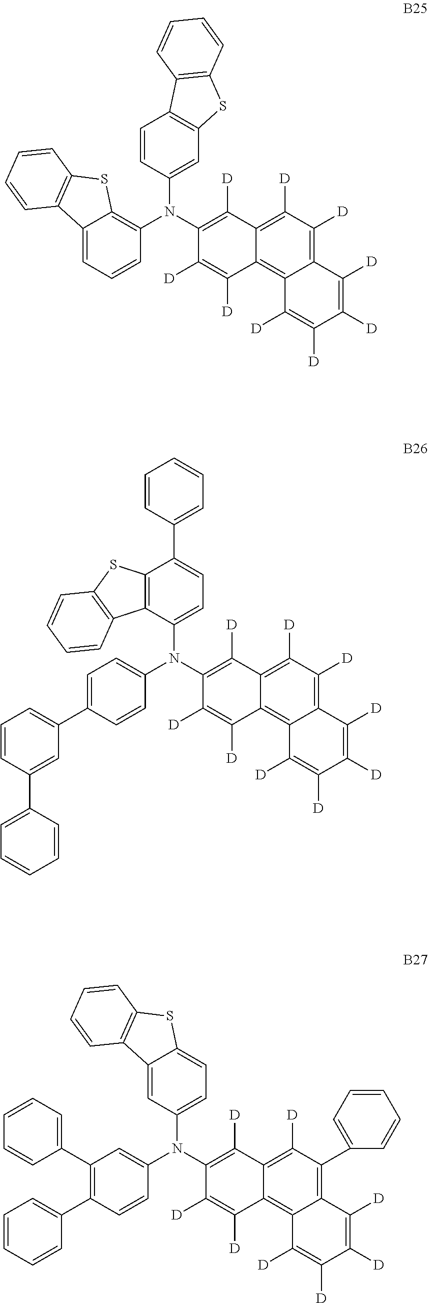 [SMILES: [2H]c1c([2H])c([2H])c2c(c1[2H])c([2H])c([2H])c1c([2H])c(N(c3ccc4c(c3)sc3ccccc34)c3cccc4c3sc3ccccc34)c([2H])c([2H])c12 ; [2H]c1c([2H])c([2H])c2c(c1[2H])c([2H])c([2H])c1c([2H])c(N(c3ccc(-c4cccc(-c5ccccc5)c4)cc3)c3ccc(-c4ccccc4)c4sc5ccccc5c34)c([2H])c([2H])c12 ; [2H]c1c([2H])c([2H])c2c(c1[2H])c(-c1ccccc1)c([2H])c1c([2H])c(N(c3ccc(-c4ccccc4)c(-c4ccccc4)c3)c3ccc4sc5ccccc5c4c3)c([2H])c([2H])c12] 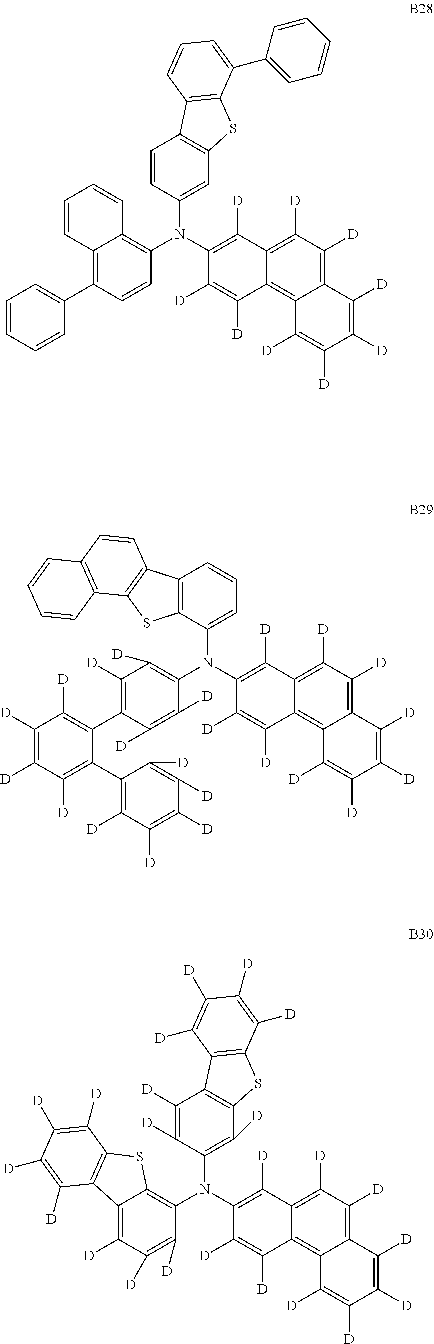 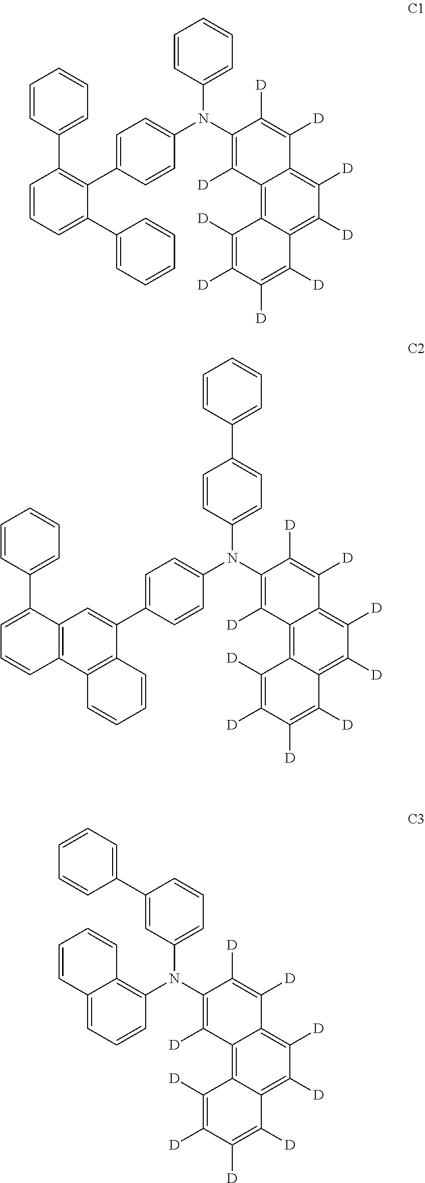 [SMILES: [2H]c1c([2H])c([2H])c2c(c1[2H])c([2H])c([2H])c1c([2H])c([2H])c(N(c3ccccc3)c3ccc(-c4c(-c5ccccc5)cccc4-c4ccccc4)cc3)c([2H])c12 ; [2H]c1c([2H])c([2H])c2c(c1[2H])c([2H])c([2H])c1c([2H])c([2H])c(N(c3ccc(-c4ccccc4)cc3)c3ccc(-c4cc5c(-c6ccccc6)cccc5c5ccccc45)cc3)c([2H])c12 ; [2H]c1c([2H])c([2H])c2c(c1[2H])c([2H])c([2H])c1c([2H])c([2H])c(N(c3cccc(-c4ccccc4)c3)c3cccc4ccccc34)c([2H])c12] 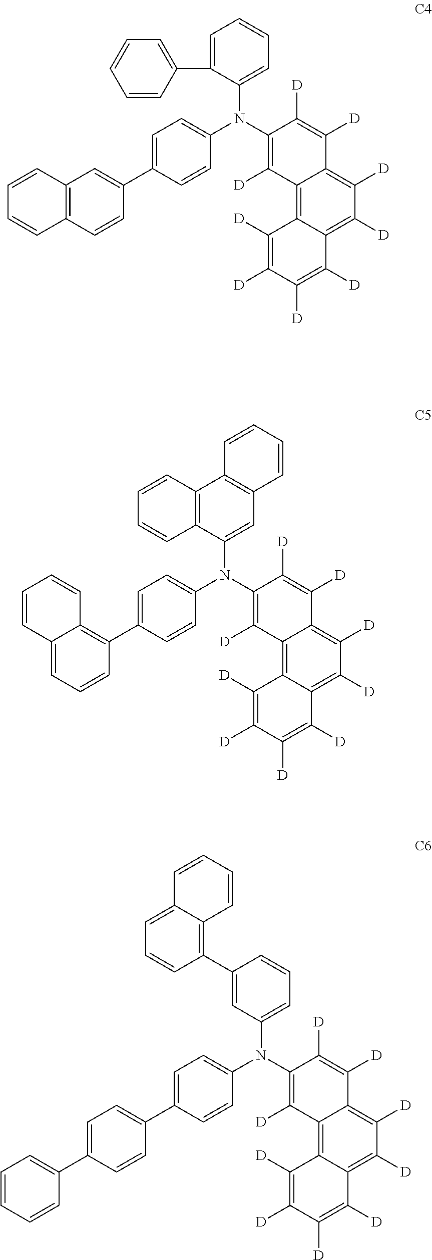 [SMILES: [2H]c1c([2H])c([2H])c2c(c1[2H])c([2H])c([2H])c1c([2H])c([2H])c(N(c3ccc(-c4ccc5ccccc5c4)cc3)c3ccccc3-c3ccccc3)c([2H])c12 ; [2H]c1c([2H])c([2H])c2c(c1[2H])c([2H])c([2H])c1c([2H])c([2H])c(N(c3ccc(-c4cccc5ccccc45)cc3)c3cc4ccccc4c4ccccc34)c([2H])c12 ; [2H]c1c([2H])c([2H])c2c(c1[2H])c([2H])c([2H])c1c([2H])c([2H])c(N(c3ccc(-c4ccc(-c5ccccc5)cc4)cc3)c3cccc(-c4cccc5ccccc45)c3)c([2H])c12] 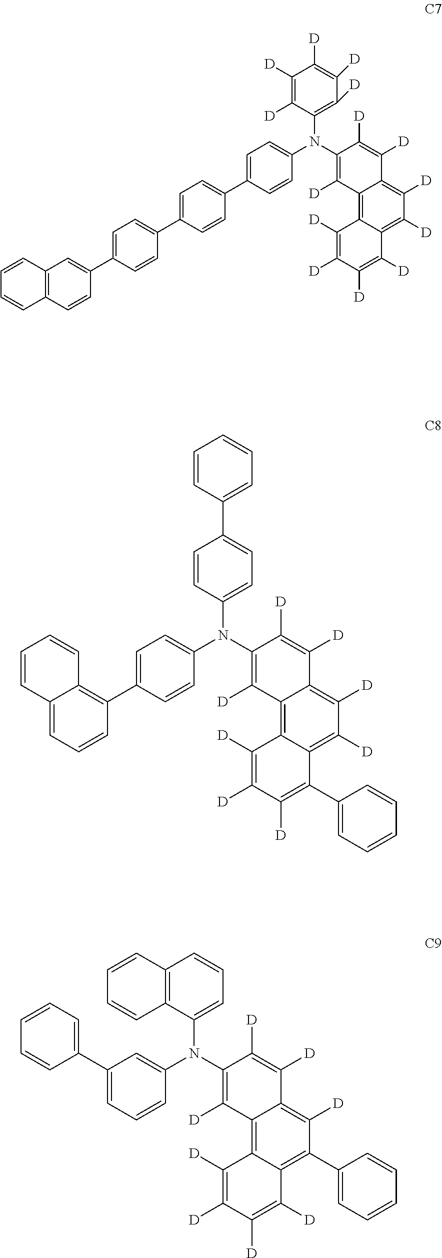 [SMILES: [2H]c1c([2H])c([2H])c(N(c2ccc(-c3ccc(-c4ccc(-c5ccc6ccccc6c5)cc4)cc3)cc2)c2c([2H])c([2H])c3c([2H])c([2H])c4c([2H])c([2H])c([2H])c([2H])c4c3c2[2H])c([2H])c1[2H] ; [2H]c1c(N(c2ccc(-c3ccccc3)cc2)c2ccc(-c3cccc4ccccc34)cc2)c([2H])c2c(c1[2H])c([2H])c([2H])c1c(-c3ccccc3)c([2H])c([2H])c([2H])c12 ; [2H]c1c(N(c2cccc(-c3ccccc3)c2)c2cccc3ccccc23)c([2H])c2c(c1[2H])c([2H])c(-c1ccccc1)c1c([2H])c([2H])c([2H])c([2H])c12] 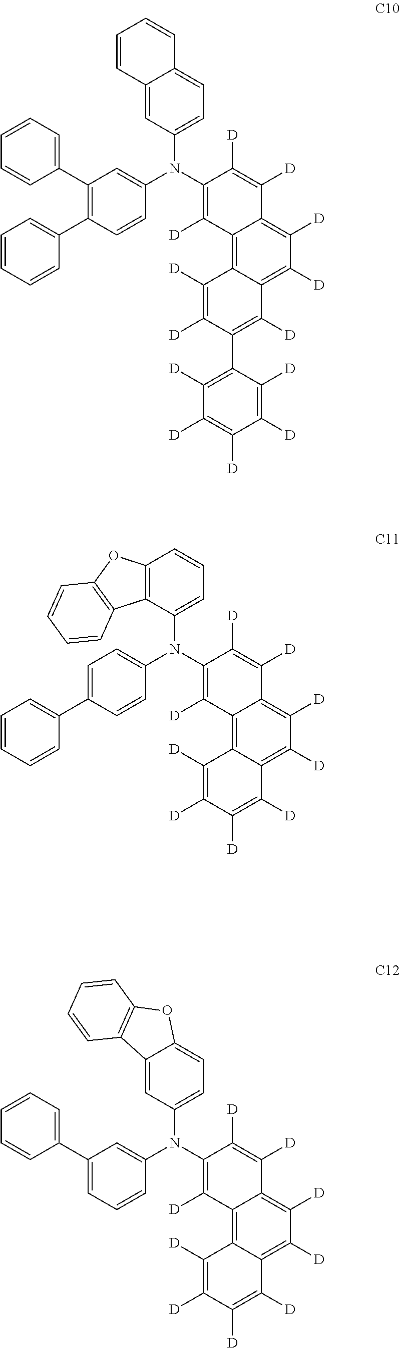 [SMILES: [2H]c1c([2H])c([2H])c(-c2c([2H])c([2H])c3c(c2[2H])c([2H])c([2H])c2c([2H])c([2H])c(N(c4ccc(-c5ccccc5)c(-c5ccccc5)c4)c4ccc5ccccc5c4)c([2H])c23)c([2H])c1[2H] ; [2H]c1c([2H])c([2H])c2c(c1[2H])c([2H])c([2H])c1c([2H])c([2H])c(N(c3ccc(-c4ccccc4)cc3)c3cccc4oc5ccccc5c34)c([2H])c12 ; [2H]c1c([2H])c([2H])c2c(c1[2H])c([2H])c([2H])c1c([2H])c([2H])c(N(c3cccc(-c4ccccc4)c3)c3ccc4oc5ccccc5c4c3)c([2H])c12] 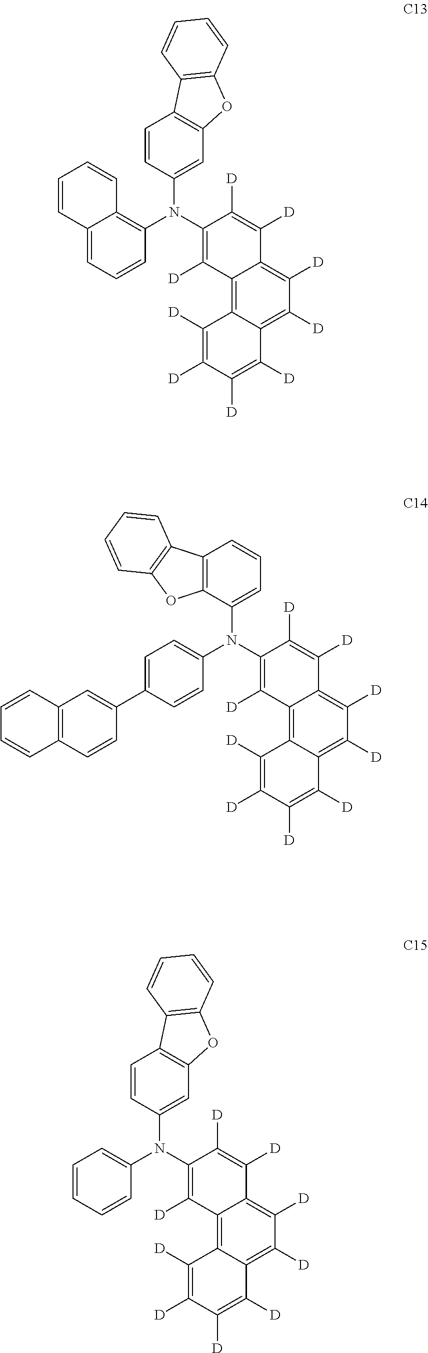 [SMILES: [2H]c1c([2H])c([2H])c2c(c1[2H])c([2H])c([2H])c1c([2H])c([2H])c(N(c3ccc4c(c3)oc3ccccc34)c3cccc4ccccc34)c([2H])c12 ; [2H]c1c([2H])c([2H])c2c(c1[2H])c([2H])c([2H])c1c([2H])c([2H])c(N(c3ccc(-c4ccc5ccccc5c4)cc3)c3cccc4c3oc3ccccc34)c([2H])c12 ; [2H]c1c([2H])c([2H])c2c(c1[2H])c([2H])c([2H])c1c([2H])c([2H])c(N(c3ccccc3)c3ccc4c(c3)oc3ccccc34)c([2H])c12] 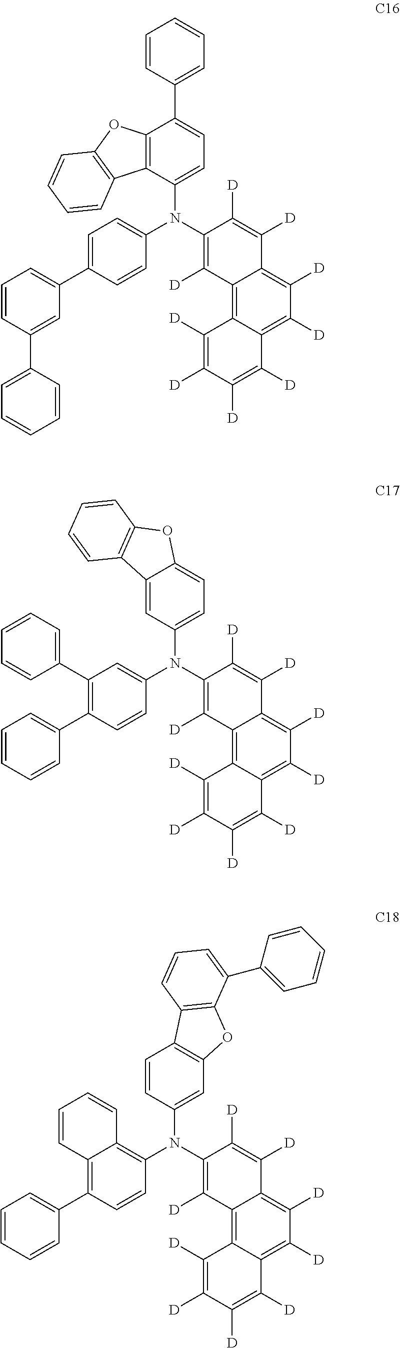 [SMILES: [2H]c1c([2H])c([2H])c2c(c1[2H])c([2H])c([2H])c1c([2H])c([2H])c(N(c3ccc(-c4cccc(-c5ccccc5)c4)cc3)c3ccc(-c4ccccc4)c4oc5ccccc5c34)c([2H])c12 ; [2H]c1c([2H])c([2H])c2c(c1[2H])c([2H])c([2H])c1c([2H])c([2H])c(N(c3ccc(-c4ccccc4)c(-c4ccccc4)c3)c3ccc4oc5ccccc5c4c3)c([2H])c12 ; [2H]c1c([2H])c([2H])c2c(c1[2H])c([2H])c([2H])c1c([2H])c([2H])c(N(c3ccc4c(c3)oc3c(-c5ccccc5)cccc34)c3ccc(-c4ccccc4)c4ccccc34)c([2H])c12] 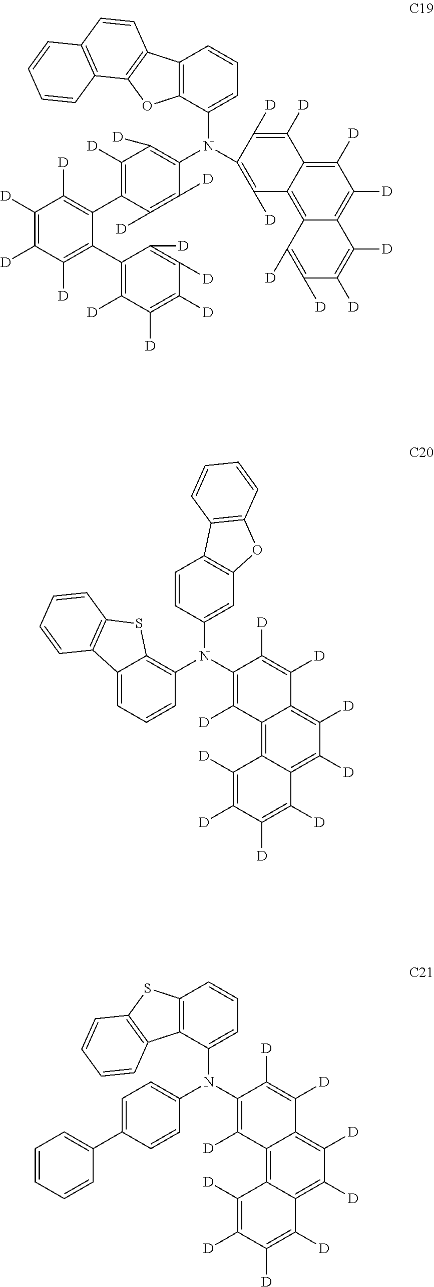 [SMILES: [2H]c1c([2H])c([2H])c(-c2c([2H])c([2H])c([2H])c([2H])c2-c2c([2H])c([2H])c(N(c3c([2H])c([2H])c4c([2H])c([2H])c5c([2H])c([2H])c([2H])c([2H])c5c4c3[2H])c3cccc4c3oc3c5ccccc5ccc43)c([2H])c2[2H])c([2H])c1[2H] ; [2H]c1c([2H])c([2H])c2c(c1[2H])c([2H])c([2H])c1c([2H])c([2H])c(N(c3ccc4c(c3)oc3ccccc34)c3cccc4c3sc3ccccc34)c([2H])c12 ; [2H]c1c([2H])c([2H])c2c(c1[2H])c([2H])c([2H])c1c([2H])c([2H])c(N(c3ccc(-c4ccccc4)cc3)c3cccc4sc5ccccc5c34)c([2H])c12] 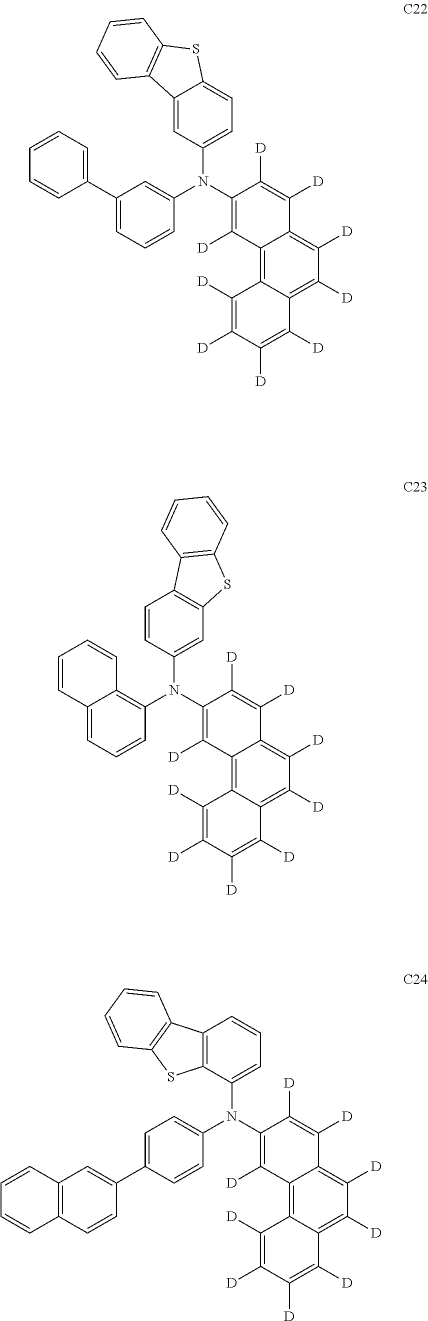 [SMILES: [2H]c1c([2H])c([2H])c2c(c1[2H])c([2H])c([2H])c1c([2H])c([2H])c(N(c3cccc(-c4ccccc4)c3)c3ccc4sc5ccccc5c4c3)c([2H])c12 ; [2H]c1c([2H])c([2H])c2c(c1[2H])c([2H])c([2H])c1c([2H])c([2H])c(N(c3ccc4c(c3)sc3ccccc34)c3cccc4ccccc34)c([2H])c12 ; [2H]c1c([2H])c([2H])c2c(c1[2H])c([2H])c([2H])c1c([2H])c([2H])c(N(c3ccc(-c4ccc5ccccc5c4)cc3)c3cccc4c3sc3ccccc34)c([2H])c12] 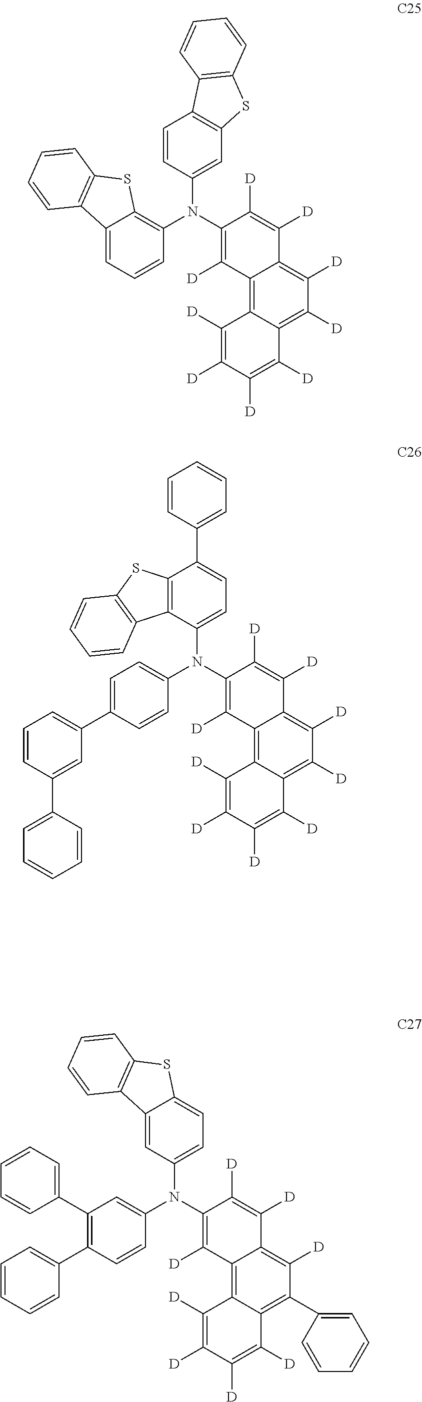 [SMILES: [2H]c1c([2H])c([2H])c2c(c1[2H])c([2H])c([2H])c1c([2H])c([2H])c(N(c3ccc4c(c3)sc3ccccc34)c3cccc4c3sc3ccccc34)c([2H])c12 ; [2H]c1c([2H])c([2H])c2c(c1[2H])c([2H])c([2H])c1c([2H])c([2H])c(N(c3ccc(-c4cccc(-c5ccccc5)c4)cc3)c3ccc(-c4ccccc4)c4sc5ccccc5c34)c([2H])c12 ; [2H]c1c(N(c2ccc(-c3ccccc3)c(-c3ccccc3)c2)c2ccc3sc4ccccc4c3c2)c([2H])c2c(c1[2H])c([2H])c(-c1ccccc1)c1c([2H])c([2H])c([2H])c([2H])c12] 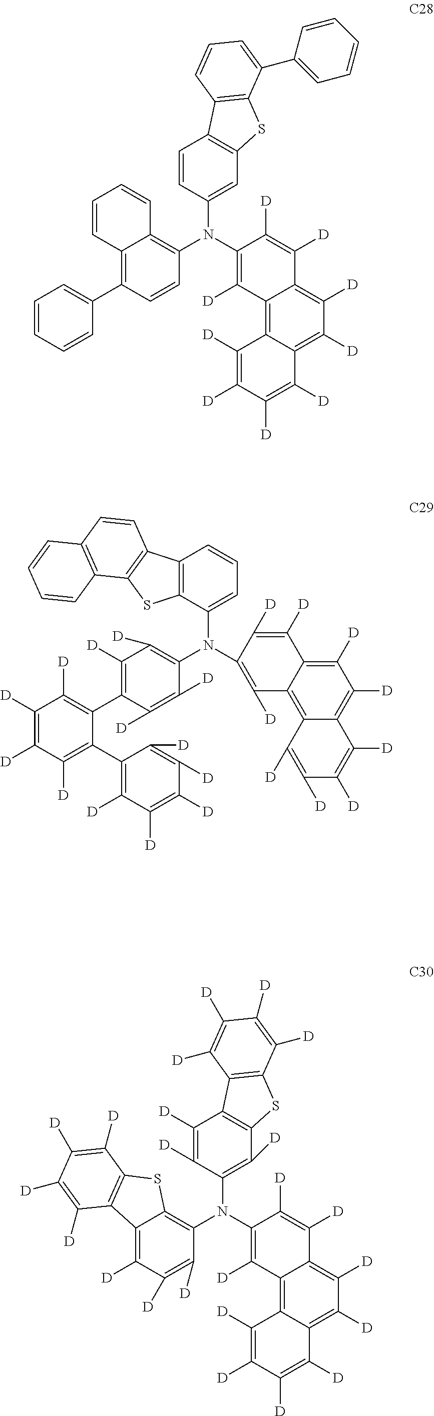 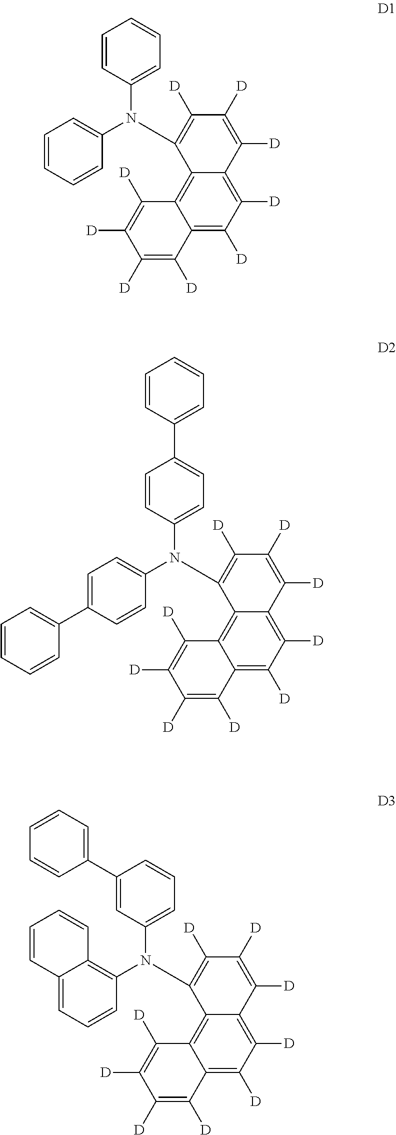 [SMILES: [2H]c1c([2H])c([2H])c2c(c1[2H])c([2H])c([2H])c1c([2H])c([2H])c([2H])c(N(c3ccccc3)c3ccccc3)c12 ; [2H]c1c([2H])c([2H])c2c(c1[2H])c([2H])c([2H])c1c([2H])c([2H])c([2H])c(N(c3ccc(-c4ccccc4)cc3)c3ccc(-c4ccccc4)cc3)c12 ; [2H]c1c([2H])c([2H])c2c(c1[2H])c([2H])c([2H])c1c([2H])c([2H])c([2H])c(N(c3cccc(-c4ccccc4)c3)c3cccc4ccccc34)c12] 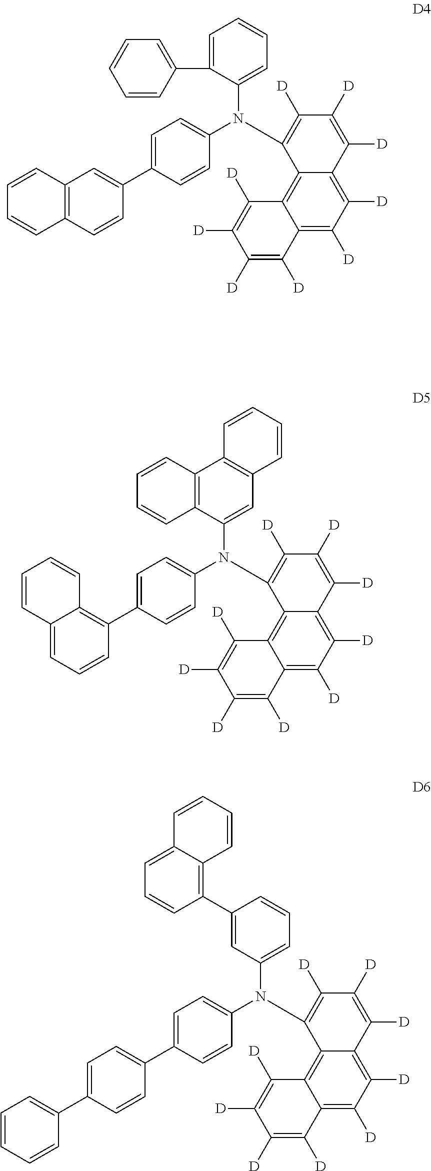 [SMILES: [2H]c1c([2H])c([2H])c2c(c1[2H])c([2H])c([2H])c1c([2H])c([2H])c([2H])c(N(c3ccc(-c4ccc5ccccc5c4)cc3)c3ccccc3-c3ccccc3)c12 ; [2H]c1c([2H])c([2H])c2c(c1[2H])c([2H])c([2H])c1c([2H])c([2H])c([2H])c(N(c3ccc(-c4cccc5ccccc45)cc3)c3cc4ccccc4c4ccccc34)c12 ; [2H]c1c([2H])c([2H])c2c(c1[2H])c([2H])c([2H])c1c([2H])c([2H])c([2H])c(N(c3ccc(-c4ccc(-c5ccccc5)cc4)cc3)c3cccc(-c4cccc5ccccc45)c3)c12] 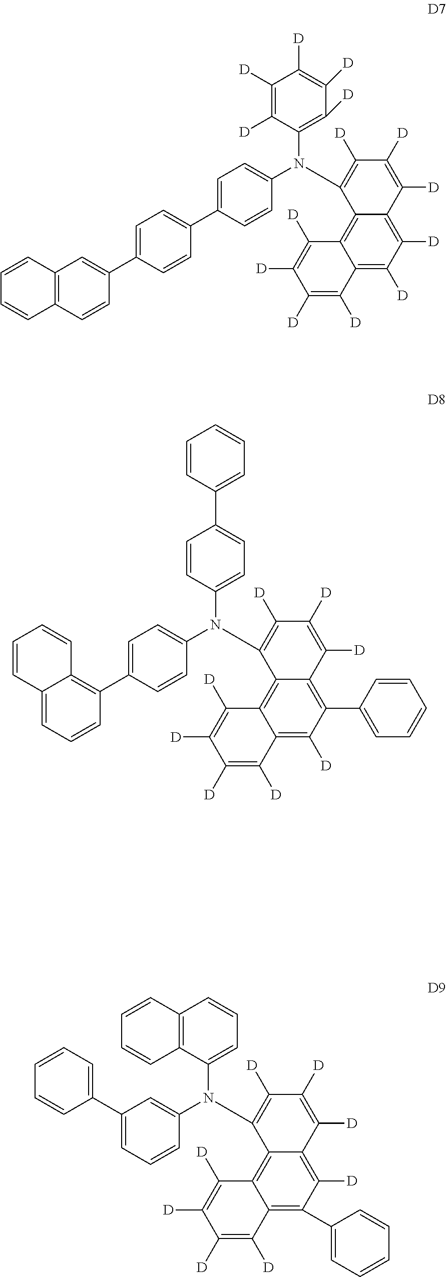 [SMILES: [2H]c1c([2H])c([2H])c(N(c2ccc(-c3ccc(-c4ccc5ccccc5c4)cc3)cc2)c2c([2H])c([2H])c([2H])c3c([2H])c([2H])c4c([2H])c([2H])c([2H])c([2H])c4c23)c([2H])c1[2H] ; [2H]c1c([2H])c([2H])c2c(c1[2H])c([2H])c(-c1ccccc1)c1c([2H])c([2H])c([2H])c(N(c3ccc(-c4ccccc4)cc3)c3ccc(-c4cccc5ccccc45)cc3)c12 ; [2H]c1c([2H])c([2H])c2c(c1[2H])c(-c1ccccc1)c([2H])c1c([2H])c([2H])c([2H])c(N(c3cccc(-c4ccccc4)c3)c3cccc4ccccc34)c12] 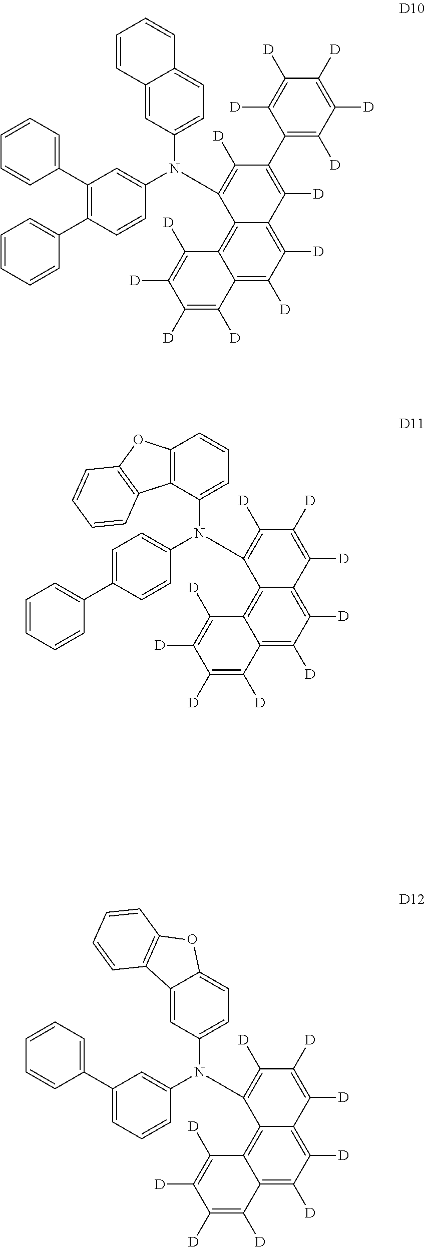 [SMILES: [2H]c1c([2H])c([2H])c(-c2c([2H])c(N(c3ccc(-c4ccccc4)c(-c4ccccc4)c3)c3ccc4ccccc4c3)c3c(c2[2H])c([2H])c([2H])c2c([2H])c([2H])c([2H])c([2H])c23)c([2H])c1[2H] ; [2H]c1c([2H])c([2H])c2c(c1[2H])c([2H])c([2H])c1c([2H])c([2H])c([2H])c(N(c3ccc(-c4ccccc4)cc3)c3cccc4oc5ccccc5c34)c12 ; [2H]c1c([2H])c([2H])c2c(c1[2H])c([2H])c([2H])c1c([2H])c([2H])c([2H])c(N(c3cccc(-c4ccccc4)c3)c3ccc4oc5ccccc5c4c3)c12] 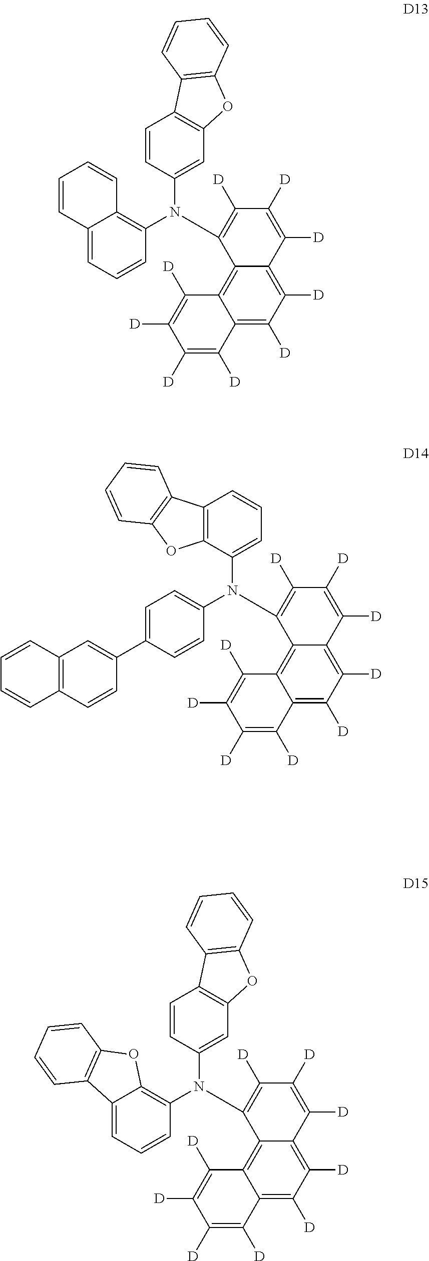 [SMILES: [2H]c1c([2H])c([2H])c2c(c1[2H])c([2H])c([2H])c1c([2H])c([2H])c([2H])c(N(c3ccc4c(c3)oc3ccccc34)c3cccc4ccccc34)c12 ; [2H]c1c([2H])c([2H])c2c(c1[2H])c([2H])c([2H])c1c([2H])c([2H])c([2H])c(N(c3ccc(-c4ccc5ccccc5c4)cc3)c3cccc4c3oc3ccccc34)c12 ; [2H]c1c([2H])c([2H])c2c(c1[2H])c([2H])c([2H])c1c([2H])c([2H])c([2H])c(N(c3ccc4c(c3)oc3ccccc34)c3cccc4c3oc3ccccc34)c12] 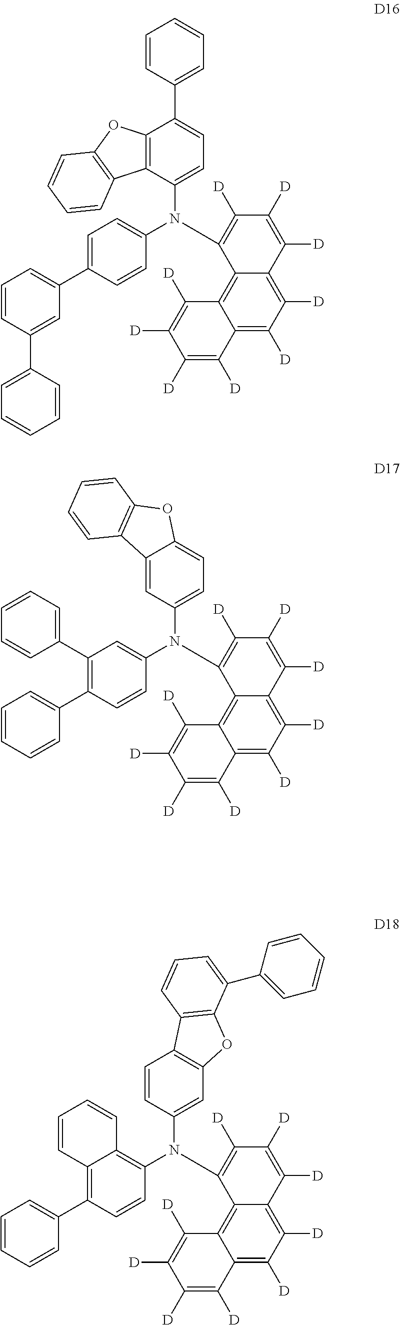 [SMILES: [2H]c1c([2H])c([2H])c2c(c1[2H])c([2H])c([2H])c1c([2H])c([2H])c([2H])c(N(c3ccc(-c4cccc(-c5ccccc5)c4)cc3)c3ccc(-c4ccccc4)c4oc5ccccc5c34)c12 ; [2H]c1c([2H])c([2H])c2c(c1[2H])c([2H])c([2H])c1c([2H])c([2H])c([2H])c(N(c3ccc(-c4ccccc4)c(-c4ccccc4)c3)c3ccc4oc5ccccc5c4c3)c12 ; [2H]c1c([2H])c([2H])c2c(c1[2H])c([2H])c([2H])c1c([2H])c([2H])c([2H])c(N(c3ccc4c(c3)oc3c(-c5ccccc5)cccc34)c3ccc(-c4ccccc4)c4ccccc34)c12] 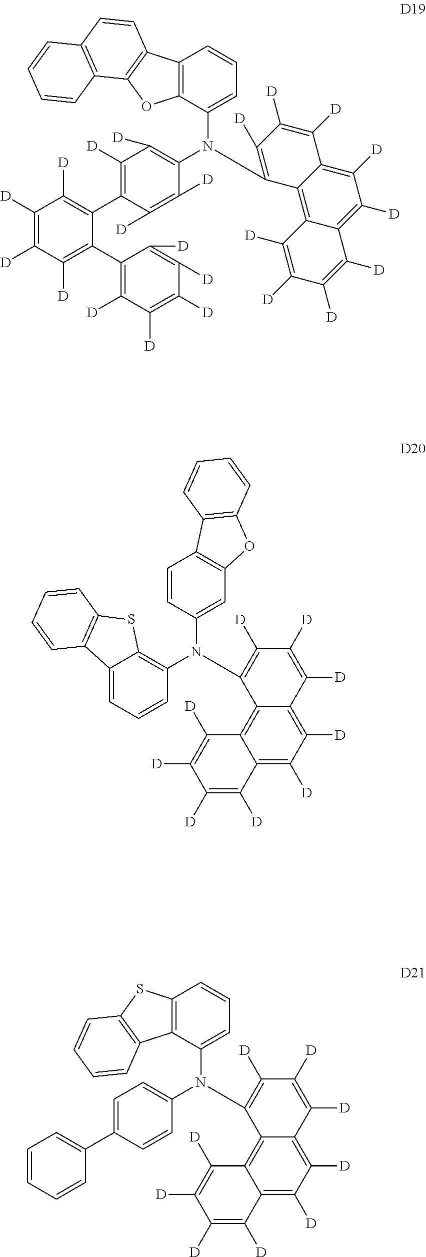 [SMILES: [2H]c1c([2H])c([2H])c(-c2c([2H])c([2H])c([2H])c([2H])c2-c2c([2H])c([2H])c(N(c3cccc4c3oc3c5ccccc5ccc43)c3c([2H])c([2H])c([2H])c4c([2H])c([2H])c5c([2H])c([2H])c([2H])c([2H])c5c34)c([2H])c2[2H])c([2H])c1[2H] ; [2H]c1c([2H])c([2H])c2c(c1[2H])c([2H])c([2H])c1c([2H])c([2H])c([2H])c(N(c3ccc4c(c3)oc3ccccc34)c3cccc4c3sc3ccccc34)c12 ; [2H]c1c([2H])c([2H])c2c(c1[2H])c([2H])c([2H])c1c([2H])c([2H])c([2H])c(N(c3ccc(-c4ccccc4)cc3)c3cccc4sc5ccccc5c34)c12] 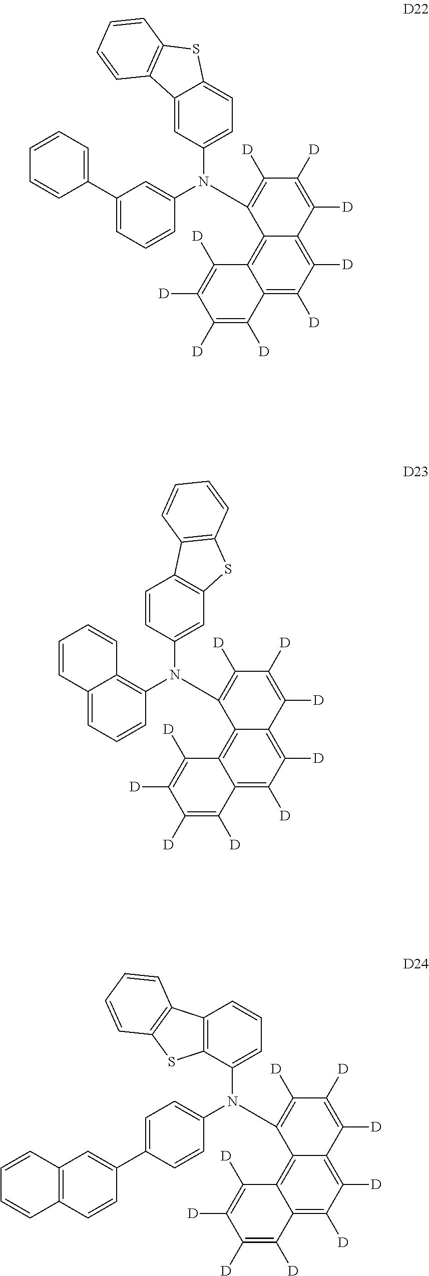 [SMILES: [2H]c1c([2H])c([2H])c2c(c1[2H])c([2H])c([2H])c1c([2H])c([2H])c([2H])c(N(c3cccc(-c4ccccc4)c3)c3ccc4sc5ccccc5c4c3)c12 ; [2H]c1c([2H])c([2H])c2c(c1[2H])c([2H])c([2H])c1c([2H])c([2H])c([2H])c(N(c3ccc4c(c3)sc3ccccc34)c3cccc4ccccc34)c12 ; [2H]c1c([2H])c([2H])c2c(c1[2H])c([2H])c([2H])c1c([2H])c([2H])c([2H])c(N(c3ccc(-c4ccc5ccccc5c4)cc3)c3cccc4c3sc3ccccc34)c12] 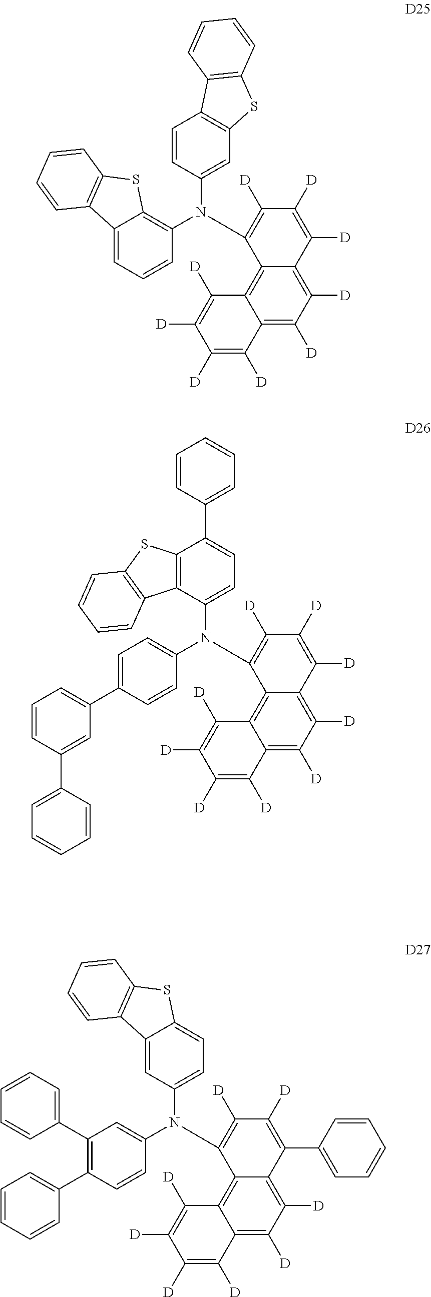 [SMILES: [2H]c1c([2H])c([2H])c2c(c1[2H])c([2H])c([2H])c1c([2H])c([2H])c([2H])c(N(c3ccc4c(c3)sc3ccccc34)c3cccc4c3sc3ccccc34)c12 ; [2H]c1c([2H])c([2H])c2c(c1[2H])c([2H])c([2H])c1c([2H])c([2H])c([2H])c(N(c3ccc(-c4cccc(-c5ccccc5)c4)cc3)c3ccc(-c4ccccc4)c4sc5ccccc5c34)c12 ; [2H]c1c([2H])c([2H])c2c(c1[2H])c([2H])c([2H])c1c(-c3ccccc3)c([2H])c([2H])c(N(c3ccc(-c4ccccc4)c(-c4ccccc4)c3)c3ccc4sc5ccccc5c4c3)c12] 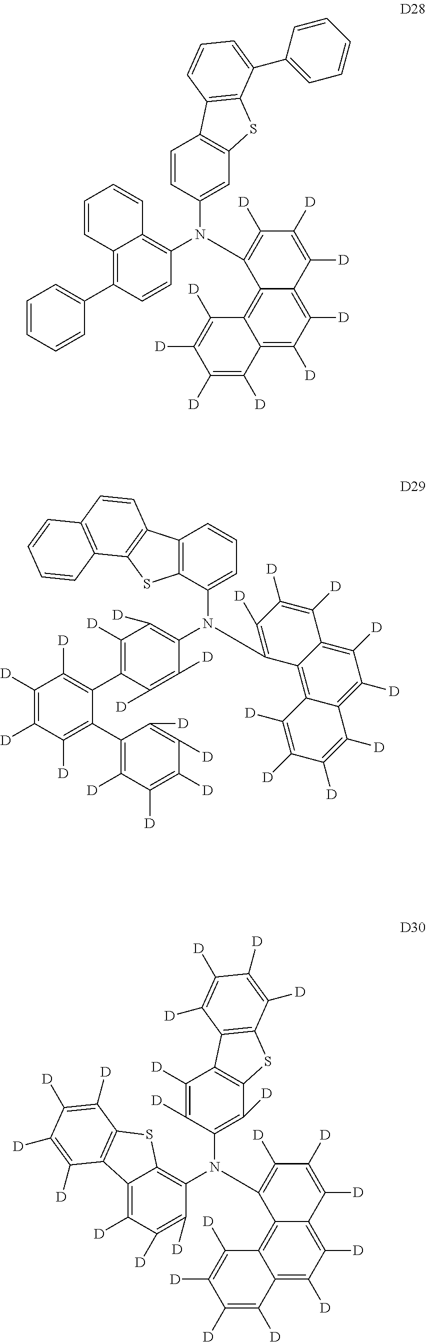 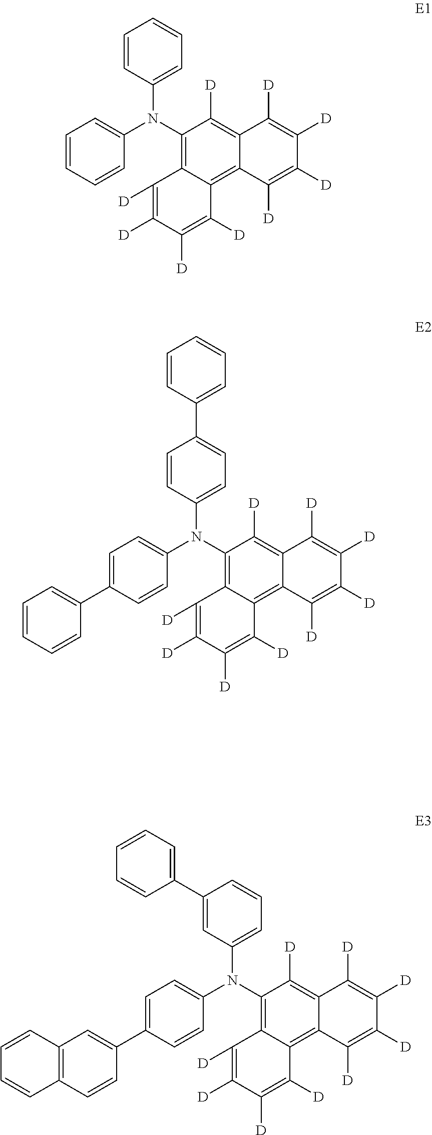 [SMILES: [2H]c1c([2H])c([2H])c2c(c1[2H])c([2H])c(N(c1ccccc1)c1ccccc1)c1c([2H])c([2H])c([2H])c([2H])c12 ; [2H]c1c([2H])c([2H])c2c(c1[2H])c([2H])c(N(c1ccc(-c3ccccc3)cc1)c1ccc(-c3ccccc3)cc1)c1c([2H])c([2H])c([2H])c([2H])c12 ; [2H]c1c([2H])c([2H])c2c(c1[2H])c([2H])c(N(c1ccc(-c3ccc4ccccc4c3)cc1)c1cccc(-c3ccccc3)c1)c1c([2H])c([2H])c([2H])c([2H])c12] 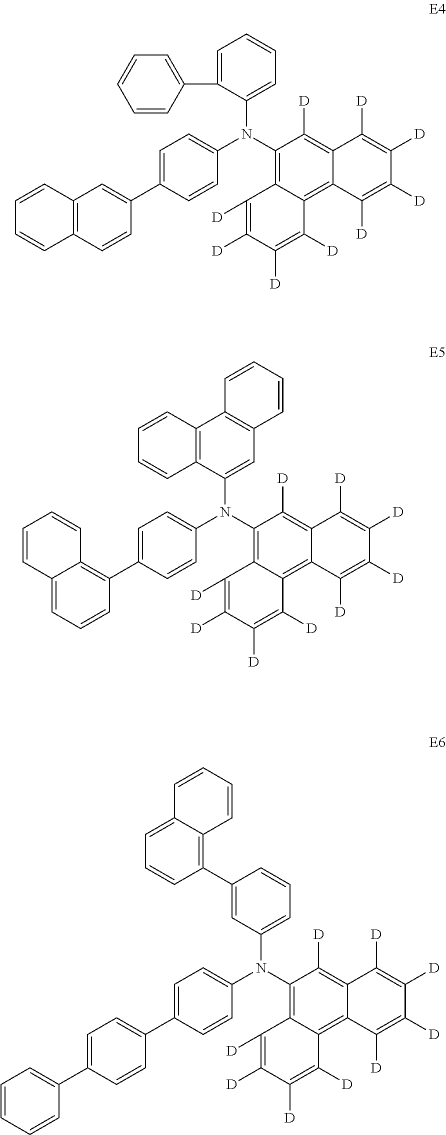 [SMILES: [2H]c1c([2H])c([2H])c2c(c1[2H])c([2H])c(N(c1ccc(-c3ccc4ccccc4c3)cc1)c1ccccc1-c1ccccc1)c1c([2H])c([2H])c([2H])c([2H])c12 ; [2H]c1c([2H])c([2H])c2c(c1[2H])c([2H])c(N(c1ccc(-c3cccc4ccccc34)cc1)c1cc3ccccc3c3ccccc13)c1c([2H])c([2H])c([2H])c([2H])c12 ; [2H]c1c([2H])c([2H])c2c(c1[2H])c([2H])c(N(c1ccc(-c3ccc(-c4ccccc4)cc3)cc1)c1cccc(-c3cccc4ccccc34)c1)c1c([2H])c([2H])c([2H])c([2H])c12] 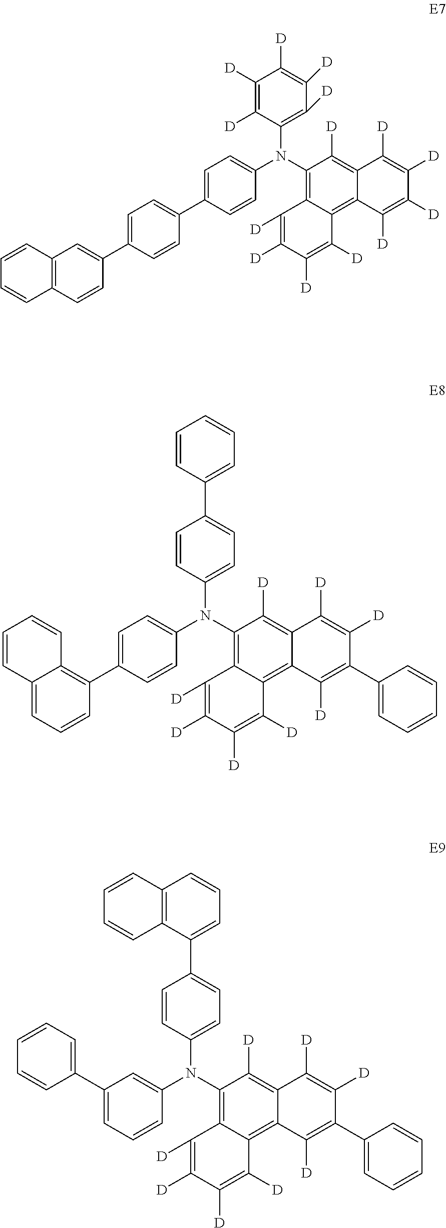 [SMILES: [2H]c1c([2H])c([2H])c(N(c2ccc(-c3ccc(-c4ccc5ccccc5c4)cc3)cc2)c2c([2H])c3c([2H])c([2H])c([2H])c([2H])c3c3c([2H])c([2H])c([2H])c([2H])c23)c([2H])c1[2H] ; [2H]c1c(-c2ccccc2)c([2H])c2c(c1[2H])c([2H])c(N(c1ccc(-c3ccccc3)cc1)c1ccc(-c3cccc4ccccc34)cc1)c1c([2H])c([2H])c([2H])c([2H])c12 ; [2H]c1c(-c2ccccc2)c([2H])c2c(c1[2H])c([2H])c(N(c1ccc(-c3cccc4ccccc34)cc1)c1cccc(-c3ccccc3)c1)c1c([2H])c([2H])c([2H])c([2H])c12] 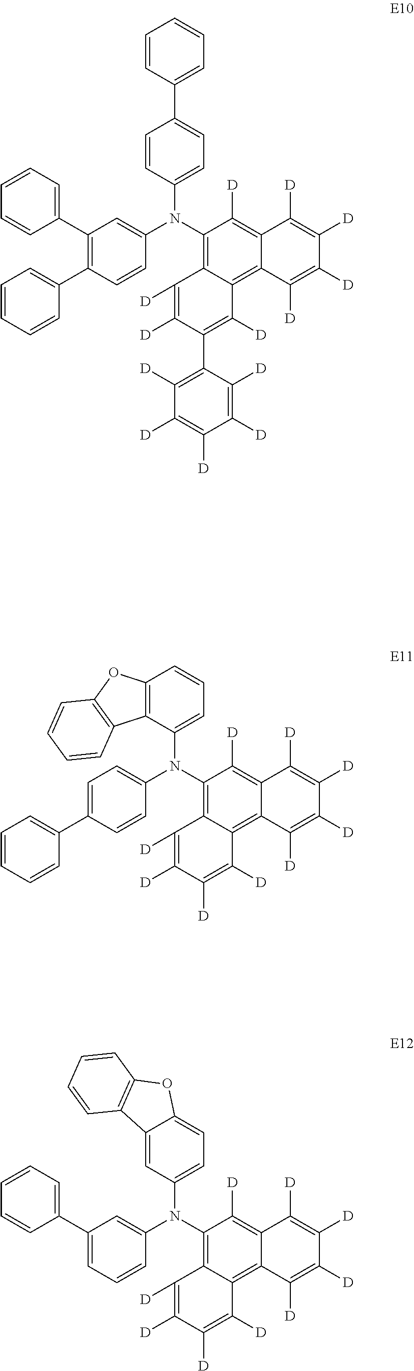 [SMILES: [2H]c1c([2H])c([2H])c(-c2c([2H])c([2H])c3c(N(c4ccc(-c5ccccc5)cc4)c4ccc(-c5ccccc5)c(-c5ccccc5)c4)c([2H])c4c([2H])c([2H])c([2H])c([2H])c4c3c2[2H])c([2H])c1[2H] ; [2H]c1c([2H])c([2H])c2c(c1[2H])c([2H])c(N(c1ccc(-c3ccccc3)cc1)c1cccc3oc4ccccc4c13)c1c([2H])c([2H])c([2H])c([2H])c12 ; [2H]c1c([2H])c([2H])c2c(c1[2H])c([2H])c(N(c1cccc(-c3ccccc3)c1)c1ccc3oc4ccccc4c3c1)c1c([2H])c([2H])c([2H])c([2H])c12] 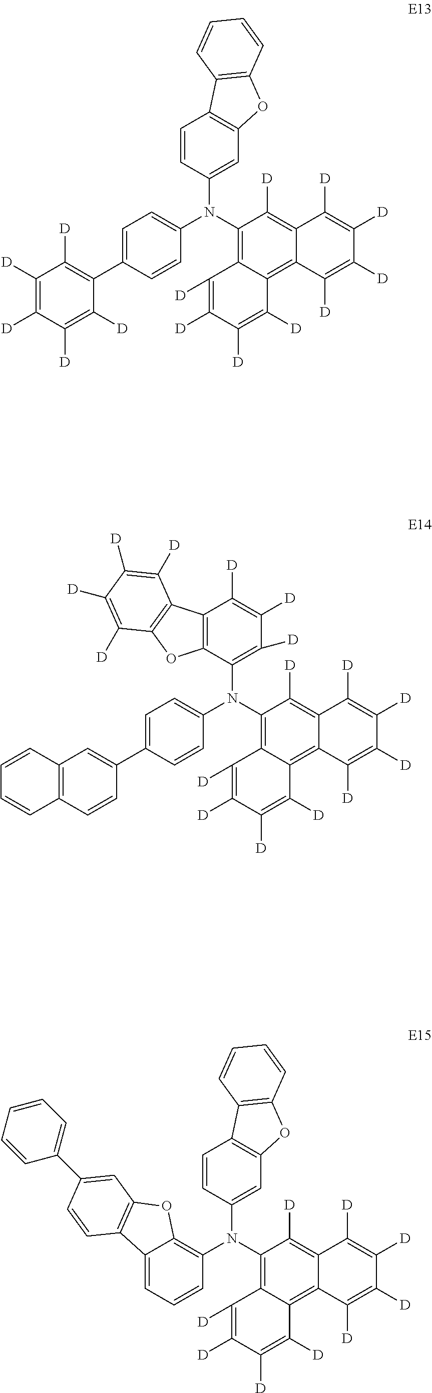 [SMILES: [2H]c1c([2H])c([2H])c(-c2ccc(N(c3ccc4c(c3)oc3ccccc34)c3c([2H])c4c([2H])c([2H])c([2H])c([2H])c4c4c([2H])c([2H])c([2H])c([2H])c34)cc2)c([2H])c1[2H] ; [2H]c1c([2H])c([2H])c2c(oc3c(N(c4ccc(-c5ccc6ccccc6c5)cc4)c4c([2H])c5c([2H])c([2H])c([2H])c([2H])c5c5c([2H])c([2H])c([2H])c([2H])c45)c([2H])c([2H])c([2H])c32)c1[2H] ; [2H]c1c([2H])c([2H])c2c(c1[2H])c([2H])c(N(c1ccc3c(c1)oc1ccccc13)c1cccc3c1oc1cc(-c4ccccc4)ccc13)c1c([2H])c([2H])c([2H])c([2H])c12] 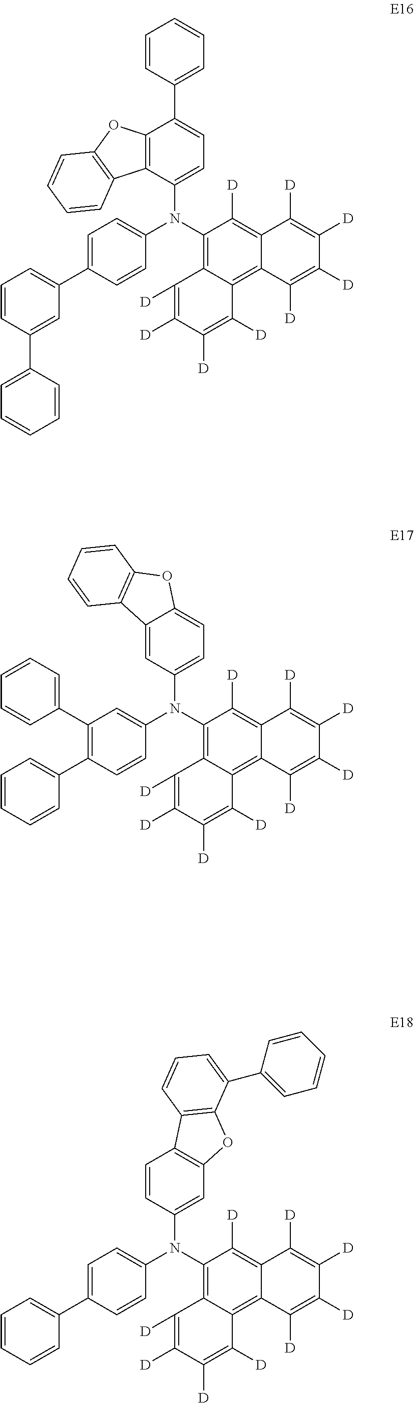 [SMILES: [2H]c1c([2H])c([2H])c2c(c1[2H])c([2H])c(N(c1ccc(-c3cccc(-c4ccccc4)c3)cc1)c1ccc(-c3ccccc3)c3oc4ccccc4c13)c1c([2H])c([2H])c([2H])c([2H])c12 ; [2H]c1c([2H])c([2H])c2c(c1[2H])c([2H])c(N(c1ccc(-c3ccccc3)c(-c3ccccc3)c1)c1ccc3oc4ccccc4c3c1)c1c([2H])c([2H])c([2H])c([2H])c12 ; [2H]c1c([2H])c([2H])c2c(c1[2H])c([2H])c(N(c1ccc(-c3ccccc3)cc1)c1ccc3c(c1)oc1c(-c4ccccc4)cccc13)c1c([2H])c([2H])c([2H])c([2H])c12] 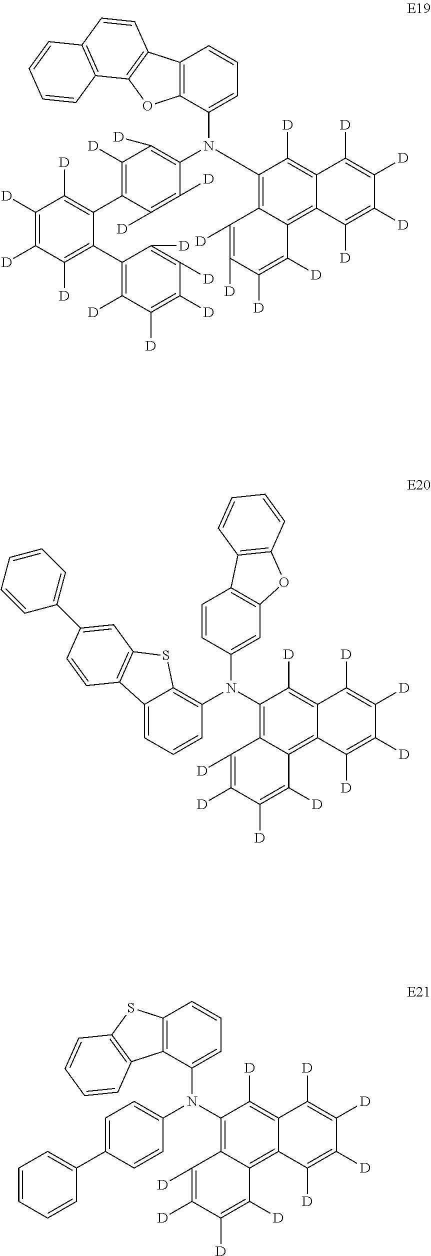 [SMILES: [2H]c1c([2H])c([2H])c(-c2c([2H])c([2H])c([2H])c([2H])c2-c2c([2H])c([2H])c(N(c3cccc4c3oc3c5ccccc5ccc43)c3c([2H])c4c([2H])c([2H])c([2H])c([2H])c4c4c([2H])c([2H])c([2H])c([2H])c34)c([2H])c2[2H])c([2H])c1[2H] ; [2H]c1c([2H])c([2H])c2c(c1[2H])c([2H])c(N(c1ccc3c(c1)oc1ccccc13)c1cccc3c1sc1cc(-c4ccccc4)ccc13)c1c([2H])c([2H])c([2H])c([2H])c12 ; [2H]c1c([2H])c([2H])c2c(c1[2H])c([2H])c(N(c1ccc(-c3ccccc3)cc1)c1cccc3sc4ccccc4c13)c1c([2H])c([2H])c([2H])c([2H])c12] 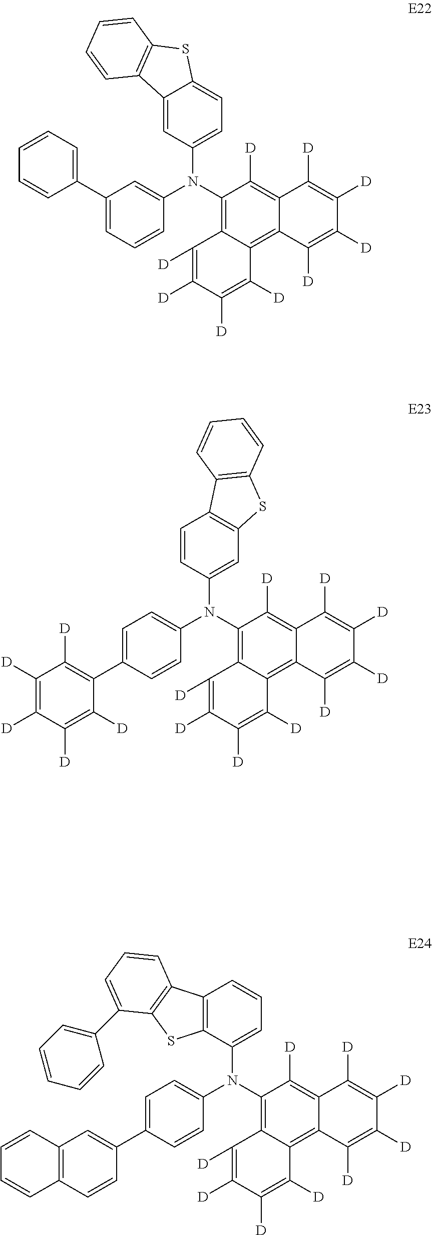 [SMILES: [2H]c1c([2H])c([2H])c2c(c1[2H])c([2H])c(N(c1cccc(-c3ccccc3)c1)c1ccc3sc4ccccc4c3c1)c1c([2H])c([2H])c([2H])c([2H])c12 ; [2H]c1c([2H])c([2H])c(-c2ccc(N(c3ccc4c(c3)sc3ccccc34)c3c([2H])c4c([2H])c([2H])c([2H])c([2H])c4c4c([2H])c([2H])c([2H])c([2H])c34)cc2)c([2H])c1[2H] ; [2H]c1c([2H])c([2H])c2c(c1[2H])c([2H])c(N(c1ccc(-c3ccc4ccccc4c3)cc1)c1cccc3c1sc1c(-c4ccccc4)cccc13)c1c([2H])c([2H])c([2H])c([2H])c12] 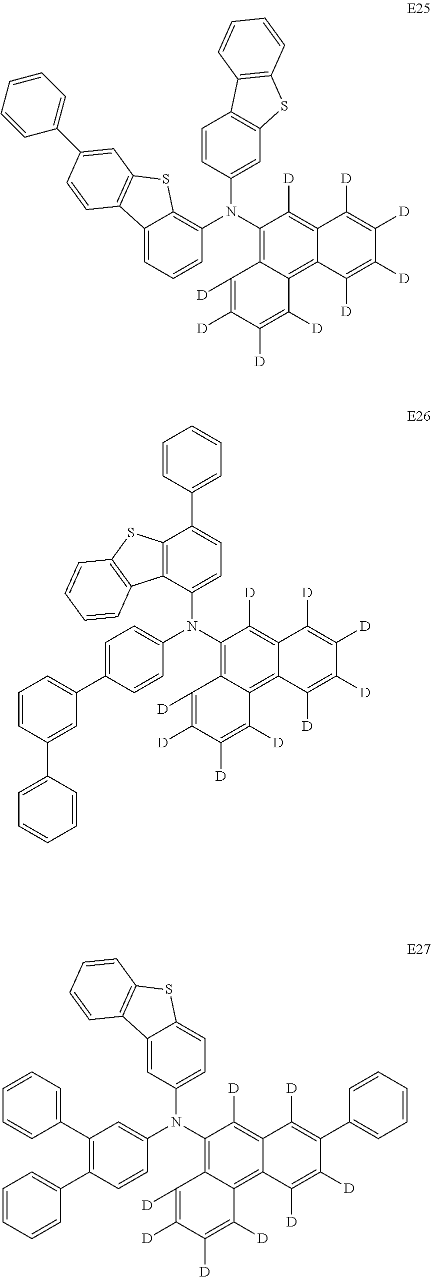 [SMILES: [2H]c1c([2H])c([2H])c2c(c1[2H])c([2H])c(N(c1ccc3c(c1)sc1ccccc13)c1cccc3c1sc1cc(-c4ccccc4)ccc13)c1c([2H])c([2H])c([2H])c([2H])c12 ; [2H]c1c([2H])c([2H])c2c(c1[2H])c([2H])c(N(c1ccc(-c3cccc(-c4ccccc4)c3)cc1)c1ccc(-c3ccccc3)c3sc4ccccc4c13)c1c([2H])c([2H])c([2H])c([2H])c12 ; [2H]c1c([2H])c([2H])c2c(c1[2H])c(N(c1ccc(-c3ccccc3)c(-c3ccccc3)c1)c1ccc3sc4ccccc4c3c1)c([2H])c1c([2H])c(-c3ccccc3)c([2H])c([2H])c12] 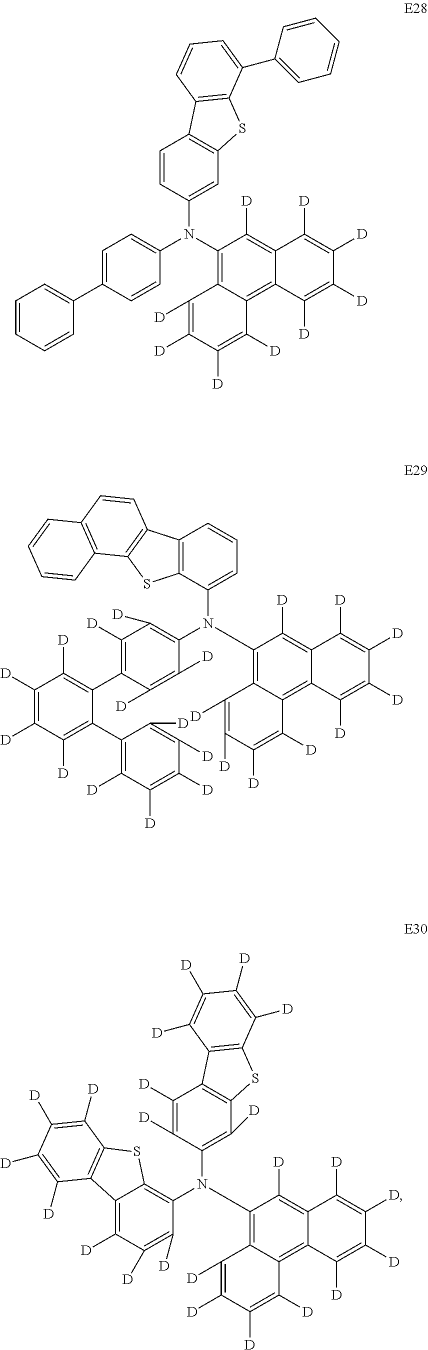
in Compound Group 1, D being deuterium.

11. An amine compound represented by Formula 1 and Formula 2: 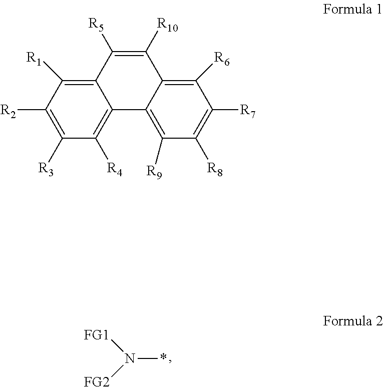
wherein,
in Formula 1, at least one selected from among R₁ to R₅ is an amine group represented by Formula 2, at least one selected from among the remaining R₁ to R₅, and R₆ to R₁₀ is deuterium, and the remaining that is not deuterium is a substituted or unsubstituted aryl group having ring-forming carbons of 6 to 40,
in Formula 2, FG1 and FG2 are each independently a substituted or unsubstituted aryl group having ring-forming carbons of 6 to 40 except for a fluorenyl group or a substituted or unsubstituted heteroaryl group comprising 1 to 3 hetero atoms and having ring-forming carbons of 5 to 40, and FG1 and FG2 comprise no alkyl group as a substituent,
when R₂ is the amine group represented by Formula 2:
a case where FG1 and FG2 are each a substituted or unsubstituted aryl groups having ring-forming carbons of 6 to 15, at the same time, is excluded; and
a case where FG1 or FG2 is a benzonaphthofuranyl group substituted with an aryl group is excluded,
when R₃ is the amine group represented by Formula 2:
a case where FG1 and FG2 are each a substituted or unsubstituted aryl groups having ring-forming carbons of 6 to 9, at the same time, is excluded; and
when one among FG1 and FG2 is an aryl group having ring-forming carbons of 26 or more, the other is an aryl group having a ring-forming carbons of 6 to 15 or a substituted or unsubstituted heteroaryl group having a ring-forming carbons of 5 to 40, and
when R₅ is the amine group represented by Formula 2:
when FG1 or FG2 is a 4-dibenzofuranyl group or a 4-dibenzothiophenyl group, at least one hydrogen of the 4-dibenzofuranyl group or the 4-dibenzothiophenyl group is substituted with a substituent; and
cases where FG1 or FG2 is a naphthyl group or a benzonaphthofuranyl group substituted with an aryl group are excluded.

12. The amine compound of claim 11, wherein the amine compound is represented by Formulae 1A to 1E: 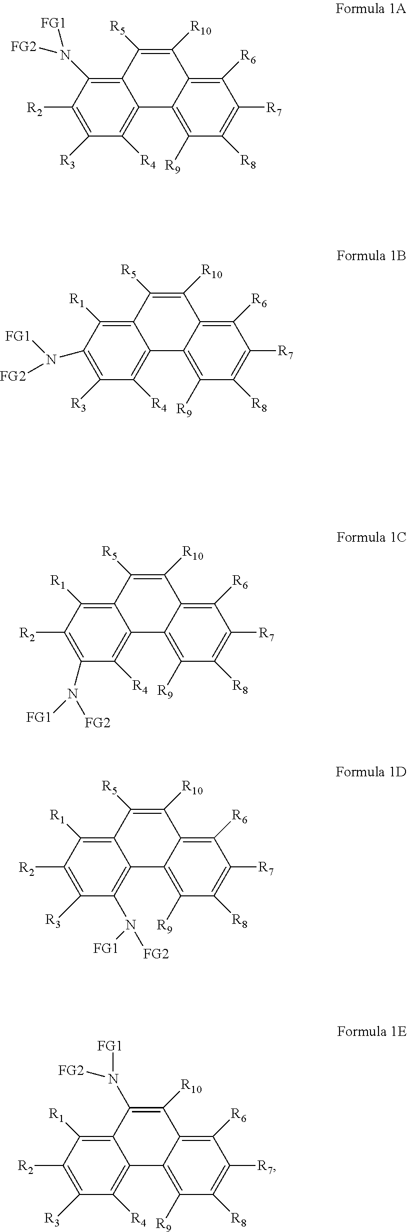
in Formula 1A to Formula 1E,
R₁ to R₁₀, FG1, and FG2 each being the same as defined in Formula 1 and Formula 2.

13. The amine compound of claim 11, wherein in Formula 1:
the remaining among R₁ to R₅ that is not the amine group represented by Formula 2, and R₆ to R₁₀ are all deuterium(s), or
any one selected from among the remaining of R₁ to R₅ that is not the amine group represented by Formula 2, and R₆ to R₁₀ is a substituted or unsubstituted phenyl group, and the remaining are deuterium(s).

14. The amine compound of claim 11, wherein at least one hydrogen of FG1 and FG2, except those in R₁ to R₅ and R₆ to R₁₀, is substituted with deuterium.

15. The amine compound of claim 11, wherein the amine compound is a monoamine compound comprising no additional amino group as a substituent.

16. The amine compound of claim 11, wherein FG1 and FG2 are each independently a substituted or unsubstituted phenyl group, a substituted or unsubstituted biphenyl group, a substituted or unsubstituted terphenyl group, a substituted or unsubstituted naphthyl group, a substituted or unsubstituted phenanthryl group, a substituted or unsubstituted dibenzofuranyl group, a substituted or unsubstituted dibenzothiophenyl group, a substituted or unsubstituted benzonaphthofuranyl group, or a substituted or unsubstituted benzonaphthothiophenyl group.

17. The amine compound of claim 11, wherein the amine compound is any one selected from among compounds in Compound Group 1:
Compound Group 1 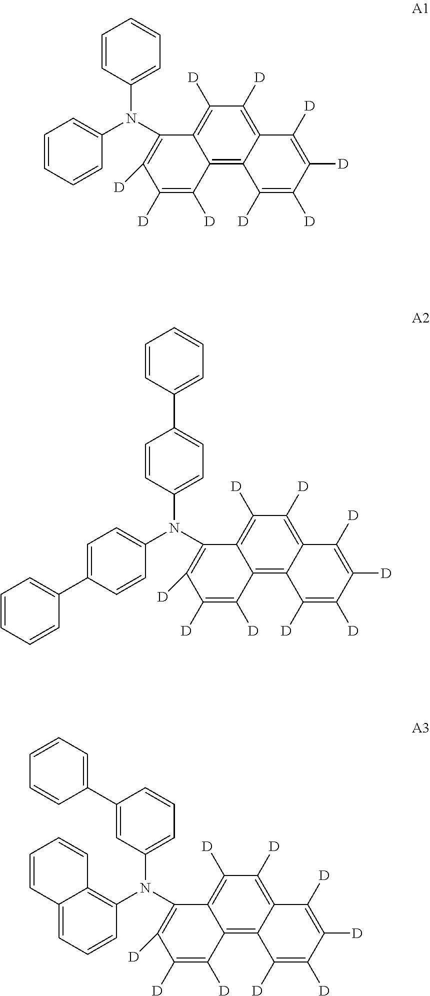 [SMILES: [2H]c1c([2H])c([2H])c2c(c1[2H])c([2H])c([2H])c1c(N(c3ccccc3)c3ccccc3)c([2H])c([2H])c([2H])c12 ; [2H]c1c([2H])c([2H])c2c(c1[2H])c([2H])c([2H])c1c(N(c3ccc(-c4ccccc4)cc3)c3ccc(-c4ccccc4)cc3)c([2H])c([2H])c([2H])c12 ; [2H]c1c([2H])c([2H])c2c(c1[2H])c([2H])c([2H])c1c(N(c3cccc(-c4ccccc4)c3)c3cccc4ccccc34)c([2H])c([2H])c([2H])c12] 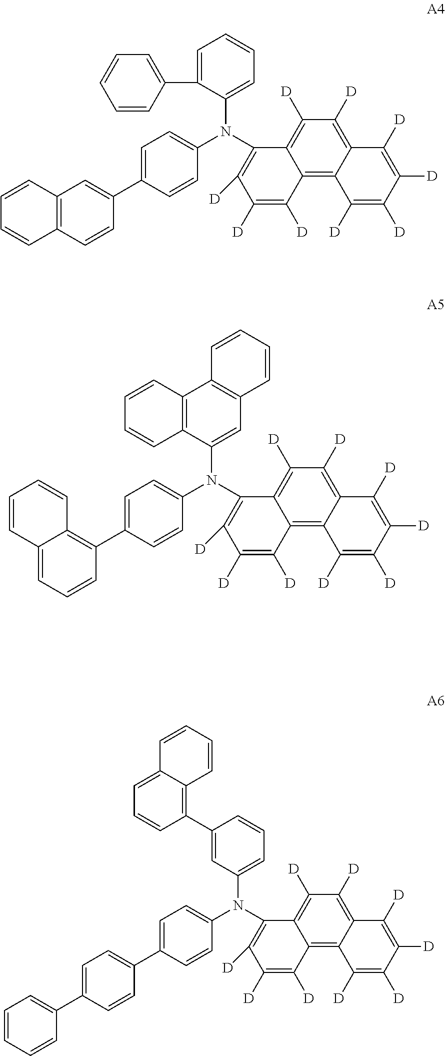 [SMILES: [2H]c1c([2H])c([2H])c2c(c1[2H])c([2H])c([2H])c1c(N(c3ccc(-c4ccc5ccccc5c4)cc3)c3ccccc3-c3ccccc3)c([2H])c([2H])c([2H])c12 ; [2H]c1c([2H])c([2H])c2c(c1[2H])c([2H])c([2H])c1c(N(c3ccc(-c4cccc5ccccc45)cc3)c3cc4ccccc4c4ccccc34)c([2H])c([2H])c([2H])c12 ; [2H]c1c([2H])c([2H])c2c(c1[2H])c([2H])c([2H])c1c(N(c3ccc(-c4ccc(-c5ccccc5)cc4)cc3)c3cccc(-c4cccc5ccccc45)c3)c([2H])c([2H])c([2H])c12] 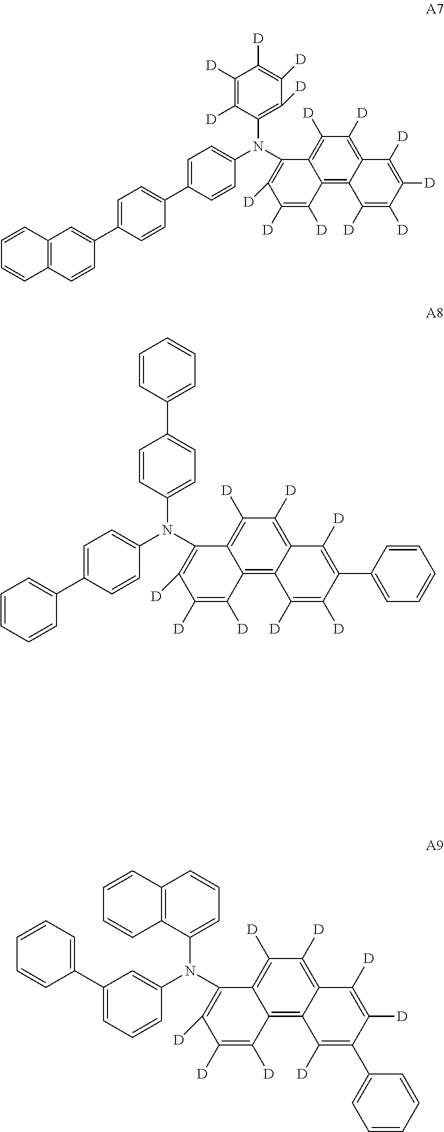 [SMILES: [2H]c1c([2H])c([2H])c(N(c2ccc(-c3ccc(-c4ccc5ccccc5c4)cc3)cc2)c2c([2H])c([2H])c([2H])c3c2c([2H])c([2H])c2c([2H])c([2H])c([2H])c([2H])c23)c([2H])c1[2H] ; [2H]c1c([2H])c([2H])c2c(c([2H])c([2H])c3c([2H])c(-c4ccccc4)c([2H])c([2H])c32)c1N(c1ccc(-c2ccccc2)cc1)c1ccc(-c2ccccc2)cc1 ; [2H]c1c(-c2ccccc2)c([2H])c2c(c1[2H])c([2H])c([2H])c1c(N(c3cccc(-c4ccccc4)c3)c3cccc4ccccc34)c([2H])c([2H])c([2H])c12] 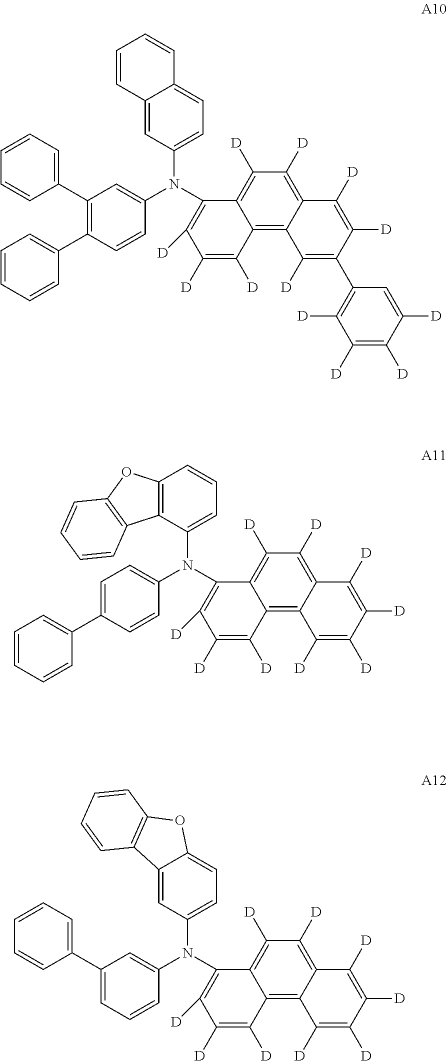 [SMILES: [2H]c1cc(-c2c([2H])c([2H])c3c([2H])c([2H])c4c(N(c5ccc(-c6ccccc6)c(-c6ccccc6)c5)c5ccc6ccccc6c5)c([2H])c([2H])c([2H])c4c3c2[2H])c([2H])c([2H])c1[2H] ; [2H]c1c([2H])c([2H])c2c(c1[2H])c([2H])c([2H])c1c(N(c3ccc(-c4ccccc4)cc3)c3cccc4oc5ccccc5c34)c([2H])c([2H])c([2H])c12 ; [2H]c1c([2H])c([2H])c2c(c1[2H])c([2H])c([2H])c1c(N(c3cccc(-c4ccccc4)c3)c3ccc4oc5ccccc5c4c3)c([2H])c([2H])c([2H])c12] 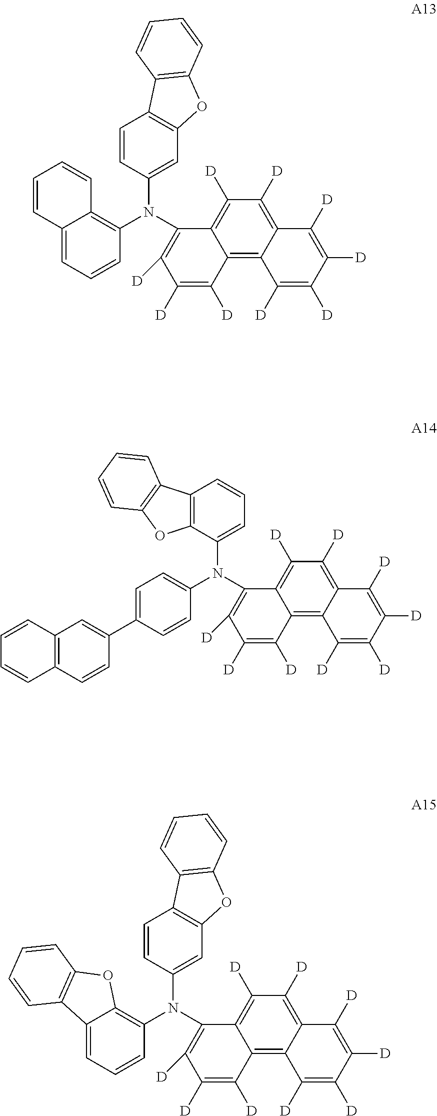 [SMILES: [2H]c1c([2H])c([2H])c2c(c1[2H])c([2H])c([2H])c1c(N(c3ccc4c(c3)oc3ccccc34)c3cccc4ccccc34)c([2H])c([2H])c([2H])c12 ; [2H]c1c([2H])c([2H])c2c(c1[2H])c([2H])c([2H])c1c(N(c3ccc(-c4ccc5ccccc5c4)cc3)c3cccc4c3oc3ccccc34)c([2H])c([2H])c([2H])c12 ; [2H]c1c([2H])c([2H])c2c(c1[2H])c([2H])c([2H])c1c(N(c3ccc4c(c3)oc3ccccc34)c3cccc4c3oc3ccccc34)c([2H])c([2H])c([2H])c12] 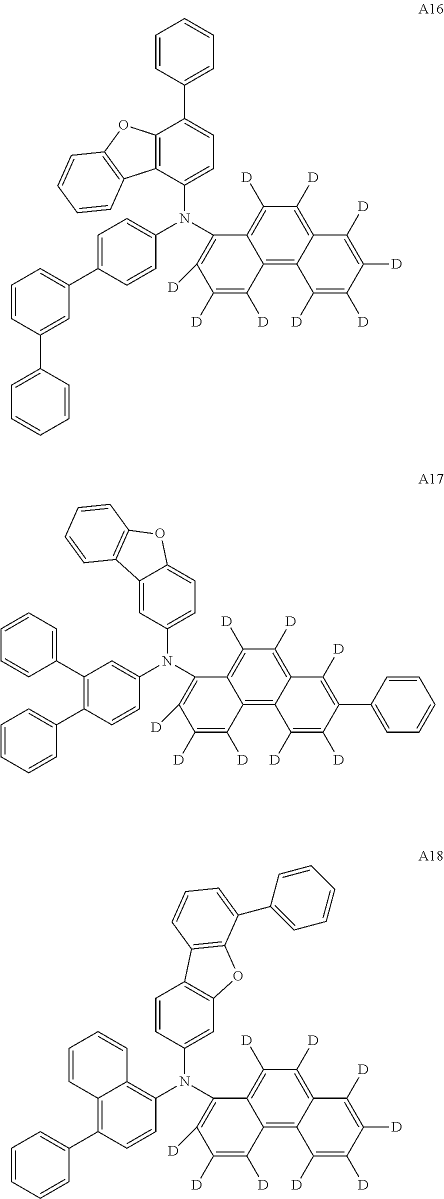 [SMILES: [2H]c1c([2H])c([2H])c2c(c1[2H])c([2H])c([2H])c1c(N(c3ccc(-c4cccc(-c5ccccc5)c4)cc3)c3ccc(-c4ccccc4)c4oc5ccccc5c34)c([2H])c([2H])c([2H])c12 ; [2H]c1c([2H])c([2H])c2c(c([2H])c([2H])c3c([2H])c(-c4ccccc4)c([2H])c([2H])c32)c1N(c1ccc(-c2ccccc2)c(-c2ccccc2)c1)c1ccc2oc3ccccc3c2c1 ; [2H]c1c([2H])c([2H])c2c(c1[2H])c([2H])c([2H])c1c(N(c3ccc4c(c3)oc3c(-c5ccccc5)cccc34)c3ccc(-c4ccccc4)c4ccccc34)c([2H])c([2H])c([2H])c12] 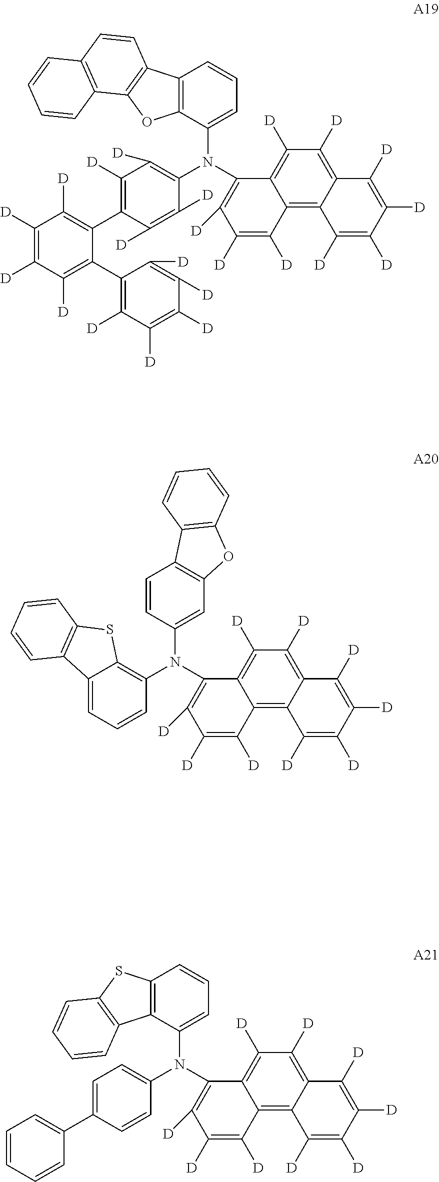 [SMILES: [2H]c1c([2H])c([2H])c(-c2c([2H])c([2H])c([2H])c([2H])c2-c2c([2H])c([2H])c(N(c3cccc4c3oc3c5ccccc5ccc43)c3c([2H])c([2H])c([2H])c4c3c([2H])c([2H])c3c([2H])c([2H])c([2H])c([2H])c34)c([2H])c2[2H])c([2H])c1[2H] ; [2H]c1c([2H])c([2H])c2c(c1[2H])c([2H])c([2H])c1c(N(c3ccc4c(c3)oc3ccccc34)c3cccc4c3sc3ccccc34)c([2H])c([2H])c([2H])c12 ; [2H]c1c([2H])c([2H])c2c(c1[2H])c([2H])c([2H])c1c(N(c3ccc(-c4ccccc4)cc3)c3cccc4sc5ccccc5c34)c([2H])c([2H])c([2H])c12] 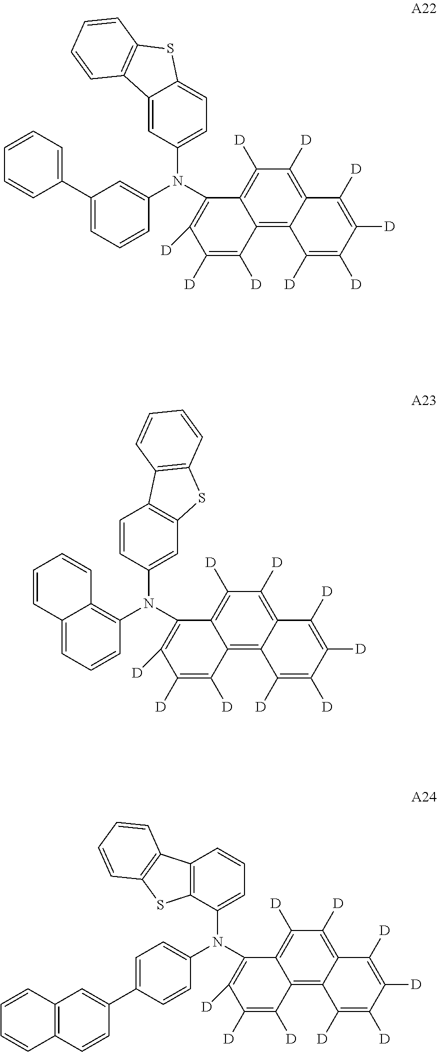 [SMILES: [2H]c1c([2H])c([2H])c2c(c1[2H])c([2H])c([2H])c1c(N(c3cccc(-c4ccccc4)c3)c3ccc4sc5ccccc5c4c3)c([2H])c([2H])c([2H])c12 ; [2H]c1c([2H])c([2H])c2c(c1[2H])c([2H])c([2H])c1c(N(c3ccc4c(c3)sc3ccccc34)c3cccc4ccccc34)c([2H])c([2H])c([2H])c12 ; [2H]c1c([2H])c([2H])c2c(c1[2H])c([2H])c([2H])c1c(N(c3ccc(-c4ccc5ccccc5c4)cc3)c3cccc4c3sc3ccccc34)c([2H])c([2H])c([2H])c12] 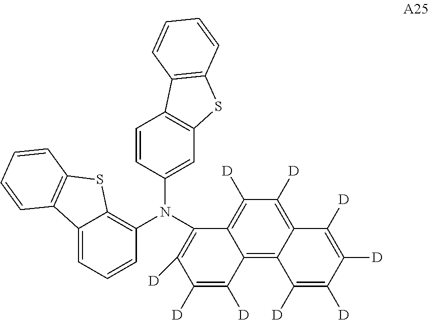 [SMILES: [2H]c1c([2H])c([2H])c2c(c1[2H])c([2H])c([2H])c1c(N(c3ccc4c(c3)sc3ccccc34)c3cccc4c3sc3ccccc34)c([2H])c([2H])c([2H])c12] 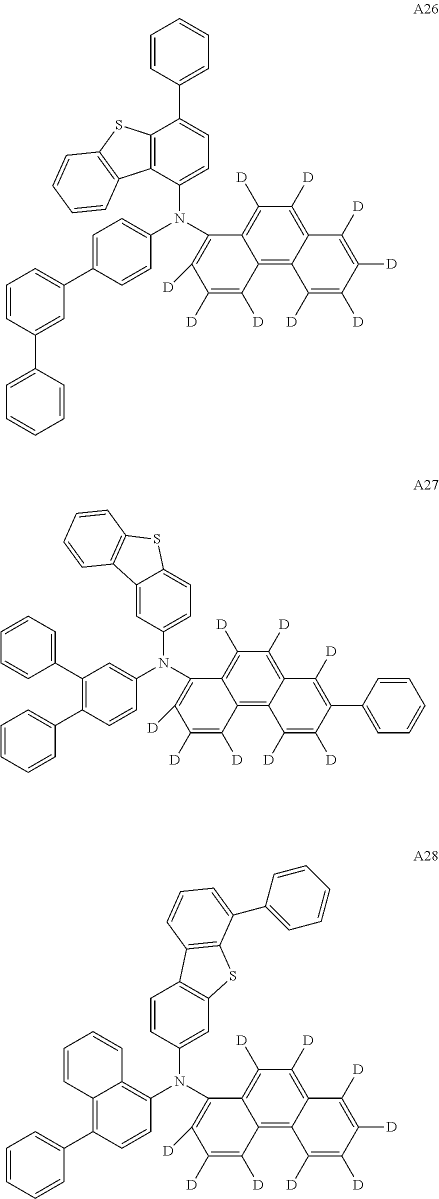 [SMILES: [2H]c1c([2H])c([2H])c2c(c1[2H])c([2H])c([2H])c1c(N(c3ccc(-c4cccc(-c5ccccc5)c4)cc3)c3ccc(-c4ccccc4)c4sc5ccccc5c34)c([2H])c([2H])c([2H])c12 ; [2H]c1c([2H])c([2H])c2c(c([2H])c([2H])c3c([2H])c(-c4ccccc4)c([2H])c([2H])c32)c1N(c1ccc(-c2ccccc2)c(-c2ccccc2)c1)c1ccc2sc3ccccc3c2c1 ; [2H]c1c([2H])c([2H])c2c(c1[2H])c([2H])c([2H])c1c(N(c3ccc4c(c3)sc3c(-c5ccccc5)cccc34)c3ccc(-c4ccccc4)c4ccccc34)c([2H])c([2H])c([2H])c12] 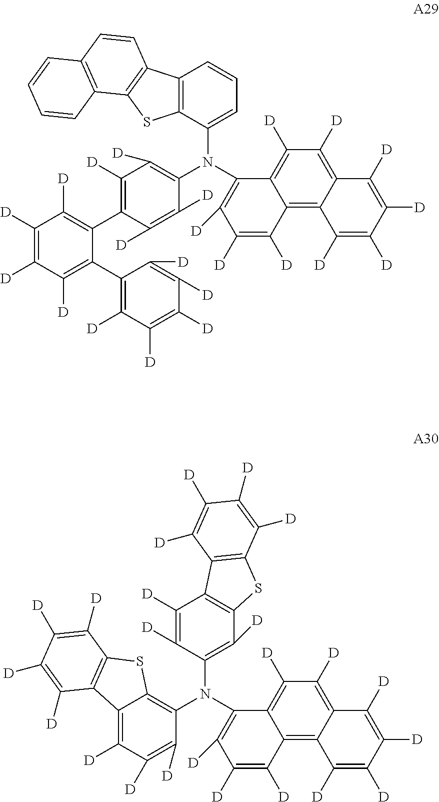 [SMILES: [2H]c1c([2H])c([2H])c(-c2c([2H])c([2H])c([2H])c([2H])c2-c2c([2H])c([2H])c(N(c3cccc4c3sc3c5ccccc5ccc43)c3c([2H])c([2H])c([2H])c4c3c([2H])c([2H])c3c([2H])c([2H])c([2H])c([2H])c34)c([2H])c2[2H])c([2H])c1[2H] ; [2H]c1c([2H])c([2H])c2c(sc3c([2H])c(N(c4c([2H])c([2H])c([2H])c5c4sc4c([2H])c([2H])c([2H])c([2H])c45)c4c([2H])c([2H])c([2H])c5c4c([2H])c([2H])c4c([2H])c([2H])c([2H])c([2H])c45)c([2H])c([2H])c32)c1[2H]] 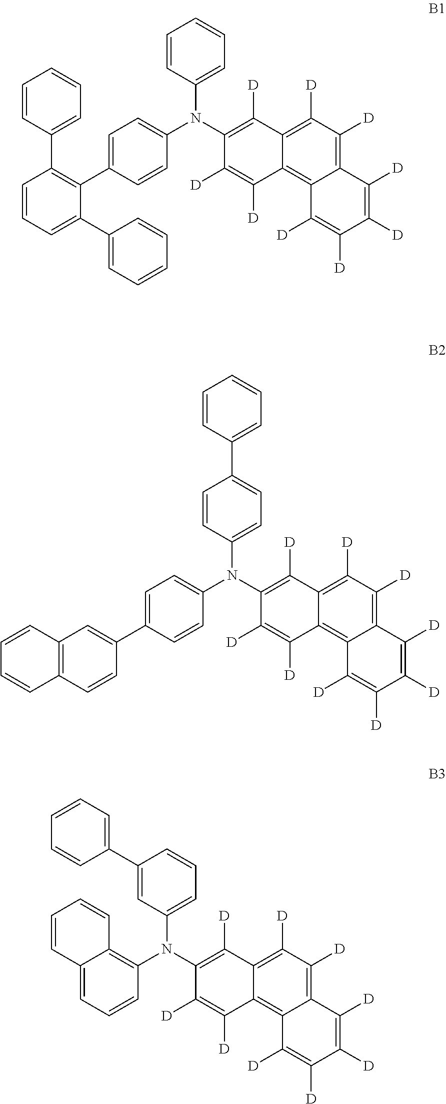 [SMILES: [2H]c1c([2H])c([2H])c2c(c1[2H])c([2H])c([2H])c1c([2H])c(N(c3ccccc3)c3ccc(-c4c(-c5ccccc5)cccc4-c4ccccc4)cc3)c([2H])c([2H])c12 ; [2H]c1c([2H])c([2H])c2c(c1[2H])c([2H])c([2H])c1c([2H])c(N(c3ccc(-c4ccccc4)cc3)c3ccc(-c4ccc5ccccc5c4)cc3)c([2H])c([2H])c12 ; [2H]c1c([2H])c([2H])c2c(c1[2H])c([2H])c([2H])c1c([2H])c(N(c3cccc(-c4ccccc4)c3)c3cccc4ccccc34)c([2H])c([2H])c12] 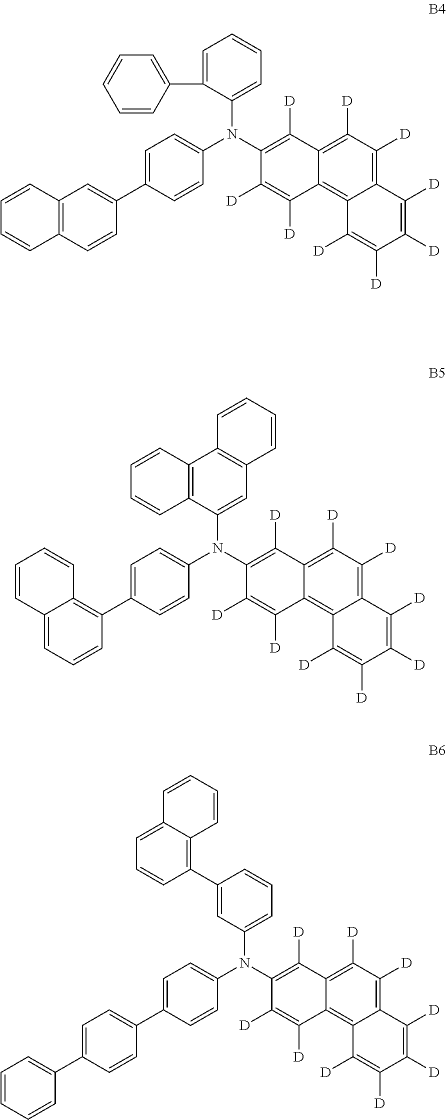 [SMILES: [2H]c1c([2H])c([2H])c2c(c1[2H])c([2H])c([2H])c1c([2H])c(N(c3ccc(-c4ccc5ccccc5c4)cc3)c3ccccc3-c3ccccc3)c([2H])c([2H])c12 ; [2H]c1c([2H])c([2H])c2c(c1[2H])c([2H])c([2H])c1c([2H])c(N(c3ccc(-c4cccc5ccccc45)cc3)c3cc4ccccc4c4ccccc34)c([2H])c([2H])c12 ; [2H]c1c([2H])c([2H])c2c(c1[2H])c([2H])c([2H])c1c([2H])c(N(c3ccc(-c4ccc(-c5ccccc5)cc4)cc3)c3cccc(-c4cccc5ccccc45)c3)c([2H])c([2H])c12] 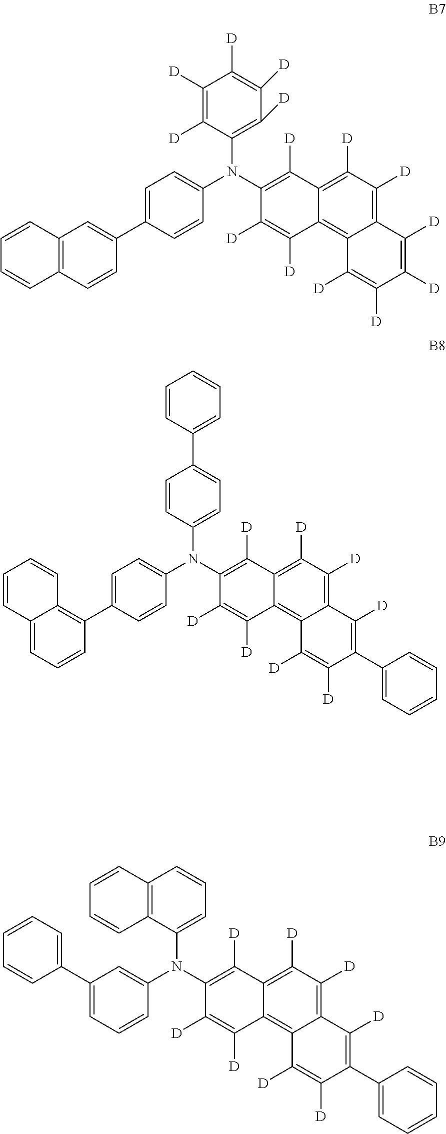 [SMILES: [2H]c1c([2H])c([2H])c(N(c2ccc(-c3ccc4ccccc4c3)cc2)c2c([2H])c([2H])c3c(c2[2H])c([2H])c([2H])c2c([2H])c([2H])c([2H])c([2H])c23)c([2H])c1[2H] ; [2H]c1c(-c2ccccc2)c([2H])c2c([2H])c([2H])c3c([2H])c(N(c4ccc(-c5ccccc5)cc4)c4ccc(-c5cccc6ccccc56)cc4)c([2H])c([2H])c3c2c1[2H] ; [2H]c1c(-c2ccccc2)c([2H])c2c([2H])c([2H])c3c([2H])c(N(c4cccc(-c5ccccc5)c4)c4cccc5ccccc45)c([2H])c([2H])c3c2c1[2H]] 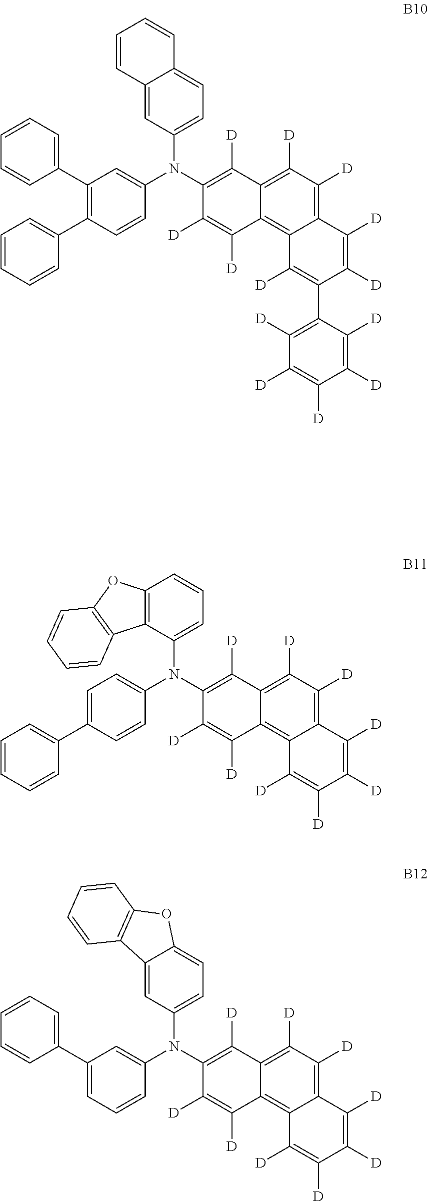 [SMILES: [2H]c1c([2H])c([2H])c(-c2c([2H])c([2H])c3c([2H])c([2H])c4c([2H])c(N(c5ccc(-c6ccccc6)c(-c6ccccc6)c5)c5ccc6ccccc6c5)c([2H])c([2H])c4c3c2[2H])c([2H])c1[2H] ; [2H]c1c([2H])c([2H])c2c(c1[2H])c([2H])c([2H])c1c([2H])c(N(c3ccc(-c4ccccc4)cc3)c3cccc4oc5ccccc5c34)c([2H])c([2H])c12 ; [2H]c1c([2H])c([2H])c2c(c1[2H])c([2H])c([2H])c1c([2H])c(N(c3cccc(-c4ccccc4)c3)c3ccc4oc5ccccc5c4c3)c([2H])c([2H])c12] 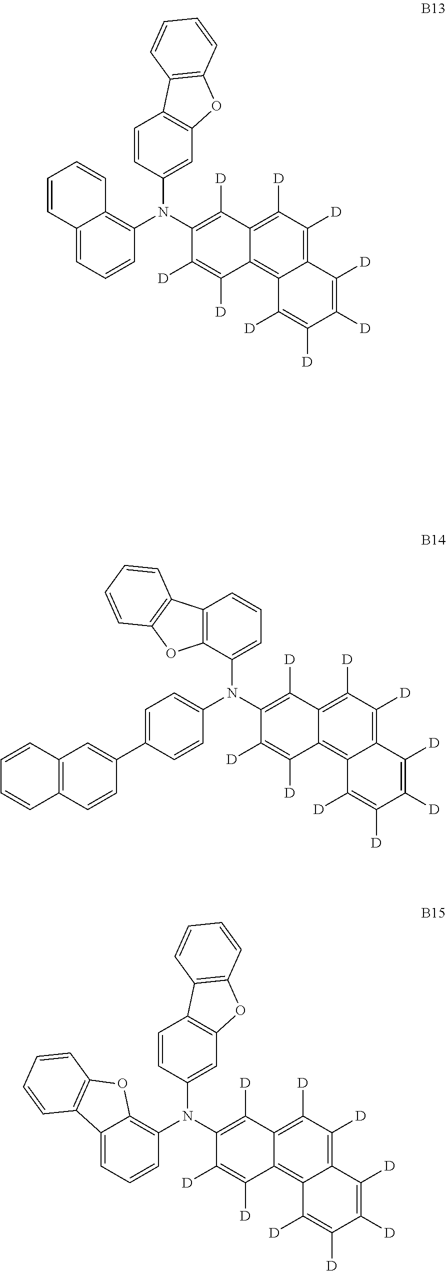 [SMILES: [2H]c1c([2H])c([2H])c2c(c1[2H])c([2H])c([2H])c1c([2H])c(N(c3ccc4c(c3)oc3ccccc34)c3cccc4ccccc34)c([2H])c([2H])c12 ; [2H]c1c([2H])c([2H])c2c(c1[2H])c([2H])c([2H])c1c([2H])c(N(c3ccc(-c4ccc5ccccc5c4)cc3)c3cccc4c3oc3ccccc34)c([2H])c([2H])c12 ; [2H]c1c([2H])c([2H])c2c(c1[2H])c([2H])c([2H])c1c([2H])c(N(c3ccc4c(c3)oc3ccccc34)c3cccc4c3oc3ccccc34)c([2H])c([2H])c12] 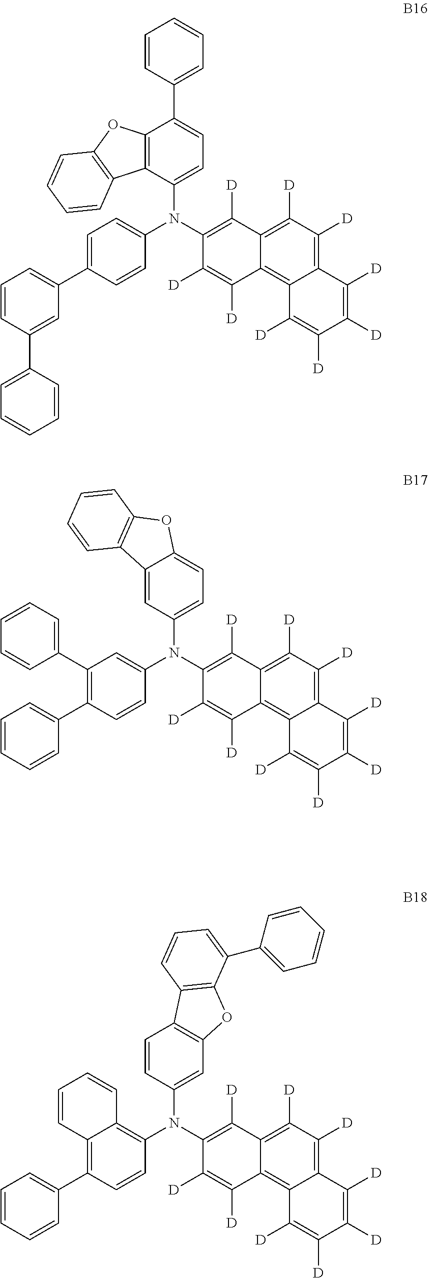 [SMILES: [2H]c1c([2H])c([2H])c2c(c1[2H])c([2H])c([2H])c1c([2H])c(N(c3ccc(-c4cccc(-c5ccccc5)c4)cc3)c3ccc(-c4ccccc4)c4oc5ccccc5c34)c([2H])c([2H])c12 ; [2H]c1c([2H])c([2H])c2c(c1[2H])c([2H])c([2H])c1c([2H])c(N(c3ccc(-c4ccccc4)c(-c4ccccc4)c3)c3ccc4oc5ccccc5c4c3)c([2H])c([2H])c12 ; [2H]c1c([2H])c([2H])c2c(c1[2H])c([2H])c([2H])c1c([2H])c(N(c3ccc4c(c3)oc3c(-c5ccccc5)cccc34)c3ccc(-c4ccccc4)c4ccccc34)c([2H])c([2H])c12] 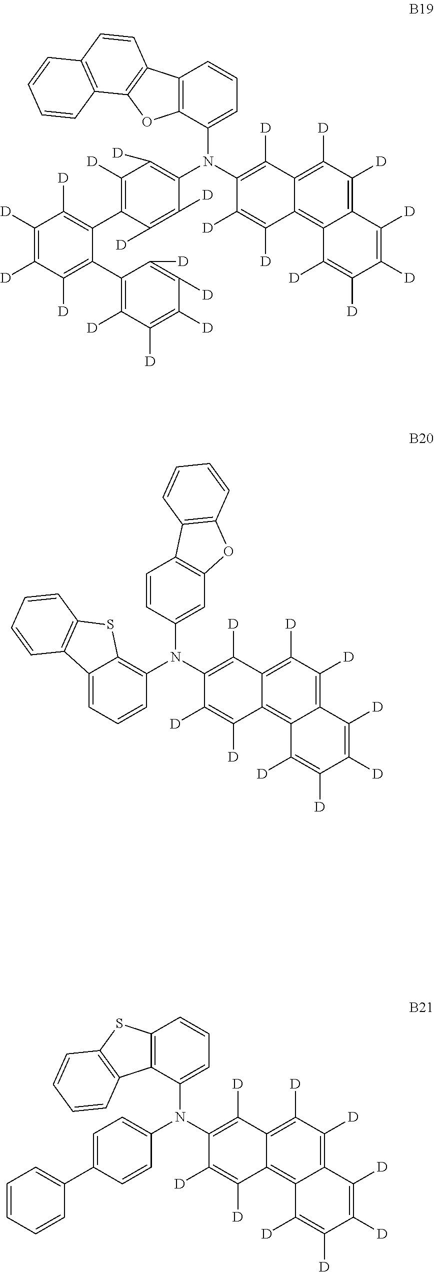 [SMILES: [2H]c1c([2H])c([2H])c(-c2c([2H])c([2H])c([2H])c([2H])c2-c2c([2H])c([2H])c(N(c3c([2H])c([2H])c4c(c3[2H])c([2H])c([2H])c3c([2H])c([2H])c([2H])c([2H])c34)c3cccc4c3oc3c5ccccc5ccc43)c([2H])c2[2H])c([2H])c1[2H] ; [2H]c1c([2H])c([2H])c2c(c1[2H])c([2H])c([2H])c1c([2H])c(N(c3ccc4c(c3)oc3ccccc34)c3cccc4c3sc3ccccc34)c([2H])c([2H])c12 ; [2H]c1c([2H])c([2H])c2c(c1[2H])c([2H])c([2H])c1c([2H])c(N(c3ccc(-c4ccccc4)cc3)c3cccc4sc5ccccc5c34)c([2H])c([2H])c12] 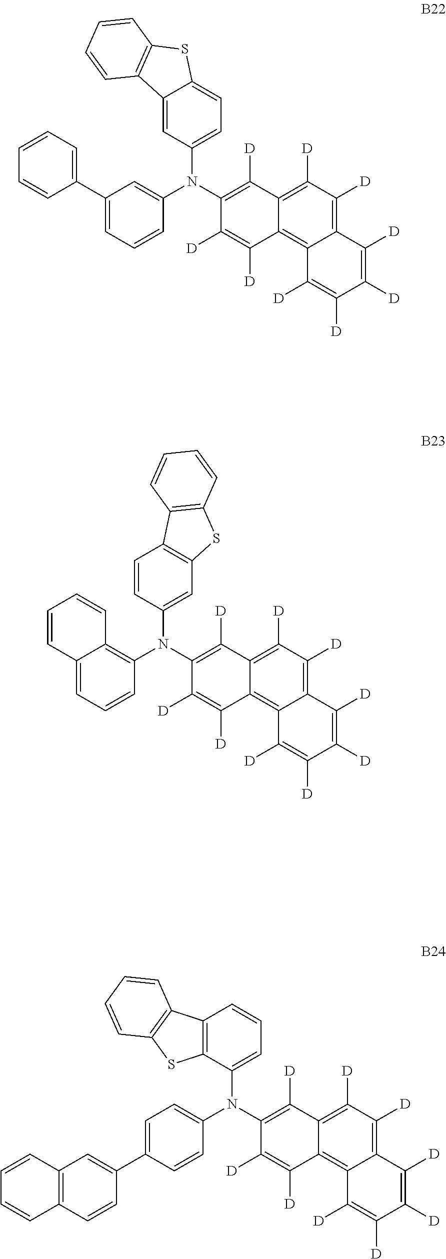 [SMILES: [2H]c1c([2H])c([2H])c2c(c1[2H])c([2H])c([2H])c1c([2H])c(N(c3cccc(-c4ccccc4)c3)c3ccc4sc5ccccc5c4c3)c([2H])c([2H])c12 ; [2H]c1c([2H])c([2H])c2c(c1[2H])c([2H])c([2H])c1c([2H])c(N(c3ccc4c(c3)sc3ccccc34)c3cccc4ccccc34)c([2H])c([2H])c12 ; [2H]c1c([2H])c([2H])c2c(c1[2H])c([2H])c([2H])c1c([2H])c(N(c3ccc(-c4ccc5ccccc5c4)cc3)c3cccc4c3sc3ccccc34)c([2H])c([2H])c12] 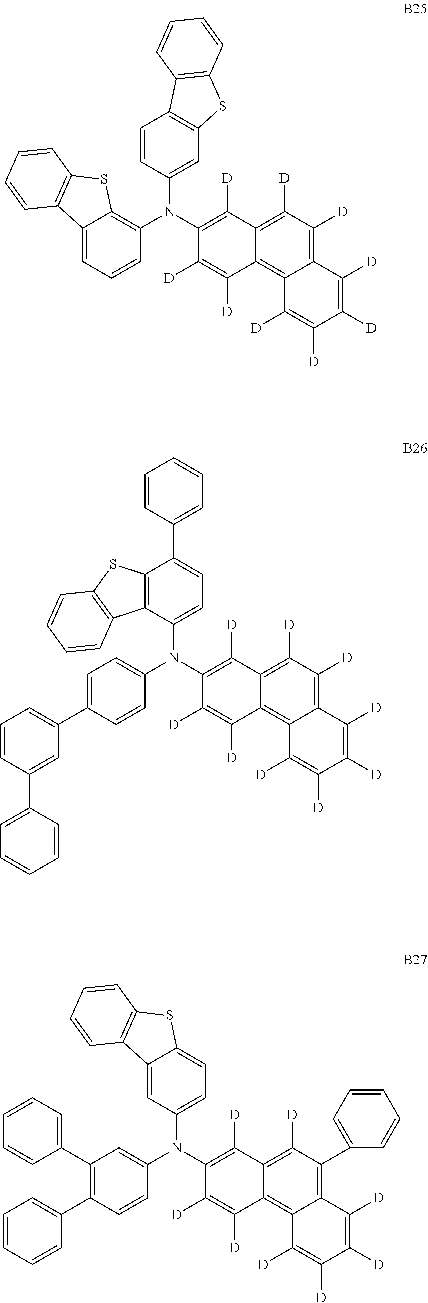 [SMILES: [2H]c1c([2H])c([2H])c2c(c1[2H])c([2H])c([2H])c1c([2H])c(N(c3ccc4c(c3)sc3ccccc34)c3cccc4c3sc3ccccc34)c([2H])c([2H])c12 ; [2H]c1c([2H])c([2H])c2c(c1[2H])c([2H])c([2H])c1c([2H])c(N(c3ccc(-c4cccc(-c5ccccc5)c4)cc3)c3ccc(-c4ccccc4)c4sc5ccccc5c34)c([2H])c([2H])c12 ; [2H]c1c([2H])c([2H])c2c(c1[2H])c(-c1ccccc1)c([2H])c1c([2H])c(N(c3ccc(-c4ccccc4)c(-c4ccccc4)c3)c3ccc4sc5ccccc5c4c3)c([2H])c([2H])c12] 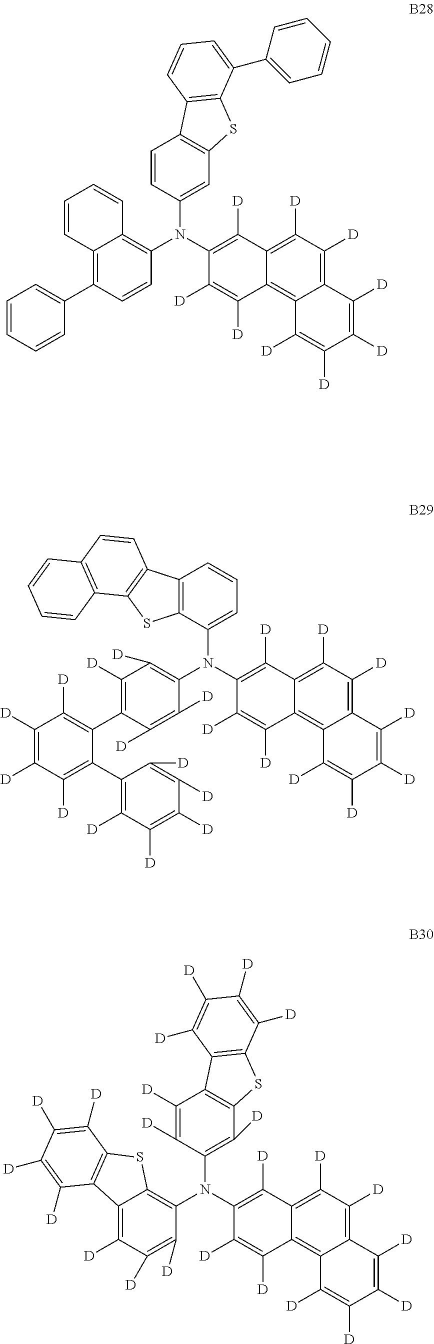 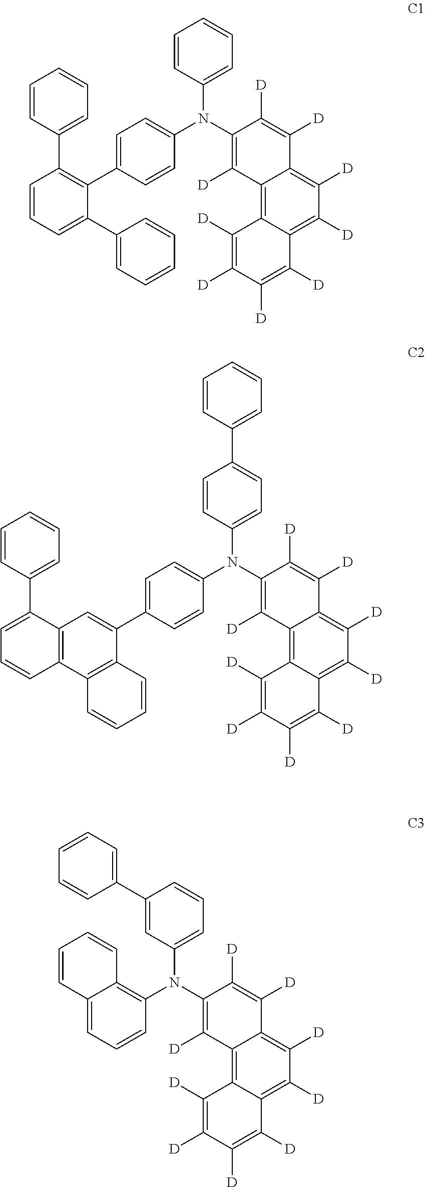 [SMILES: [2H]c1c([2H])c([2H])c2c(c1[2H])c([2H])c([2H])c1c([2H])c([2H])c(N(c3ccccc3)c3ccc(-c4c(-c5ccccc5)cccc4-c4ccccc4)cc3)c([2H])c12 ; [2H]c1c([2H])c([2H])c2c(c1[2H])c([2H])c([2H])c1c([2H])c([2H])c(N(c3ccc(-c4ccccc4)cc3)c3ccc(-c4cc5c(-c6ccccc6)cccc5c5ccccc45)cc3)c([2H])c12 ; [2H]c1c([2H])c([2H])c2c(c1[2H])c([2H])c([2H])c1c([2H])c([2H])c(N(c3cccc(-c4ccccc4)c3)c3cccc4ccccc34)c([2H])c12] 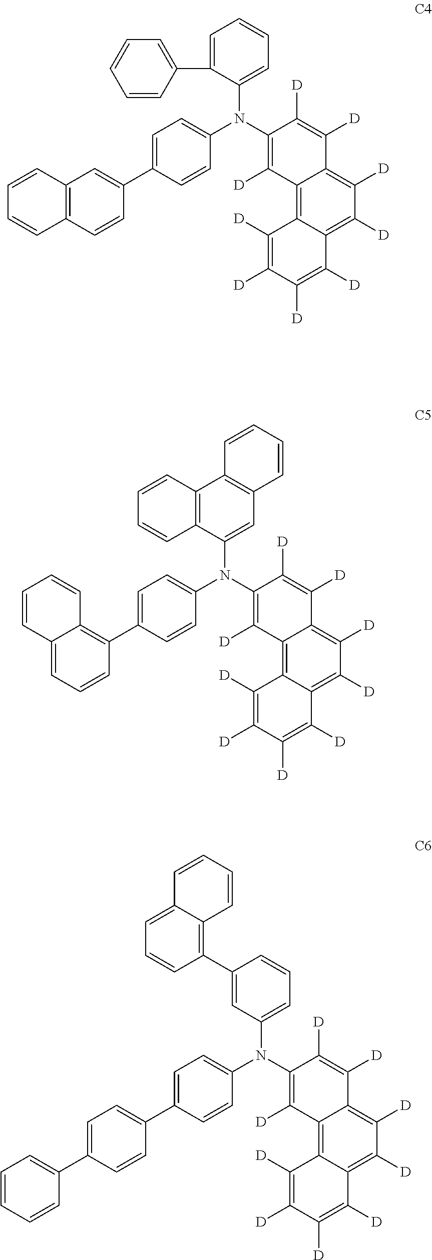 [SMILES: [2H]c1c([2H])c([2H])c2c(c1[2H])c([2H])c([2H])c1c([2H])c([2H])c(N(c3ccc(-c4ccc5ccccc5c4)cc3)c3ccccc3-c3ccccc3)c([2H])c12 ; [2H]c1c([2H])c([2H])c2c(c1[2H])c([2H])c([2H])c1c([2H])c([2H])c(N(c3ccc(-c4cccc5ccccc45)cc3)c3cc4ccccc4c4ccccc34)c([2H])c12 ; [2H]c1c([2H])c([2H])c2c(c1[2H])c([2H])c([2H])c1c([2H])c([2H])c(N(c3ccc(-c4ccc(-c5ccccc5)cc4)cc3)c3cccc(-c4cccc5ccccc45)c3)c([2H])c12] 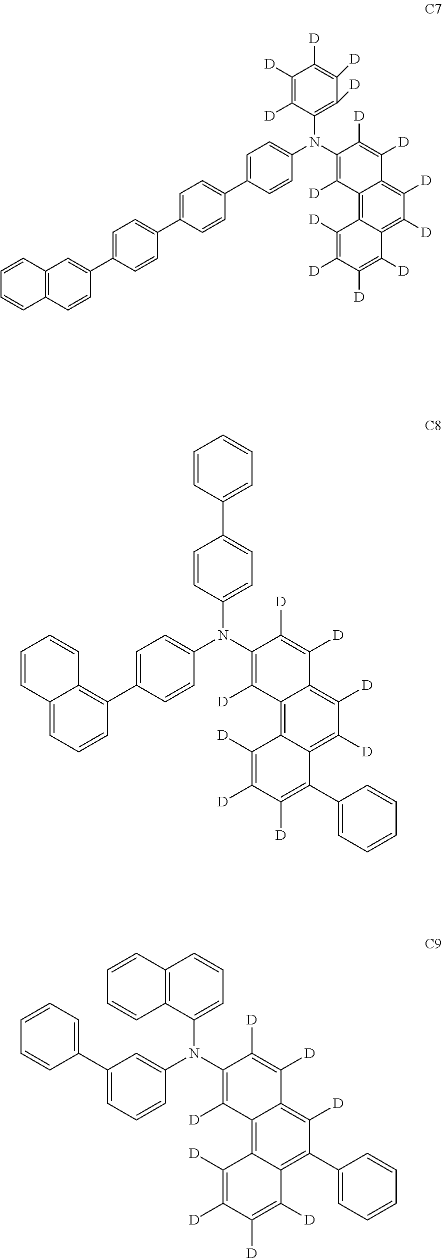 [SMILES: [2H]c1c([2H])c([2H])c(N(c2ccc(-c3ccc(-c4ccc(-c5ccc6ccccc6c5)cc4)cc3)cc2)c2c([2H])c([2H])c3c([2H])c([2H])c4c([2H])c([2H])c([2H])c([2H])c4c3c2[2H])c([2H])c1[2H] ; [2H]c1c(N(c2ccc(-c3ccccc3)cc2)c2ccc(-c3cccc4ccccc34)cc2)c([2H])c2c(c1[2H])c([2H])c([2H])c1c(-c3ccccc3)c([2H])c([2H])c([2H])c12 ; [2H]c1c(N(c2cccc(-c3ccccc3)c2)c2cccc3ccccc23)c([2H])c2c(c1[2H])c([2H])c(-c1ccccc1)c1c([2H])c([2H])c([2H])c([2H])c12] 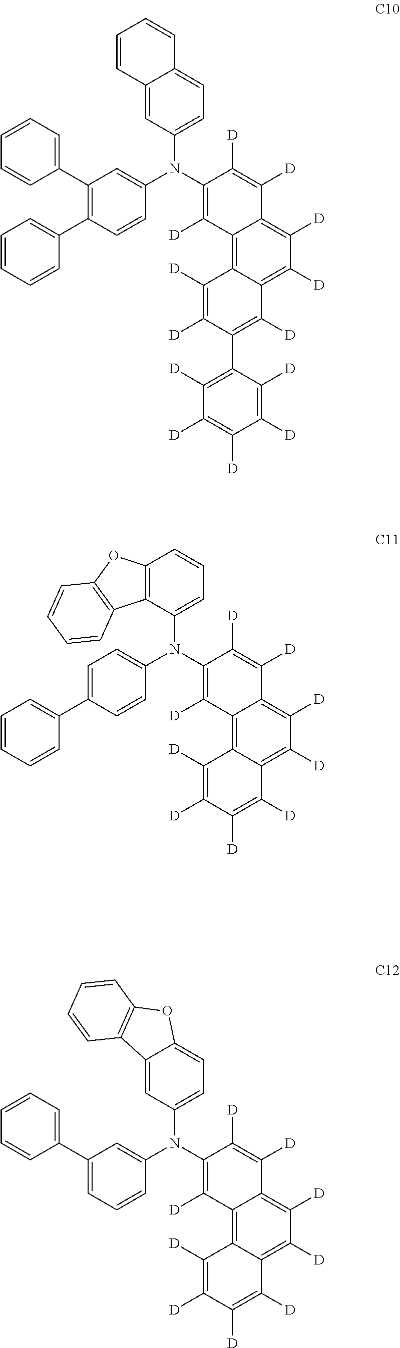 [SMILES: [2H]c1c([2H])c([2H])c(-c2c([2H])c([2H])c3c(c2[2H])c([2H])c([2H])c2c([2H])c([2H])c(N(c4ccc(-c5ccccc5)c(-c5ccccc5)c4)c4ccc5ccccc5c4)c([2H])c23)c([2H])c1[2H] ; [2H]c1c([2H])c([2H])c2c(c1[2H])c([2H])c([2H])c1c([2H])c([2H])c(N(c3ccc(-c4ccccc4)cc3)c3cccc4oc5ccccc5c34)c([2H])c12 ; [2H]c1c([2H])c([2H])c2c(c1[2H])c([2H])c([2H])c1c([2H])c([2H])c(N(c3cccc(-c4ccccc4)c3)c3ccc4oc5ccccc5c4c3)c([2H])c12] 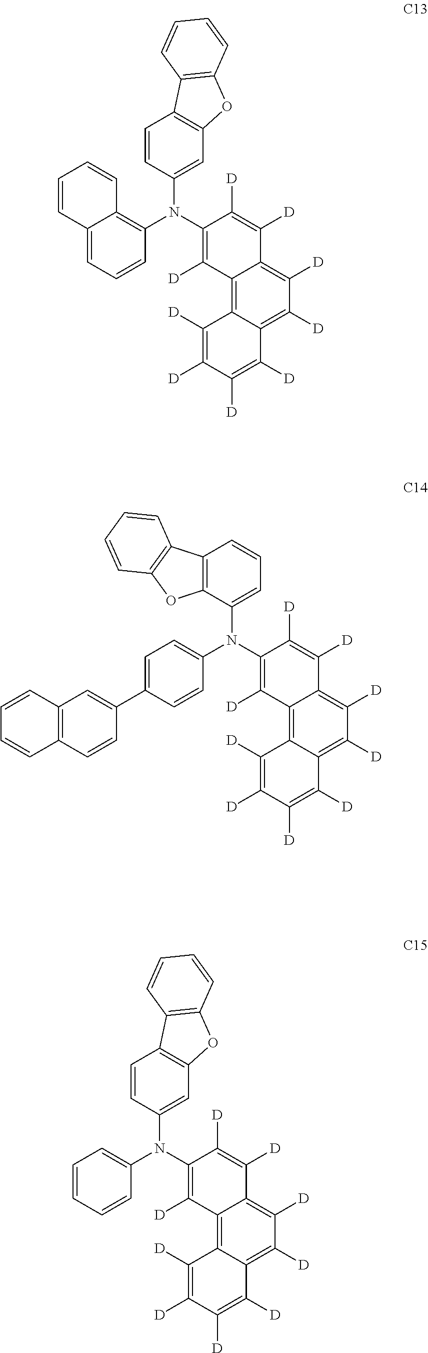 [SMILES: [2H]c1c([2H])c([2H])c2c(c1[2H])c([2H])c([2H])c1c([2H])c([2H])c(N(c3ccc4c(c3)oc3ccccc34)c3cccc4ccccc34)c([2H])c12 ; [2H]c1c([2H])c([2H])c2c(c1[2H])c([2H])c([2H])c1c([2H])c([2H])c(N(c3ccc(-c4ccc5ccccc5c4)cc3)c3cccc4c3oc3ccccc34)c([2H])c12 ; [2H]c1c([2H])c([2H])c2c(c1[2H])c([2H])c([2H])c1c([2H])c([2H])c(N(c3ccccc3)c3ccc4c(c3)oc3ccccc34)c([2H])c12] 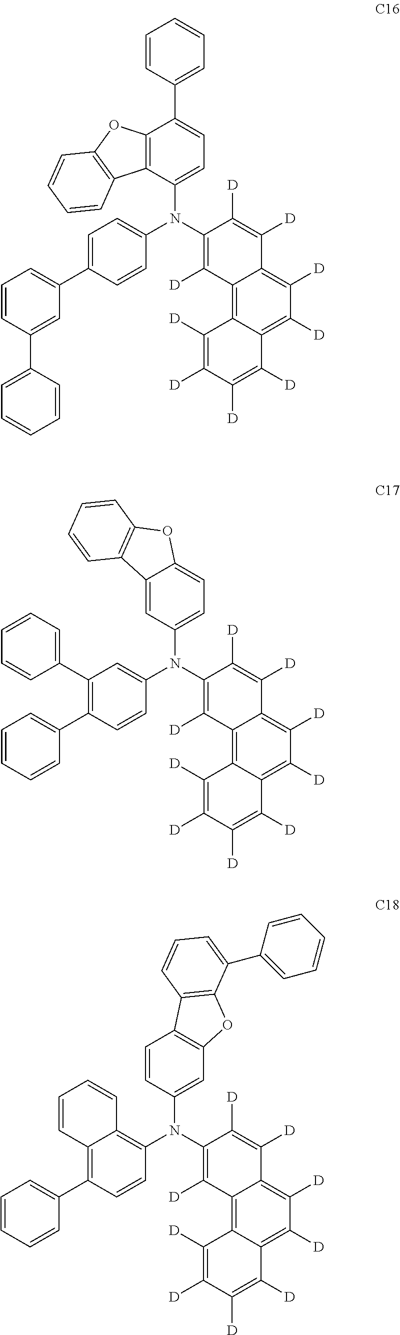 [SMILES: [2H]c1c([2H])c([2H])c2c(c1[2H])c([2H])c([2H])c1c([2H])c([2H])c(N(c3ccc(-c4cccc(-c5ccccc5)c4)cc3)c3ccc(-c4ccccc4)c4oc5ccccc5c34)c([2H])c12 ; [2H]c1c([2H])c([2H])c2c(c1[2H])c([2H])c([2H])c1c([2H])c([2H])c(N(c3ccc(-c4ccccc4)c(-c4ccccc4)c3)c3ccc4oc5ccccc5c4c3)c([2H])c12 ; [2H]c1c([2H])c([2H])c2c(c1[2H])c([2H])c([2H])c1c([2H])c([2H])c(N(c3ccc4c(c3)oc3c(-c5ccccc5)cccc34)c3ccc(-c4ccccc4)c4ccccc34)c([2H])c12] 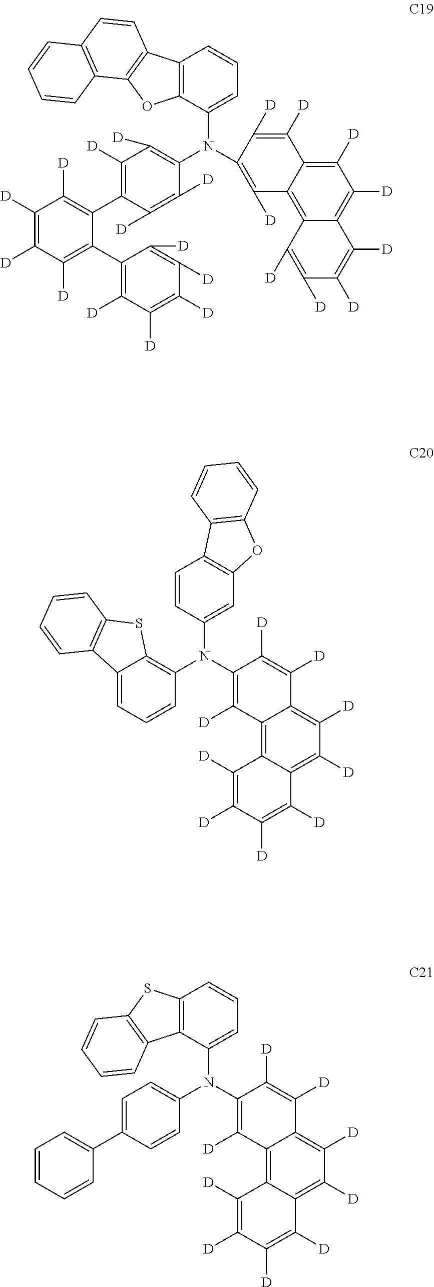 [SMILES: [2H]c1c([2H])c([2H])c(-c2c([2H])c([2H])c([2H])c([2H])c2-c2c([2H])c([2H])c(N(c3c([2H])c([2H])c4c([2H])c([2H])c5c([2H])c([2H])c([2H])c([2H])c5c4c3[2H])c3cccc4c3oc3c5ccccc5ccc43)c([2H])c2[2H])c([2H])c1[2H] ; [2H]c1c([2H])c([2H])c2c(c1[2H])c([2H])c([2H])c1c([2H])c([2H])c(N(c3ccc4c(c3)oc3ccccc34)c3cccc4c3sc3ccccc34)c([2H])c12 ; [2H]c1c([2H])c([2H])c2c(c1[2H])c([2H])c([2H])c1c([2H])c([2H])c(N(c3ccc(-c4ccccc4)cc3)c3cccc4sc5ccccc5c34)c([2H])c12] 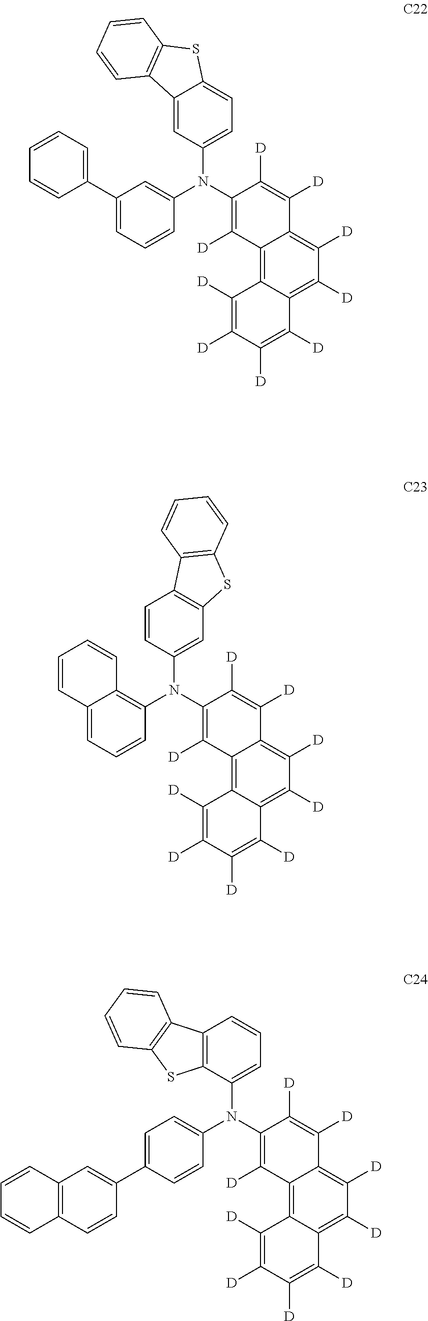 [SMILES: [2H]c1c([2H])c([2H])c2c(c1[2H])c([2H])c([2H])c1c([2H])c([2H])c(N(c3cccc(-c4ccccc4)c3)c3ccc4sc5ccccc5c4c3)c([2H])c12 ; [2H]c1c([2H])c([2H])c2c(c1[2H])c([2H])c([2H])c1c([2H])c([2H])c(N(c3ccc4c(c3)sc3ccccc34)c3cccc4ccccc34)c([2H])c12 ; [2H]c1c([2H])c([2H])c2c(c1[2H])c([2H])c([2H])c1c([2H])c([2H])c(N(c3ccc(-c4ccc5ccccc5c4)cc3)c3cccc4c3sc3ccccc34)c([2H])c12] 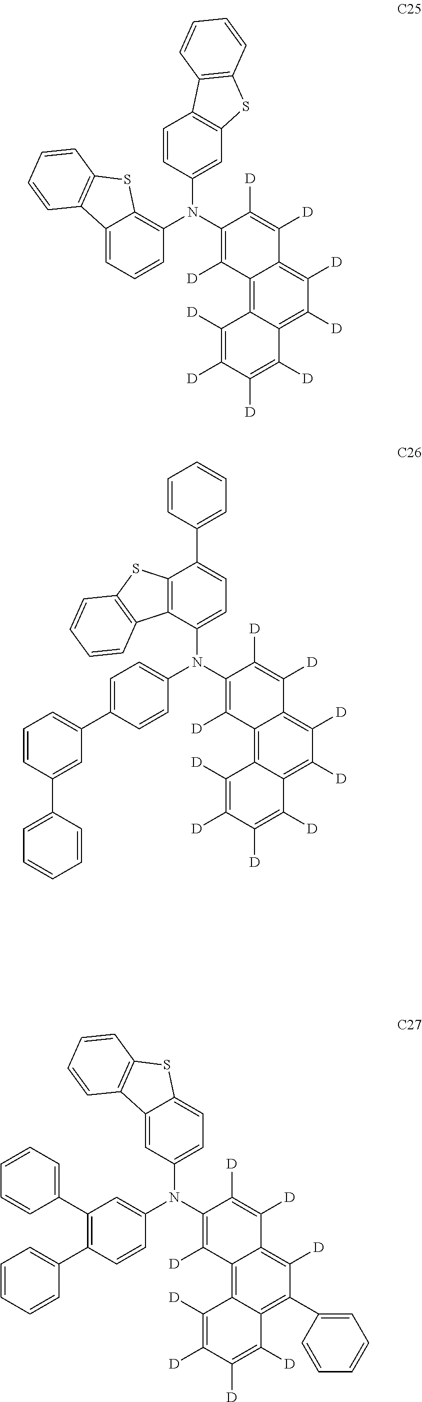 [SMILES: [2H]c1c([2H])c([2H])c2c(c1[2H])c([2H])c([2H])c1c([2H])c([2H])c(N(c3ccc4c(c3)sc3ccccc34)c3cccc4c3sc3ccccc34)c([2H])c12 ; [2H]c1c([2H])c([2H])c2c(c1[2H])c([2H])c([2H])c1c([2H])c([2H])c(N(c3ccc(-c4cccc(-c5ccccc5)c4)cc3)c3ccc(-c4ccccc4)c4sc5ccccc5c34)c([2H])c12 ; [2H]c1c(N(c2ccc(-c3ccccc3)c(-c3ccccc3)c2)c2ccc3sc4ccccc4c3c2)c([2H])c2c(c1[2H])c([2H])c(-c1ccccc1)c1c([2H])c([2H])c([2H])c([2H])c12] 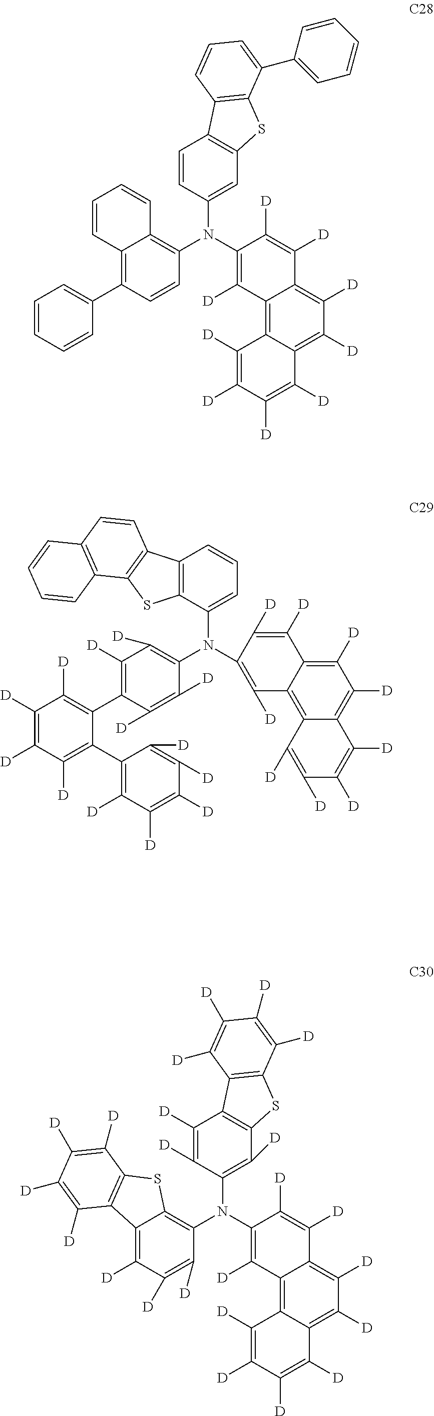 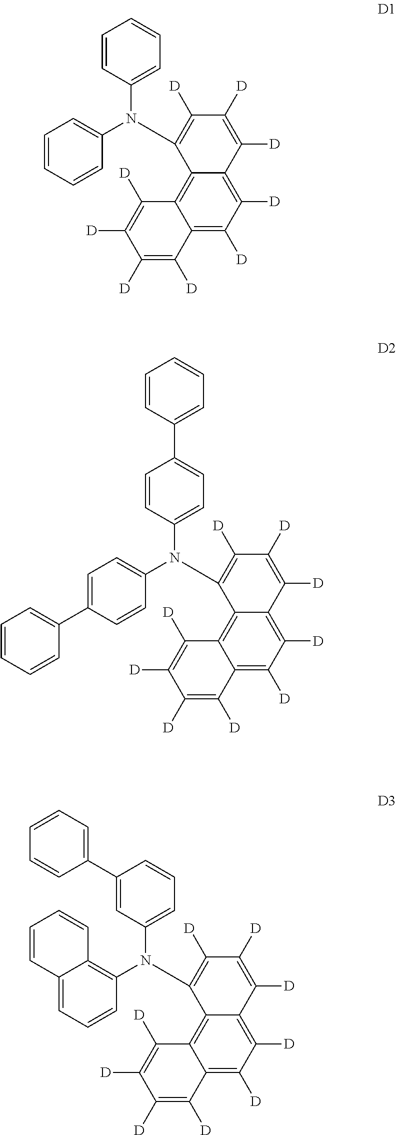 [SMILES: [2H]c1c([2H])c([2H])c2c(c1[2H])c([2H])c([2H])c1c([2H])c([2H])c([2H])c(N(c3ccccc3)c3ccccc3)c12 ; [2H]c1c([2H])c([2H])c2c(c1[2H])c([2H])c([2H])c1c([2H])c([2H])c([2H])c(N(c3ccc(-c4ccccc4)cc3)c3ccc(-c4ccccc4)cc3)c12 ; [2H]c1c([2H])c([2H])c2c(c1[2H])c([2H])c([2H])c1c([2H])c([2H])c([2H])c(N(c3cccc(-c4ccccc4)c3)c3cccc4ccccc34)c12] 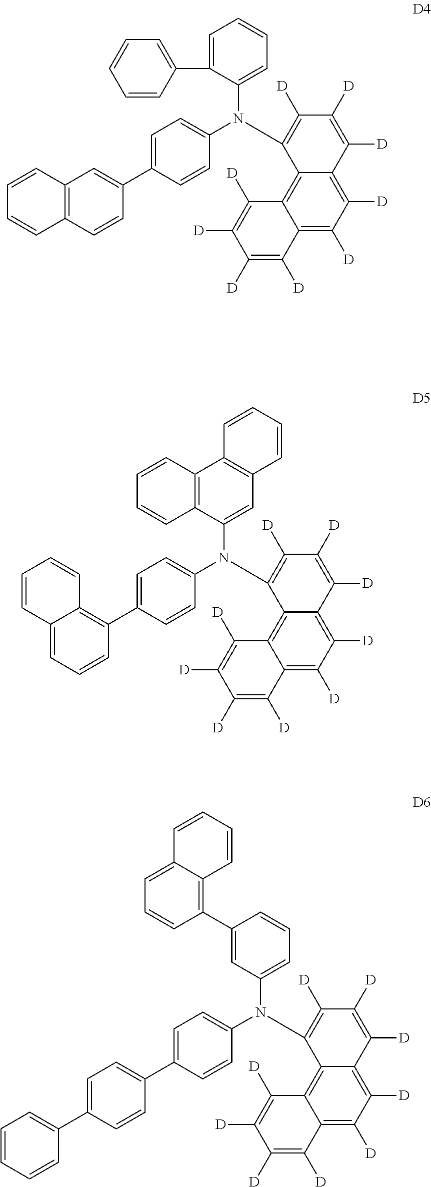 [SMILES: [2H]c1c([2H])c([2H])c2c(c1[2H])c([2H])c([2H])c1c([2H])c([2H])c([2H])c(N(c3ccc(-c4ccc5ccccc5c4)cc3)c3ccccc3-c3ccccc3)c12 ; [2H]c1c([2H])c([2H])c2c(c1[2H])c([2H])c([2H])c1c([2H])c([2H])c([2H])c(N(c3ccc(-c4cccc5ccccc45)cc3)c3cc4ccccc4c4ccccc34)c12 ; [2H]c1c([2H])c([2H])c2c(c1[2H])c([2H])c([2H])c1c([2H])c([2H])c([2H])c(N(c3ccc(-c4ccc(-c5ccccc5)cc4)cc3)c3cccc(-c4cccc5ccccc45)c3)c12] 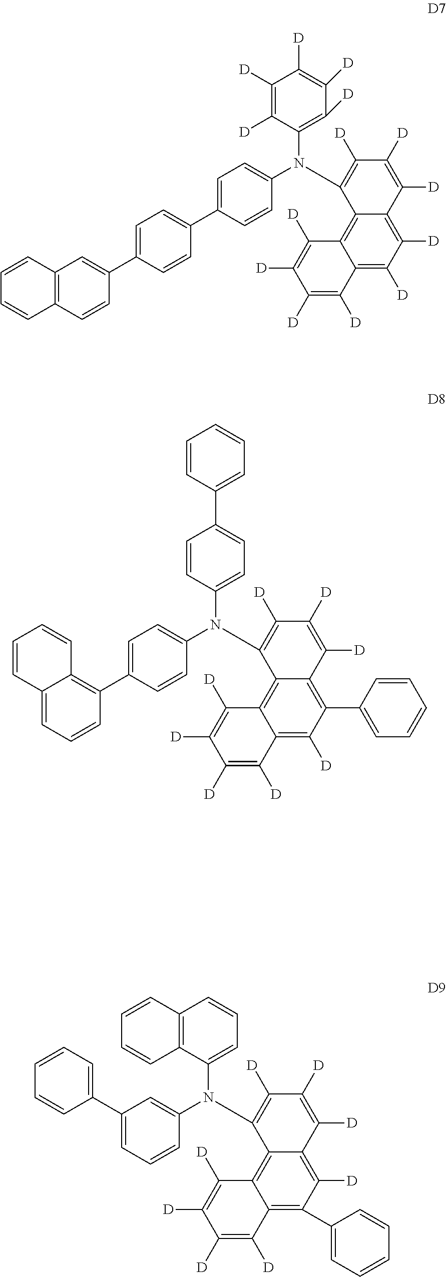 [SMILES: [2H]c1c([2H])c([2H])c(N(c2ccc(-c3ccc(-c4ccc5ccccc5c4)cc3)cc2)c2c([2H])c([2H])c([2H])c3c([2H])c([2H])c4c([2H])c([2H])c([2H])c([2H])c4c23)c([2H])c1[2H] ; [2H]c1c([2H])c([2H])c2c(c1[2H])c([2H])c(-c1ccccc1)c1c([2H])c([2H])c([2H])c(N(c3ccc(-c4ccccc4)cc3)c3ccc(-c4cccc5ccccc45)cc3)c12 ; [2H]c1c([2H])c([2H])c2c(c1[2H])c(-c1ccccc1)c([2H])c1c([2H])c([2H])c([2H])c(N(c3cccc(-c4ccccc4)c3)c3cccc4ccccc34)c12] 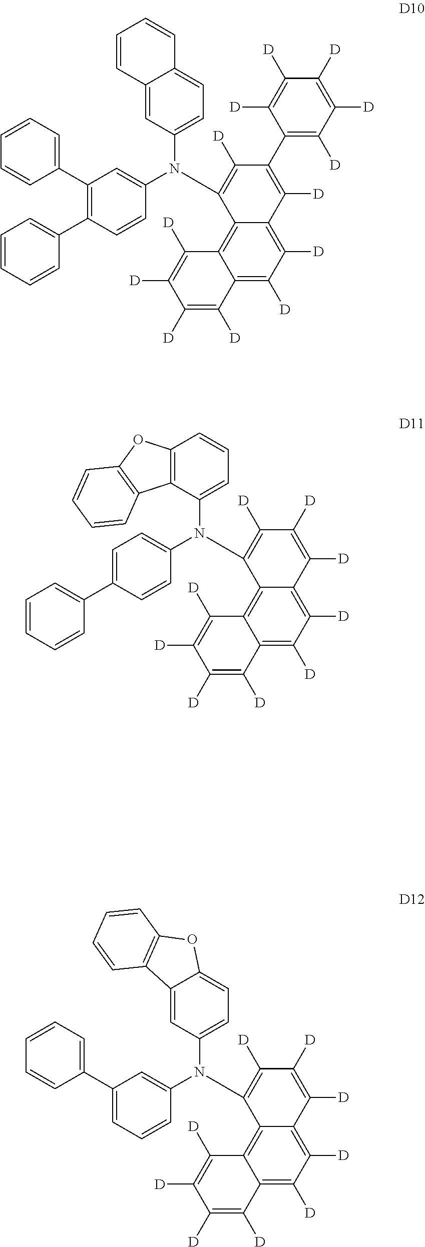 [SMILES: [2H]c1c([2H])c([2H])c(-c2c([2H])c(N(c3ccc(-c4ccccc4)c(-c4ccccc4)c3)c3ccc4ccccc4c3)c3c(c2[2H])c([2H])c([2H])c2c([2H])c([2H])c([2H])c([2H])c23)c([2H])c1[2H] ; [2H]c1c([2H])c([2H])c2c(c1[2H])c([2H])c([2H])c1c([2H])c([2H])c([2H])c(N(c3ccc(-c4ccccc4)cc3)c3cccc4oc5ccccc5c34)c12 ; [2H]c1c([2H])c([2H])c2c(c1[2H])c([2H])c([2H])c1c([2H])c([2H])c([2H])c(N(c3cccc(-c4ccccc4)c3)c3ccc4oc5ccccc5c4c3)c12] 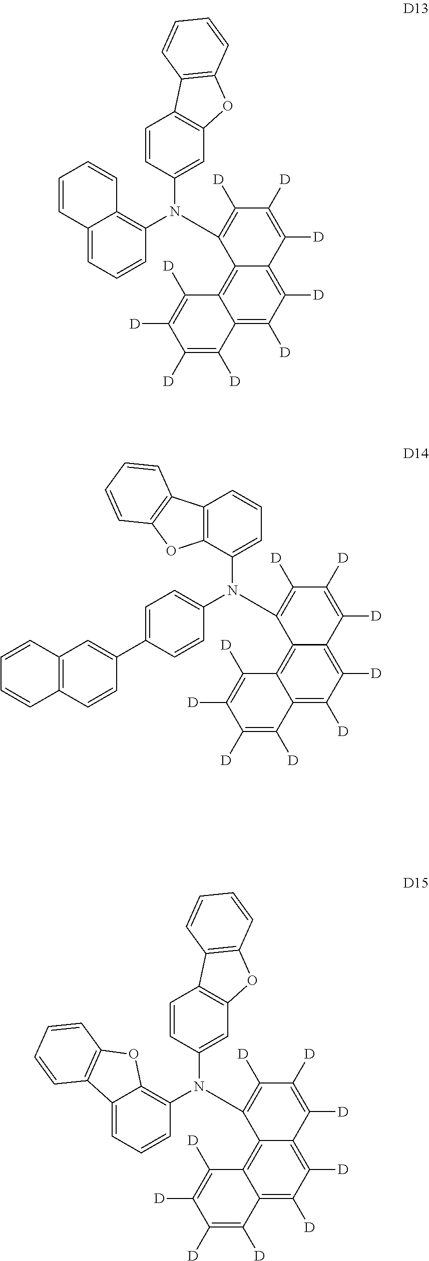 [SMILES: [2H]c1c([2H])c([2H])c2c(c1[2H])c([2H])c([2H])c1c([2H])c([2H])c([2H])c(N(c3ccc4c(c3)oc3ccccc34)c3cccc4ccccc34)c12 ; [2H]c1c([2H])c([2H])c2c(c1[2H])c([2H])c([2H])c1c([2H])c([2H])c([2H])c(N(c3ccc(-c4ccc5ccccc5c4)cc3)c3cccc4c3oc3ccccc34)c12 ; [2H]c1c([2H])c([2H])c2c(c1[2H])c([2H])c([2H])c1c([2H])c([2H])c([2H])c(N(c3ccc4c(c3)oc3ccccc34)c3cccc4c3oc3ccccc34)c12] 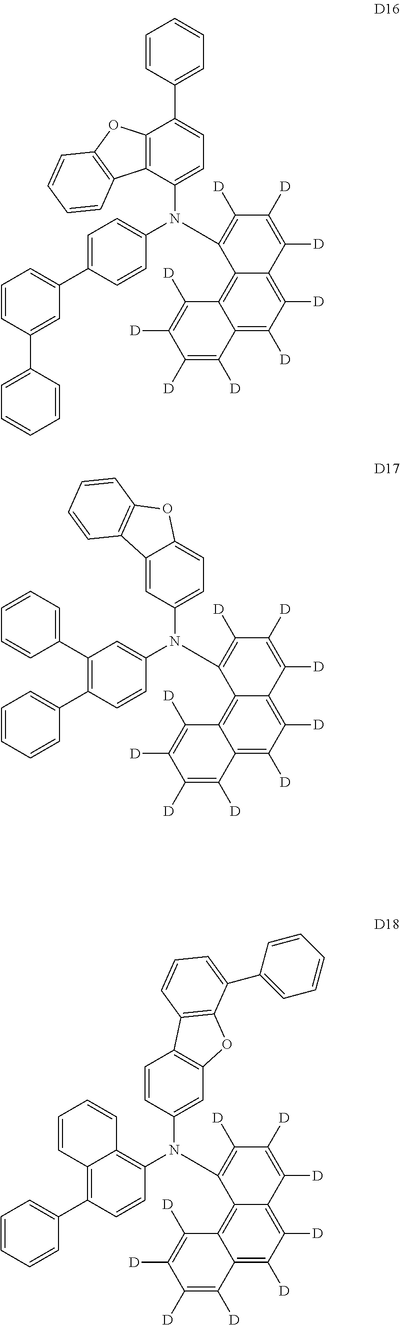 [SMILES: [2H]c1c([2H])c([2H])c2c(c1[2H])c([2H])c([2H])c1c([2H])c([2H])c([2H])c(N(c3ccc(-c4cccc(-c5ccccc5)c4)cc3)c3ccc(-c4ccccc4)c4oc5ccccc5c34)c12 ; [2H]c1c([2H])c([2H])c2c(c1[2H])c([2H])c([2H])c1c([2H])c([2H])c([2H])c(N(c3ccc(-c4ccccc4)c(-c4ccccc4)c3)c3ccc4oc5ccccc5c4c3)c12 ; [2H]c1c([2H])c([2H])c2c(c1[2H])c([2H])c([2H])c1c([2H])c([2H])c([2H])c(N(c3ccc4c(c3)oc3c(-c5ccccc5)cccc34)c3ccc(-c4ccccc4)c4ccccc34)c12] 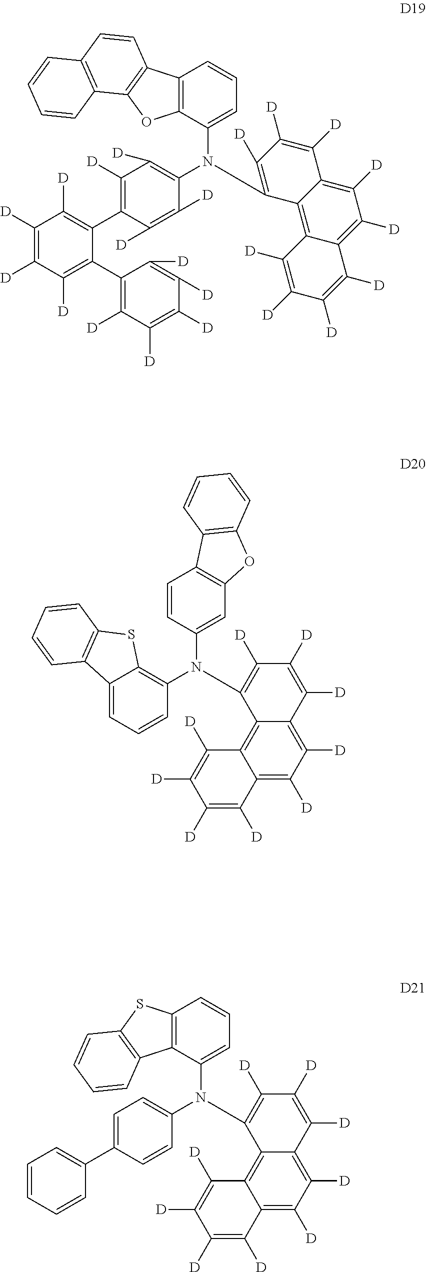 [SMILES: [2H]c1c([2H])c([2H])c(-c2c([2H])c([2H])c([2H])c([2H])c2-c2c([2H])c([2H])c(N(c3cccc4c3oc3c5ccccc5ccc43)c3c([2H])c([2H])c([2H])c4c([2H])c([2H])c5c([2H])c([2H])c([2H])c([2H])c5c34)c([2H])c2[2H])c([2H])c1[2H] ; [2H]c1c([2H])c([2H])c2c(c1[2H])c([2H])c([2H])c1c([2H])c([2H])c([2H])c(N(c3ccc4c(c3)oc3ccccc34)c3cccc4c3sc3ccccc34)c12 ; [2H]c1c([2H])c([2H])c2c(c1[2H])c([2H])c([2H])c1c([2H])c([2H])c([2H])c(N(c3ccc(-c4ccccc4)cc3)c3cccc4sc5ccccc5c34)c12] 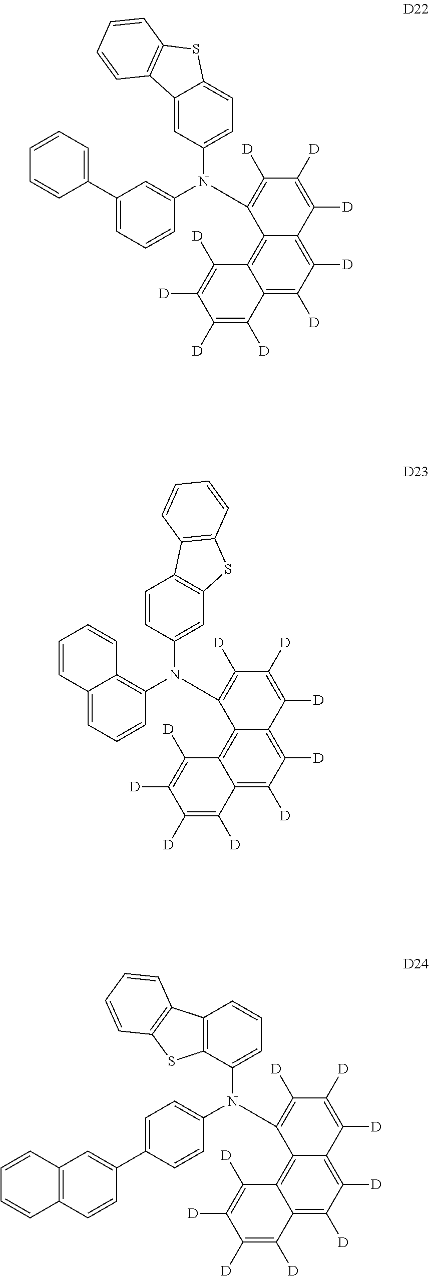 [SMILES: [2H]c1c([2H])c([2H])c2c(c1[2H])c([2H])c([2H])c1c([2H])c([2H])c([2H])c(N(c3cccc(-c4ccccc4)c3)c3ccc4sc5ccccc5c4c3)c12 ; [2H]c1c([2H])c([2H])c2c(c1[2H])c([2H])c([2H])c1c([2H])c([2H])c([2H])c(N(c3ccc4c(c3)sc3ccccc34)c3cccc4ccccc34)c12 ; [2H]c1c([2H])c([2H])c2c(c1[2H])c([2H])c([2H])c1c([2H])c([2H])c([2H])c(N(c3ccc(-c4ccc5ccccc5c4)cc3)c3cccc4c3sc3ccccc34)c12] 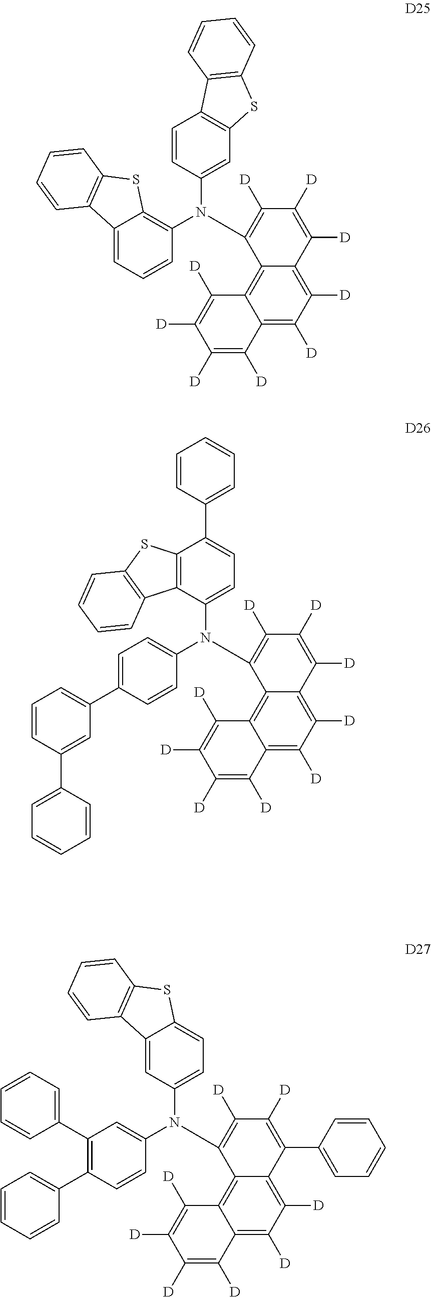 [SMILES: [2H]c1c([2H])c([2H])c2c(c1[2H])c([2H])c([2H])c1c([2H])c([2H])c([2H])c(N(c3ccc4c(c3)sc3ccccc34)c3cccc4c3sc3ccccc34)c12 ; [2H]c1c([2H])c([2H])c2c(c1[2H])c([2H])c([2H])c1c([2H])c([2H])c([2H])c(N(c3ccc(-c4cccc(-c5ccccc5)c4)cc3)c3ccc(-c4ccccc4)c4sc5ccccc5c34)c12 ; [2H]c1c([2H])c([2H])c2c(c1[2H])c([2H])c([2H])c1c(-c3ccccc3)c([2H])c([2H])c(N(c3ccc(-c4ccccc4)c(-c4ccccc4)c3)c3ccc4sc5ccccc5c4c3)c12] 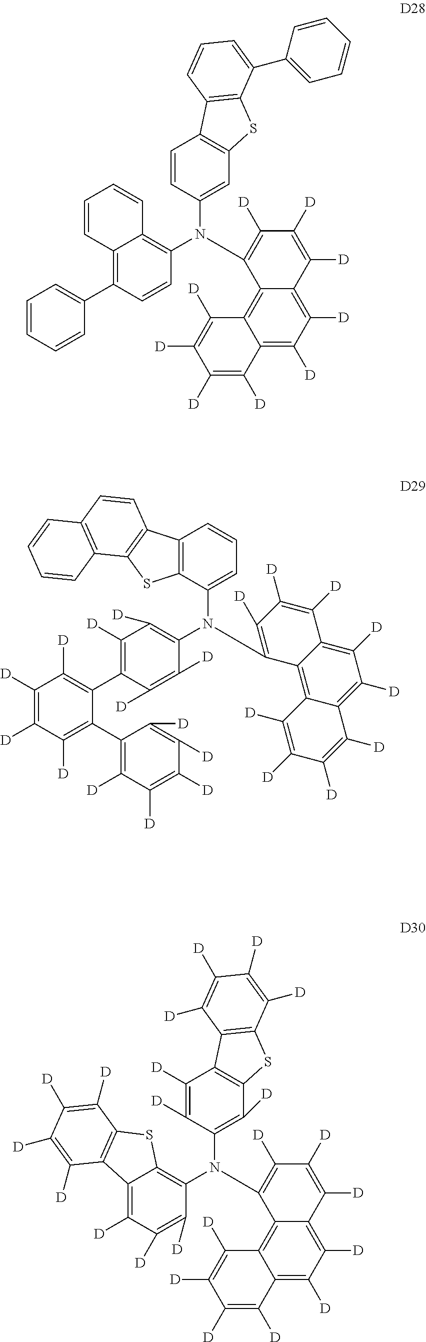 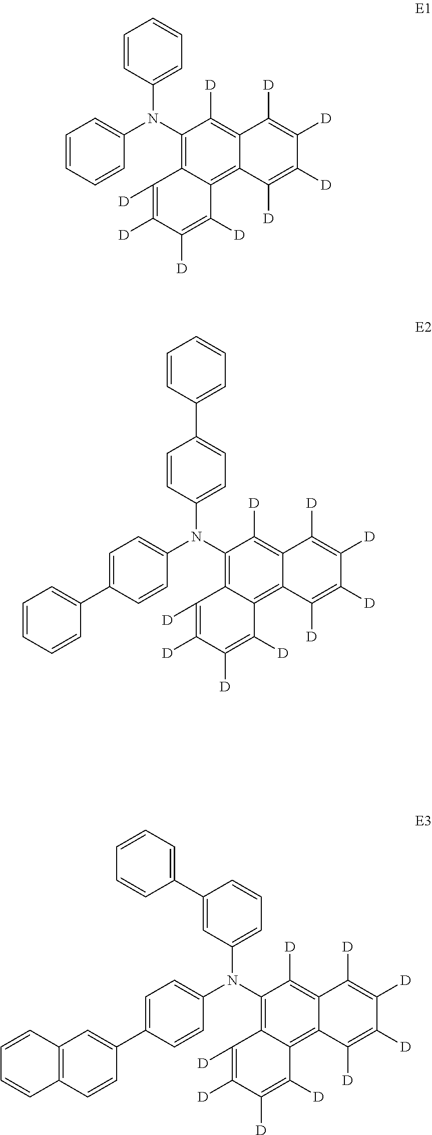 [SMILES: [2H]c1c([2H])c([2H])c2c(c1[2H])c([2H])c(N(c1ccccc1)c1ccccc1)c1c([2H])c([2H])c([2H])c([2H])c12 ; [2H]c1c([2H])c([2H])c2c(c1[2H])c([2H])c(N(c1ccc(-c3ccccc3)cc1)c1ccc(-c3ccccc3)cc1)c1c([2H])c([2H])c([2H])c([2H])c12 ; [2H]c1c([2H])c([2H])c2c(c1[2H])c([2H])c(N(c1ccc(-c3ccc4ccccc4c3)cc1)c1cccc(-c3ccccc3)c1)c1c([2H])c([2H])c([2H])c([2H])c12] 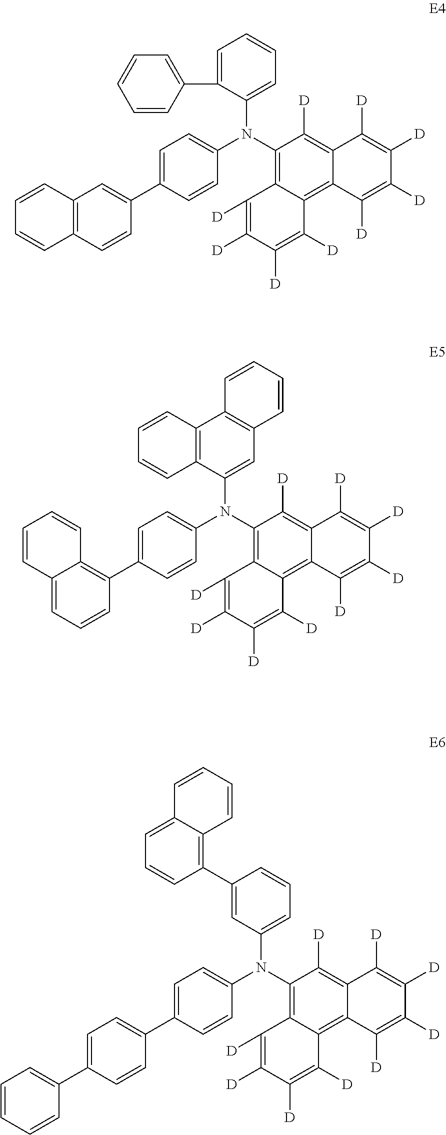 [SMILES: [2H]c1c([2H])c([2H])c2c(c1[2H])c([2H])c(N(c1ccc(-c3ccc4ccccc4c3)cc1)c1ccccc1-c1ccccc1)c1c([2H])c([2H])c([2H])c([2H])c12 ; [2H]c1c([2H])c([2H])c2c(c1[2H])c([2H])c(N(c1ccc(-c3cccc4ccccc34)cc1)c1cc3ccccc3c3ccccc13)c1c([2H])c([2H])c([2H])c([2H])c12 ; [2H]c1c([2H])c([2H])c2c(c1[2H])c([2H])c(N(c1ccc(-c3ccc(-c4ccccc4)cc3)cc1)c1cccc(-c3cccc4ccccc34)c1)c1c([2H])c([2H])c([2H])c([2H])c12] 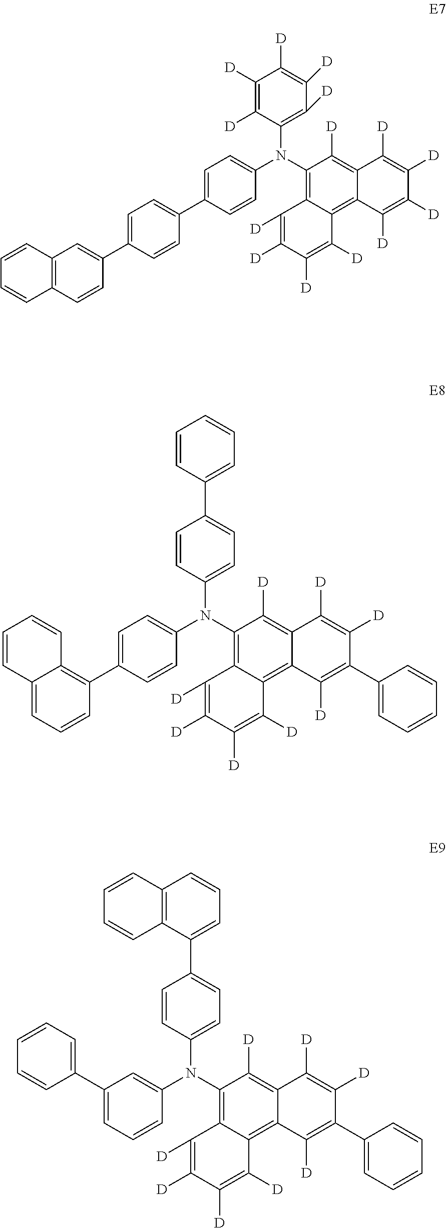 [SMILES: [2H]c1c([2H])c([2H])c(N(c2ccc(-c3ccc(-c4ccc5ccccc5c4)cc3)cc2)c2c([2H])c3c([2H])c([2H])c([2H])c([2H])c3c3c([2H])c([2H])c([2H])c([2H])c23)c([2H])c1[2H] ; [2H]c1c(-c2ccccc2)c([2H])c2c(c1[2H])c([2H])c(N(c1ccc(-c3ccccc3)cc1)c1ccc(-c3cccc4ccccc34)cc1)c1c([2H])c([2H])c([2H])c([2H])c12 ; [2H]c1c(-c2ccccc2)c([2H])c2c(c1[2H])c([2H])c(N(c1ccc(-c3cccc4ccccc34)cc1)c1cccc(-c3ccccc3)c1)c1c([2H])c([2H])c([2H])c([2H])c12] 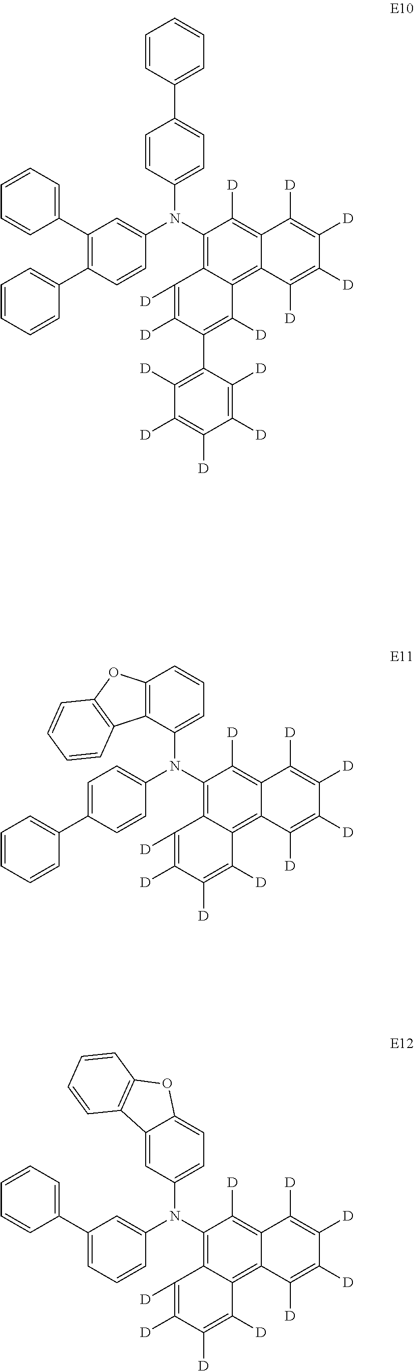 [SMILES: [2H]c1c([2H])c([2H])c(-c2c([2H])c([2H])c3c(N(c4ccc(-c5ccccc5)cc4)c4ccc(-c5ccccc5)c(-c5ccccc5)c4)c([2H])c4c([2H])c([2H])c([2H])c([2H])c4c3c2[2H])c([2H])c1[2H] ; [2H]c1c([2H])c([2H])c2c(c1[2H])c([2H])c(N(c1ccc(-c3ccccc3)cc1)c1cccc3oc4ccccc4c13)c1c([2H])c([2H])c([2H])c([2H])c12 ; [2H]c1c([2H])c([2H])c2c(c1[2H])c([2H])c(N(c1cccc(-c3ccccc3)c1)c1ccc3oc4ccccc4c3c1)c1c([2H])c([2H])c([2H])c([2H])c12] 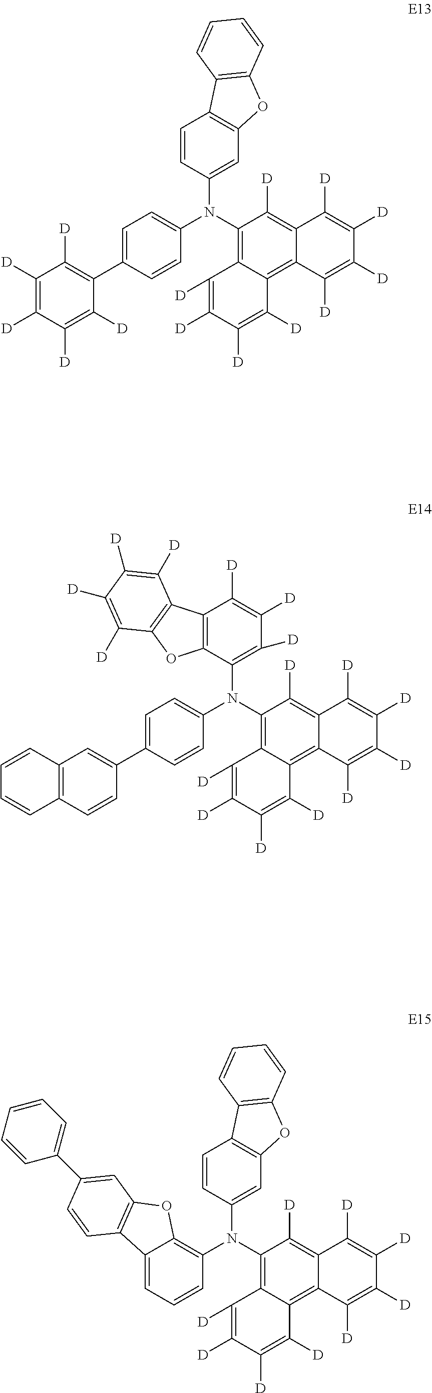 [SMILES: [2H]c1c([2H])c([2H])c(-c2ccc(N(c3ccc4c(c3)oc3ccccc34)c3c([2H])c4c([2H])c([2H])c([2H])c([2H])c4c4c([2H])c([2H])c([2H])c([2H])c34)cc2)c([2H])c1[2H] ; [2H]c1c([2H])c([2H])c2c(oc3c(N(c4ccc(-c5ccc6ccccc6c5)cc4)c4c([2H])c5c([2H])c([2H])c([2H])c([2H])c5c5c([2H])c([2H])c([2H])c([2H])c45)c([2H])c([2H])c([2H])c32)c1[2H] ; [2H]c1c([2H])c([2H])c2c(c1[2H])c([2H])c(N(c1ccc3c(c1)oc1ccccc13)c1cccc3c1oc1cc(-c4ccccc4)ccc13)c1c([2H])c([2H])c([2H])c([2H])c12] 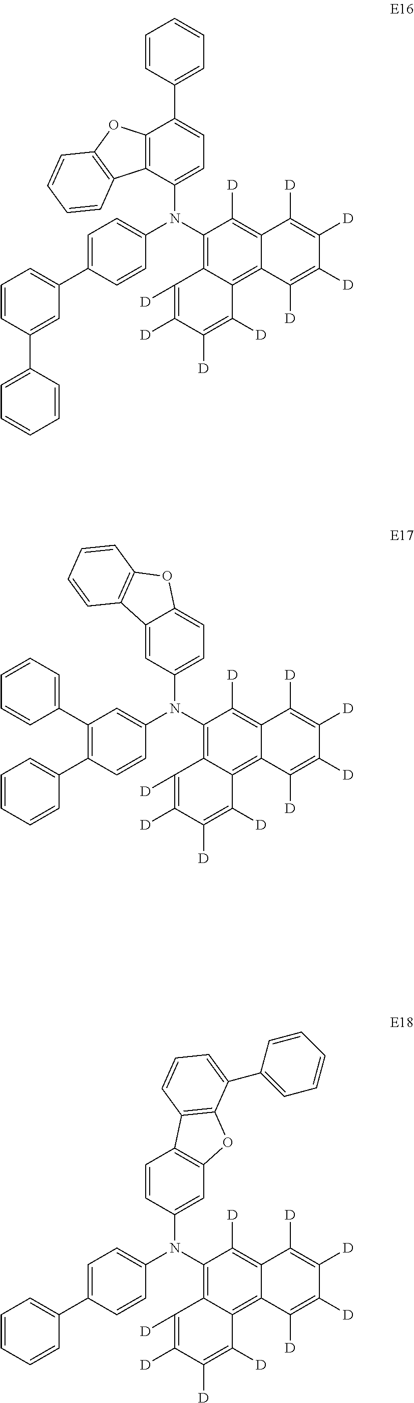 [SMILES: [2H]c1c([2H])c([2H])c2c(c1[2H])c([2H])c(N(c1ccc(-c3cccc(-c4ccccc4)c3)cc1)c1ccc(-c3ccccc3)c3oc4ccccc4c13)c1c([2H])c([2H])c([2H])c([2H])c12 ; [2H]c1c([2H])c([2H])c2c(c1[2H])c([2H])c(N(c1ccc(-c3ccccc3)c(-c3ccccc3)c1)c1ccc3oc4ccccc4c3c1)c1c([2H])c([2H])c([2H])c([2H])c12 ; [2H]c1c([2H])c([2H])c2c(c1[2H])c([2H])c(N(c1ccc(-c3ccccc3)cc1)c1ccc3c(c1)oc1c(-c4ccccc4)cccc13)c1c([2H])c([2H])c([2H])c([2H])c12] 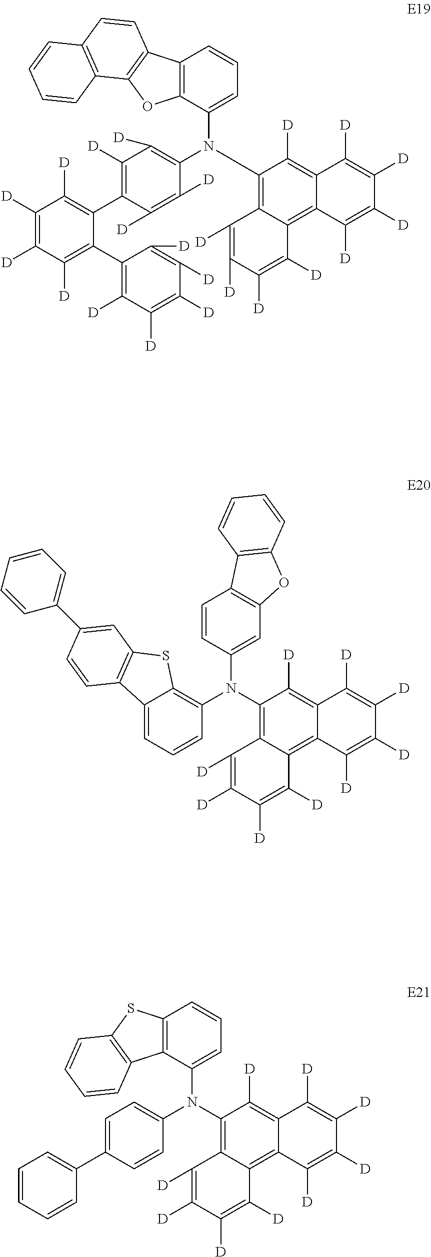 [SMILES: [2H]c1c([2H])c([2H])c(-c2c([2H])c([2H])c([2H])c([2H])c2-c2c([2H])c([2H])c(N(c3cccc4c3oc3c5ccccc5ccc43)c3c([2H])c4c([2H])c([2H])c([2H])c([2H])c4c4c([2H])c([2H])c([2H])c([2H])c34)c([2H])c2[2H])c([2H])c1[2H] ; [2H]c1c([2H])c([2H])c2c(c1[2H])c([2H])c(N(c1ccc3c(c1)oc1ccccc13)c1cccc3c1sc1cc(-c4ccccc4)ccc13)c1c([2H])c([2H])c([2H])c([2H])c12 ; [2H]c1c([2H])c([2H])c2c(c1[2H])c([2H])c(N(c1ccc(-c3ccccc3)cc1)c1cccc3sc4ccccc4c13)c1c([2H])c([2H])c([2H])c([2H])c12] 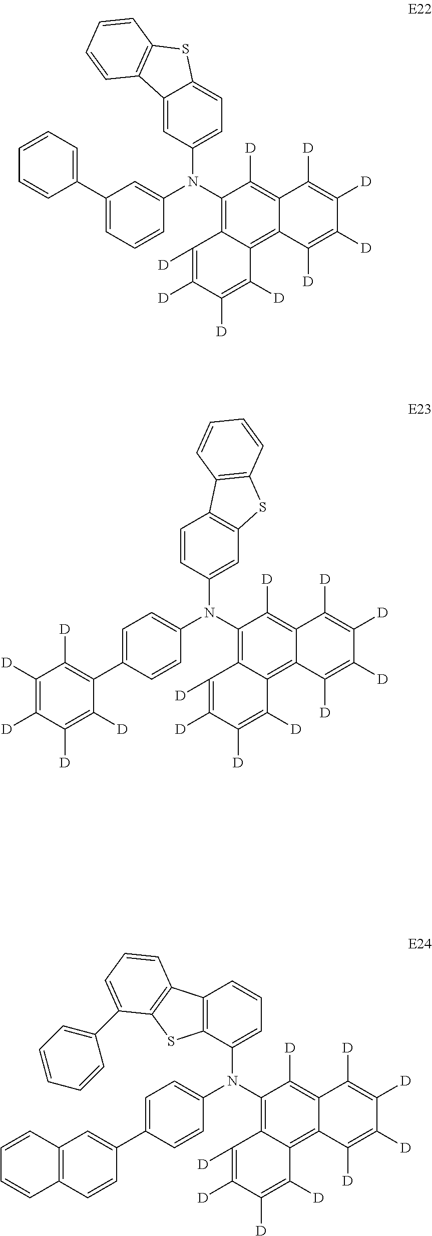 [SMILES: [2H]c1c([2H])c([2H])c2c(c1[2H])c([2H])c(N(c1cccc(-c3ccccc3)c1)c1ccc3sc4ccccc4c3c1)c1c([2H])c([2H])c([2H])c([2H])c12 ; [2H]c1c([2H])c([2H])c(-c2ccc(N(c3ccc4c(c3)sc3ccccc34)c3c([2H])c4c([2H])c([2H])c([2H])c([2H])c4c4c([2H])c([2H])c([2H])c([2H])c34)cc2)c([2H])c1[2H] ; [2H]c1c([2H])c([2H])c2c(c1[2H])c([2H])c(N(c1ccc(-c3ccc4ccccc4c3)cc1)c1cccc3c1sc1c(-c4ccccc4)cccc13)c1c([2H])c([2H])c([2H])c([2H])c12] 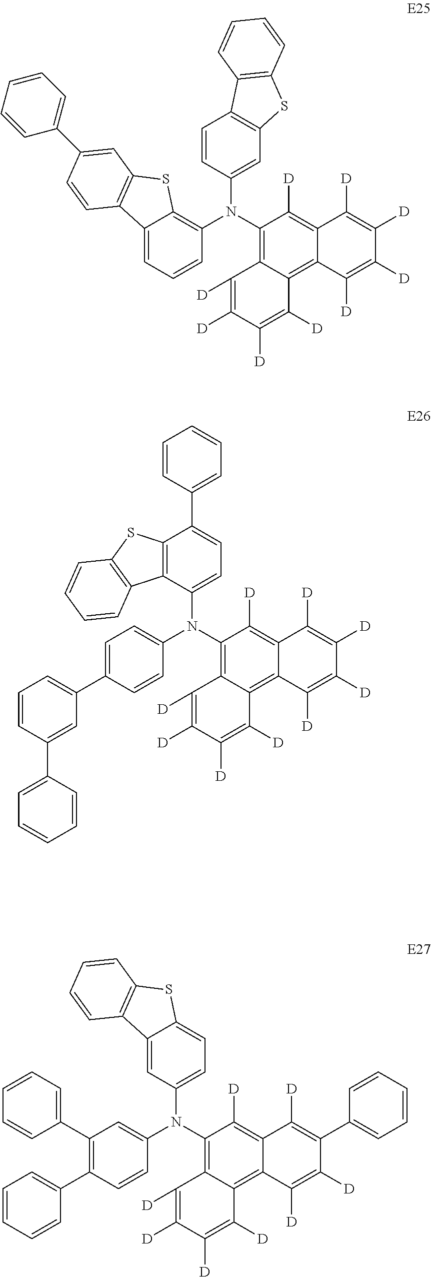 [SMILES: [2H]c1c([2H])c([2H])c2c(c1[2H])c([2H])c(N(c1ccc3c(c1)sc1ccccc13)c1cccc3c1sc1cc(-c4ccccc4)ccc13)c1c([2H])c([2H])c([2H])c([2H])c12 ; [2H]c1c([2H])c([2H])c2c(c1[2H])c([2H])c(N(c1ccc(-c3cccc(-c4ccccc4)c3)cc1)c1ccc(-c3ccccc3)c3sc4ccccc4c13)c1c([2H])c([2H])c([2H])c([2H])c12 ; [2H]c1c([2H])c([2H])c2c(c1[2H])c(N(c1ccc(-c3ccccc3)c(-c3ccccc3)c1)c1ccc3sc4ccccc4c3c1)c([2H])c1c([2H])c(-c3ccccc3)c([2H])c([2H])c12] 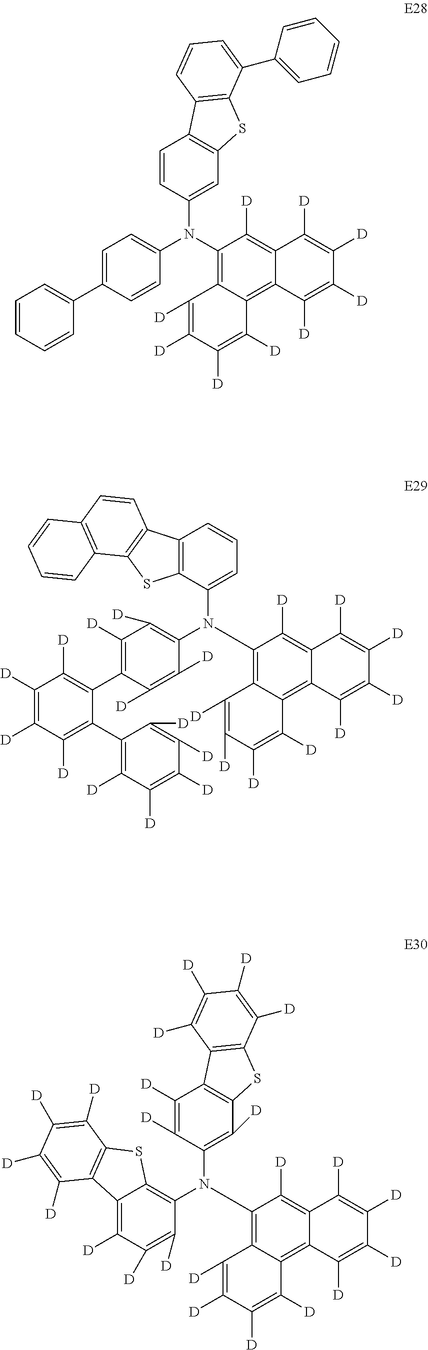
in Compound Group 1, D being deuterium.

18. An electronic apparatus comprising:
a base layer;
a circuit layer on the base layer; and
a display element layer on the circuit layer and comprising a light-emitting element,
wherein the light-emitting element comprises:
a first electrode;
a second electrode on the first electrode;
a light-emitting layer between the first electrode and the second electrode; and
a hole transport region between the first electrode and the light-emitting layer and comprising an amine compound represented by Formula 1 and Formula 2: 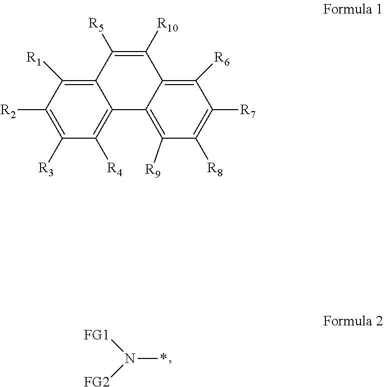
wherein,
in Formula 1, at least one selected from among R₁ to R₅ is an amine group represented by Formula 2, and at least one selected from among the remaining of R₁ to R₅, and R₆ to R₁₀ is deuterium, and the remaining that is not deuterium is a substituted or unsubstituted aryl group having ring-forming carbons of 6 to 40,
in Formula 2, FG1 and FG2 are each independently a substituted or unsubstituted aryl group having ring-forming carbons of 6 to 40 except for a fluorenyl group or a substituted or unsubstituted heteroaryl group having ring-forming carbons of 5 to 40 and containing 1 to 3 hetero atoms, and FG1 and FG2 comprise no alkyl group as a substituent,
when R₂ is the amine group represented by Formula 2:
a case where FG1 and FG2 are each a substituted or unsubstituted aryl group having ring-forming carbons of 6 to 15, at the same time, is excluded; and
a case where FG1 or FG2 is a benzonaphthofuranyl group substituted with an aryl group is excluded,
when R₃ is the amine group represented by Formula 2:
a case where FG1 and FG2 are each a substituted or unsubstituted aryl group having ring-forming carbons of 6 to 9, at the same time, is excluded; and
when one among FG1 and FG2 is an aryl group having ring-forming carbons of 26 or more, the other is an aryl group having ring-forming carbons of 6 to 15 or a substituted or unsubstituted heteroaryl group having ring-forming carbons or 5 to 40, and
when R₅ is the amine group represented by Formula 2:
when FG1 or FG2 is a 4-dibenzofuranyl group or a 4-dibenzothiophenyl group, at least one hydrogen of the 4-dibenzofuranyl group or the 4-dibenzothiophenyl group is substituted with a substituent; and
cases where FG1 or FG2 is a naphthyl group or a benzonaphthofuranyl group substituted with an aryl group are excluded.

19. The electronic apparatus of claim 18, wherein the light-emitting element is to emit blue light or green light.

20. The electronic apparatus of claim 18, further comprising a light control layer comprising a quantum dot.
